SEQUENCE LISTING (266 sequences)

SEQ ID NO: 1 - protein, 7 aa, synthetic construct
GSGSGSG

SEQ ID NO: 2 - protein, 5 aa, synthetic construct
GGGGS

SEQ ID NO: 3 - protein, 5 aa, synthetic construct
GSGSG

SEQ ID NO: 4 - protein, 4 aa, synthetic construct
GGGG

SEQ ID NO: 5-7 - skipped (3 entries)

SEQ ID NO: 8 - protein, 4 aa, synthetic construct
GSGS

SEQ ID NO: 9 - protein, 4 aa, synthetic construct
GGGS

SEQ ID NO: 10-11 - skipped (2 entries)

SEQ ID NO: 12 - protein, 6 aa, synthetic construct
GGSGGS

SEQ ID NO: 13 - skipped

SEQ ID NO: 14 - protein, 6 aa, synthetic construct
GGGGGG

SEQ ID NO: 15 - protein, 9 aa, synthetic construct
GGGGGGGGG

SEQ ID NO: 16 - protein, 12 aa, synthetic construct
GGGGGGGGGGGG

SEQ ID NO: 17 - protein, 15 aa, synthetic construct
GGGGGGGGGGGGGGG

SEQ ID NO: 18 - skipped

SEQ ID NO: 19 - protein, 6 aa, synthetic construct
GGSGGS

SEQ ID NO: 20 - protein, 9 aa, synthetic construct
GGSGGSGGS

SEQ ID NO: 21 - protein, 12 aa, synthetic construct
GGSGGSGGSGGS

SEQ ID NO: 22 - protein, 15 aa, synthetic construct
GGSGGSGGSGGSGGS

SEQ ID NO: 23 - skipped

SEQ ID NO: 24 - protein, 6 aa, synthetic construct
GSGGSG

SEQ ID NO: 25 - protein, 9 aa, synthetic construct
GSGGSGGSG

SEQ ID NO: 26 - protein, 12 aa, synthetic construct
GSGGSGGSGGSG

SEQ ID NO: 27 - protein, 15 aa, synthetic construct
GSGGSGGSGGSGGSG

SEQ ID NO: 28 - protein, 5 aa, synthetic construct
  source 1..5: note = The entire sequence of amino acids 1-5 can be repeated one or more times
GGGGS

SEQ ID NO: 29 - protein, 10 aa, synthetic construct
GGGGSGGGGS

SEQ ID NO: 30 - protein, 15 aa, synthetic construct
GGGGSGGGGSGGGGS

SEQ ID NO: 31 - protein, 20 aa, synthetic construct
GGGGSGGGGSGGGGSGGGGS

SEQ ID NO: 32 - protein, 25 aa, synthetic construct
GGGGSGGGGSGGGGSGGGGSGGGGS

SEQ ID NO: 33 - protein, 62 aa, synthetic construct
FNSSINNIHEMEIQLKDALEKNQQWLVYDQQREVYVKGLLAKIFELEKKTETAAHSLPHSLP

SEQ ID NO: 34 - protein, 468 aa, synthetic construct
MGWSCIILFLVATATGVHSEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 234 aa, synthetic construct
MGWSCIILFLVATATGSWAQSELTQPRSVSGSPGQSVTISCTGTSRDVGGYNYVSWYQQHPGKAPKLIIHDVIERSSGVPDRFSGSKSGNTASLTISGLQAEDEADYYCWSFAGSYYVFGTGTDVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 36 - protein, 1376 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSALPGNPDHREMGETLPEEVGEYRQPSGGSVPVSPGPPSGLEPTSSSPYGGGSEQKLISEEDLGGGSFNSSINNIHEMEIQLKDALEKNQQWLVYDQQREVYVKGLLAKIFELEKKTETAAHSLP

SEQ ID NO: 37 - protein, 511 aa, synthetic construct
MATGSRTSLLLAFGLLCLPWLQEGSAELAGIGILTVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 38 - protein, 485 aa, synthetic construct
ELAGIGILTVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 39 - protein, 573 aa, synthetic construct
MATGSRTSLLLAFGLLCLPWLQEGSAELAGIGILTVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKVGGGSFNSSINNIHEMEIQLKDALEKNQQWLVYDQQREVYVKGLLAKIFELEKKTETAAHSLP

SEQ ID NO: 40 - protein, 547 aa, synthetic construct
ELAGIGILTVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKVGGGSFNSSINNIHEMEIQLKDALEKNQQWLVYDQQREVYVKGLLAKIFELEKKTETAAHSLP

SEQ ID NO: 41 - protein, 511 aa, synthetic construct
MATGSRTSLLLAFGLLCLPWLQEGSAELAGIGILTVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 42 - protein, 485 aa, synthetic construct
ELAGIGILTVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 43 - protein, 573 aa, synthetic construct
MATGSRTSLLLAFGLLCLPWLQEGSAELAGIGILTVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKVGGGSFNSSINNIHEMEIQLKDALEKNQQWLVYDQQREVYVKGLLAKIFELEKKTETAAHSLP

SEQ ID NO: 44 - protein, 547 aa, synthetic construct
ELAGIGILTVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKVGGGSFNSSINNIHEMEIQLKDALEKNQQWLVYDQQREVYVKGLLAKIFELEKKTETAAHSLP

SEQ ID NO: 45 - protein, 510 aa, synthetic construct
MATGSRTSLLLAFGLLCLPWLQEGSASLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 46 - protein, 484 aa, synthetic construct
SLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 47 - protein, 572 aa, synthetic construct
MATGSRTSLLLAFGLLCLPWLQEGSASLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKVGGGSFNSSINNIHEMEIQLKDALEKNQQWLVYDQQREVYVKGLLAKIFELEKKTETAAHSLP

SEQ ID NO: 48 - protein, 546 aa, synthetic construct
SLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKVGGGSFNSSINNIHEMEIQLKDALEKNQQWLVYDQQREVYVKGLLAKIFELEKKTETAAHSLP

SEQ ID NO: 49 - protein, 510 aa, synthetic construct
MATGSRTSLLLAFGLLCLPWLQEGSASLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 50 - protein, 484 aa, synthetic construct
SLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 51 - protein, 572 aa, synthetic construct
MATGSRTSLLLAFGLLCLPWLQEGSASLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKVGGGSFNSSINNIHEMEIQLKDALEKNQQWLVYDQQREVYVKGLLAKIFELEKKTETAAHSLP

SEQ ID NO: 52 - protein, 546 aa, synthetic construct
SLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKVGGGSFNSSINNIHEMEIQLKDALEKNQQWLVYDQQREVYVKGLLAKIFELEKKTETAAHSLP

SEQ ID NO: 53 - protein, 119 aa, synthetic construct
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 54 - protein, 99 aa, synthetic construct [sequence omitted]

SEQ ID NO: 55 - protein, 149 aa, synthetic construct
MATGSRTSLLLAFGLLCLPWLQEGSASLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 56 - protein, 123 aa, synthetic construct
SLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 57 - protein, 486 aa, synthetic construct
MATGSRTSLLLAFGLLCLPWLQEGSAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 58 - protein, 460 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 59 - protein, 548 aa, synthetic construct
MATGSRTSLLLAFGLLCLPWLQEGSAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKVGGGSFNSSINNIHEMEIQLKDALEKNQQWLVYDQQREVYVKGLLAKIFELEKKTETAAHSLP

SEQ ID NO: 60 - protein, 522 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKVGGGSFNSSINNIHEMEIQLKDALEKNQQWLVYDQQREVYVKGLLAKIFELEKKTETAAHSLP

SEQ ID NO: 61 - protein, 367 aa, synthetic construct
MATGSRTSLLLAFGLLCLPWLQEGSAGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 62 - protein, 339 aa, synthetic construct
HSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 63 - protein, 429 aa, synthetic construct
MATGSRTSLLLAFGLLCLPWLQEGSAGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKVGGGSFNSSINNIHEMEIQLKDALEKNQQWLVYDQQREVYVKGLLAKIFELEKKTETAAHSLP

SEQ ID NO: 64 - protein, 401 aa, synthetic construct
HSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKVGGGSFNSSINNIHEMEIQLKDALEKNQQWLVYDQQREVYVKGLLAKIFELEKKTETAAHSLP

SEQ ID NO: 65 - protein, 516 aa, synthetic construct
MATGSRTSLLLAFGLLCLPWLQEGSAKYNKANVFLGGGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRWKLPPSTVSNTVIIAVLVVLGAAIVTGAVVAFVMKMRRNTGGKGVNYALAPGSQTSDLSLPDGKVMVHDPHSLA

SEQ ID NO: 66 - protein, 490 aa, synthetic construct
KYNKANVFLGGGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRWKLPPSTVSNTVIIAVLVVLGAAIVTGAVVAFVMKMRRNTGGKGVNYALAPGSQTSDLSLPDGKVMVHDPHSLA

SEQ ID NO: 67 - protein, 578 aa, synthetic construct
MATGSRTSLLLAFGLLCLPWLQEGSAKYNKANVFLGGGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRWKLPPSTVSNTVIIAVLVVLGAAIVTGAVVAFVMKMRRNTGGKGVNYALAPGSQTSDLSLPDGKVMVHDPHSLAGGGSFNSSINNIHEMEIQLKDALEKNQQWLVYDQQREVYVKGLLAKIFELEKKTETAAHSLP

SEQ ID NO: 68 - protein, 552 aa, synthetic construct
KYNKANVFLGGGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRWKLPPSTVSNTVIIAVLVVLGAAIVTGAVVAFVMKMRRNTGGKGVNYALAPGSQTSDLSLPDGKVMVHDPHSLAGGGSFNSSINNIHEMEIQLKDALEKNQQWLVYDQQREVYVKGLLAKIFELEKKTETAAHSLP

SEQ ID NO: 69 - protein, 516 aa, synthetic construct
MATGSRTSLLLAFGLLCLPWLQEGSAVYLKTNVFLGGGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRWKLPPSTVSNTVIIAVLVVLGAAIVTGAVVAFVMKMRRNTGGKGVNYALAPGSQTSDLSLPDGKVMVHDPHSLA

SEQ ID NO: 70 - protein, 490 aa, synthetic construct
VYLKTNVFLGGGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRWKLPPSTVSNTVIIAVLVVLGAAIVTGAVVAFVMKMRRNTGGKGVNYALAPGSQTSDLSLPDGKVMVHDPHSLA

SEQ ID NO: 71 - protein, 578 aa, synthetic construct
MATGSRTSLLLAFGLLCLPWLQEGSAVYLKTNVFLGGGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRWKLPPSTVSNTVIIAVLVVLGAAIVTGAVVAFVMKMRRNTGGKGVNYALAPGSQTSDLSLPDGKVMVHDPHSLAGGGSFNSSINNIHEMEIQLKDALEKNQQWLVYDQQREVYVKGLLAKIFELEKKTETAAHSLP

SEQ ID NO: 72 - protein, 552 aa, synthetic construct
VYLKTNVFLGGGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRWKLPPSTVSNTVIIAVLVVLGAAIVTGAVVAFVMKMRRNTGGKGVNYALAPGSQTSDLSLPDGKVMVHDPHSLAGGGSFNSSINNIHEMEIQLKDALEKNQQWLVYDQQREVYVKGLLAKIFELEKKTETAAHSLP

SEQ ID NO: 73 - protein, 507 aa, synthetic construct
MATGSRTSLLLAFGLLCLPWLQEGSASLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLLRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 74 - protein, 481 aa, synthetic construct
SLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLLRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 75 - protein, 569 aa, synthetic construct
MATGSRTSLLLAFGLLCLPWLQEGSASLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLLRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKAGGGSFNSSINNIHEMEIQLKDALEKNQQWLVYDQQREVYVKGLLAKIFELEKKTETAAHSLP

SEQ ID NO: 76 - protein, 543 aa, synthetic construct
SLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLLRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKAGGGSFNSSINNIHEMEIQLKDALEKNQQWLVYDQQREVYVKGLLAKIFELEKKTETAAHSLP

SEQ ID NO: 77 - protein, 517 aa, synthetic construct
MATGSRTSLLLAFGLLCLPWLQEGSASLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLA

SEQ ID NO: 78 - protein, 491 aa, synthetic construct
SLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLA

SEQ ID NO: 79 - protein, 579 aa, synthetic construct
MATGSRTSLLLAFGLLCLPWLQEGSASLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLAGGGSFNSSINNIHEMEIQLKDALEKNQQWLVYDQQREVYVKGLLAKIFELEKKTETAAHSLP

SEQ ID NO: 80 - protein, 553 aa, synthetic construct
SLLMWITQVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLAGGGSFNSSINNIHEMEIQLKDALEKNQQWLVYDQQREVYVKGLLAKIFELEKKTETAAHSLP

SEQ ID NO: 81 - protein, 466 aa, synthetic construct
IQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRWKLPPSTVSNTVIIAVLVVLGAAIVTGAVVAFVMKMRRNTGGKGVNYALAPGSQTSDLSLPDGKVMVHDPHSLA

SEQ ID NO: 82 - protein, 347 aa, synthetic construct
GPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRWKLPPSTVSNTVIIAVLVVLGAAIVTGAVVAFVMKMRRNTGGKGVNYALAPGSQTSDLSLPDGKVMVHDPHSLA

SEQ ID NO: 83 - protein, 457 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLLRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 84 - protein, 336 aa, synthetic construct
HSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLLRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 85 - protein, 467 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLA

SEQ ID NO: 86 - protein, 346 aa, synthetic construct
HSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLA

SEQ ID NO: 87 - DNA, 1407 bp, synthetic construct
  source 1..1407: mol_type = other DNA
atgggatggtcatgtatcatcctttttctagtagcaactgcaaccggtgtacattctgaagtacagttgctggagagtggaggaggattggtgcaaccgggaggctcccttaggttgagttgtgccgcgtccggttttactttttccacatatcaaatgtcttgggtgcggcaagctccaggtaagggattggagtgggtttcaggaatcgtgtcttccggcggaagtacagcttacgctgactcagtgaagggtaggttcacaatttcacgcgacaatagcaaaaacacgctctacctccagatgaacagtctcagagctgaagacacggcggtgtattattgcgccggagagttgttgccctactacgggatggatgtttggggacagggcaccaccgtaacagtctctagcgcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 88 - DNA, 705 bp, synthetic construct
  source 1..705: mol_type = other DNA
atgggatggtcatgtatcatcctttttctagtagcaactgcaaccggttcctgggcccaaagcgaacttactcagccaagatctgttagcggttcccccggccagtcagtaacaatcagttgcaccggcacctctagagatgtcggtggctataattatgtatcatggtatcagcaacacccaggtaaggcacctaagctgattattcacgacgttattgaacggagttcaggggtgcctgatcgcttttctggctccaaatccggaaacaccgctagtctgacaatcagtgggcttcaggcggaggatgaagccgactactattgctggtcctttgctggcagctattacgttttcggcactgggacagacgtgactgttctcggtcagcccaaggctgccccctcggtcactctgttcccaccctcgagtgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 89 - DNA, 4131 bp, synthetic construct
  source 1..4131: mol_type = other DNA
atgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcaccacacggcgtggtgtttctgcacgtgacctacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccttggtacatctggctgggctttatcgccggactgattgccatcgtgatggtcacaatcatgctgtgttgcatgaccagctgctgtagctgcctgaagggctgttgtagctgtggctctgcccttcctggcaaccctgatcacagagagatgggcgagacactgcctgaggaagtgggcgagtacagacagccttctggcggctctgtgcctgtgtctcctggacctccttctggcctggaacctacaagcagcagcccttacggcggaggctctgaacaaaaactcatctcagaagaggatctgggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgccttga

SEQ ID NO: 90 - DNA, 1536 bp, synthetic construct
  source 1..1536: mol_type = other DNA
atggcgaccgggagtcgaacatcattgttgcttgcatttgggctcctttgcctgccgtggctccaagaagggtccgccgagttggcaggaattggtatacttacagttggtggcggtggctccggtggaggtggcagtggaggtggcggtagcattcaacgaactccaaagatacaggtatacagtagacaccctgccgaaaacggaaaaagtaacttccttaattgctatgtttccggatttcacccttcagacatagaggtagacttgctgaagaatggtgaaaggatagagaaagtggagcactctgaccttagtttcagtaaggactggagtttctatctgctgtactacaccgaatttaccccaactgaaaaagacgagtacgcatgtcgggtgaaccacgttacgctcagtcagccaaagatagtgaaatgggatcgagatatgggtggaggtggtagcggtggaggtgggagcggagggggtggatctggaggtggagggtctggcagccatagtatgcgatactttttcacaagcgtttctcggccgggccgcggcgaacctagatttatagcagtaggttacgtcgatgacactcagttcgtccggtttgatagcgatgctgcaagtcaaaggatggaaccccgggcaccttggattgagcaggagggccctgaatattgggatggcgaaacgcgcaaagtgaaggcgcacagccaaacacatcgcgtggacttggggacccttcgagggtactacaatcagagcgaagctggaagccacacggtacaaagaatgtacgggtgtgacgtcgggtccgactggcgctttctcagaggttaccatcagtatgcttatgatggtaaggactacattgcactgaaggaggatcttcgatcctggaccgctgcggacatggcggcccaaacaacaaagcacaaatgggaagctgcccacgtcgctgaacagttgcgcgcgtatctggaaggtacgtgtgttgaatggctgagacggtacctcgaaaacggcaaagaaacgctccagaggacggatgcaccgaaaacgcacatgacgcaccatgccgtttctgatcatgaagcgacacttcgctgctgggcgctttctttctatcccgccgaaattacgcttacgtggcagcgagatggcgaggaccagactcaagacaccgaactggtcgaaacgcgacctgcaggggacgggacgttccaaaaatgggcggccgtggtggttccaagtgggcaagaacagcggtacacctgccacgtccagcacgagggtctcccgaaacctctcacacttcggtgggaaccttcttcccaacctacaatcccaatcgtcgggataatcgctggtctggttttgttcggggcagtcattaccggagctgttgtagccgccgtgatgtggcgaagaaaaagctctgatcgaaaggggggcagctactcacaagccgcaagcagtgacagcgctcagggcagcgacgtgagcttgacagcttgcaaggtatga

SEQ ID NO: 91 - DNA, 1722 bp, synthetic construct
  source 1..1722: mol_type = other DNA
atggcgaccgggagtcgaacatcattgttgcttgcatttgggctcctttgcctgccgtggctccaagaagggtccgccgagttggcaggaattggtatacttacagttggtggcggtggctccggtggaggtggcagtggaggtggcggtagcattcaacgaactccaaagatacaggtatacagtagacaccctgccgaaaacggaaaaagtaacttccttaattgctatgtttccggatttcacccttcagacatagaggtagacttgctgaagaatggtgaaaggatagagaaagtggagcactctgaccttagtttcagtaaggactggagtttctatctgctgtactacaccgaatttaccccaactgaaaaagacgagtacgcatgtcgggtgaaccacgttacgctcagtcagccaaagatagtgaaatgggatcgagatatgggtggaggtggtagcggtggaggtgggagcggagggggtggatctggaggtggagggtctggcagccatagtatgcgatactttttcacaagcgtttctcggccgggccgcggcgaacctagatttatagcagtaggttacgtcgatgacactcagttcgtccggtttgatagcgatgctgcaagtcaaaggatggaaccccgggcaccttggattgagcaggagggccctgaatattgggatggcgaaacgcgcaaagtgaaggcgcacagccaaacacatcgcgtggacttggggacccttcgagggtactacaatcagagcgaagctggaagccacacggtacaaagaatgtacgggtgtgacgtcgggtccgactggcgctttctcagaggttaccatcagtatgcttatgatggtaaggactacattgcactgaaggaggatcttcgatcctggaccgctgcggacatggcggcccaaacaacaaagcacaaatgggaagctgcccacgtcgctgaacagttgcgcgcgtatctggaaggtacgtgtgttgaatggctgagacggtacctcgaaaacggcaaagaaacgctccagaggacggatgcaccgaaaacgcacatgacgcaccatgccgtttctgatcatgaagcgacacttcgctgctgggcgctttctttctatcccgccgaaattacgcttacgtggcagcgagatggcgaggaccagactcaagacaccgaactggtcgaaacgcgacctgcaggggacgggacgttccaaaaatgggcggccgtggtggttccaagtgggcaagaacagcggtacacctgccacgtccagcacgagggtctcccgaaacctctcacacttcggtgggaaccttcttcccaacctacaatcccaatcgtcgggataatcgctggtctggttttgttcggggcagtcattaccggagctgttgtagccgccgtgatgtggcgaagaaaaagctctgatcgaaaggggggcagctactcacaagccgcaagcagtgacagcgctcagggcagcgacgtgagcttgacagcttgcaaggtaggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgccttga

SEQ ID NO: 92 - DNA, 1536 bp, synthetic construct
  source 1..1536: mol_type = other DNA
atggcgaccgggagtcgaacatcattgttgcttgcatttgggctcctttgcctgccgtggctccaagaagggtccgccgagttggcaggaattggtatacttacagttggtggcggtggctccggtggaggtggcagtggaggtggcggtagcattcaacgaactccaaagatacaggtatacagtagacaccctgccgaaaacggaaaaagtaacttccttaattgctatgtttccggatttcacccttcagacatagaggtagacttgctgaagaatggtgaaaggatagagaaagtggagcactctgaccttagtttcagtaaggactggagtttctatctgctgtactacaccgaatttaccccaactgaaaaagacgagtacgcatgtcgggtgaaccacgttacgctcagtcagccaaagatagtgaaatgggatcgagatatgggtggaggtggtagcggtggaggtgggagcggagggggtggatctggaggtggagggtctggcagccatagtatgcgatactttttcacaagcgtttctcggccgggccgcggcgaacctagatttatagcagtaggttacgtcgatgacactcagttcgtccggtttgatagcgatgctgcaagtcaaaggatggaaccccgggcaccttggattgagcaggagggccctgaatattgggatggcgaaacgcgcaaagtgaaggcgcacagccaaacacatcgcgtggacttggggacccttcgagggtgctacaatcagagcgaagctggaagccacacggtacaaagaatgtacgggtgtgacgtcgggtccgactggcgctttctcagaggttaccatcagtatgcttatgatggtaaggactacattgcactgaaggaggatcttcgatcctggaccgctgcggacatgtgcgcccaaacaacaaagcacaaatgggaagctgcccacgtcgctgaacagttgcgcgcgtatctggaaggtacgtgtgttgaatggctgagacggtacctcgaaaacggcaaagaaacgctccagaggacggatgcaccgaaaacgcacatgacgcaccatgccgtttctgatcatgaagcgacacttcgctgctgggcgctttctttctatcccgccgaaattacgcttacgtggcagcgagatggcgaggaccagactcaagacaccgaactggtcgaaacgcgacctgcaggggacgggacgttccaaaaatgggcggccgtggtggttccaagtgggcaagaacagcggtacacctgccacgtccagcacgagggtctcccgaaacctctcacacttcggtgggaaccttcttcccaacctacaatcccaatcgtcgggataatcgctggtctggttttgttcggggcagtcattaccggagctgttgtagccgccgtgatgtggcgaagaaaaagctctgatcgaaaggggggcagctactcacaagccgcaagcagtgacagcgctcagggcagcgacgtgagcttgacagcttgcaaggtatga

SEQ ID NO: 93 - DNA, 1722 bp, synthetic construct
  source 1..1722: mol_type = other DNA
atggcgaccgggagtcgaacatcattgttgcttgcatttgggctcctttgcctgccgtggctccaagaagggtccgccgagttggcaggaattggtatacttacagttggtggcggtggctccggtggaggtggcagtggaggtggcggtagcattcaacgaactccaaagatacaggtatacagtagacaccctgccgaaaacggaaaaagtaacttccttaattgctatgtttccggatttcacccttcagacatagaggtagacttgctgaagaatggtgaaaggatagagaaagtggagcactctgaccttagtttcagtaaggactggagtttctatctgctgtactacaccgaatttaccccaactgaaaaagacgagtacgcatgtcgggtgaaccacgttacgctcagtcagccaaagatagtgaaatgggatcgagatatgggtggaggtggtagcggtggaggtgggagcggagggggtggatctggaggtggagggtctggcagccatagtatgcgatactttttcacaagcgtttctcggccgggccgcggcgaacctagatttatagcagtaggttacgtcgatgacactcagttcgtccggtttgatagcgatgctgcaagtcaaaggatggaaccccgggcaccttggattgagcaggagggccctgaatattgggatggcgaaacgcgcaaagtgaaggcgcacagccaaacacatcgcgtggacttggggacccttcgagggtgctacaatcagagcgaagctggaagccacacggtacaaagaatgtacgggtgtgacgtcgggtccgactggcgctttctcagaggttaccatcagtatgcttatgatggtaaggactacattgcactgaaggaggatcttcgatcctggaccgctgcggacatgtgcgcccaaacaacaaagcacaaatgggaagctgcccacgtcgctgaacagttgcgcgcgtatctggaaggtacgtgtgttgaatggctgagacggtacctcgaaaacggcaaagaaacgctccagaggacggatgcaccgaaaacgcacatgacgcaccatgccgtttctgatcatgaagcgacacttcgctgctgggcgctttctttctatcccgccgaaattacgcttacgtggcagcgagatggcgaggaccagactcaagacaccgaactggtcgaaacgcgacctgcaggggacgggacgttccaaaaatgggcggccgtggtggttccaagtgggcaagaacagcggtacacctgccacgtccagcacgagggtctcccgaaacctctcacacttcggtgggaaccttcttcccaacctacaatcccaatcgtcgggataatcgctggtctggttttgttcggggcagtcattaccggagctgttgtagccgccgtgatgtggcgaagaaaaagctctgatcgaaaggggggcagctactcacaagccgcaagcagtgacagcgctcagggcagcgacgtgagcttgacagcttgcaaggtaggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgccttga

SEQ ID NO: 94 - DNA, 1533 bp, synthetic construct
  source 1..1533: mol_type = other DNA
atggcgaccgggagtcgaacatcattgttgcttgcatttgggctcctttgcctgccgtggctccaagaagggtccgcctccctgcttatgtggattacgcaagttggtggcggtggctccggtggaggtggcagtggaggtggcggtagcattcaacgaactccaaagatacaggtatacagtagacaccctgccgaaaacggaaaaagtaacttccttaattgctatgtttccggatttcacccttcagacatagaggtagacttgctgaagaatggtgaaaggatagagaaagtggagcactctgaccttagtttcagtaaggactggagtttctatctgctgtactacaccgaatttaccccaactgaaaaagacgagtacgcatgtcgggtgaaccacgttacgctcagtcagccaaagatagtgaaatgggatcgagatatgggtggaggtggtagcggtggaggtgggagcggagggggtggatctggaggtggagggtctggcagccatagtatgcgatactttttcacaagcgtttctcggccgggccgcggcgaacctagatttatagcagtaggttacgtcgatgacactcagttcgtccggtttgatagcgatgctgcaagtcaaaggatggaaccccgggcaccttggattgagcaggagggccctgaatattgggatggcgaaacgcgcaaagtgaaggcgcacagccaaacacatcgcgtggacttggggacccttcgagggtactacaatcagagcgaagctggaagccacacggtacaaagaatgtacgggtgtgacgtcgggtccgactggcgctttctcagaggttaccatcagtatgcttatgatggtaaggactacattgcactgaaggaggatcttcgatcctggaccgctgcggacatggcggcccaaacaacaaagcacaaatgggaagctgcccacgtcgctgaacagttgcgcgcgtatctggaaggtacgtgtgttgaatggctgagacggtacctcgaaaacggcaaagaaacgctccagaggacggatgcaccgaaaacgcacatgacgcaccatgccgtttctgatcatgaagcgacacttcgctgctgggcgctttctttctatcccgccgaaattacgcttacgtggcagcgagatggcgaggaccagactcaagacaccgaactggtcgaaacgcgacctgcaggggacgggacgttccaaaaatgggcggccgtggtggttccaagtgggcaagaacagcggtacacctgccacgtccagcacgagggtctcccgaaacctctcacacttcggtgggaaccttcttcccaacctacaatcccaatcgtcgggataatcgctggtctggttttgttcggggcagtcattaccggagctgttgtagccgccgtgatgtggcgaagaaaaagctctgatcgaaaggggggcagctactcacaagccgcaagcagtgacagcgctcagggcagcgacgtgagcttgacagcttgcaaggtatga

SEQ ID NO: 95 - DNA, 1719 bp, synthetic construct
  source 1..1719: mol_type = other DNA
atggcgaccgggagtcgaacatcattgttgcttgcatttgggctcctttgcctgccgtggctccaagaagggtccgcctccctgcttatgtggattacgcaagttggtggcggtggctccggtggaggtggcagtggaggtggcggtagcattcaacgaactccaaagatacaggtatacagtagacaccctgccgaaaacggaaaaagtaacttccttaattgctatgtttccggatttcacccttcagacatagaggtagacttgctgaagaatggtgaaaggatagagaaagtggagcactctgaccttagtttcagtaaggactggagtttctatctgctgtactacaccgaatttaccccaactgaaaaagacgagtacgcatgtcgggtgaaccacgttacgctcagtcagccaaagatagtgaaatgggatcgagatatgggtggaggtggtagcggtggaggtgggagcggagggggtggatctggaggtggagggtctggcagccatagtatgcgatactttttcacaagcgtttctcggccgggccgcggcgaacctagatttatagcagtaggttacgtcgatgacactcagttcgtccggtttgatagcgatgctgcaagtcaaaggatggaaccccgggcaccttggattgagcaggagggccctgaatattgggatggcgaaacgcgcaaagtgaaggcgcacagccaaacacatcgcgtggacttggggacccttcgagggtactacaatcagagcgaagctggaagccacacggtacaaagaatgtacgggtgtgacgtcgggtccgactggcgctttctcagaggttaccatcagtatgcttatgatggtaaggactacattgcactgaaggaggatcttcgatcctggaccgctgcggacatggcggcccaaacaacaaagcacaaatgggaagctgcccacgtcgctgaacagttgcgcgcgtatctggaaggtacgtgtgttgaatggctgagacggtacctcgaaaacggcaaagaaacgctccagaggacggatgcaccgaaaacgcacatgacgcaccatgccgtttctgatcatgaagcgacacttcgctgctgggcgctttctttctatcccgccgaaattacgcttacgtggcagcgagatggcgaggaccagactcaagacaccgaactggtcgaaacgcgacctgcaggggacgggacgttccaaaaatgggcggccgtggtggttccaagtgggcaagaacagcggtacacctgccacgtccagcacgagggtctcccgaaacctctcacacttcggtgggaaccttcttcccaacctacaatcccaatcgtcgggataatcgctggtctggttttgttcggggcagtcattaccggagctgttgtagccgccgtgatgtggcgaagaaaaagctctgatcgaaaggggggcagctactcacaagccgcaagcagtgacagcgctcagggcagcgacgtgagcttgacagcttgcaaggtaggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgccttga

SEQ ID NO: 96 - DNA, 1533 bp, synthetic construct
  source 1..1533: mol_type = other DNA
atggcgaccgggagtcgaacatcattgttgcttgcatttgggctcctttgcctgccgtggctccaagaagggtccgcctccctgcttatgtggattacgcaagttggtggcggtggctccggtggaggtggcagtggaggtggcggtagcattcaacgaactccaaagatacaggtatacagtagacaccctgccgaaaacggaaaaagtaacttccttaattgctatgtttccggatttcacccttcagacatagaggtagacttgctgaagaatggtgaaaggatagagaaagtggagcactctgaccttagtttcagtaaggactggagtttctatctgctgtactacaccgaatttaccccaactgaaaaagacgagtacgcatgtcgggtgaaccacgttacgctcagtcagccaaagatagtgaaatgggatcgagatatgggtggaggtggtagcggtggaggtgggagcggagggggtggatctggaggtggagggtctggcagccatagtatgcgatactttttcacaagcgtttctcggccgggccgcggcgaacctagatttatagcagtaggttacgtcgatgacactcagttcgtccggtttgatagcgatgctgcaagtcaaaggatggaaccccgggcaccttggattgagcaggagggccctgaatattgggatggcgaaacgcgcaaagtgaaggcgcacagccaaacacatcgcgtggacttggggacccttcgagggtgctacaatcagagcgaagctggaagccacacggtacaaagaatgtacgggtgtgacgtcgggtccgactggcgctttctcagaggttaccatcagtatgcttatgatggtaaggactacattgcactgaaggaggatcttcgatcctggaccgctgcggacatgtgcgcccaaacaacaaagcacaaatgggaagctgcccacgtcgctgaacagttgcgcgcgtatctggaaggtacgtgtgttgaatggctgagacggtacctcgaaaacggcaaagaaacgctccagaggacggatgcaccgaaaacgcacatgacgcaccatgccgtttctgatcatgaagcgacacttcgctgctgggcgctttctttctatcccgccgaaattacgcttacgtggcagcgagatggcgaggaccagactcaagacaccgaactggtcgaaacgcgacctgcaggggacgggacgttccaaaaatgggcggccgtggtggttccaagtgggcaagaacagcggtacacctgccacgtccagcacgagggtctcccgaaacctctcacacttcggtgggaaccttcttcccaacctacaatcccaatcgtcgggataatcgctggtctggttttgttcggggcagtcattaccggagctgttgtagccgccgtgatgtggcgaagaaaaagctctgatcgaaaggggggcagctactcacaagccgcaagcagtgacagcgctcagggcagcgacgtgagcttgacagcttgcaaggtatga

SEQ ID NO: 97 - DNA, 1719 bp, synthetic construct
  source 1..1719: mol_type = other DNA
atggcgaccgggagtcgaacatcattgttgcttgcatttgggctcctttgcctgccgtggctccaagaagggtccgcctccctgcttatgtggattacgcaagttggtggcggtggctccggtggaggtggcagtggaggtggcggtagcattcaacgaactccaaagatacaggtatacagtagacaccctgccgaaaacggaaaaagtaacttccttaattgctatgtttccggatttcacccttcagacatagaggtagacttgctgaagaatggtgaaaggatagagaaagtggagcactctgaccttagtttcagtaaggactggagtttctatctgctgtactacaccgaatttaccccaactgaaaaagacgagtacgcatgtcgggtgaaccacgttacgctcagtcagccaaagatagtgaaatgggatcgagatatgggtggaggtggtagcggtggaggtgggagcggagggggtggatctggaggtggagggtctggcagccatagtatgcgatactttttcacaagcgtttctcggccgggccgcggcgaacctagatttatagcagtaggttacgtcgatgacactcagttcgtccggtttgatagcgatgctgcaagtcaaaggatggaaccccgggcaccttggattgagcaggagggccctgaatattgggatggcgaaacgcgcaaagtgaaggcgcacagccaaacacatcgcgtggacttggggacccttcgagggtgctacaatcagagcgaagctggaagccacacggtacaaagaatgtacgggtgtgacgtcgggtccgactggcgctttctcagaggttaccatcagtatgcttatgatggtaaggactacattgcactgaaggaggatcttcgatcctggaccgctgcggacatgtgcgcccaaacaacaaagcacaaatgggaagctgcccacgtcgctgaacagttgcgcgcgtatctggaaggtacgtgtgttgaatggctgagacggtacctcgaaaacggcaaagaaacgctccagaggacggatgcaccgaaaacgcacatgacgcaccatgccgtttctgatcatgaagcgacacttcgctgctgggcgctttctttctatcccgccgaaattacgcttacgtggcagcgagatggcgaggaccagactcaagacaccgaactggtcgaaacgcgacctgcaggggacgggacgttccaaaaatgggcggccgtggtggttccaagtgggcaagaacagcggtacacctgccacgtccagcacgagggtctcccgaaacctctcacacttcggtgggaaccttcttcccaacctacaatcccaatcgtcgggataatcgctggtctggttttgttcggggcagtcattaccggagctgttgtagccgccgtgatgtggcgaagaaaaagctctgatcgaaaggggggcagctactcacaagccgcaagcagtgacagcgctcagggcagcgacgtgagcttgacagcttgcaaggtaggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgccttga

SEQ ID NO: 98 - DNA, 360 bp, synthetic construct
  source 1..360: mol_type = other DNA
atgtcacgcagtgtggcgcttgcggttcttgcgctgcttagtctttcagggctggaggccattcaacgaactccaaagatacaggtatacagtagacaccctgccgaaaacggaaaaagtaacttccttaattgctatgtttccggatttcacccttcagacatagaggtagacttgctgaagaatggtgaaaggatagagaaagtggagcactctgaccttagtttcagtaaggactggagtttctatctgctgtactacaccgaatttaccccaactgaaaaagacgagtacgcatgtcgggtgaaccacgttacgctcagtcagccaaagatagtgaaatgggatcgagatatgtga

SEQ ID NO: 99 - DNA, 450 bp, synthetic construct
  source 1..450: mol_type = other DNA
atggcgaccgggagtcgaacatcattgttgcttgcatttgggctcctttgcctgccgtggctccaagaagggtccgcctccctgcttatgtggattacgcaagttggtggcggtggctccggtggaggtggcagtggaggtggcggtagcattcaacgaactccaaagatacaggtatacagtagacaccctgccgaaaacggaaaaagtaacttccttaattgctatgtttccggatttcacccttcagacatagaggtagacttgctgaagaatggtgaaaggatagagaaagtggagcactctgaccttagtttcagtaaggactggagtttctatctgctgtactacaccgaatttaccccaactgaaaaagacgagtacgcatgtcgggtgaaccacgttacgctcagtcagccaaagatagtgaaatgggatcgagatatgtga

SEQ ID NO: 100 - DNA, 1461 bp, synthetic construct
  source 1..1461: mol_type = other DNA
atggcgaccgggagtcgaacatcattgttgcttgcatttgggctcctttgcctgccgtggctccaagaagggtccgccattcaacgaactccaaagatacaggtatacagtagacaccctgccgaaaacggaaaaagtaacttccttaattgctatgtttccggatttcacccttcagacatagaggtagacttgctgaagaatggtgaaaggatagagaaagtggagcactctgaccttagtttcagtaaggactggagtttctatctgctgtactacaccgaatttaccccaactgaaaaagacgagtacgcatgtcgggtgaaccacgttacgctcagtcagccaaagatagtgaaatgggatcgagatatgggtggaggtggtagcggtggaggtgggagcggagggggtggatctggaggtggagggtctggcagccatagtatgcgatactttttcacaagcgtttctcggccgggccgcggcgaacctagatttatagcagtaggttacgtcgatgacactcagttcgtccggtttgatagcgatgctgcaagtcaaaggatggaaccccgggcaccttggattgagcaggagggccctgaatattgggatggcgaaacgcgcaaagtgaaggcgcacagccaaacacatcgcgtggacttggggacccttcgagggtgctacaatcagagcgaagctggaagccacacggtacaaagaatgtacgggtgtgacgtcgggtccgactggcgctttctcagaggttaccatcagtatgcttatgatggtaaggactacattgcactgaaggaggatcttcgatcctggaccgctgcggacatgtgcgcccaaacaacaaagcacaaatgggaagctgcccacgtcgctgaacagttgcgcgcgtatctggaaggtacgtgtgttgaatggctgagacggtacctcgaaaacggcaaagaaacgctccagaggacggatgcaccgaaaacgcacatgacgcaccatgccgtttctgatcatgaagcgacacttcgctgctgggcgctttctttctatcccgccgaaattacgcttacgtggcagcgagatggcgaggaccagactcaagacaccgaactggtcgaaacgcgacctgcaggggacgggacgttccaaaaatgggcggccgtggtggttccaagtgggcaagaacagcggtacacctgccacgtccagcacgagggtctcccgaaacctctcacacttcggtgggaaccttcttcccaacctacaatcccaatcgtcgggataatcgctggtctggttttgttcggggcagtcattaccggagctgttgtagccgccgtgatgtggcgaagaaaaagctctgatcgaaaggggggcagctactcacaagccgcaagcagtgacagcgctcagggcagcgacgtgagcttgacagcttgcaaggtatga

SEQ ID NO: 101 - DNA, 1647 bp, synthetic construct
  source 1..1647: mol_type = other DNA
atggcgaccgggagtcgaacatcattgttgcttgcatttgggctcctttgcctgccgtggctccaagaagggtccgccattcaacgaactccaaagatacaggtatacagtagacaccctgccgaaaacggaaaaagtaacttccttaattgctatgtttccggatttcacccttcagacatagaggtagacttgctgaagaatggtgaaaggatagagaaagtggagcactctgaccttagtttcagtaaggactggagtttctatctgctgtactacaccgaatttaccccaactgaaaaagacgagtacgcatgtcgggtgaaccacgttacgctcagtcagccaaagatagtgaaatgggatcgagatatgggtggaggtggtagcggtggaggtgggagcggagggggtggatctggaggtggagggtctggcagccatagtatgcgatactttttcacaagcgtttctcggccgggccgcggcgaacctagatttatagcagtaggttacgtcgatgacactcagttcgtccggtttgatagcgatgctgcaagtcaaaggatggaaccccgggcaccttggattgagcaggagggccctgaatattgggatggcgaaacgcgcaaagtgaaggcgcacagccaaacacatcgcgtggacttggggacccttcgagggtgctacaatcagagcgaagctggaagccacacggtacaaagaatgtacgggtgtgacgtcgggtccgactggcgctttctcagaggttaccatcagtatgcttatgatggtaaggactacattgcactgaaggaggatcttcgatcctggaccgctgcggacatgtgcgcccaaacaacaaagcacaaatgggaagctgcccacgtcgctgaacagttgcgcgcgtatctggaaggtacgtgtgttgaatggctgagacggtacctcgaaaacggcaaagaaacgctccagaggacggatgcaccgaaaacgcacatgacgcaccatgccgtttctgatcatgaagcgacacttcgctgctgggcgctttctttctatcccgccgaaattacgcttacgtggcagcgagatggcgaggaccagactcaagacaccgaactggtcgaaacgcgacctgcaggggacgggacgttccaaaaatgggcggccgtggtggttccaagtgggcaagaacagcggtacacctgccacgtccagcacgagggtctcccgaaacctctcacacttcggtgggaaccttcttcccaacctacaatcccaatcgtcgggataatcgctggtctggttttgttcggggcagtcattaccggagctgttgtagccgccgtgatgtggcgaagaaaaagctctgatcgaaaggggggcagctactcacaagccgcaagcagtgacagcgctcagggcagcgacgtgagcttgacagcttgcaaggtaggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgccttga

SEQ ID NO: 102 - DNA, 1104 bp, synthetic construct
  source 1..1104: mol_type = other DNA
atggcgaccgggagtcgaacatcattgttgcttgcatttgggctcctttgcctgccgtggctccaagaagggtccgccggcagccatagtatgcgatactttttcacaagcgtttctcggccgggccgcggcgaacctagatttatagcagtaggttacgtcgatgacactcagttcgtccggtttgatagcgatgctgcaagtcaaaggatggaaccccgggcaccttggattgagcaggagggccctgaatattgggatggcgaaacgcgcaaagtgaaggcgcacagccaaacacatcgcgtggacttggggacccttcgagggtgctacaatcagagcgaagctggaagccacacggtacaaagaatgtacgggtgtgacgtcgggtccgactggcgctttctcagaggttaccatcagtatgcttatgatggtaaggactacattgcactgaaggaggatcttcgatcctggaccgctgcggacatgtgcgcccaaacaacaaagcacaaatgggaagctgcccacgtcgctgaacagttgcgcgcgtatctggaaggtacgtgtgttgaatggctgagacggtacctcgaaaacggcaaagaaacgctccagaggacggatgcaccgaaaacgcacatgacgcaccatgccgtttctgatcatgaagcgacacttcgctgctgggcgctttctttctatcccgccgaaattacgcttacgtggcagcgagatggcgaggaccagactcaagacaccgaactggtcgaaacgcgacctgcaggggacgggacgttccaaaaatgggcggccgtggtggttccaagtgggcaagaacagcggtacacctgccacgtccagcacgagggtctcccgaaacctctcacacttcggtgggaaccttcttcccaacctacaatcccaatcgtcgggataatcgctggtctggttttgttcggggcagtcattaccggagctgttgtagccgccgtgatgtggcgaagaaaaagctctgatcgaaaggggggcagctactcacaagccgcaagcagtgacagcgctcagggcagcgacgtgagcttgacagcttgcaaggtatga

SEQ ID NO: 103 - DNA, 1290 bp, synthetic construct
  source 1..1290: mol_type = other DNA
atggcgaccgggagtcgaacatcattgttgcttgcatttgggctcctttgcctgccgtggctccaagaagggtccgccggcagccatagtatgcgatactttttcacaagcgtttctcggccgggccgcggcgaacctagatttatagcagtaggttacgtcgatgacactcagttcgtccggtttgatagcgatgctgcaagtcaaaggatggaaccccgggcaccttggattgagcaggagggccctgaatattgggatggcgaaacgcgcaaagtgaaggcgcacagccaaacacatcgcgtggacttggggacccttcgagggtgctacaatcagagcgaagctggaagccacacggtacaaagaatgtacgggtgtgacgtcgggtccgactggcgctttctcagaggttaccatcagtatgcttatgatggtaaggactacattgcactgaaggaggatcttcgatcctggaccgctgcggacatgtgcgcccaaacaacaaagcacaaatgggaagctgcccacgtcgctgaacagttgcgcgcgtatctggaaggtacgtgtgttgaatggctgagacggtacctcgaaaacggcaaagaaacgctccagaggacggatgcaccgaaaacgcacatgacgcaccatgccgtttctgatcatgaagcgacacttcgctgctgggcgctttctttctatcccgccgaaattacgcttacgtggcagcgagatggcgaggaccagactcaagacaccgaactggtcgaaacgcgacctgcaggggacgggacgttccaaaaatgggcggccgtggtggttccaagtgggcaagaacagcggtacacctgccacgtccagcacgagggtctcccgaaacctctcacacttcggtgggaaccttcttcccaacctacaatcccaatcgtcgggataatcgctggtctggttttgttcggggcagtcattaccggagctgttgtagccgccgtgatgtggcgaagaaaaagctctgatcgaaaggggggcagctactcacaagccgcaagcagtgacagcgctcagggcagcgacgtgagcttgacagcttgcaaggtaggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgccttga

SEQ ID NO: 104 - DNA, 1551 bp, synthetic construct
  source 1..1551: mol_type = other DNA
atggcgaccgggagtcgaacatcattgttgcttgcatttgggctcctttgcctgccgtggctccaagaagggtccgccaagtataataaggcgaatgtattcctgggtggcggtggctccggtggaggtggcagtggaggtggcggtagcatacagaaaactccgcagatacaggtctattccaggcatccaccagaaaacggaaaaccaaatatattgaattgttatgtaacccagtttcaccctcctcacatcgagatccagatgctgaagaatggtaagaaaatccctaaagtggagatgtccgacatgtccttctccaaagactggagcttttacatattggcgcataccgaattcactcccacggaaactgacacgtatgcctgtagggtgaaacatgcatcaatggcagagccgaaaacagtgtactgggaccgggatatgggtggaggtggtagcggtggaggtgggagcggagggggtggatctggaggtggagggtctggtccgcactccctccgatattttgtaactgccgtctctcgacctggactgggcgaaccgaggttcatagccgtggggtacgtggacgacacgcagtttgtaagattcgacagtgatgcggataatcccagattcgagccaagagccccgtggatggagcaggaggggccggagtactgggaggaacagacacaacgagcaaaatcagacgagcagtggtttcgagttagccttcgcaccgcgcagcgatactataaccaatctaaagggggcagccatacgttccagaggatgttcgggtgtgacgtcggttcagattggcggctcttgcggggctaccagcaattcgcgtatgatggaagagactacatcgccctgaacgaagatttgaagacatggaccgctgcggatacggcggcgctcattaccagaagaaagtgggaacaggccggagatgccgaatattatcgagcctatttggagggagaatgtgtggagtggctccgccgctacctggaacttggtaatgaaaccctgcttaggactgactcccctaaagcccatgtcacgtaccatccgaggtctcaggttgacgtaacgttgaggtgctgggcgctcgggttctaccccgcagatatcaccctgacttggcagctcaacggcgaagatcttactcaggacatggagttggtggaaaccagacccgccggggatggaacgttccaaaaatgggccgctgttgtagtgccgctcggcaaagaacaaaattacacatgtcatgtccaccacaagggtctgccagaaccgctgacgctgagatggaaactccctccttctacagtttccaacacagtcataattgctgtcctcgttgtccttggtgcagctatagtcacaggcgcagtagtagcctttgtgatgaagatgcggcggaatactgggggtaaaggggttaattacgcactcgcacctgggtctcagaccagtgacttgagcctgcctgatggtaaggtcatggtgcatgaccctcatagcttggcatga

SEQ ID NO: 105 - DNA, 1737 bp, synthetic construct
  source 1..1737: mol_type = other DNA
atggcgaccgggagtcgaacatcattgttgcttgcatttgggctcctttgcctgccgtggctccaagaagggtccgccaagtataataaggcgaatgtattcctgggtggcggtggctccggtggaggtggcagtggaggtggcggtagcatacagaaaactccgcagatacaggtctattccaggcatccaccagaaaacggaaaaccaaatatattgaattgttatgtaacccagtttcaccctcctcacatcgagatccagatgctgaagaatggtaagaaaatccctaaagtggagatgtccgacatgtccttctccaaagactggagcttttacatattggcgcataccgaattcactcccacggaaactgacacgtatgcctgtagggtgaaacatgcatcaatggcagagccgaaaacagtgtactgggaccgggatatgggtggaggtggtagcggtggaggtgggagcggagggggtggatctggaggtggagggtctggtccgcactccctccgatattttgtaactgccgtctctcgacctggactgggcgaaccgaggttcatagccgtggggtacgtggacgacacgcagtttgtaagattcgacagtgatgcggataatcccagattcgagccaagagccccgtggatggagcaggaggggccggagtactgggaggaacagacacaacgagcaaaatcagacgagcagtggtttcgagttagccttcgcaccgcgcagcgatactataaccaatctaaagggggcagccatacgttccagaggatgttcgggtgtgacgtcggttcagattggcggctcttgcggggctaccagcaattcgcgtatgatggaagagactacatcgccctgaacgaagatttgaagacatggaccgctgcggatacggcggcgctcattaccagaagaaagtgggaacaggccggagatgccgaatattatcgagcctatttggagggagaatgtgtggagtggctccgccgctacctggaacttggtaatgaaaccctgcttaggactgactcccctaaagcccatgtcacgtaccatccgaggtctcaggttgacgtaacgttgaggtgctgggcgctcgggttctaccccgcagatatcaccctgacttggcagctcaacggcgaagatcttactcaggacatggagttggtggaaaccagacccgccggggatggaacgttccaaaaatgggccgctgttgtagtgccgctcggcaaagaacaaaattacacatgtcatgtccaccacaagggtctgccagaaccgctgacgctgagatggaaactccctccttctacagtttccaacacagtcataattgctgtcctcgttgtccttggtgcagctatagtcacaggcgcagtagtagcctttgtgatgaagatgcggcggaatactgggggtaaaggggttaattacgcactcgcacctgggtctcagaccagtgacttgagcctgcctgatggtaaggtcatggtgcatgaccctcatagcttggcaggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgccttga

SEQ ID NO: 106 - DNA, 1551 bp, synthetic construct
  source 1..1551: mol_type = other DNA
atggcgaccgggagtcgaacatcattgttgcttgcatttgggctcctttgcctgccgtggctccaagaagggtccgccgtgtatctcaagacgaacgtattcctcggtggcggtggctccggtggaggtggcagtggaggtggcggtagcatacagaaaactccgcagatacaggtctattccaggcatccaccagaaaacggaaaaccaaatatattgaattgttatgtaacccagtttcaccctcctcacatcgagatccagatgctgaagaatggtaagaaaatccctaaagtggagatgtccgacatgtccttctccaaagactggagcttttacatattggcgcataccgaattcactcccacggaaactgacacgtatgcctgtagggtgaaacatgcatcaatggcagagccgaaaacagtgtactgggaccgggatatgggtggaggtggtagcggtggaggtgggagcggagggggtggatctggaggtggagggtctggtccgcactccctccgatattttgtaactgccgtctctcgacctggactgggcgaaccgaggttcatagccgtggggtacgtggacgacacgcagtttgtaagattcgacagtgatgcggataatcccagattcgagccaagagccccgtggatggagcaggaggggccggagtactgggaggaacagacacaacgagcaaaatcagacgagcagtggtttcgagttagccttcgcaccgcgcagcgatactataaccaatctaaagggggcagccatacgttccagaggatgttcgggtgtgacgtcggttcagattggcggctcttgcggggctaccagcaattcgcgtatgatggaagagactacatcgccctgaacgaagatttgaagacatggaccgctgcggatacggcggcgctcattaccagaagaaagtgggaacaggccggagatgccgaatattatcgagcctatttggagggagaatgtgtggagtggctccgccgctacctggaacttggtaatgaaaccctgcttaggactgactcccctaaagcccatgtcacgtaccatccgaggtctcaggttgacgtaacgttgaggtgctgggcgctcgggttctaccccgcagatatcaccctgacttggcagctcaacggcgaagatcttactcaggacatggagttggtggaaaccagacccgccggggatggaacgttccaaaaatgggccgctgttgtagtgccgctcggcaaagaacaaaattacacatgtcatgtccaccacaagggtctgccagaaccgctgacgctgagatggaaactccctccttctacagtttccaacacagtcataattgctgtcctcgttgtccttggtgcagctatagtcacaggcgcagtagtagcctttgtgatgaagatgcggcggaatactgggggtaaaggggttaattacgcactcgcacctgggtctcagaccagtgacttgagcctgcctgatggtaaggtcatggtgcatgaccctcatagcttggcatga

SEQ ID NO: 107 - DNA, 1737 bp, synthetic construct
  source 1..1737: mol_type = other DNA
atggcgaccgggagtcgaacatcattgttgcttgcatttgggctcctttgcctgccgtggctccaagaagggtccgccgtgtatctcaagacgaacgtattcctcggtggcggtggctccggtggaggtggcagtggaggtggcggtagcatacagaaaactccgcagatacaggtctattccaggcatccaccagaaaacggaaaaccaaatatattgaattgttatgtaacccagtttcaccctcctcacatcgagatccagatgctgaagaatggtaagaaaatccctaaagtggagatgtccgacatgtccttctccaaagactggagcttttacatattggcgcataccgaattcactcccacggaaactgacacgtatgcctgtagggtgaaacatgcatcaatggcagagccgaaaacagtgtactgggaccgggatatgggtggaggtggtagcggtggaggtgggagcggagggggtggatctggaggtggagggtctggtccgcactccctccgatattttgtaactgccgtctctcgacctggactgggcgaaccgaggttcatagccgtggggtacgtggacgacacgcagtttgtaagattcgacagtgatgcggataatcccagattcgagccaagagccccgtggatggagcaggaggggccggagtactgggaggaacagacacaacgagcaaaatcagacgagcagtggtttcgagttagccttcgcaccgcgcagcgatactataaccaatctaaagggggcagccatacgttccagaggatgttcgggtgtgacgtcggttcagattggcggctcttgcggggctaccagcaattcgcgtatgatggaagagactacatcgccctgaacgaagatttgaagacatggaccgctgcggatacggcggcgctcattaccagaagaaagtgggaacaggccggagatgccgaatattatcgagcctatttggagggagaatgtgtggagtggctccgccgctacctggaacttggtaatgaaaccctgcttaggactgactcccctaaagcccatgtcacgtaccatccgaggtctcaggttgacgtaacgttgaggtgctgggcgctcgggttctaccccgcagatatcaccctgacttggcagctcaacggcgaagatcttactcaggacatggagttggtggaaaccagacccgccggggatggaacgttccaaaaatgggccgctgttgtagtgccgctcggcaaagaacaaaattacacatgtcatgtccaccacaagggtctgccagaaccgctgacgctgagatggaaactccctccttctacagtttccaacacagtcataattgctgtcctcgttgtccttggtgcagctatagtcacaggcgcagtagtagcctttgtgatgaagatgcggcggaatactgggggtaaaggggttaattacgcactcgcacctgggtctcagaccagtgacttgagcctgcctgatggtaaggtcatggtgcatgaccctcatagcttggcaggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgccttga

SEQ ID NO: 108 - DNA, 1524 bp, synthetic construct
  source 1..1524: mol_type = other DNA
atggcgaccgggagtcgaacatcattgttgcttgcatttgggctcctttgcctgccgtggctccaagaagggtccgcctccctgcttatgtggattacgcaagttggtggcggtggctccggtggaggtggcagtggaggtggcggtagcattcaacgaactccaaagatacaggtatacagtagacaccctgccgaaaacggaaaaagtaacttccttaattgctatgtttccggatttcacccttcagacatagaggtagacttgctgaagaatggtgaaaggatagagaaagtggagcactctgaccttagtttcagtaaggactggagtttctatctgctgtactacaccgaatttaccccaactgaaaaagacgagtacgcatgtcgggtgaaccacgttacgctcagtcagccaaagatagtgaaatgggatcgagatatgggtggaggtggtagcggtggaggtgggagcggagggggtggatctggaggtggagggtctggcagccatagtatgcgatactttttcacaagcgtttctcggccgggccgcggcgaacctagatttatagcagtaggttacgtcgatgacactcagttcgtccggtttgatagcgatgctgcaagtcaaaggatggaaccccgggcaccttggattgagcaggagggccctgaatattgggatggcgaaacgcgcaaagtgaaggcgcacagccaaacacatcgcgtggacttggggacccttcgagggtgctacaatcagagcgaagctggaagccacacggtacaaagaatgtacgggtgtgacgtcgggtccgactggcgctttctcagaggttaccatcagtatgcttatgatggtaaggactacattgcactgaaggaggatcttcgatcctggaccgctgcggacatgtgcgcccaaacaacaaagcacaaatgggaagctgcccacgtcgctgaacagttgcgcgcgtatctggaaggtacgtgtgttgaatggctgagacggtacctcgaaaacggcaaagaaacgctcttgaggacggactcacccaaggctcacgtgactcatcaccctcgaagtaaaggagaagtcactctgagatgttgggcgctcgggttttatccagcggacatcacattgacttggcagctgaacggtgaggagctgacacaagatatggaattggtggaaacacgacctgctggagatggtacttttcaaaaatgggcgtctgtggtagtccctctgggtaaagaacaaaactatacatgccgagtttaccatgaggggcttccggagcccctcaccctgcggtgggaacctccaccatccacggatagttacatggtcatcgtcgctgtcctcggagtgctcggtgcgatggcgatcataggtgcagttgttgcgttcgtaatgaagaggaggcggaacaccgggggcaaagggggagattacgcactcgccccaggtagccaatcatctgagatgtccttgagggactgcaaggcttga

SEQ ID NO: 109 - DNA, 1710 bp, synthetic construct
  source 1..1710: mol_type = other DNA
atggcgaccgggagtcgaacatcattgttgcttgcatttgggctcctttgcctgccgtggctccaagaagggtccgcctccctgcttatgtggattacgcaagttggtggcggtggctccggtggaggtggcagtggaggtggcggtagcattcaacgaactccaaagatacaggtatacagtagacaccctgccgaaaacggaaaaagtaacttccttaattgctatgtttccggatttcacccttcagacatagaggtagacttgctgaagaatggtgaaaggatagagaaagtggagcactctgaccttagtttcagtaaggactggagtttctatctgctgtactacaccgaatttaccccaactgaaaaagacgagtacgcatgtcgggtgaaccacgttacgctcagtcagccaaagatagtgaaatgggatcgagatatgggtggaggtggtagcggtggaggtgggagcggagggggtggatctggaggtggagggtctggcagccatagtatgcgatactttttcacaagcgtttctcggccgggccgcggcgaacctagatttatagcagtaggttacgtcgatgacactcagttcgtccggtttgatagcgatgctgcaagtcaaaggatggaaccccgggcaccttggattgagcaggagggccctgaatattgggatggcgaaacgcgcaaagtgaaggcgcacagccaaacacatcgcgtggacttggggacccttcgagggtgctacaatcagagcgaagctggaagccacacggtacaaagaatgtacgggtgtgacgtcgggtccgactggcgctttctcagaggttaccatcagtatgcttatgatggtaaggactacattgcactgaaggaggatcttcgatcctggaccgctgcggacatgtgcgcccaaacaacaaagcacaaatgggaagctgcccacgtcgctgaacagttgcgcgcgtatctggaaggtacgtgtgttgaatggctgagacggtacctcgaaaacggcaaagaaacgctcttgaggacggactcacccaaggctcacgtgactcatcaccctcgaagtaaaggagaagtcactctgagatgttgggcgctcgggttttatccagcggacatcacattgacttggcagctgaacggtgaggagctgacacaagatatggaattggtggaaacacgacctgctggagatggtacttttcaaaaatgggcgtctgtggtagtccctctgggtaaagaacaaaactatacatgccgagtttaccatgaggggcttccggagcccctcaccctgcggtgggaacctccaccatccacggatagttacatggtcatcgtcgctgtcctcggagtgctcggtgcgatggcgatcataggtgcagttgttgcgttcgtaatgaagaggaggcggaacaccgggggcaaagggggagattacgcactcgccccaggtagccaatcatctgagatgtccttgagggactgcaaggctggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgccttga

SEQ ID NO: 110 - DNA, 1554 bp, synthetic construct
  source 1..1554: mol_type = other DNA
atggcgaccgggagtcgaacatcattgttgcttgcatttgggctcctttgcctgccgtggctccaagaagggtccgcctccctgcttatgtggattacgcaagttggtggcggtggctccggtggaggtggcagtggaggtggcggtagcattcaacgaactccaaagatacaggtatacagtagacaccctgccgaaaacggaaaaagtaacttccttaattgctatgtttccggatttcacccttcagacatagaggtagacttgctgaagaatggtgaaaggatagagaaagtggagcactctgaccttagtttcagtaaggactggagtttctatctgctgtactacaccgaatttaccccaactgaaaaagacgagtacgcatgtcgggtgaaccacgttacgctcagtcagccaaagatagtgaaatgggatcgagatatgggtggaggtggtagcggtggaggtgggagcggagggggtggatctggaggtggagggtctggcagccatagtatgcgatactttttcacaagcgtttctcggccgggccgcggcgaacctagatttatagcagtaggttacgtcgatgacactcagttcgtccggtttgatagcgatgctgcaagtcaaaggatggaaccccgggcaccttggattgagcaggagggccctgaatattgggatggcgaaacgcgcaaagtgaaggcgcacagccaaacacatcgcgtggacttggggacccttcgagggtgctacaatcagagcgaagctggaagccacacggtacaaagaatgtacgggtgtgacgtcgggtccgactggcgctttctcagaggttaccatcagtatgcttatgatggtaaggactacattgcactgaaggaggatcttcgatcctggaccgctgcggacatgtgcgcccaaacaacaaagcacaaatgggaagctgcccacgtcgctgaacagttgcgcgcgtatctggaaggtacgtgtgttgaatggctgagacggtacctcgaaaacggcaaagaaacgctcctgagaaccgacagtccgaaggctcacgttacacatcattctcgacctgaagataaagtaacacttagatgttgggccttgggcttttatcccgcggacataactctgacatggcagcttaacggtgaggaactgatccaggacatggagctggttgaaacaaggcctgccggagatgggaccttccagaaatgggcttctgtagtagttccgcttggaaaggaacaatattatacttgccatgtttatcatcaagggctgccagaacccctgacgcttcgctgggagcctccaccctcaactgttagcaatatggctaccgtagcagttttggtcgtactcggtgctgccatagtgacgggagcggtggttgcattcgtgatgaagatgagaagacggaatacgggagggaaggggggagactacgccctggcaccaggttctcaaacaagtgatctcagcctcccggactgcaaggtgatggttcacgacccacatagtctggcttga

SEQ ID NO: 111 - DNA, 1740 bp, synthetic construct
  source 1..1740: mol_type = other DNA
atggcgaccgggagtcgaacatcattgttgcttgcatttgggctcctttgcctgccgtggctccaagaagggtccgcctccctgcttatgtggattacgcaagttggtggcggtggctccggtggaggtggcagtggaggtggcggtagcattcaacgaactccaaagatacaggtatacagtagacaccctgccgaaaacggaaaaagtaacttccttaattgctatgtttccggatttcacccttcagacatagaggtagacttgctgaagaatggtgaaaggatagagaaagtggagcactctgaccttagtttcagtaaggactggagtttctatctgctgtactacaccgaatttaccccaactgaaaaagacgagtacgcatgtcgggtgaaccacgttacgctcagtcagccaaagatagtgaaatgggatcgagatatgggtggaggtggtagcggtggaggtgggagcggagggggtggatctggaggtggagggtctggcagccatagtatgcgatactttttcacaagcgtttctcggccgggccgcggcgaacctagatttatagcagtaggttacgtcgatgacactcagttcgtccggtttgatagcgatgctgcaagtcaaaggatggaaccccgggcaccttggattgagcaggagggccctgaatattgggatggcgaaacgcgcaaagtgaaggcgcacagccaaacacatcgcgtggacttggggacccttcgagggtgctacaatcagagcgaagctggaagccacacggtacaaagaatgtacgggtgtgacgtcgggtccgactggcgctttctcagaggttaccatcagtatgcttatgatggtaaggactacattgcactgaaggaggatcttcgatcctggaccgctgcggacatgtgcgcccaaacaacaaagcacaaatgggaagctgcccacgtcgctgaacagttgcgcgcgtatctggaaggtacgtgtgttgaatggctgagacggtacctcgaaaacggcaaagaaacgctcctgagaaccgacagtccgaaggctcacgttacacatcattctcgacctgaagataaagtaacacttagatgttgggccttgggcttttatcccgcggacataactctgacatggcagcttaacggtgaggaactgatccaggacatggagctggttgaaacaaggcctgccggagatgggaccttccagaaatgggcttctgtagtagttccgcttggaaaggaacaatattatacttgccatgtttatcatcaagggctgccagaacccctgacgcttcgctgggagcctccaccctcaactgttagcaatatggctaccgtagcagttttggtcgtactcggtgctgccatagtgacgggagcggtggttgcattcgtgatgaagatgagaagacggaatacgggagggaaggggggagactacgccctggcaccaggttctcaaacaagtgatctcagcctcccggactgcaaggtgatggttcacgacccacatagtctggctggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgccttga

SEQ ID NO: 112 - protein, 259 aa, synthetic construct
MATGSRTSLLLAFGLLCLPWLQEGSAEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPL

SEQ ID NO: 113 - protein, 233 aa, synthetic construct
EDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPL

SEQ ID NO: 114 - protein, 291 aa, synthetic construct
MATGSRTSLLLAFGLLCLPWLQEGSAASFEAQGALANIAVDKAGGGGSGGGGSGGGGSEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPL

SEQ ID NO: 115 - protein, 265 aa, synthetic construct
ASFEAQGALANIAVDKAGGGGSGGGGSGGGGSEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPL

SEQ ID NO: 116 - protein, 321 aa, synthetic construct
MATGSRTSLLLAFGLLCLPWLQEGSAEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPLGGGSFNSSINNIHEMEIQLKDALEKNQQWLVYDQQREVYVKGLLAKIFELEKKTETAAHSLP

SEQ ID NO: 117 - protein, 295 aa, synthetic construct
EDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPLGGGSFNSSINNIHEMEIQLKDALEKNQQWLVYDQQREVYVKGLLAKIFELEKKTETAAHSLP

SEQ ID NO: 118 - protein, 353 aa, synthetic construct
MATGSRTSLLLAFGLLCLPWLQEGSAASFEAQGALANIAVDKAGGGGSGGGGSGGGGSEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPLGGGSFNSSINNIHEMEIQLKDALEKNQQWLVYDQQREVYVKGLLAKIFELEKKTETAAHSLP

SEQ ID NO: 119 - protein, 327 aa, synthetic construct
ASFEAQGALANIAVDKAGGGGSGGGGSGGGGSEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPLGGGSFNSSINNIHEMEIQLKDALEKNQQWLVYDQQREVYVKGLLAKIFELEKKTETAAHSLP

SEQ ID NO: 120 - protein, 264 aa, synthetic construct
MATGSRTSLLLAFGLLCLPWLQEGSAGDSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQ

SEQ ID NO: 121 - protein, 238 aa, synthetic construct
GDSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQ

SEQ ID NO: 122 - protein, 293 aa, synthetic construct
MATGSRTSLLLAFGLLCLPWLQEGSAASFEAQGALANIAVDKAGGGGSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQ

SEQ ID NO: 123 - protein, 267 aa, synthetic construct
ASFEAQGALANIAVDKAGGGGSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQ

SEQ ID NO: 124 - protein, 326 aa, synthetic construct
MATGSRTSLLLAFGLLCLPWLQEGSAGDSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQGGGSFNSSINNIHEMEIQLKDALEKNQQWLVYDQQREVYVKGLLAKIFELEKKTETAAHSLP

SEQ ID NO: 125 - protein, 297 aa, synthetic construct
ERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQGGGSFNSSINNIHEMEIQLKDALEKNQQWLVYDQQREVYVKGLLAKIFELEKKTETAAHSLP

SEQ ID NO: 126 - protein, 355 aa, synthetic construct
MATGSRTSLLLAFGLLCLPWLQEGSAASFEAQGALANIAVDKAGGGGSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQGGGSFNSSINNIHEMEIQLKDALEKNQQWLVYDQQREVYVKGLLAKIFELEKKTETAAHSLP

SEQ ID NO: 127 - protein, 329 aa, synthetic construct
ASFEAQGALANIAVDKAGGGGSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQGGGSFNSSINNIHEMEIQLKDALEKNQQWLVYDQQREVYVKGLLAKIFELEKKTETAAHSLP

SEQ ID NO: 128 - protein, 524 aa, synthetic construct
MPRSRALILGVLALTTMLSLCGGEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETGGGGSGGGGSQQGRLDKLTITSQNLQLESLRMKLPKSAASFEAQGALANIAVDKAGGGSSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQ

SEQ ID NO: 129 - protein, 501 aa, synthetic construct
EDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETGGGGSGGGGSQQGRLDKLTITSQNLQLESLRMKLPKSAASFEAQGALANIAVDKAGGGSSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQ

SEQ ID NO: 130 - protein, 586 aa, synthetic construct
MPRSRALILGVLALTTMLSLCGGEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETGGGGSGGGGSQQGRLDKLTITSQNLQLESLRMKLPKSAASFEAQGALANIAVDKAGGGSSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQGGGSFNSSINNIHEMEIQLKDALEKNQQWLVYDQQREVYVKGLLAKIFELEKKTETAAHSLP

SEQ ID NO: 131 - protein, 563 aa, synthetic construct
EDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETGGGGSGGGGSQQGRLDKLTITSQNLQLESLRMKLPKSAASFEAQGALANIAVDKAGGGSSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQGGGSFNSSINNIHEMEIQLKDALEKNQQWLVYDQQREVYVKGLLAKIFELEKKTETAAHSLP

SEQ ID NO: 132 - protein, 286 aa, synthetic construct
MATGSRTSLLLAFGLLCLPWLQEGSAGWYRSPFSRVVHGGGGSGGGGSGGGGSEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPL

SEQ ID NO: 133 - protein, 260 aa, synthetic construct
GWYRSPFSRVVHGGGGSGGGGSGGGGSEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPL

SEQ ID NO: 134 - protein, 348 aa, synthetic construct
MATGSRTSLLLAFGLLCLPWLQEGSAGWYRSPFSRVVHGGGGSGGGGSGGGGSEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPLGGGSFNSSINNIHEMEIQLKDALEKNQQWLVYDQQREVYVKGLLAKIFELEKKTETAAHSLP

SEQ ID NO: 135 - protein, 322 aa, synthetic construct
GWYRSPFSRVVHGGGGSGGGGSGGGGSEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPLGGGSFNSSINNIHEMEIQLKDALEKNQQWLVYDQQREVYVKGLLAKIFELEKKTETAAHSLP

SEQ ID NO: 136 - protein, 288 aa, synthetic construct
MATGSRTSLLLAFGLLCLPWLQEGSAGWYRSPFSRVVHGGGGSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQ

SEQ ID NO: 137 - protein, 262 aa, synthetic construct
GWYRSPFSRVVHGGGGSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQ

SEQ ID NO: 138 - protein, 350 aa, synthetic construct
MATGSRTSLLLAFGLLCLPWLQEGSAGWYRSPFSRVVHGGGGSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQGGGSFNSSINNIHEMEIQLKDALEKNQQWLVYDQQREVYVKGLLAKIFELEKKTETAAHSLP

SEQ ID NO: 139 - protein, 324 aa, synthetic construct
GWYRSPFSRVVHGGGGSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQGGGSFNSSINNIHEMEIQLKDALEKNQQWLVYDQQREVYVKGLLAKIFELEKKTETAAHSLP

SEQ ID NO: 140 - protein, 519 aa, synthetic construct
MPRSRALILGVLALTTMLSLCGGEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETGGGGSGGGGSQQGRLDKLTITSQNLQLESLRMKLPKSAGWYRSPFSRVVHGGGSSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQ

SEQ ID NO: 141 - protein, 496 aa, synthetic construct
EDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETGGGGSGGGGSQQGRLDKLTITSQNLQLESLRMKLPKSAGWYRSPFSRVVHGGGSSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQ

SEQ ID NO: 142 - protein, 581 aa, synthetic construct
MPRSRALILGVLALTTMLSLCGGEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETGGGGSGGGGSQQGRLDKLTITSQNLQLESLRMKLPKSAGWYRSPFSRVVHGGGSSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQGGGSFNSSINNIHEMEIQLKDALEKNQQWLVYDQQREVYVKGLLAKIFELEKKTETAAHSLP

SEQ ID NO: 143 - protein, 558 aa, synthetic construct
EDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETGGGGSGGGGSQQGRLDKLTITSQNLQLESLRMKLPKSAGWYRSPFSRVVHGGGSSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQGGGSFNSSINNIHEMEIQLKDALEKNQQWLVYDQQREVYVKGLLAKIFELEKKTETAAHSLP

SEQ ID NO: 144 - protein, 256 aa, synthetic construct
MPRSRALILGVLALTTMLSLCGGEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPL

SEQ ID NO: 145 - protein, 233 aa, synthetic construct; identical to SEQ ID NO: 113

SEQ ID NO: 146 - protein, 288 aa, synthetic construct
MPRSRALILGVLALTTMLSLCGGASFEAQGALANIAVDKAGGGGSGGGGSGGGGSEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPL

SEQ ID NO: 147 - protein, 265 aa, synthetic construct; identical to SEQ ID NO: 115

SEQ ID NO: 148 - protein, 318 aa, synthetic construct
MPRSRALILGVLALTTMLSLCGGEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPLGGGSFNSSMNSIHEKEMQLKDALEKNQQWLVYDQQREAYVKGLLAKIFELEKRTETAAASLT

SEQ ID NO: 149 - protein, 295 aa, synthetic construct
EDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPLGGGSFNSSMNSIHEKEMQLKDALEKNQQWLVYDQQREAYVKGLLAKIFELEKRTETAAASLT

SEQ ID NO: 150 - protein, 350 aa, synthetic construct
MPRSRALILGVLALTTMLSLCGGASFEAQGALANIAVDKAGGGGSGGGGSGGGGSEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPLGGGSFNSSMNSIHEKEMQLKDALEKNQQWLVYDQQREAYVKGLLAKIFELEKRTETAAASLT

SEQ ID NO: 151 - protein, 327 aa, synthetic construct
ASFEAQGALANIAVDKAGGGGSGGGGSGGGGSEDDIEADHVGTYGISVYQSPGDIGQYTFEFDGDELFYVDLDKKETVWMLPEFGQLASFDPQGGLQNIAVVKHNLGVLTKRSNSTPATNEAPQATVFPKSPVLLGQPNTLICFVDNIFPPVINITWLRNSKSVADGVYETSFFVNRDYSFHKLSYLTFIPSDDDIYDCKVEHWGLEEPVLKHWEPEIPAPMSELTETVVCALGLSVGLVGIVVGTIFIIQGLRSGGTSRHPGPLGGGSFNSSMNSIHEKEMQLKDALEKNQQWLVYDQQREAYVKGLLAKIFELEKRTETAAASLT

SEQ ID NO: 152 - protein, 265 aa, synthetic construct
MALQIPSLLLSAAVVVLMVLSSPGTEGGDSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQ

SEQ ID NO: 153 - protein, 238 aa, synthetic construct; identical to SEQ ID NO: 121

SEQ ID NO: 154 - protein, 294 aa, synthetic construct
MALQIPSLLLSAAVVVLMVLSSPGTEGASFEAQGALANIAVDKAGGGGSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQ

SEQ ID NO: 155 - protein, 267 aa, synthetic construct; identical to SEQ ID NO: 123

SEQ ID NO: 156 - protein, 327 aa, synthetic construct
MALQIPSLLLSAAVVVLMVLSSPGTEGGDSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQGGGSFNSSMNSIHEKEMQLKDALEKNQQWLVYDQQREAYVKGLLAKIFELEKRTETAAASLT

SEQ ID NO: 157 - protein, 300 aa, synthetic construct
GDSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQGGGSFNSSMNSIHEKEMQLKDALEKNQQWLVYDQQREAYVKGLLAKIFELEKRTETAAASLT

SEQ ID NO: 158 - protein, 356 aa, synthetic construct
MALQIPSLLLSAAVVVLMVLSSPGTEGASFEAQGALANIAVDKAGGGGSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQGGGSFNSSMNSIHEKEMQLKDALEKNQQWLVYDQQREAYVKGLLAKIFELEKRTETAAASLT

SEQ ID NO: 159 - protein, 329 aa, synthetic construct
ASFEAQGALANIAVDKAGGGGSGGGGSGGGGSERHFVYQFMGECYFTNGTQRIRYVTRYIYNREEYVRYDSDVGEHRAVTELGRPDAEYWNSQPEILERTRAELDTVCRHNYEGPETHTSLRRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESAWSKMLSGIGGCVLGVIFLGLGLFIRHRSQKGPRGPPPAGLLQGGGSFNSSMNSIHEKEMQLKDALEKNQQWLVYDQQREAYVKGLLAKIFELEKRTETAAASLT

SEQ ID NO: 160 - DNA, 777 bp, synthetic construct
  source 1..777: mol_type = other DNA
atggcgaccgggagtcgaacatcattgttgcttgcatttgggctcctttgcctgccgtggctccaagaagggtccgccgaggacgacatagaggccgaccatgtggggacatacggtattagtgtttaccagtcaccaggggacataggacaatatacgttcgaattcgatggtgatgagctcttctatgtagacctcgataaaaaagaaacggtttggatgctgcccgagttcggtcagcttgcttcctttgaccctcagggaggcctgcaaaatatagcagtggtcaaacacaacctcggtgtattgaccaagcgatccaatagcactccggcaacgaacgaagcgccccaagcaaccgtgtttccaaagtcacctgtactgcttgggcagccgaacactctcatctgtttcgttgacaacatcttccctcctgtcattaacatcacatggctgcgcaatagcaagtccgtggctgatggcgtgtacgaaacaagttttttcgtaaacagagattacagctttcataagctctcctaccttacattcattccttccgacgacgacatctatgattgtaaagtcgaacactggggtctcgaagagccagttctcaagcattgggagcctgaaatcccggcccccatgagtgaactcaccgaaactgtagtttgtgccctgggacttagtgttggtctcgtaggaattgttgttggcacgatttttatcatccagggtctgcggtctgggggcacttcccgccatcctggccccctt

SEQ ID NO: 161 - DNA, 873 bp, synthetic construct
  source 1..873: mol_type = other DNA
atggcgaccgggagtcgaacatcattgttgcttgcatttgggctcctttgcctgccgtggctccaagaagggtccgccgcgagtttcgaagcgcaaggagccctcgcgaatattgcggttgataaggctggcggagggggttcaggtggaggaggttcaggaggcggtggttcagaggacgacatagaggccgaccatgtggggacatacggtattagtgtttaccagtcaccaggggacataggacaatatacgttcgaattcgatggtgatgagctcttctatgtagacctcgataaaaaagaaacggtttggatgctgcccgagttcggtcagcttgcttcctttgaccctcagggaggcctgcaaaatatagcagtggtcaaacacaacctcggtgtattgaccaagcgatccaatagcactccggcaacgaacgaagcgccccaagcaaccgtgtttccaaagtcacctgtactgcttgggcagccgaacactctcatctgtttcgttgacaacatcttccctcctgtcattaacatcacatggctgcgcaatagcaagtccgtggctgatggcgtgtacgaaacaagttttttcgtaaacagagattacagctttcataagctctcctaccttacattcattccttccgacgacgacatctatgattgtaaagtcgaacactggggtctcgaagagccagttctcaagcattgggagcctgaaatcccggcccccatgagtgaactcaccgaaactgtagtttgtgccctgggacttagtgttggtctcgtaggaattgttgttggcacgatttttatcatccagggtctgcggtctgggggcacttcccgccatcctggccccctt

SEQ ID NO: 162 - DNA, 963 bp, synthetic construct
  source 1..963: mol_type = other DNA
atggcgaccgggagtcgaacatcattgttgcttgcatttgggctcctttgcctgccgtggctccaagaagggtccgccgaggacgacatagaggccgaccatgtggggacatacggtattagtgtttaccagtcaccaggggacataggacaatatacgttcgaattcgatggtgatgagctcttctatgtagacctcgataaaaaagaaacggtttggatgctgcccgagttcggtcagcttgcttcctttgaccctcagggaggcctgcaaaatatagcagtggtcaaacacaacctcggtgtattgaccaagcgatccaatagcactccggcaacgaacgaagcgccccaagcaaccgtgtttccaaagtcacctgtactgcttgggcagccgaacactctcatctgtttcgttgacaacatcttccctcctgtcattaacatcacatggctgcgcaatagcaagtccgtggctgatggcgtgtacgaaacaagttttttcgtaaacagagattacagctttcataagctctcctaccttacattcattccttccgacgacgacatctatgattgtaaagtcgaacactggggtctcgaagagccagttctcaagcattgggagcctgaaatcccggcccccatgagtgaactcaccgaaactgtagtttgtgccctgggacttagtgttggtctcgtaggaattgttgttggcacgatttttatcatccagggtctgcggtctgggggcacttcccgccatcctggcccccttggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgcct

SEQ ID NO: 163 - DNA, 1059 bp, synthetic construct
  source 1..1059: mol_type = other DNA
atggcgaccgggagtcgaacatcattgttgcttgcatttgggctcctttgcctgccgtggctccaagaagggtccgccgcgagtttcgaagcgcaaggagccctcgcgaatattgcggttgataaggctggcggagggggttcaggtggaggaggttcaggaggcggtggttcagaggacgacatagaggccgaccatgtggggacatacggtattagtgtttaccagtcaccaggggacataggacaatatacgttcgaattcgatggtgatgagctcttctatgtagacctcgataaaaaagaaacggtttggatgctgcccgagttcggtcagcttgcttcctttgaccctcagggaggcctgcaaaatatagcagtggtcaaacacaacctcggtgtattgaccaagcgatccaatagcactccggcaacgaacgaagcgccccaagcaaccgtgtttccaaagtcacctgtactgcttgggcagccgaacactctcatctgtttcgttgacaacatcttccctcctgtcattaacatcacatggctgcgcaatagcaagtccgtggctgatggcgtgtacgaaacaagttttttcgtaaacagagattacagctttcataagctctcctaccttacattcattccttccgacgacgacatctatgattgtaaagtcgaacactggggtctcgaagagccagttctcaagcattgggagcctgaaatcccggcccccatgagtgaactcaccgaaactgtagtttgtgccctgggacttagtgttggtctcgtaggaattgttgttggcacgatttttatcatccagggtctgcggtctgggggcacttcccgccatcctggcccccttggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgcct

SEQ ID NO: 164 - DNA, 792 bp, synthetic construct
  source 1..792: mol_type = other DNA
atggcgaccgggagtcgaacatcattgttgcttgcatttgggctcctttgcctgccgtggctccaagaagggtccgccggggattctgaacgccattttgtatatcagttcatgggcgagtgttattttacaaacgggacacaaaggattcgatatgtgaccagatatatctataacagagaagagtatgtccgatatgattccgacgtgggcgaacaccgcgcggtcacagagcttggcaggccggacgcggaatactggaattcacaaccagaaatactggagcgcacacgggcagagcttgacactgtgtgcagacacaattacgaaggacccgaaacacataccagcttgcggcgcctggaacaacctaacgtcgtaatctccctgagccgaactgaagctcttaaccaccacaatactctggtctgttctgttaccgatttctacccagcaaaaataaaagtacgctggtttagaaacggccaggaggaaacagtcggcgtaagtagcacgcagcttatccgaaacggggattggacattccaggtgttggttatgcttgaaatgaccccacggaggggagaggtgtacacatgccacgtagaacacccctcactgaagtcacccattaccgtcgaatggcgagcccagtcagagtctgcatggtctaaaatgctcagcggaataggtggctgcgtattgggagttattttccttggacttggcttgtttatcaggcatcggtctcagaaggggccccgggggccacccccagcgggactgttgcaa

SEQ ID NO: 165 - DNA, 879 bp, synthetic construct
  source 1..879: mol_type = other DNA
atggcgaccgggagtcgaacatcattgttgcttgcatttgggctcctttgcctgccgtggctccaagaagggtccgccgcgagtttcgaagcgcaaggagccctcgcgaatattgcggttgataaggctggcggagggggttcaggtggaggaggttcaggaggcggtggttcagaacgccattttgtatatcagttcatgggcgagtgttattttacaaacgggacacaaaggattcgatatgtgaccagatatatctataacagagaagagtatgtccgatatgattccgacgtgggcgaacaccgcgcggtcacagagcttggcaggccggacgcggaatactggaattcacaaccagaaatactggagcgcacacgggcagagcttgacactgtgtgcagacacaattacgaaggacccgaaacacataccagcttgcggcgcctggaacaacctaacgtcgtaatctccctgagccgaactgaagctcttaaccaccacaatactctggtctgttctgttaccgatttctacccagcaaaaataaaagtacgctggtttagaaacggccaggaggaaacagtcggcgtaagtagcacgcagcttatccgaaacggggattggacattccaggtgttggttatgcttgaaatgaccccacggaggggagaggtgtacacatgccacgtagaacacccctcactgaagtcacccattaccgtcgaatggcgagcccagtcagagtctgcatggtctaaaatgctcagcggaataggtggctgcgtattgggagttattttccttggacttggcttgtttatcaggcatcggtctcagaaggggccccgggggccacccccagcgggactgttgcaa

SEQ ID NO: 166 - DNA, 978 bp, synthetic construct
  source 1..978: mol_type = other DNA
atggcgaccgggagtcgaacatcattgttgcttgcatttgggctcctttgcctgccgtggctccaagaagggtccgccggggattctgaacgccattttgtatatcagttcatgggcgagtgttattttacaaacgggacacaaaggattcgatatgtgaccagatatatctataacagagaagagtatgtccgatatgattccgacgtgggcgaacaccgcgcggtcacagagcttggcaggccggacgcggaatactggaattcacaaccagaaatactggagcgcacacgggcagagcttgacactgtgtgcagacacaattacgaaggacccgaaacacataccagcttgcggcgcctggaacaacctaacgtcgtaatctccctgagccgaactgaagctcttaaccaccacaatactctggtctgttctgttaccgatttctacccagcaaaaataaaagtacgctggtttagaaacggccaggaggaaacagtcggcgtaagtagcacgcagcttatccgaaacggggattggacattccaggtgttggttatgcttgaaatgaccccacggaggggagaggtgtacacatgccacgtagaacacccctcactgaagtcacccattaccgtcgaatggcgagcccagtcagagtctgcatggtctaaaatgctcagcggaataggtggctgcgtattgggagttattttccttggacttggcttgtttatcaggcatcggtctcagaaggggccccgggggccacccccagcgggactgttgcaaggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgcct

SEQ ID NO: 167 - DNA, 1065 bp, synthetic construct
  source 1..1065: mol_type = other DNA
atggcgaccgggagtcgaacatcattgttgcttgcatttgggctcctttgcctgccgtggctccaagaagggtccgccgcgagtttcgaagcgcaaggagccctcgcgaatattgcggttgataaggctggcggagggggttcaggtggaggaggttcaggaggcggtggttcagaacgccattttgtatatcagttcatgggcgagtgttattttacaaacgggacacaaaggattcgatatgtgaccagatatatctataacagagaagagtatgtccgatatgattccgacgtgggcgaacaccgcgcggtcacagagcttggcaggccggacgcggaatactggaattcacaaccagaaatactggagcgcacacgggcagagcttgacactgtgtgcagacacaattacgaaggacccgaaacacataccagcttgcggcgcctggaacaacctaacgtcgtaatctccctgagccgaactgaagctcttaaccaccacaatactctggtctgttctgttaccgatttctacccagcaaaaataaaagtacgctggtttagaaacggccaggaggaaacagtcggcgtaagtagcacgcagcttatccgaaacggggattggacattccaggtgttggttatgcttgaaatgaccccacggaggggagaggtgtacacatgccacgtagaacacccctcactgaagtcacccattaccgtcgaatggcgagcccagtcagagtctgcatggtctaaaatgctcagcggaataggtggctgcgtattgggagttattttccttggacttggcttgtttatcaggcatcggtctcagaaggggccccgggggccacccccagcgggactgttgcaaggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgcct

SEQ ID NO: 168 - DNA, 1572 bp, synthetic construct
  source 1..1572: mol_type = other DNA
atgccccgcagccgagcgcttatactcggcgttctggcgctcacaactatgctttcattgtgtggtggtgaggacgacatagaagccgatcacgtcgggacatacggaataagcgtgtatcagtcacctggtgacataggacaatatacattcgagtttgatggagatgaacttttctacgttgaccttgataaaaaagaaactgtatggatgttgccggaatttggtcaactggcgtcttttgacccccaaggaggccttcagaacatagcggtagtaaaacacaaccttggagtattgacgaaacgctccaactctaccccggcgacaaatgaagcgccgcaggccaccgtattcccaaaatcacctgtattgctcggtcagcctaataccctcatctgctttgtagataatatatttcccccggtcataaacatcacgtggctcagaaatagtaaaagcgtagccgacggtgtatatgaaacctcattcttcgtcaaccgcgactactcattccacaaattgagctatttgacgttcattccctctgacgatgacatttacgactgcaaagttgaacactggggtctggaagaaccagtgctcaagcactgggagccagagattccggcaccgatgtctgagttgactgaaacgggcggtggtgggagtgggggcgggggtagccagcagggccggcttgataagctgacaataacatctcaaaatttgcaactcgagtctctcagaatgaaactgccgaagtcagccgcgagttttgaagcgcaaggggcattggcaaacattgcagttgacaaagctggtggtgggtccagtgggggaggggggtctgggggaggtggatccgagcggcactttgtgtaccagtttatgggcgaatgttactttaccaacggaactcagaggattcgctatgttacgcgatatatctacaaccgagaggagtatgtccggtatgactccgatgtgggggagcacagggctgtcaccgaactcggacgccccgacgcggaatattggaatagccaacccgaaatactcgagaggacccgggcagagctggacacggtctgtcggcacaactatgaaggaccagaaacgcacacgagcctgcggcggctggagcagccaaatgtagtgataagtctgtcccgcacggaggctttgaaccatcacaacacattggtctgttcagttaccgatttctatcccgcgaaaatcaaggttcgatggttccgaaacgggcaggaagagactgtaggtgtttcttctacacaactgatccggaacggggattggacgttccaagtccttgtcatgttggaaatgactcccaggaggggtgaagtctatacctgtcatgtggagcatccctcactgaaaagcccaattacagtcgagtggagagcccagtctgaaagcgcctggtcaaaaatgctgtctggtataggaggttgtgtactcggggtcattttcctgggactgggtctgtttatccgccatcgaagtcagaaaggccctagaggtccaccccctgccgggcttctccag

SEQ ID NO: 169 - DNA, 1758 bp, synthetic construct
  source 1..1758: mol_type = other DNA
atgccccgcagccgagcgcttatactcggcgttctggcgctcacaactatgctttcattgtgtggtggtgaggacgacatagaagccgatcacgtcgggacatacggaataagcgtgtatcagtcacctggtgacataggacaatatacattcgagtttgatggagatgaacttttctacgttgaccttgataaaaaagaaactgtatggatgttgccggaatttggtcaactggcgtcttttgacccccaaggaggccttcagaacatagcggtagtaaaacacaaccttggagtattgacgaaacgctccaactctaccccggcgacaaatgaagcgccgcaggccaccgtattcccaaaatcacctgtattgctcggtcagcctaataccctcatctgctttgtagataatatatttcccccggtcataaacatcacgtggctcagaaatagtaaaagcgtagccgacggtgtatatgaaacctcattcttcgtcaaccgcgactactcattccacaaattgagctatttgacgttcattccctctgacgatgacatttacgactgcaaagttgaacactggggtctggaagaaccagtgctcaagcactgggagccagagattccggcaccgatgtctgagttgactgaaacgggcggtggtgggagtgggggcgggggtagccagcagggccggcttgataagctgacaataacatctcaaaatttgcaactcgagtctctcagaatgaaactgccgaagtcagccgcgagttttgaagcgcaaggggcattggcaaacattgcagttgacaaagctggtggtgggtccagtgggggaggggggtctgggggaggtggatccgagcggcactttgtgtaccagtttatgggcgaatgttactttaccaacggaactcagaggattcgctatgttacgcgatatatctacaaccgagaggagtatgtccggtatgactccgatgtgggggagcacagggctgtcaccgaactcggacgccccgacgcggaatattggaatagccaacccgaaatactcgagaggacccgggcagagctggacacggtctgtcggcacaactatgaaggaccagaaacgcacacgagcctgcggcggctggagcagccaaatgtagtgataagtctgtcccgcacggaggctttgaaccatcacaacacattggtctgttcagttaccgatttctatcccgcgaaaatcaaggttcgatggttccgaaacgggcaggaagagactgtaggtgtttcttctacacaactgatccggaacggggattggacgttccaagtccttgtcatgttggaaatgactcccaggaggggtgaagtctatacctgtcatgtggagcatccctcactgaaaagcccaattacagtcgagtggagagcccagtctgaaagcgcctggtcaaaaatgctgtctggtataggaggttgtgtactcggggtcattttcctgggactgggtctgtttatccgccatcgaagtcagaaaggccctagaggtccaccccctgccgggcttctccagggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgcct

SEQ ID NO: 170 - DNA, 858 bp, synthetic construct
  source 1..858: mol_type = other DNA
atggcgaccgggagtcgaacatcattgttgcttgcatttgggctcctttgcctgccgtggctccaagaagggtccgccggatggtatcggtctccgttcagcagagttgtgcacggcggagggggttcaggtggaggaggttcaggaggcggtggttcagaggacgacatagaggccgaccatgtggggacatacggtattagtgtttaccagtcaccaggggacataggacaatatacgttcgaattcgatggtgatgagctcttctatgtagacctcgataaaaaagaaacggtttggatgctgcccgagttcggtcagcttgcttcctttgaccctcagggaggcctgcaaaatatagcagtggtcaaacacaacctcggtgtattgaccaagcgatccaatagcactccggcaacgaacgaagcgccccaagcaaccgtgtttccaaagtcacctgtactgcttgggcagccgaacactctcatctgtttcgttgacaacatcttccctcctgtcattaacatcacatggctgcgcaatagcaagtccgtggctgatggcgtgtacgaaacaagttttttcgtaaacagagattacagctttcataagctctcctaccttacattcattccttccgacgacgacatctatgattgtaaagtcgaacactggggtctcgaagagccagttctcaagcattgggagcctgaaatcccggcccccatgagtgaactcaccgaaactgtagtttgtgccctgggacttagtgttggtctcgtaggaattgttgttggcacgatttttatcatccagggtctgcggtctgggggcacttcccgccatcctggccccctt

SEQ ID NO: 171 - DNA, 1044 bp, synthetic construct
  source 1..1044: mol_type = other DNA
atggcgaccgggagtcgaacatcattgttgcttgcatttgggctcctttgcctgccgtggctccaagaagggtccgccggatggtatcggtctccgttcagcagagttgtgcacggcggagggggttcaggtggaggaggttcaggaggcggtggttcagaggacgacatagaggccgaccatgtggggacatacggtattagtgtttaccagtcaccaggggacataggacaatatacgttcgaattcgatggtgatgagctcttctatgtagacctcgataaaaaagaaacggtttggatgctgcccgagttcggtcagcttgcttcctttgaccctcagggaggcctgcaaaatatagcagtggtcaaacacaacctcggtgtattgaccaagcgatccaatagcactccggcaacgaacgaagcgccccaagcaaccgtgtttccaaagtcacctgtactgcttgggcagccgaacactctcatctgtttcgttgacaacatcttccctcctgtcattaacatcacatggctgcgcaatagcaagtccgtggctgatggcgtgtacgaaacaagttttttcgtaaacagagattacagctttcataagctctcctaccttacattcattccttccgacgacgacatctatgattgtaaagtcgaacactggggtctcgaagagccagttctcaagcattgggagcctgaaatcccggcccccatgagtgaactcaccgaaactgtagtttgtgccctgggacttagtgttggtctcgtaggaattgttgttggcacgatttttatcatccagggtctgcggtctgggggcacttcccgccatcctggcccccttggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgcct

SEQ ID NO: 172 - DNA, 864 bp, synthetic construct
  source 1..864: mol_type = other DNA
atggcgaccgggagtcgaacatcattgttgcttgcatttgggctcctttgcctgccgtggctccaagaagggtccgccggatggtatcggtctccgttcagcagagttgtgcacggcggagggggttcaggtggaggaggttcaggaggcggtggttcagaacgccattttgtatatcagttcatgggcgagtgttattttacaaacgggacacaaaggattcgatatgtgaccagatatatctataacagagaagagtatgtccgatatgattccgacgtgggcgaacaccgcgcggtcacagagcttggcaggccggacgcggaatactggaattcacaaccagaaatactggagcgcacacgggcagagcttgacactgtgtgcagacacaattacgaaggacccgaaacacataccagcttgcggcgcctggaacaacctaacgtcgtaatctccctgagccgaactgaagctcttaaccaccacaatactctggtctgttctgttaccgatttctacccagcaaaaataaaagtacgctggtttagaaacggccaggaggaaacagtcggcgtaagtagcacgcagcttatccgaaacggggattggacattccaggtgttggttatgcttgaaatgaccccacggaggggagaggtgtacacatgccacgtagaacacccctcactgaagtcacccattaccgtcgaatggcgagcccagtcagagtctgcatggtctaaaatgctcagcggaataggtggctgcgtattgggagttattttccttggacttggcttgtttatcaggcatcggtctcagaaggggccccgggggccacccccagcgggactgttgcaa

SEQ ID NO: 173 - DNA, 1050 bp, synthetic construct
  source 1..1050: mol_type = other DNA
atggcgaccgggagtcgaacatcattgttgcttgcatttgggctcctttgcctgccgtggctccaagaagggtccgccggatggtatcggtctccgttcagcagagttgtgcacggcggagggggttcaggtggaggaggttcaggaggcggtggttcagaacgccattttgtatatcagttcatgggcgagtgttattttacaaacgggacacaaaggattcgatatgtgaccagatatatctataacagagaagagtatgtccgatatgattccgacgtgggcgaacaccgcgcggtcacagagcttggcaggccggacgcggaatactggaattcacaaccagaaatactggagcgcacacgggcagagcttgacactgtgtgcagacacaattacgaaggacccgaaacacataccagcttgcggcgcctggaacaacctaacgtcgtaatctccctgagccgaactgaagctcttaaccaccacaatactctggtctgttctgttaccgatttctacccagcaaaaataaaagtacgctggtttagaaacggccaggaggaaacagtcggcgtaagtagcacgcagcttatccgaaacggggattggacattccaggtgttggttatgcttgaaatgaccccacggaggggagaggtgtacacatgccacgtagaacacccctcactgaagtcacccattaccgtcgaatggcgagcccagtcagagtctgcatggtctaaaatgctcagcggaataggtggctgcgtattgggagttattttccttggacttggcttgtttatcaggcatcggtctcagaaggggccccgggggccacccccagcgggactgttgcaaggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgcct

SEQ ID NO: 174 - DNA, 1557 bp, synthetic construct
  source 1..1557: mol_type = other DNA
atgccccgcagccgagcgcttatactcggcgttctggcgctcacaactatgctttcattgtgtggtggtgaggacgacatagaagccgatcacgtcgggacatacggaataagcgtgtatcagtcacctggtgacataggacaatatacattcgagtttgatggagatgaacttttctacgttgaccttgataaaaaagaaactgtatggatgttgccggaatttggtcaactggcgtcttttgacccccaaggaggccttcagaacatagcggtagtaaaacacaaccttggagtattgacgaaacgctccaactctaccccggcgacaaatgaagcgccgcaggccaccgtattcccaaaatcacctgtattgctcggtcagcctaataccctcatctgctttgtagataatatatttcccccggtcataaacatcacgtggctcagaaatagtaaaagcgtagccgacggtgtatatgaaacctcattcttcgtcaaccgcgactactcattccacaaattgagctatttgacgttcattccctctgacgatgacatttacgactgcaaagttgaacactggggtctggaagaaccagtgctcaagcactgggagccagagattccggcaccgatgtctgagttgactgaaacgggcggtggtgggagtgggggcgggggtagccagcagggccggcttgataagctgacaataacatctcaaaatttgcaactcgagtctctcagaatgaaactgccgaagtcagccggatggtatcggtctccgttcagcagagttgtgcacggtggtgggtccagtgggggaggggggtctgggggaggtggatccgagcggcactttgtgtaccagtttatgggcgaatgttactttaccaacggaactcagaggattcgctatgttacgcgatatatctacaaccgagaggagtatgtccggtatgactccgatgtgggggagcacagggctgtcaccgaactcggacgccccgacgcggaatattggaatagccaacccgaaatactcgagaggacccgggcagagctggacacggtctgtcggcacaactatgaaggaccagaaacgcacacgagcctgcggcggctggagcagccaaatgtagtgataagtctgtcccgcacggaggctttgaaccatcacaacacattggtctgttcagttaccgatttctatcccgcgaaaatcaaggttcgatggttccgaaacgggcaggaagagactgtaggtgtttcttctacacaactgatccggaacggggattggacgttccaagtccttgtcatgttggaaatgactcccaggaggggtgaagtctatacctgtcatgtggagcatccctcactgaaaagcccaattacagtcgagtggagagcccagtctgaaagcgcctggtcaaaaatgctgtctggtataggaggttgtgtactcggggtcattttcctgggactgggtctgtttatccgccatcgaagtcagaaaggccctagaggtccaccccctgccgggcttctccag

SEQ ID NO: 175 - DNA, 1743 bp, synthetic construct
  source 1..1743: mol_type = other DNA
atgccccgcagccgagcgcttatactcggcgttctggcgctcacaactatgctttcattgtgtggtggtgaggacgacatagaagccgatcacgtcgggacatacggaataagcgtgtatcagtcacctggtgacataggacaatatacattcgagtttgatggagatgaacttttctacgttgaccttgataaaaaagaaactgtatggatgttgccggaatttggtcaactggcgtcttttgacccccaaggaggccttcagaacatagcggtagtaaaacacaaccttggagtattgacgaaacgctccaactctaccccggcgacaaatgaagcgccgcaggccaccgtattcccaaaatcacctgtattgctcggtcagcctaataccctcatctgctttgtagataatatatttcccccggtcataaacatcacgtggctcagaaatagtaaaagcgtagccgacggtgtatatgaaacctcattcttcgtcaaccgcgactactcattccacaaattgagctatttgacgttcattccctctgacgatgacatttacgactgcaaagttgaacactggggtctggaagaaccagtgctcaagcactgggagccagagattccggcaccgatgtctgagttgactgaaacgggcggtggtgggagtgggggcgggggtagccagcagggccggcttgataagctgacaataacatctcaaaatttgcaactcgagtctctcagaatgaaactgccgaagtcagccggatggtatcggtctccgttcagcagagttgtgcacggtggtgggtccagtgggggaggggggtctgggggaggtggatccgagcggcactttgtgtaccagtttatgggcgaatgttactttaccaacggaactcagaggattcgctatgttacgcgatatatctacaaccgagaggagtatgtccggtatgactccgatgtgggggagcacagggctgtcaccgaactcggacgccccgacgcggaatattggaatagccaacccgaaatactcgagaggacccgggcagagctggacacggtctgtcggcacaactatgaaggaccagaaacgcacacgagcctgcggcggctggagcagccaaatgtagtgataagtctgtcccgcacggaggctttgaaccatcacaacacattggtctgttcagttaccgatttctatcccgcgaaaatcaaggttcgatggttccgaaacgggcaggaagagactgtaggtgtttcttctacacaactgatccggaacggggattggacgttccaagtccttgtcatgttggaaatgactcccaggaggggtgaagtctatacctgtcatgtggagcatccctcactgaaaagcccaattacagtcgagtggagagcccagtctgaaagcgcctggtcaaaaatgctgtctggtataggaggttgtgtactcggggtcattttcctgggactgggtctgtttatccgccatcgaagtcagaaaggccctagaggtccaccccctgccgggcttctccagggcggaggctctttcaacagcagcatcaacaacatccacgagatggaaatccagctgaaggacgccctggaaaagaaccagcagtggctggtgtacgatcagcagcgcgaggtgtacgtgaaaggcctgctggccaagatcttcgagctggaaaaaaagaccgagacagccgctcacagcctgcct

SEQ ID NO: 176 - DNA, 1025 bp, synthetic construct
  source 1..1025: mol_type = other DNA
taatacgactcactataaggaaataagagagaaaagaagagtaagaagaaatataagagccaccgatatgcctaggagtcgagcccttatattgggcgtactcgccctcaccacaatgttgtctctctgcgggggagaggacgacatagaagcagatcacgttggtacatatggcatcagcgtgtaccagagtcctggggatattggtcaatacacattcgagttcgacggagatgagcttttttacgtcgatctcgacaaaaaggaaactgtttggatgctccctgaatttggccaacttgcaagcttcgaccctcagggcggcttgcagaatatcgctgtggtcaaacataatctcggtgtactcactaagagaagcaacagcactcctgctaccaacgaagccccacaagccaccgtgtttccaaagagcccagtccttctcggccagccaaacactcttatctgcttcgtcgacaacatcttccctcccgtaataaatataacttggctgcgaaactccaaaagcgtggctgacggggtctatgagactagcttcttcgtcaatcgagactactcattccataaattgtcatacctgacatttatcccctcagacgatgatatatacgactgtaaggtcgaacattggggactcgaagagcccgtcctgaagcattgggagcccgaaattcctgctcctatgtctgagttgactgagacagtagtctgcgctcttggcctcagcgtgggtctggtcgggatagtagtcggcaccatcttcataatccaagggctgaggagcggagggacctcccggcaccctgggcctctgtgaatcgctgccttctgcggggcttgccttctggccatgcccttcttctctcccttgcacctgtacctcttggtctttgaataaagcctgagtaggaagtgagggtctagaaaaaaaaaaaaaaaaaaaaaaaaaaaaaaaaaaaaaaaaaaaaaaaaaaaaaaacgatcaataaccgacactcctgcgt

SEQ ID NO: 177 - DNA, 1121 bp, synthetic construct
  source 1..1121: mol_type = other DNA
taatacgactcactataaggaaataagagagaaaagaagagtaagaagaaatataagagccaccgatatgcctaggagtcgagcccttatattgggcgtactcgccctcaccacaatgttgtctctctgcgggggagcttcttttgaagcacagggtgcacttgcaaacatcgccgtcgacaaggccggaggaggaggatccggaggtggtggctctggcggcggtggttcagaggacgacatagaagcagatcacgttggtacatatggcatcagcgtgtaccagagtcctggggatattggtcaatacacattcgagttcgacggagatgagcttttttacgtcgatctcgacaaaaaggaaactgtttggatgctccctgaatttggccaacttgcaagcttcgaccctcagggcggcttgcagaatatcgctgtggtcaaacataatctcggtgtactcactaagagaagcaacagcactcctgctaccaacgaagccccacaagccaccgtgtttccaaagagcccagtccttctcggccagccaaacactcttatctgcttcgtcgacaacatcttccctcccgtaataaatataacttggctgcgaaactccaaaagcgtggctgacggggtctatgagactagcttcttcgtcaatcgagactactcattccataaattgtcatacctgacatttatcccctcagacgatgatatatacgactgtaaggtcgaacattggggactcgaagagcccgtcctgaagcattgggagcccgaaattcctgctcctatgtctgagttgactgagacagtagtctgcgctcttggcctcagcgtgggtctggtcgggatagtagtcggcaccatcttcataatccaagggctgaggagcggagggacctcccggcaccctgggcctctgtgaatcgctgccttctgcggggcttgccttctggccatgcccttcttctctcccttgcacctgtacctcttggtctttgaataaagcctgagtaggaagtgagggtctagaaaaaaaaaaaaaaaaaaaaaaaaaaaaaaaaaaaaaaaaaaaaaaaaaaaaaaacgatcaataaccgacactcctgcgt

SEQ ID NO: 178 - DNA, 1211 bp, synthetic construct
  source 1..1211: mol_type = other DNA
taatacgactcactataaggaaataagagagaaaagaagagtaagaagaaatataagagccaccgatatgcctaggagtcgagcccttatattgggcgtactcgccctcaccacaatgttgtctctctgcgggggagaggacgacatagaagcagatcacgttggtacatatggcatcagcgtgtaccagagtcctggggatattggtcaatacacattcgagttcgacggagatgagcttttttacgtcgatctcgacaaaaaggaaactgtttggatgctccctgaatttggccaacttgcaagcttcgaccctcagggcggcttgcagaatatcgctgtggtcaaacataatctcggtgtactcactaagagaagcaacagcactcctgctaccaacgaagccccacaagccaccgtgtttccaaagagcccagtccttctcggccagccaaacactcttatctgcttcgtcgacaacatcttccctcccgtaataaatataacttggctgcgaaactccaaaagcgtggctgacggggtctatgagactagcttcttcgtcaatcgagactactcattccataaattgtcatacctgacatttatcccctcagacgatgatatatacgactgtaaggtcgaacattggggactcgaagagcccgtcctgaagcattgggagcccgaaattcctgctcctatgtctgagttgactgagacagtagtctgcgctcttggcctcagcgtgggtctggtcgggatagtagtcggcaccatcttcataatccaagggctgaggagcggagggacctcccggcaccctgggcctctgggaggtgggtcattcaactccagtatgaactccatacatgaaaaggagatgcagttgaaagacgctcttgaaaaaaaccagcaatggctcgtttatgaccagcaacgcgaagcatacgtgaaggggttgcttgctaaaattttcgagttggaaaagcgcactgaaactgcagcagcttcactcacttgaatcgctgccttctgcggggcttgccttctggccatgcccttcttctctcccttgcacctgtacctcttggtctttgaataaagcctgagtaggaagtgagggtctagaaaaaaaaaaaaaaaaaaaaaaaaaaaaaaaaaaaaaaaaaaaaaaaaaaaaaaacgatcaataaccgacactcctgcgt

SEQ ID NO: 179 - DNA, 1307 bp, synthetic construct
  source 1..1307: mol_type = other DNA
taatacgactcactataaggaaataagagagaaaagaagagtaagaagaaatataagagccaccgatatgcctaggagtcgagcccttatattgggcgtactcgccctcaccacaatgttgtctctctgcgggggagcttcttttgaagcacagggtgcacttgcaaacatcgccgtcgacaaggccggaggaggaggatccggaggtggtggctctggcggcggtggttcagaggacgacatagaagcagatcacgttggtacatatggcatcagcgtgtaccagagtcctggggatattggtcaatacacattcgagttcgacggagatgagcttttttacgtcgatctcgacaaaaaggaaactgtttggatgctccctgaatttggccaacttgcaagcttcgaccctcagggcggcttgcagaatatcgctgtggtcaaacataatctcggtgtactcactaagagaagcaacagcactcctgctaccaacgaagccccacaagccaccgtgtttccaaagagcccagtccttctcggccagccaaacactcttatctgcttcgtcgacaacatcttccctcccgtaataaatataacttggctgcgaaactccaaaagcgtggctgacggggtctatgagactagcttcttcgtcaatcgagactactcattccataaattgtcatacctgacatttatcccctcagacgatgatatatacgactgtaaggtcgaacattggggactcgaagagcccgtcctgaagcattgggagcccgaaattcctgctcctatgtctgagttgactgagacagtagtctgcgctcttggcctcagcgtgggtctggtcgggatagtagtcggcaccatcttcataatccaagggctgaggagcggagggacctcccggcaccctgggcctctgggaggtgggtcattcaactccagtatgaactccatacatgaaaaggagatgcagttgaaagacgctcttgaaaaaaaccagcaatggctcgtttatgaccagcaacgcgaagcatacgtgaaggggttgcttgctaaaattttcgagttggaaaagcgcactgaaactgcagcagcttcactcacttgaatcgctgccttctgcggggcttgccttctggccatgcccttcttctctcccttgcacctgtacctcttggtctttgaataaagcctgagtaggaagtgagggtctagaaaaaaaaaaaaaaaaaaaaaaaaaaaaaaaaaaaaaaaaaaaaaaaaaaaaaaacgatcaataaccgacactcctgcgt

SEQ ID NO: 180 - DNA, 1052 bp, synthetic construct
  source 1..1052: mol_type = other DNA
taatacgactcactataaggaaataagagagaaaagaagagtaagaagaaatataagagccaccgatatggctttgcagattccatcccttctcctttctgccgcagtcgttgtgctgatggttctctcaagtcctggaacagagggtggcgactccgaacgccattttgtgtaccagttcatgggggaatgctacttcaccaacggtacacagcggattagatacgtcaccagatatatctacaatagggaggagtatgtgagatatgacagtgacgtgggagaacatagggcagtgaccgagttgggaagaccagatgcagaatactggaacagccagcctgaaatcctcgaacgaacacgagccgaacttgatactgtgtgcaggcacaactacgaagggccagagacccacaccagtctgcggcgcctcgaacaaccaaacgtcgtaattagtctgagtagaaccgaggcactcaaccaccataacactctggtgtgctcagtcacagatttctaccctgctaagatcaaggtgagatggttccggaacggtcaagaggagacagttggcgttagtagcacccagctgataagaaatggggattggacttttcaggtcctggtgatgcttgaaatgacacccaggagaggtgaggtttacacttgtcatgtcgagcatcctagtctgaagtctcctattacagtagagtggcgcgctcagagcgagagtgcatggtcaaagatgctgagcggcataggaggatgtgtcctgggggtcattttcctcggcctcggattgttcatccgccatcggtcccaaaaaggcccacgagggccaccccccgctggtcttctgcaatgaatcgctgccttctgcggggcttgccttctggccatgcccttcttctctcccttgcacctgtacctcttggtctttgaataaagcctgagtaggaagtgagggtctagaaaaaaaaaaaaaaaaaaaaaaaaaaaaaaaaaaaaaaaaaaaaaaaaaaaaaaacgatcaataaccgacactcctgcgt

SEQ ID NO: 181 - DNA, 1139 bp, synthetic construct
  source 1..1139: mol_type = other DNA
taatacgactcactataaggaaataagagagaaaagaagagtaagaagaaatataagagccaccgatatggctttgcagattccatcccttctcctttctgccgcagtcgttgtgctgatggttctctcaagtcctggaacagagggtgcttcttttgaagcacagggtgcacttgcaaacatcgccgtcgacaaggccggaggaggaggatccggaggtggtggctctggcggcggtggttcagaacgccattttgtgtaccagttcatgggggaatgctacttcaccaacggtacacagcggattagatacgtcaccagatatatctacaatagggaggagtatgtgagatatgacagtgacgtgggagaacatagggcagtgaccgagttgggaagaccagatgcagaatactggaacagccagcctgaaatcctcgaacgaacacgagccgaacttgatactgtgtgcaggcacaactacgaagggccagagacccacaccagtctgcggcgcctcgaacaaccaaacgtcgtaattagtctgagtagaaccgaggcactcaaccaccataacactctggtgtgctcagtcacagatttctaccctgctaagatcaaggtgagatggttccggaacggtcaagaggagacagttggcgttagtagcacccagctgataagaaatggggattggacttttcaggtcctggtgatgcttgaaatgacacccaggagaggtgaggtttacacttgtcatgtcgagcatcctagtctgaagtctcctattacagtagagtggcgcgctcagagcgagagtgcatggtcaaagatgctgagcggcataggaggatgtgtcctgggggtcattttcctcggcctcggattgttcatccgccatcggtcccaaaaaggcccacgagggccaccccccgctggtcttctgcaatgaatcgctgccttctgcggggcttgccttctggccatgcccttcttctctcccttgcacctgtacctcttggtctttgaataaagcctgagtaggaagtgagggtctagaaaaaaaaaaaaaaaaaaaaaaaaaaaaaaaaaaaaaaaaaaaaaaaaaaaaaaacgatcaataaccgacactcctgcgt

SEQ ID NO: 182 - DNA, 1238 bp, synthetic construct
  source 1..1238: mol_type = other DNA
taatacgactcactataaggaaataagagagaaaagaagagtaagaagaaatataagagccaccgatatggctttgcagattccatcccttctcctttctgccgcagtcgttgtgctgatggttctctcaagtcctggaacagagggtggcgactccgaacgccattttgtgtaccagttcatgggggaatgctacttcaccaacggtacacagcggattagatacgtcaccagatatatctacaatagggaggagtatgtgagatatgacagtgacgtgggagaacatagggcagtgaccgagttgggaagaccagatgcagaatactggaacagccagcctgaaatcctcgaacgaacacgagccgaacttgatactgtgtgcaggcacaactacgaagggccagagacccacaccagtctgcggcgcctcgaacaaccaaacgtcgtaattagtctgagtagaaccgaggcactcaaccaccataacactctggtgtgctcagtcacagatttctaccctgctaagatcaaggtgagatggttccggaacggtcaagaggagacagttggcgttagtagcacccagctgataagaaatggggattggacttttcaggtcctggtgatgcttgaaatgacacccaggagaggtgaggtttacacttgtcatgtcgagcatcctagtctgaagtctcctattacagtagagtggcgcgctcagagcgagagtgcatggtcaaagatgctgagcggcataggaggatgtgtcctgggggtcattttcctcggcctcggattgttcatccgccatcggtcccaaaaaggcccacgagggccaccccccgctggtcttctgcaaggaggtgggtcattcaactccagtatgaactccatacatgaaaaggagatgcagttgaaagacgctcttgaaaaaaaccagcaatggctcgtttatgaccagcaacgcgaagcatacgtgaaggggttgcttgctaaaattttcgagttggaaaagcgcactgaaactgcagcagcttcactcacttgaatcgctgccttctgcggggcttgccttctggccatgcccttcttctctcccttgcacctgtacctcttggtctttgaataaagcctgagtaggaagtgagggtctagaaaaaaaaaaaaaaaaaaaaaaaaaaaaaaaaaaaaaaaaaaaaaaaaaaaaaaacgatcaataaccgacactcctgcgt

SEQ ID NO: 183 - DNA, 1325 bp, synthetic construct
  source 1..1325: mol_type = other DNA
taatacgactcactataaggaaataagagagaaaagaagagtaagaagaaatataagagccaccgatatggctttgcagattccatcccttctcctttctgccgcagtcgttgtgctgatggttctctcaagtcctggaacagagggtgcttcttttgaagcacagggtgcacttgcaaacatcgccgtcgacaaggccggaggaggaggatccggaggtggtggctctggcggcggtggttcagaacgccattttgtgtaccagttcatgggggaatgctacttcaccaacggtacacagcggattagatacgtcaccagatatatctacaatagggaggagtatgtgagatatgacagtgacgtgggagaacatagggcagtgaccgagttgggaagaccagatgcagaatactggaacagccagcctgaaatcctcgaacgaacacgagccgaacttgatactgtgtgcaggcacaactacgaagggccagagacccacaccagtctgcggcgcctcgaacaaccaaacgtcgtaattagtctgagtagaaccgaggcactcaaccaccataacactctggtgtgctcagtcacagatttctaccctgctaagatcaaggtgagatggttccggaacggtcaagaggagacagttggcgttagtagcacccagctgataagaaatggggattggacttttcaggtcctggtgatgcttgaaatgacacccaggagaggtgaggtttacacttgtcatgtcgagcatcctagtctgaagtctcctattacagtagagtggcgcgctcagagcgagagtgcatggtcaaagatgctgagcggcataggaggatgtgtcctgggggtcattttcctcggcctcggattgttcatccgccatcggtcccaaaaaggcccacgagggccaccccccgctggtcttctgcaaggaggtgggtcattcaactccagtatgaactccatacatgaaaaggagatgcagttgaaagacgctcttgaaaaaaaccagcaatggctcgtttatgaccagcaacgcgaagcatacgtgaaggggttgcttgctaaaattttcgagttggaaaagcgcactgaaactgcagcagcttcactcacttgaatcgctgccttctgcggggcttgccttctggccatgcccttcttctctcccttgcacctgtacctcttggtctttgaataaagcctgagtaggaagtgagggtctagaaaaaaaaaaaaaaaaaaaaaaaaaaaaaaaaaaaaaaaaaaaaaaaaaaaaaaacgatcaataaccgacactcctgcgt

SEQ ID NO: 184 - protein, 10 aa, synthetic construct
ELAGIGILTV

SEQ ID NO: 185 - protein, 9 aa, synthetic construct
SLLMWITQV

SEQ ID NO: 186 - protein, 17 aa, synthetic construct
ASFEAQGALANIAVDKA

SEQ ID NO: 187 - protein, 12 aa, synthetic construct
GWYRSPFSRVVH

SEQ ID NO: 188 - protein, 17 aa, synthetic construct
QATQKITYRISGVGIDQ

SEQ ID NO: 189 - protein, 17 aa, synthetic construct
PFGIFVVDKNTGDINIT

SEQ ID NO: 190 - protein, 17 aa, synthetic construct
HLNSKIAFKIVSQEPAG

SEQ ID NO: 191 - protein, 17 aa, synthetic construct
GTPMFLLSRNTGEVRTL

SEQ ID NO: 192 - protein, 17 aa, synthetic construct
QCECNIKVKDVNDNFPM

SEQ ID NO: 193 - protein, 17 aa, synthetic construct
SVKLSIAVKNKAEFHQS

SEQ ID NO: 194 - protein, 17 aa, synthetic construct
NVREGIAFRPASKTFTV

SEQ ID NO: 195 - protein, 17 aa, synthetic construct
RDSTFIVNKTITAEVLA

SEQ ID NO: 196 - protein, 17 aa, synthetic construct
LLSRNTGEVRTLTNSLD

SEQ ID NO: 197 - protein, 9 aa, synthetic construct
VRSRRCLRL

SEQ ID NO: 198 - protein, 15 aa, synthetic construct
GQVELGGGGIVEQCC

SEQ ID NO: 199 - protein, 15 aa, synthetic construct
GQVELGGGTPIESHQ

SEQ ID NO: 200 - protein, 15 aa, synthetic construct
GQVELGGGNAVEVLK

SEQ ID NO: 201 - protein, 15 aa, synthetic construct
GQVELGGGSSPETLI

SEQ ID NO: 202 - protein, 14 aa, synthetic construct
SLQPLALEAEDLQV

SEQ ID NO: 203 - protein, 14 aa, synthetic construct
SLQPLALGIVEQCC

SEQ ID NO: 204 - protein, 14 aa, synthetic construct
SLQPLALNAVEVLK

SEQ ID NO: 205 - protein, 14 aa, synthetic construct
SLQPLALSSPETLI

SEQ ID NO: 206 - protein, 14 aa, synthetic construct
SHLVEALYLVCGER

SEQ ID NO: 207 - protein, 14 aa, synthetic construct
SHLVEALYLVCGEE

SEQ ID NO: 208 - protein, 13 aa, synthetic construct
LVEALYLVCGERG

SEQ ID NO: 209 - protein, 13 aa, synthetic construct
LVEALYLVCGEEG

SEQ ID NO: 210 - protein, 15 aa, synthetic construct
GQVELGGGNAVEVLK

SEQ ID NO: 211 - protein, 15 aa, synthetic construct
GQVELGGGSSPETLI

SEQ ID NO: 212 - protein, 15 aa, synthetic construct
GQVELGGGGIVEQCC

SEQ ID NO: 213 - protein, 15 aa, synthetic construct
GQVELGGGTPIESHQ

SEQ ID NO: 214 - protein, 16 aa, synthetic construct
VCGERGFFEELVARSE

SEQ ID NO: 215 - protein, 16 aa, synthetic construct
HLVEALYLEELVARSE

SEQ ID NO: 216 - protein, 16 aa, synthetic construct
ICSLYQLEFVNQHLCG

SEQ ID NO: 217 - protein, 16 aa, synthetic construct
SLQKRGIVEELVARSE

SEQ ID NO: 218 - protein, 16 aa, synthetic construct
CSLYQLENSVPHFSDE

SEQ ID NO: 219 - protein, 16 aa, synthetic construct
QPLALEGSALSSQHQA

SEQ ID NO: 220 - protein, 12 aa, synthetic construct
PGAGSLQPLALE

SEQ ID NO: 221 - protein, 11 aa, synthetic construct
SLQPLALEGSL

SEQ ID NO: 222 - protein, 9 aa, synthetic construct
QPLALEGSL

SEQ ID NO: 223 - protein, 10 aa, synthetic construct
QPLALEGSLQ

SEQ ID NO: 224 - protein, 10 aa, synthetic construct
GAGSLQPLAL

SEQ ID NO: 225 - protein, 11 aa, synthetic construct
QVELGGGPGAG

SEQ ID NO: 226 - protein, 10 aa, synthetic construct
AEDLQVGQVE

SEQ ID NO: 227 - protein, 12 aa, synthetic construct
SLQPLALEGSLQ

SEQ ID NO: 228 - protein, 7 aa, synthetic construct
LALEGSL

SEQ ID NO: 229 - protein, 9 aa, synthetic construct
AGSLQPLAL

SEQ ID NO: 230 - protein, 10 aa, synthetic construct
SLQPLALEGS

SEQ ID NO: 231 - protein, 10 aa, synthetic construct
VELGGGPGAG

SEQ ID NO: 232 - protein, 12 aa, synthetic construct
EDLQVGQVELGG

SEQ ID NO: 233 - protein, 9 aa, synthetic construct
VELGGGPGA

SEQ ID NO: 234 - protein, 11 aa, synthetic construct
QVELGGGPGAG

SEQ ID NO: 235 - protein, 9 aa, synthetic construct
QVELGGGPG

SEQ ID NO: 236 - protein, 10 aa, synthetic construct
AGSLQPLALE

SEQ ID NO: 237 - protein, 9 aa, synthetic construct
AGSLQPLAL

SEQ ID NO: 238 - protein, 11 aa, synthetic construct
SLQPLALEGSL

SEQ ID NO: 239 - protein, 16 aa, synthetic construct
VGQVELGGGPIVEQCC

SEQ ID NO: 240 - protein, 16 aa, synthetic construct
VGQVELGGGPALEGSL

SEQ ID NO: 241 - protein, 15 aa, synthetic construct
VELGGGPGAEAEDLQ

SEQ ID NO: 242 - protein, 16 aa, synthetic construct
LQPLALEGSLLALEGS

SEQ ID NO: 243 - protein, 16 aa, synthetic construct
QPLALEGLQVGQVELG

SEQ ID NO: 244 - protein, 16 aa, synthetic construct
EAEDLQVGLALEGSLQ

SEQ ID NO: 245 - protein, 9 aa, synthetic construct
KYNKANVFL

SEQ ID NO: 246 - protein, 9 aa, synthetic construct
VYLKTNVFL

SEQ ID NO: 247 - protein, 9 aa, synthetic construct
VLFGLGFAI

SEQ ID NO: 248 - protein, 9 aa, synthetic construct
NLFLFLFAV

SEQ ID NO: 249 - protein, 9 aa, synthetic construct
FLFAVGFYL

SEQ ID NO: 250 - protein, 9 aa, synthetic construct
YLLLRVLNI

SEQ ID NO: 251 - protein, 12 aa, synthetic construct
MASPGSGFWSFG

SEQ ID NO: 252 - protein, 12 aa, synthetic construct
DSENPGTARAWC

SEQ ID NO: 253 - protein, 12 aa, synthetic construct
TARAWCQVAQKF

SEQ ID NO: 254 - protein, 12 aa, synthetic construct
ADWLTSTANTNM

SEQ ID NO: 255 - protein, 12 aa, synthetic construct
PVFVLLEYVTLK

SEQ ID NO: 256 - protein, 12 aa, synthetic construct
KGMAALPRLIAF

SEQ ID NO: 257 - protein, 12 aa, synthetic construct
AVADICKKYKIW

SEQ ID NO: 258 - protein, 12 aa, synthetic construct
WGGGLLMSRKHK

SEQ ID NO: 259 - protein, 12 aa, synthetic construct
MSRKHKWKLSGV

SEQ ID NO: 260 - protein, 12 aa, synthetic construct
QCGRHVDVFKLW

SEQ ID NO: 261 - protein, 12 aa, synthetic construct
QCGRHVDVFKLW

SEQ ID NO: 262 - protein, 20 aa, synthetic construct
EYVTLKKMREIIGWPGGSGD

SEQ ID NO: 263 - protein, 20 aa, synthetic construct
AALGIGTDSVILIKCDERGK

SEQ ID NO: 264 - protein, 13 aa, synthetic construct
NFIRMVISNPAAT

SEQ ID NO: 265 - protein, 20 aa, synthetic construct
YVVKSFDRSTKVIDFHYPNE

SEQ ID NO: 266 - protein, 20 aa, synthetic construct
LPRLIAFTSEHSHFSLKKGA

(19) United States
(12) Patent Application Publication
(10) Pub. No.: US 2026/0284164 A1
(43) Pub. Date: Sep. 24, 2026

(54) IMMUNOREGULATORY ANTIGEN-PRESENTING VESICLES

(71) Applicant: California Institute of Technology, Pasadena, CA (US)

(72) Inventors: Blade A. Olson, Pasadena, CA (US); Stephen L. Mayo, Pasadena, CA (US); Richard M. Murray, Pasadena, CA (US)

(21) Appl. No.: 19/176,162

(22) Filed: Apr. 11, 2025

Related U.S. Application Data
(60) Provisional application No. 63/632,954, filed on Apr. 11, 2024.
(60) Provisional application No. 63/728,287, filed on Dec. 5, 2024.

(51) Int. Cl. A61K 39/00; A61P 37/04

(52) U.S. Cl. CPC A61K 39/001111; A61P 37/04; A61K 2039/55555

(57) ABSTRACT

Disclosed herein include methods, compositions, and kits suitable for use in modulating immune responses and treating diseases or disorders. There are provided, in some embodiments, nucleic acid compositions (e.g., mRNA vaccines, DNA vaccines) comprising one or more polynucleotides encoding components of an antigen presentation complex (APC). The APC can comprise an APC-assembly domain, one or more antigen presentation polypeptide(s) and a presenting peptide. A plurality of APCs can be capable of self-assembling into an antigen-presenting vesicle (APV) secreted from a cell in which the APCs are expressed. There are provided, in some embodiments, populations of APVs.

### RELATED APPLICATIONS

This application claims the benefit under 35 U.S.C. § 119(e) of U.S. Provisional Patent Application Ser. No. 63/632,954, filed Apr. 11, 2024, and U.S. Provisional Patent Application Ser. No. 63/728,287, filed Dec. 5, 2024. The entire contents of these applications are hereby expressly incorporated by reference in their entireties.

### STATEMENT REGARDING FEDERALLY SPONSORED R&D

This invention was made with government support under Grant No. W911NF-19-2-0026 awarded by the Army. The government has certain rights in the invention.

### REFERENCE TO SEQUENCE LISTING

The present application is being filed along with a Sequence Listing in electronic format. The Sequence Listing is provided as a file entitled 30KJ-810005-US_SequenceListing, created Apr. 10, 2025, which is 325 kilobytes in size. The information in the electronic format of the Sequence Listing is incorporated herein by reference in its entirety.

### BACKGROUND

### Field

The present disclosure relates generally to the field of immune medicine, in particular, to self-assembling vesicles for treating and regulating immune responses against diseases and disorders.

### Description of the Related Art

The release of B cell-derived extracellular vesicles (EVs) with functional peptide:MHC (pMHC) complexes on their surface was first reported about thirty years ago. Since then, EVs that were previously labeled as simple cellular “debris” have been increasingly recognized for their immunoregulatory potential as a simpler, cell-free alternative to cell-based immunotherapies. Naturally occurring pMHC-displaying antigen-presenting vesicles (APVs) in particular have been demonstrated to orchestrate immune responses either directly or with the aid of antigen-presenting cells. Research teams have ventured to engineer more complex versions of these antigen-presenting vesicles, such as whole mammalian cells conjugated with immunostimulatory molecules, nanoparticles coated with membranes derived from antigen-presenting cells via extrusion or sonication, or engineered extracellular blebs from dendritic cells. These previous approaches demonstrated the therapeutic potential of APVs, but production is often low yield or requires the patient's own cells to be harvested and processed before treatment can be administered. Significant attention has been given to manufacturing APVs via microfluidics, but existing microfluidic vesicles suffer from short half-lives and are susceptible to rupture due to osmotic changes and the turbulent flow of the lymphatic and cardiovascular systems. Polymer-coated iron-oxide nanoparticles conjugated with pMHCI have shown remarkable therapeutic potential for autoimmune diseases such as type 1 diabetes, but the stiff spherical structure of the nanoparticle scaffold reduces the potential surface area for ideal pMHC:T cell receptor (TCR) interactions; additionally, pMHC complexes are rigidly adhered to the nanoparticle surface, limiting pMHC movement and clustering considered important for the formation of a T cell stimulating immunological synapse. Thus, engineered APVs that would ideally recreate the endogenous form of APVs, while maximizing the concentration of proteins that are critical for immunoregulation, are still in need.

### SUMMARY

Disclosed herein include compositions. In some embodiments, the composition comprises: a nucleic acid composition comprising one or more polynucleotides encoding components of an antigen presentation complex (APC). In some embodiments, the components of the APC are capable of assembling into an antigen-presenting vesicle (APV) budded from a cell in which the APC components are expressed. In some embodiments, the APV comprises a plurality of APCs. In some embodiments, the APC comprises an APC-assembly domain, one or more antigen presentation polypeptide(s) and a presenting peptide.

Disclosed herein include compositions. In some embodiments, the composition comprises: a population of an antigen-presenting vesicles (APVs). In some embodiments, each of the APVs comprises a plurality of antigen presentation complexes (APCs). In some embodiments, the APC comprises an APC-assembly domain, one or more antigen presentation polypeptide(s) and a presenting peptide.

In some embodiments, the APVs are derived from expression of the nucleic acid composition disclosed herein in a cell. In some embodiments, the APC comprises or consists of components of the APC.

In some embodiments, the APC comprises all or a portion of a major histocompatibility complex (MHC), or a derivative thereof. In some embodiments, the APC comprises all or a portion of MHC class I (MHC-I) or MHC class II (MHC-II). In some embodiments, the MHC comprises human leukocyte antigen (HLA) and/or mouse MHC. In some embodiments, the antigen presentation polypeptide(s) are selected from MIC-I α chain, beta-2 microglobulin (β2M), MHC-II α chain, MHC-II β chain, a classical HLA Class I molecule, a non-classical HLA Class I molecule, a classical HLA Class II molecule, a non-classical HLA Class II molecule, HLA-A, HLA-B, HLA-C, HLA-E, HLA-F, HLA-G, HLA-DRA, HLA-DRB1, HLA-DRB3, HLA-DRB4, HLA-DRB5, HLA-DQA1, HLA-DQB1, HLA-DPA1, HLA-DPB1, HLA-DMA, HLA-DMB, HLA-DOA, HLA-DOB, derivatives thereof, homologs thereof, or any combination thereof. In some embodiments, the HLA class I is selected from an HLA allele selected from HLA-A*01:01, HLA-A*02:01, HLA-A*02:03, HLA-A*02:06, HLA-A*03:01, HLA-A*11:01, HLA-A*23:01, HLA-A*24:02, HLA-A*26:01, HLA-A*30:01, HLA-A*30:02, HLA-A*31:01, HLA-A*32:01, HLA-A*33:01, HLA-A*68:01, HLA-A*68:02, HLA-B*07:02, HLA-B*08:01, HLA-B*15:01, HLA-B*35:01, HLA-B*40:01, HLA-B*44:02, HLA-B*44:03, HLA-B*51:01, HLA-B*53:01, HLA-B*57:01, and HLA-B*58:01. In some embodiments, the HLA class II is selected from an HLA allele selected from HLA-DRB1*01:01, HLA-DRB1*15:01, HLA-DRB1*03:01, HLA-DRB1*04:01, HLA-DRB1*11:01, HLA-DRB1*13:01, HLA-DRB1*07:01, HLA-DRB1*01:01, HLA-DRB1*01:02, HLA-DRB1*04:02, HLA-DRB1*04:05, HLA-DRB1*04:07, HLA-DRB1*04:08, HLA-DRB1*08:04, HLA-DRB1*09:01, HLA-DRB1*10:01, HLA-DRB1*11:02, HLA-DRB1*11:03, HLA-DRB1*11:04, HLA-DRB1*15:02, HLA-DRB1*15:03, HLA-DRB1*16:01, HLA-DRB1*16:02, HLA-DRB3*02:02, HLA-DRB3*03:01, HLA-DRB5*01:01, HLA-DQA1*01:01/DQB1*05:01, HLA-DQA1*05:01/DQB1*02:01, HLA-DQA1*01:02/DQB1*05:02, HLA-DQA1*01:02/DQB1*06:02, HLA-DQA1*03:01/DQB1*03:02, HLA-DQA1*01:02/DQB1*06:04, HLA-DQA1*05:01/DQB1*03:01, HLA-DQA1*02:01/DQB1*02:02, HLA-DQA1*03:01/DQB1*03:01, HLA-DQA1*02:01/DQB1*03:03, HLA-DQA1*03:03/DQB1*03:03, HLA-DPA1*01:03/DPB1*01:01, HLA-DPA1*01:03/DPB1*02:01, HLA-DPA1*01:03/DPB1*03:01, HLA-DPA1*01:03/DPB1*04:01, HLA-DPA1*01:03/DPB1*04:02, HLA-DPA1*01:03/DPB1*05:01, HLA-DPA1*02:01/DPB1*01:01, HLA-DPA1*02:01/DPB1*02:01, HLA-DPA1*02:01/DPB1*03:01, HLA-DPA1*02:01/DPB1*04:01, HLA-DPA1*02:01/DPB1*04:02, HLA-DPA1*02:01/DPB1*05:01, HLA-DPA1*02:01/DPB1*06:01, HLA-DPA1*02:01/DPB1*09:01, HLA-DPA1*02:01/DPB1*11:01, HLA-DPA1*02:01/DPB1*13:01, HLA-DPA1*02:01/DPB1*14:01, HLA-DPA1*02:01/DPB1*15:01, and HLA-DPA1*02:01/DPB1*17:01. In some embodiments, at least two components of the APC are derived from different species. In some embodiments, the APC comprises at least one mutation at positions equivalent to Y84 and A139 of HLA-A, optionally Y84A and/or A139C.

In some embodiments, the antigen presentation polypeptide(s) is capable of forming a binding site that binds the presenting peptide. In some embodiments, the presence of the antigen presentation polypeptide(s) does not induce an immune response in a subject, optionally a subject from which the cell was derived. In some embodiments, the presence of the antigen presentation polypeptide(s) induces an immune response in a subject. In some embodiments, the immune response is induced from a subject from which the cell was derived. In some embodiments, the antigen presentation polypeptide(s) comprises an amino acid sequence having at least 65%, 70%, 75%, 80%, 85%, 90%, 95%, 98%, or 99% sequence identity to an amino acid sequence selected from SEQ ID NOs: 53-54, 57-58, 61-62, 81-86, 112-113, 120-121, 144-145, and 152-153.

In some embodiments, the cell is: a cell of a subject; an in vivo cell, an ex vivo cell, or an in situ cell; and/or an adherent cell or a suspension cell. In some embodiments, the cell: is not an immune cell; or is an immune cell.

In some embodiments, the APC-assembly domain recruits one or more endosomal sorting complex required for transport (ESCRT) proteins to the cytoplasmic tail of the APC. In some embodiments, the recruitment of ESCRT proteins via the APC-assembly domain induces the self-assembly and budding of APV. In some embodiments, the cytoplasmic portion of the APC comprises the APC-assembly domain. In some embodiments, the cytoplasmic tail of the APC comprises the APC-assembly domain. In some embodiments, the APC-assembly domain interacts with the ESCRT proteins TSG101, NEDD4, and/or ALIX. In some embodiments, the APC-assembly domain comprises or is derived from: the ESCRT and ALIX binding region (EABR) of the human CEP55 protein, optionally residues 160-217; a nonhuman protein, optionally a nonmammalian protein, further optionally a chicken protein, a mouse protein, a lizard protein, a reptile protein, a hamster protein, or a goldfish protein; Syntenin-1, rat Galectin-3 (rGalectin-3), Hrs, and/or CD2AP; a viral protein, optionally a fragment of a viral protein, further optionally a retroviral protein, herpes simplex viral protein, vaccinia viral protein, hepadnaviral protein, togaviral protein, flaviviral protein, arenaviral protein, coronaviral protein, orthomyxoviral protein, paramyxoviral protein, bunyaviral protein, bornaviral protein, rhabdoviral protein or filoviral protein, optionally a Gag protein, further optionally derived from EIAV, HTLV-1, MLV, or MPMV, optionally EIAV p9 and/or HIV-1 p6; and/or an Ebola protein, optionally EBOV VP40. In some embodiments, the APC-assembly domain comprises an amino acid sequence having at least 65%, 70%, 75%, 80%, 85%, 90%, 95%, 98%, or 99% sequence identity to an amino acid sequence of SEQ ID NO: 33.

In some embodiments, the presenting peptide, or a portion thereof, is displayed on the surface of the APV. In some embodiments, the presenting peptide: is about 1 amino acid to about 10000 amino acids in length, optionally 5-20 amino acids in length, further optionally 5, 6, 7, 8, 9, 10, 11, or 12 amino acids in length; comprises or is derived from an antigenic protein associated with a disease or disorder, optionally an immunogenic variant and/or an immunogenic fragment of said antigenic protein; comprises or is derived from a conserved portion of said antigenic protein; and/or comprises or is derived from at least about 0.001 percent, about 0.01 percent, about 0.05 percent, about 0.1 percent, about 0.2 percent, about 0.4 percent, about 0.6 percent, about 0.8 percent, about 1 percent, about 2 percent, about 3 percent, about 4 percent, or about 5 percent of the full length of said antigenic protein.

In some embodiments, the disease or disorder is an infectious disease or disorder caused by an infectious agent, the presenting peptide comprises or is derived from an antigenic protein of said infectious agent, and the antigenic protein of said infectious agent is a pathogenic antigen. In some embodiments, the disease or disorder is a disease associated with expression of a tumor-associated antigen, and the antigenic protein is a tumor-associated antigen. In some embodiments, the disease or disorder is a disease associated with expression of a cancer-associated antigen, and the antigenic protein is a cancer-associated antigen. In some embodiments, the disease or disorder is an autoimmune disease or disorder, and the antigenic protein is an autoimmune antigen. In some embodiments, the disease or disorder is a metabolic disorder. In some embodiments, the metabolic disorder is diabetes. In some embodiments, the disease or disorder is an allergic disease or disorder, and the antigenic protein is an allergenic antigen.

In some embodiments, the infectious agent is: a bacterium, a fungus, a virus, or a protist; and/or a coronavirus (CoV). In some embodiments, the CoV comprises an alphacoronavirus, a betacoronavirus, a gammacoronavirus, or a deltacoronavirus. In some embodiments, the infectious agent is selected from Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157:H7, O111 and O104:H4, Fasciola hepatica and Fasciola gigantica, FFI prion, Filarioidea superfamily, Filoviruses, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In some embodiments, the presenting peptide is derived from one or more of MART-1; NYESO1; neuropilin (NRP); IGRP; CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some embodiments, the APC comprises one or more linker(s). In some embodiments, the one or more linker(s): comprise one or more flexible amino acid residue(s), optionally about 1 to about 50 flexible amino acid residues, further optionally the flexible amino acid residues comprise glycine, serine, or a combination thereof; is a glycine-serine (GS) linker, optionally 1-25 amino acids in length; and/or is situated between the APC-assembly domain and the presenting peptide, between the APC-assembly domain and the antigen presentation polypeptide, between the antigen presentation polypeptide and the presenting peptide, and/or between two of the one or more antigen presentation polypeptides. In some embodiments, the linker comprises an amino acid sequence selected from GSGSGSG (SEQ ID NO: 1), GGGGS (SEQ ID NO: 2), GSGSG (SEQ ID NO: 3), GGGG (SEQ ID NO: 4), GGG, GG, GS, GSGS (SEQ ID NO: 8), GGGS (SEQ ID NO: 9), GGS, GTS, GGSGGS (SEQ ID NO: 12), GGG, GGGGGG (SEQ ID NO: 14), GGGGGGGGG (SEQ ID NO: 15), GGGGGGGGGGGG (SEQ ID NO: 16), GGGGGGGGGGGGGGG (SEQ ID NO: 17), GGS, GGSGGS (SEQ ID NO: 19), GGSGGSGGS (SEQ ID NO: 20), GGSGGSGGSGGS (SEQ ID NO: 21), GGSGGSGGSGGSGGS (SEQ ID NO: 22), GSG, GSGGSG (SEQ ID NO: 24), GSGGSGGSG (SEQ ID NO: 25), GSGGSGGSGGSG (SEQ ID NO: 26), GSGGSGGSGGSGGSG (SEQ ID NO: 27), (GGGGS)n (SEQ ID NO: 28), GGGGSGGGGS (SEQ ID NO: 29), GGGGSGGGGSGGGGS (SEQ ID NO: 30), GGGGSGGGGSGGGGSGGGGS (SEQ ID NO: 31), and GGGGSGGGGSGGGGSGGGGSGGGGS (SEQ ID NO: 32). In some embodiments, the linker comprises an amino acid sequence of GGGGS (SEQ ID NO: 2) or GGGS (SEQ ID NO: 9). In some embodiments, the linker comprises n GGGGS (SEQ ID NO: 2) or GGGS (SEQ ID NO: 9), and n is an integer from 1 to 10, optionally n is 3 or 4.

In some embodiments, the APC comprises a single fusion protein. In some embodiments, the single fusion protein comprises one or more linker(s), the presenting peptide, the antigen presentation polypeptide(s), and the APC-assembly domain. In some embodiments, the antigen presentation polypeptide(s) is covalently connected to the APC-assembly domain and the presenting peptide. In some embodiments, the antigen presentation polypeptide(s) is situated between the APC-assembly domain and the presenting peptide. In some embodiments, at least one of the antigen presentation polypeptide(s) is situated between the APC-assembly domain and the presenting peptide. In some embodiments, the antigen presentation polypeptide(s) comprise two or more antigen presentation polypeptides with the presenting peptide situated between said two or more antigen presentation polypeptides. In some embodiments, the antigen presentation polypeptide(s) comprise MHC-I α chain and beta-2 microglobulin (β2M). In some embodiments, the components of the APC are connected in the order from N-terminus to C-terminus: the presenting peptide, beta-2 microglobulin (β2M), MHC-I α chain and the APC-assembly domain; or the APC-assembly domain, MHC-I α chain, beta-2 microglobulin (β2M) and the presenting peptide. In some embodiments, the antigen presentation polypeptide(s) comprise MHC-II α chain and MHC-II β chain. In some embodiments, the components of the APC are connected in the order from N-terminus to C-terminus: the APC-assembly domain, MHC-II β chain, the presenting peptide and the MHC-II α chain; the APC-assembly domain, MHC-II α chain, the presenting peptide and the MHC-II β chain; the MHC-II α chain, the presenting peptide, MHC-II β chain and the APC-assembly domain; or the MHC-II β chain, the presenting peptide, MHC-II α chain and the APC-assembly domain.

In some embodiments, the APC comprises two or more separate polypeptides. In some embodiments, the APC comprises two separate polypeptides. In some embodiments, at least two of the two or more separate polypeptides comprise an APC-assembly domain. In some embodiments, at least one of the two or more separate polypeptides is a fusion protein. In some embodiments, the fusion protein comprises one or more linker(s), the antigen presentation polypeptide(s), and the APC-assembly domain. In some embodiments, the fusion protein comprises (i) a linker, a first antigen presentation polypeptide, and the APC-assembly domain; or (ii) a linker, a second antigen presentation polypeptide, and the presenting peptide. In some embodiments, the first antigen presentation polypeptide is covalently connected to the APC-assembly domain, and the second antigen presentation polypeptide is covalently connected to the presenting peptide, optionally via a peptide bond. In some embodiments, the first antigen presentation polypeptide is capable of binding to the second antigen presentation polypeptide. In some embodiments, the first antigen presentation polypeptide comprises MHC-I α chain and the second antigen presentation polypeptide comprises beta-2 microglobulin (β2M). In some embodiments, the first antigen presentation polypeptide comprises beta-2 microglobulin (β2M) and the second antigen presentation polypeptide comprises MHC-I α chain. In some embodiments, the first antigen presentation polypeptide comprises MHC-II α chain and the second antigen presentation polypeptide comprises MHC-II β chain. In some embodiments, the first antigen presentation polypeptide comprises MHC-II β chain and the second antigen presentation polypeptide comprises MHC-II α chain. In some embodiments, MHC-I α chain is capable of forming a binding site that binds the presenting peptide. In some embodiments, MHC-II α chain and the MHC-II β chain are capable of forming a binding site that binds the presenting peptide.

In some embodiments, the APC comprises an amino acid sequence having at least 65%, 70%, 75%, 80%, 85%, 90%, 95%, 98%, or 99% sequence identity to an amino acid sequence selected from SEQ ID NOs: 37-52, 55-56, 59-60, 63-80, 114-119, 122-143, 146-151, and 154-159.

In some embodiments, the APVs comprise one or more supplemental polypeptide(s) (e.g., immunosuppressive polypeptide(s), immunostimulatory polypeptide(s), costimulatory polypeptide(s), cell adhesion polypeptide(s), anti-phagocytic polypeptide(s), tissue-homing polypeptide(s), or any combination thereof). In some embodiments, the supplemental polypeptide(s) are selected from CD3ε, CD3ζ, CD4, CD5, CD8α, CD9, CD16, CD22, CD27, CD28, CD33, CD37, CD45, CD64, CD80, CD86, CD 134, CD137, CD152, CD 154, PD1, CARD11, CD2, CD7, CD27, CD28, CD30, CD40, CD54 (ICAM), CD3, CD3γ, CD3δ, CD25, CD39, GITR, LRRC32, Neuropilin-1, CCR6, CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, ZAP70, CD73, LFA-1, CD47, CCR7, derivatives thereof, or any combination thereof. In some embodiments, the supplemental polypeptide(s) is: (i) associated with an APC-assembly domain; and/or (ii) not associated with an APC-assembly domain. In some embodiments, the nucleic acid composition comprises one or more second polynucleotide(s) encoding the one or more supplemental polypeptide(s).

In some embodiments, the APVs comprise a lipid bilayer. In some embodiments, the APVs comprise a lipid bilayer derived from the cell from which the APVs was budded.

In some embodiments, at least two of the APCs of the plurality of APCs are different from each other with respect to the presenting peptide, and the population of APVs thereby display a plurality of disparate presenting peptides. In some embodiments, the population of APVs comprise: one or more homotypic APVs, wherein the plurality of APCs of a homotypic APV are the same as each other with respect to the presenting peptide, and wherein a homotypic APV thereby does not display a plurality of disparate presenting peptides; one or more heterotypic APVs, wherein at least two of the APCs of a heterotypic APV are different from each other with respect to the presenting peptide, and wherein a heterotypic APV thereby displays a plurality of disparate presenting peptides; a mixture of two or more homotypic APVs that differ from each other with respect to the presenting peptide of the plurality of APCs present in said two or more homotypic APVs, and wherein the population of APVs thereby displays a plurality of disparate presenting peptides; a mixture of two or more heterotypic APVs that differ from each other with respect to the presenting peptide of the plurality of APCs of said two or more heterotypic APVs; and/or a mixture of one or more homotypic APVs and one or more heterotypic APVs, and wherein the population of APVs thereby displays a plurality of disparate presenting peptides. In some embodiments, the plurality of disparate presenting peptides comprises: between about 2 and about 500 antigenic peptides that differ from each other; presenting peptide of a same protein type; and/or presenting peptide of different protein types.

In some embodiments, less than about 10% of the APVs of the population of APVs have a particle size smaller than about 30 nm; less than about 10% of the APVs of the population of APVs have a particle size exceeding about 100 nm; the average diameter of the APVs of the population of APVs range from about 30 nm to about 250 nm, from about 50 nm to about 125 nm, or from about 60 nm to about 80 nm; and/or the average diameter of the APVs of the population of APVs is about 50 nm, about 52 nm, about 54 nm, about 56 nm, about 58 nm, about 60 nm, about 62 nm, about 64 nm, about 66 nm, about 68 nm, 70 nm, about 72 nm, about 74 nm, about 76 nm, about 78 nm, about 80 nm, about 82 nm, about 84 nm, about 86 nm, about 88 nm, or about 90 nm. In some embodiments, the average is the mean, median or mode. In some embodiments, the mean is the arithmetic mean, geometric mean, and/or harmonic mean. In some embodiments, the APVs: have a minimum diameter of about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, or about 50 nm; and/or have a maximum diameter of about 250 nm, about 252 nm, about 254 nm, about 256 nm, about 258 nm, about 260 nm, about 262 nm, about 264 nm, about 266 nm, about 268 nm, about 270 nm, about 272 nm, about 274 nm, about 276 nm, about 278 nm, or about 280 nm.

In some embodiments, the APV and/or the APC is capable of inducing immunogenic responses against the presenting peptide in a subject without the administration of exogenous antigen presenting cells and/or exogenous antigen presenting components other than the APV and/or the APC.

In some embodiments, the nucleic acid composition is complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition is, comprises, or further comprises, one or more vectors. In some embodiments, at least one of the one or more vectors is a viral vector, a plasmid, a transposable element, a naked DNA vector, a lipid nanoparticle (LNP), or any combination thereof. In some embodiments, the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. In some embodiments, the transposable element is piggybac transposon or sleeping beauty transposon. In some embodiments, the polynucleotide(s) encoding components of the APC are comprised in the one or more vectors. In some embodiments, the polynucleotide(s) encoding components of the APC are comprised in the same vector and/or different vectors. In some embodiments, the polynucleotide(s) encoding components of the APC are situated on the same nucleic acid and/or different nucleic acids. In some embodiments, the one or more vectors is a DNA vaccine.

In some embodiments, the polynucleotide(s) encoding components of the APC are operably linked to one or more promoters capable of inducing transcription of said polynucleotide(s). In some embodiments, the DNA vaccine is a plasmid-based DNA vaccine, a minicircle-based DNA vaccine, a bacmid-based DNA vaccine, a minigene-based DNA vaccine, a ministring DNA (linear covalently closed DNA vector) vaccine, a closed-ended linear duplex DNA (CELiD or ceDNA) vaccine, a Doggybone™ DNA vaccine, a dumbbell shaped DNA vaccine, or a minimalistic immunological-defined gene expression (MIDGE)-vector DNA vaccine. In some embodiments, the promoter comprises a ubiquitous promoter, an inducible promoter, a tissue-specific promoter and/or a lineage-specific promoter. In some embodiments, the ubiquitous promoter is selected from a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), 0-kinesin (0-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human 0-actin (HBA) promoter, chicken 3-actin (CBA) promoter, a CAG promoter, a CASI promoter, a CBH promoter, or any combination thereof. In some embodiments, the polynucleotide(s) encoding components of the APC are operably linked to a tandem gene expression element. In some embodiments, the tandem gene expression element is an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof.

In some embodiments, the polynucleotide(s) encoding components of the APC comprises a transcript stabilization element. In some embodiments, the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof.

In some embodiments, the one or more polynucleotides comprise the nucleotide sequence of any one of SEQ ID NOs: 90-111 and 160-183 or a sequence that is at least 80%, 85%, 90%, 95%, 98%, 99%, or 100% identical to any one of SEQ ID NOs: 90-111 and 160-183.

In some embodiments, the nucleic acid composition is or comprises mRNA. In some embodiments, the mRNA is formulated in a lipid nanoparticle (LNP). In some embodiments, the mRNA comprises: a 5′ untranslated region (UTR), a 3′ UTR, and/or a cap; one or more modified nucleotides selected from pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine; and/or a modified nucleotide in place of one or more uridines. In some embodiments, the modified nucleoside is selected from pseudouridine (ψ), N 1-methyl-pseudouridine (m 1ψ), and 5-methyl-uridine (m5U).

In some embodiments, the LNP comprises: one or more of an ionizable cationic lipid, a non-cationic lipid, a sterol, and a PEG-modified lipid, optionally the non-cationic lipid is a neutral lipid; 0.5-15 mol % PEG-modified lipid, 5-25 mol % non-cationic lipid, 25-55 mol % sterol, and 20-60 mol % ionizable cationic lipid; and/or 40-55 mol % ionizable cationic lipid, 5-15 mol % neutral lipid, 35-45 mol % sterol, and 1-5 mol % PEG-modified lipid. In some embodiments, the LNP comprises: 47 mol % ionizable cationic lipid, 11.5 mol % neutral lipid, 38.5 mol % sterol, and 3.0 mol % PEG-modified lipid; 48 mol % ionizable cationic lipid, 11 mol % neutral lipid, 38.5 mol % sterol, and 2.5 mol % PEG-modified lipid; 49 mol % ionizable cationic lipid, 10.5 mol % neutral lipid, 38.5 mol % sterol, and 2.0 mol % PEG-modified lipid; 50 mol % ionizable cationic lipid, 10 mol % neutral lipid, 38.5 mol % sterol, and 1.5 mol % PEG-modified lipid; or 51 mol % ionizable cationic lipid, 9.5 mol % neutral lipid, 38.5 mol % sterol, and 1.0 mol % PEG-modified lipid. In some embodiments, the ionizable cationic lipid is heptadecan-9-yl 8 ((2 hydroxyethyl)(6 oxo 6-(undecyloxy)hexyl)amino)octanoate; the neutral lipid is 1,2 distearoyl sn glycero-3 phosphocholine (DSPC); the sterol is cholesterol; and/or the PEG-modified lipid is 1-monomethoxypolyethyleneglycol-2,3-dimyristylglycerol with polyethylene glycol of average molecular weight 2000 (PEG2000 DMG). In some embodiments, the wt/wt ratio of lipid to mRNA is from about 1:100 to about 100:1. In some embodiments, the composition is a lyophilized composition. In some embodiments, the lyophilized composition has a water content of less than about 10%.

In some embodiments, the composition is formulated or is to be formulated: as a liquid, a solid, or a combination thereof; for infusion; for injection; for intramuscular administration, intranasal administration, transdermal administration, aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection; and/or as particles. In some embodiments, the particles are iron oxide particles, liposomes, micelles, polymer complexes, cationic peptide nanoemulsions, virus-like particles (VLPs), lipid nanoparticles (LNP) and/or lipoplex (LPX) particles.

In some embodiments, the nucleic acid composition and the LNP-forming components are in separate vials. In some embodiments, the composition is a pharmaceutical composition. In some embodiments, the pharmaceutical composition further comprises one or more pharmaceutically acceptable carriers, diluents and/or excipients.

The composition can further comprises instructions for use of the composition for: stimulating an immune response in a subject in need thereof, treating or preventing a disease or disorder in a subject in need thereof, and/or treating or preventing a cancer or an autoimmune disorder in a subject in need thereof.

Disclosed herein include kits. In some embodiments, the kit comprises the composition disclosed herein. Disclosed herein include cells. In some embodiments, the cell comprises the nucleic acid composition disclosed herein.

Disclosed herein include methods of stimulating an immune response in a subject in need thereof. In some embodiments, the method comprises: administering to the subject a therapeutically effective amount of the composition or the cell disclosed herein, thereby stimulating an immune response in the subject.

Disclosed herein include methods of treating or preventing a disease or disorder in a subject in need thereof. In some embodiments, the method comprises: administering to the subject a therapeutically effective amount of the composition or the cell disclosed herein, thereby treating or preventing the disease or disorder in the subject. In some embodiments, the disease or disorder is a cancer or an autoimmune disorder. Disclosed herein include methods for treating or preventing a cancer or an autoimmune disorder in a subject in need thereof. In some embodiments, the method comprises: administering to the subject a therapeutically effective amount of the composition or the cell disclosed herein, thereby treating or preventing a cancer or the autoimmune disorder in the subject.

In some embodiments, immunogenic levels of the APC and/or APV are produced in serum of the subject at about 1 hour to about 6 months post administration of the composition. In some embodiments, a cytokine is released in the serum of the subject at about 1 hour to about 6 months post administration of the composition. In some embodiments, the cytokine is IFN-7. In some embodiments, T cells are activated in the subject at about 1 hour to about 6 months post administration of the composition; and/or T cells are induced to change from one type of T cell to another type of T cell, optionally the regulatory T cell type, in the subject at about 1 hour to about 6 months post administration of the composition.

In some embodiments, the method comprises administering to the subject at least two doses of the composition. In some embodiments, the second dose of the composition is administered to the subject at least 7 days after a first dose of the composition is administered to the subject; the method comprises administering to the subject at least 3, 4, 5, 6, 7, 8, 9, 10, 12, 15, 20, or 25, doses of the composition; and/or the method comprises administering a dose of the composion to the subject on a periodic basis, optionally at least once every hour, every two hours, every four hours, every six hours, every eight hours, every ten hours, every 12 hours, every 16 hours, every 24 hours, every 36 hours, every 48 hours, every 72 hours, every 96 hours, every 5 days, every week, every 2 weeks, every 3 weeks, every 4 weeks, every 5 weeks, every 6 weeks, every 8 weeks, every 10 weeks, every 12 weeks, every 15 weeks, every 20 weeks, every 25 weeks, every 30 weeks, every 40 weeks, every year, every 2 years, or every 3 years.

In some embodiments, the composition elicits an at least 10% higher T cell activation and/or cytokine release as compared to an approach comprising administration of a soluble version of the presenting peptide; or the composition elicits an at least 10% lower T cell activation and/or cytokine release as compared to an approach comprising administration of a soluble version of the presenting peptide. In some embodiments, administration of the composition elicits protective and long-lasting immunity against the disease or disorder associated with the presenting peptide thereof; or administration of the composition elicits protective and long-lasting immunosuppression, optionally associated with the presenting peptide.

In some embodiments, an at least as low dose of the composition is needed to generate a comparable immune response as compared to an approach comprising administration of a soluble version of the presenting peptide; or an at least as low dose of the composition is needed to generate a comparable immunosuppression as compared to an approach comprising administration of a soluble version of the presenting peptide. In some embodiments, following administration of the composition, the composition induces an at least as potent T cell activation and/or cytokine release against the presenting peptide or variants thereof as compared to an approach comprising administration of a soluble version of the presenting peptide, optionally the composition comprises a population of APVs; or the composition induces an at least as potent immunosuppression, optionally against the presenting peptide or variants thereof, as compared to an approach comprising administration of a soluble version of the presenting peptide, optionally the composition comprises a population of APVs.

In some embodiments, the subject: is a human subject; is a newborn or infant of an age of not more than 3 years, of not more than 2 years, of not more than 1.5 years, of not more than 1 year (12 months), of not more than 9 months, 6 months or 3 months, or is between 6 months and 2 years; is immunocompromised, has a pulmonary disease, and/or is 65 years of age or older; has a cancer, optionally melanoma, ovarian cancer, esophageal cancer, lung cancer, hepatocellular cancer, prostate cancer, or breast cancer; and/or has an underlying comorbid condition, optionally selected from heart disease, diabetes, and lung disease.

In some embodiments, the composition is administered in an effective amount to: elicit a robust CD4 and/or CD8 T cell response against the presenting peptide in the subject; and/or elicit a cytokine release against the presenting peptide in the subject, optionally IFN-7 release. In some embodiments, the composition is: (i) co-administered with an adjuvant; or (ii) not co-administered with an adjuvant.

In some embodiments, the disease or disorder is a blood disease, an immune disease, a neurological disease or disorder, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof, optionally a solid tumor. In some embodiments, the disease is associated with expression of a tumor-associated antigen. In some embodiments, the disease associated with expression of a tumor antigen-associated is selected from a proliferative disease, a precancerous condition, a cancer, and a non-cancer related indication associated with expression of the tumor antigen; the cancer is selected from colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers; and/or the cancer is a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

In some embodiments, administering comprises aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, infusion, subcutaneous injection, intranodal injection, intratumoral injection, intraperitoneal injection, intradermal injection, or any combination thereof. In some embodiments, the composition is administered intramuscularly. In some embodiments, the composition is administered into a deltoid region of an arm.

Disclosed herein include methods of modulating an immune cell. In some embodiments, the method comprises: contacting the immune cell with the composition disclosed herein. In some embodiments, modulating comprising activating, regulating, stimulating, and/or suppressing. Disclosed herein include method of isolating an immune cell of interest. In some embodiments, the method comprises: contacting a population of cells suspected of comprising of an immune cell of interest with the composition disclosed herein.

In some embodiments, the contacting step is performed in vitro. In some embodiments, the contacting step is performed in a compartment loaded with the composition disclosed herein, optionally a nanovial. In some embodiments, the contacting step is performed in a cavity of a particle, optionally a nanovial. In some embodiments, the particle comprises at least one opening. In some embodiments, the particle comprises a single opening. In some embodiments, the particle is composed of hydrogel. In some embodiments, a longest dimensional length of the particle is less than about 100 micrometers.

In some embodiments, the APVs are disposed on the surface of the cavity. In some embodiments, the APVs are disposed on the surface of the cavity through a binding moiety. In some embodiments, the binding moiety comprises an antibody or fragment thereof capable of specifically binding the APV. In some embodiments, the binding moiety comprises an antibody or fragment thereof capable of specifically binding to one or more selected from CD274, CD83, CD80, CD54, MHC, the presenting peptide, the APC-assembly domain, and a fragment thereof. In some embodiments, one or more capture agent(s) are disposed on the surface of the cavity. In some embodiments, the capture agent(s) comprise an antibody or fragment thereof capable of specifically binding to a cytokine. In some embodiments, the capture agent(s) comprise an antibody or fragment thereof capable of specifically binding to one or more selected from IL-2, TNF-α, IFN-γ, and fragments thereof. In some embodiments, the immune cell comprises a B cell, a plasmablast, a plasma cell, a T cell, or an NK cell.

The method can further comprise isolating immune cell(s) of interest. In some embodiments, activation of the immune cell is at least about 1.1-fold higher as compared to a method of activating the immune cell using the antigen presentation polypeptide and the presenting peptide without forming the APV. In some embodiments, level of cytokine secretion is at least about 1.1-fold higher as compared to a method of activating the immune cell using the antigen presentation polypeptide(s) and the presenting peptide without forming the APV. In some embodiments, the cytokine is IFN-γ. In some embodiments, the presence of the supplemental polypeptide(s) increases the activation of the immune cell and/or the level of cytokine secretion by at least 10%, 20%, 30%, 40%, 50%, 60%, 70%, 80% or 90%.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 A-FIG. 1F show that EABR addition to the cytoplasmic tail of SCT pMHCI promotes APV budding and release. FIG. 1A shows a schematic of membrane-bound SCT pMHCI proteins on a cell surface containing cytoplasmic tail EABR additions that induced budding of an APV comprising a lipid bilayer with embedded SCT pMHCI proteins. FIG. 1B shows a SCT pMHCI/EABR construct. The top panel of FIG. 1B shows that the SCT pMHCI protein is composed of a peptide fused to a 3×(Gly₄Ser) linker (SEQ ID NO: 30), β2M, a 4×(Gly₄Ser) linker (SEQ ID NO: 31), HLA-A*02:01 alpha chain, a Gly₃Ser spacer (SEQ ID NO: 9), and an EABR sequence. The bottom panel of FIG. 1B shows the EABR sequence. FIG. 1C displays a schematic showing production and purification of APVs. FIG. 1D shows a chromatogram representative of the typical yield of SCT pMHCI APVs from a 100 mL transfection of Expi293F cells. Inset highlights void volume peak. FIG. 1E shows the Western blot analysis detecting SCT pMHCI protein from APVs purified by ultracentrifugation on a 20% sucrose cushion from transfected HEK293T cell culture supernatants. Cells were transfected with SCT MART1/D1, SCT MART1/D1/EABR, SCT MART1/D9, SCT MART1/D9/EABR, and SARS-CoV-2 spike(S)/EPM/EABR (S/EPM/EABR) constructs. Rabbit anti-HLA-A primary antibody and Alexa488-conjugated anti-rabbit secondary antibody were used to detect SCT pMHCI protein. FIG. 1F shows immuno-EM images of SCT MART1/D1/EABR APVs purified from transfected Expi293F cell culture supernatants by ultracentrifugation and SEC. Primary rabbit anti-HLA-A antibody identical to primary antibody used in FIG. 1E. Secondary 6 nm gold-conjugated anti-rabbit antibody appears as black punctate in image. Left: representative APVs are highlighted in boxes. Scale bar, 100 nm. Right: close up of black-boxed APV. Scale bar, 20 nm.

FIG. 2 A-FIG. 2G shows that EABR addition to SCT pMHCI promoted budding of the complete, intended pMHCI complex on APVs. FIG. 2A shows indirect ELISA of APVs purified by ultracentrifugation from transfected HEK293T cells with a primary rabbit anti-HLA-A antibody and secondary HRP-conjugated anti-rabbit antibody. Cells were transfected with SCT NYESO1/D1, SCT NYESO1/D1/EABR, SCT NYESO1/D9, SCT NYESO1/D9/EABR, SCT MART1/D1, and SCT MART1/D1/EABR constructs (2 replicates per dilution). FIG. 2B shows sandwich ELISA of APVs purified by ultracentrifugation from transfected HEK293T cells using anti-NYESO1:HLA-A*02:01 (3M4E5) for capture, biotinylated 3M4E5 for detection, and HRP-conjugated streptavidin secondary. Cells were transfected with SCT NYESO1/D9/EABR, SCD D9/EABR and NYESO1 peptide (SCD/EABR+NYESO1)), HC D9/EABR and NYESO1/β2M (HC/EABR+NYESO1/β2M), SCT NYESO1/D9 (SCT), SCD D9 and NYESO1 peptide (SCD+NYESO1), HC D9 and NYESO1/β2M (HC+NYESO1/β2M) constructs (2 replicates per dilution). FIG. 2C shows dynamic light scattering measurement of SCT MART1/D1 and SCT MART1/D1/EABR APVs represented as a probability mass function of the expected diameter values for each sample. Points are connected by a simple interpolating spline. FIG. 2D displays a schematic showing full SCT pMHCI/EABR construct (SCT/EABR) and various alternative constructs used for the ELISA in FIG. 2B: NYESO1/β2M, HC, and SCD. FIG. 2E-FIG. 2G show immuno-EM micrographs of different EABR vesicles purified from transfected Expi293F cell culture supernatants by ultracentrifugation and SEC, including SARS-CoV2-S/EPM/EABR (FIG. 2E), SCT MART1/D1/EABR (FIG. 2F) and SCT MART1/D1 (FIG. 2G). Samples were stained with a primary rabbit anti-HLA-A antibody and a secondary 6 nm gold-conjugated anti-rabbit antibody that appears as black punctate in image. Scale bars, 100 nm. In FIG. 2E, SARS-CoV2-S/EPM/EABR. Spike protein coronas are visible around the edges of the spherical vesicles. A single gold nanoparticle from a secondary antibody is visible in the middle of the image. In FIG. 2F, SCT MART1/D1/EABR gold particles from secondary antibody can be seen as halos of black punctate around deflated vesicles. In FIG. 2G, SCT MART1/D1 scattered gold particles from secondary antibody can be seen as non-specific staining without the presence of vesicles.

FIG. 3A-FIG. 3B show the generalization of EABR-mediated APV release across SCT pMHCI variants. FIG. 3A shows sandwich ELISA of murine SCT pMHCI of APVs purified by ultracentrifugation from transfected HEK293T cells (2 replicates per dilution). Mouse anti-H-2Kd capture antibody, biotinylated mouse anti-H-2Kd primary antibody, and HRP-conjugated streptavidin secondary antibody. FIG. 3B shows sandwich ELISA of chimeric human/mouse SCT pMHCI protein on APVs purified by ultracentrifugation from transfected HEK293T cell culture supernatants (2 replicates per dilution). Mouse anti-NYESO1:HLA-A*02:01 capture antibody, biotinylated mouse anti-NYESO1:HLA-A*02:01 primary antibody, and HRP-conjugated streptavidin secondary antibody.

FIG. 4A-FIG. 4C show IFN-7 release from their cognate T cells in vitro induced by pMHCI/EABR APVs. FIG. 4A shows quantitative Western blot comparing known amounts of NYESO1:HLA-A*02:01 tetramer (NYESO1 tet.) standards (lanes 1-5) with various dilutions of purified NYESO1/D9/EABR APVs (NYESO1 APVs) (lanes 6-9) to determine pMHCI protein concentrations in APV samples. Mouse anti-β2M primary antibody, Alexa488-conjugated anti-mouse secondary antibody. FIG. 4B shows scans of ELISPOT wells detecting IFN-7 release from one replicate test of Donor 3's T cells after being dosed with serial dilutions of NYESO1:HLA-A*02:01 tetramers (NYESO1 Tetramer), SCT NYESO1/D9/EABR APVs, and SCT MART1/D9/EABR APVs (top three rows) and scans of ELISPOT wells detecting IFN-7 release from T cells dosed with 1.5% PHA as a positive control, wells without cells as a negative control (No cells), and T cells plated without antigen challenge (No antigen) (bottom row). FIG. 4C shows ELISPOT spot forming units for IFN-7 from T cells of three different donors dosed with SCT NYESO1/D9/EABR APVs, SCT MART1/D9/EABR APVs, NYESO1:HLA-A*02:01 tetramers (NYESO1 Tetramer), or no antigen challenge. Each data point is the response per 1×10⁶ cells. Baseline stimulation (i.e., “No antigen”—plated T cells without stimulation by antigen) was measured in triplicate for each donor and presented as a single mean in each plot with standard deviation. Because the NYESO1:HLA-A*02:01 tetramers generated larger spot sizes on average compared to the APVs, Donor 3's spot forming units were too numerous to count when dosed with more than 10 ng of NYESO1:HLA-A*02:01 tetramer, preventing direct comparison between antigen conditions at higher concentrations for this donor. Significant differences between conditions linked by vertical lines are indicated by asterisks: ns>0.05; *p<0.05; **p<0.01; ***p<0.001; ****p<0.0001.

FIG. 5 displays additional micrographs of pMHCI/EABR grids prepared by immuno-EM showing APVs. All micrographs show Expi293F cell culture supernatant after transfection with a SCT MART1/D1/EABR construct and purified by ultracentrifugation and SEC as outlined in FIG. 1C. Panels A-D in FIG. 5 show 50 mL of purified Expi293F cell culture supernatant. Panels E-I in FIG. 5 show 200 mL of purified Expi293F cell culture supernatant further diluted in pH 7.4 PBS after purification. All grids were prepared using the identical protocol as in FIG. 1F, FIG. 2C and FIG. 2E-FIG. 2F. Secondary 6 nm gold-conjugated anti-rabbit antibody bound to primary rabbit anti-HLA-A antibody appears as black punctate in the image. In panel A of FIG. 5, scale bar=200 nm. In panels B-I of FIG. 5, scale bar=100 nm.

FIG. 6A-FIG. 6B show Western blots of purified APVs that were negative for CD81 (FIG. 6A) and CD63 (FIG. 6B), including purified Expi293F SCT D1 MART1/D1/EABR APVs (XP293F D1/EABR APVs), Expi293F purified supernatant, HEK293T SCT D1 MART1/D1/EABR APVs (HEK293T D1/EABR APVs), HEK293T cell extract, and HEK293T purified supernatant.

FIG. 7A-FIG. 7B show indirect ELISA of purified Expi293F and HEK293T transfection supernatant, suggesting complete pMHCI not required for APV release. Indirect ELISA of purified Expi293F (FIG. 7A) and HEK293T (FIG. 7B) supernatant transfected with SCT MART1/D1/EABR (SCT/EABR), SCD D1/EABR (SCD/EABR), D1 HC-EABR (HC/EABR), and the non-EABR versions of the three constructs: SCT MART1/D1 (SCT), SCD D1 (SCD), and HC D1 (HC). The diagrams of SCT, SCD, and HC are in FIG. 2D. All supernatant was purified by ultracentrifugation and re-suspended in pH 7.4 PBS. Polyclonal rabbit anti-HLA-A was used for the primary antibody, and HRP-conjugated goat anti-rabbit was used for the secondary antibody (2 replicate measurements per dilution).

FIG. 8 displays sandwich ELISA of purified Expi293F transfection supernatant showing non-EABR SCT capable of vesicle formation. Sandwich ELISA of purified Expi293F supernatant transfected with: SCT NYESO1/D9/EABR (SCT/EABR); SCD D9/EABR (SCD/EABR) along with co-transfected NYESO1 peptide; HC-D9/EABR (HC/EABR) along with co-transfected NYESO1/β2M; and, the non-EABR versions of the three constructs: SCT NYESO1/D9 (SCT) and SCD D9 (SCD) with co-transfected NYESO1, and HC-D9 (HC) with co-transfected NYESO1/β2M. All supernatant was purified by ultracentrifugation and re-suspended in pH 7.4 PBS. Monoclonal human anti-NYESO1:HLA-A*02:01 complex was used for the initial capture antibody. Biotinylated monoclonal human anti-NYESO1:HLA-A*02:01 complex was used for the primary detection antibody and secondary strep-RP was then incubated in each well before developing with HRP substrate. (2 replicate measurements per dilution).

FIG. 9A-FIG. 9C show scans of ELISPOT plates detecting IFN-7 release from donor T cells from Donor 1 (FIG. 9A), Donor 2 (FIG. 9B), and Donor 3 (FIG. 9C), after being dosed with serial dilutions of SCT NY-ESO-1/D9/EABR APVs (lanes 4, 7 and 10), SCT MART1/D9/EABR APVs (lanes 5, 8 and 11), and NYESO1:HLA-A2 tetramers (lanes 6, 9 and 12); 1.5% PHA (lane 3, row A of FIG. 9A-FIG. 9C); without cells or antigen challenge (lane 2, rows A of FIG. 9A-FIG. 9C); and cells without antigen challenge (lane 1, rows A-C of FIG. 9A-FIG. 9C).

FIG. 10A-FIG. 10G shown that EABR addition to pMHCII promoted budding of the complete, intended pMHCII complex on APVs. FIG. 10A displays a schematic showing cartoon representation of naturally-occurring MHCII including the alpha and beta chains that form the full MHCII complex. A MHCII complex is termed a peptide-MHCII (pMHCII) complex when a presenting peptide is bound in the peptide-binding cleft formed by the complexed MHCII alpha and beta chains. FIG. 10B displays a schematic showing a SCT pMHCII/EABR construct (SCT-T & SCT-T/EABR) and various alternative constructs used for the ELISA in FIG. 10C-FIG. 10G. FIG. 10C shows ELISA of purified vesicles from Expi293F cells that were transfected with various DNA plasmids encoding a murine version of MHCII, I-Ab, presenting the E alpha peptide (Ea) or pMHCII single-chain trimer constructs. FIG. 10D shows ELISA of purified vesicles from HEK293T cells that were transfected with various DNA plasmids encoding a murine version of MHCII, I-Ab, presenting the E alpha peptide (Ea) or pMHCII single-chain trimer constructs. FIG. 10E shows ELISA of purified vesicles from NMuMG cells that were transfected with various mRNA transcripts encoding a murine version of MHCII, I-Ab, presenting the E alpha peptide or pMHCII single-chain trimer constructs. 10F shows ELISA of purified vesicles from Expi293F cells that were transfected with various DNA plasmids encoding a murine version of MHCII, I-Ab, presenting the multiple-sclerosis-relevant myelin oligodendrocyte peptide or pMOG peptide as well as single-chain trimer pMHCII constructs. FIG. 10G shows ELISA of purified vesicles from HEK293T cells that were transfected with various DNA plasmids encoding a murine version of MHCII, I-Ab, presenting the myelin oligodendrocyte peptide or pMOG peptide as well as single-chain trimer pMHCII constructs.

FIG. 11A-FIG. 11D show that engineered extracellular vesicles improved single cell capture and T cell activation on hydrogel nanovials compared to functionalizing the hydrogel surface with recombinant protein. FIG. 11A shows a schematic of a plasmid construct containing a membrane protein fused to a Gly₃Ser spacer (SEQ ID NO: 9), and an EABR vesicle-forming sequence (top) and a schematic of membrane-bound protein on a cell surface containing cytoplasmic tail EABR additions that induced budding of a vesicle comprising a lipid bilayer with embedded membrane proteins containing the EABR sequence. FIG. 11B shows a schematic showing production and purification of the engineered EABR-mediated extracellular vesicles. FIG. 11C shows post-fabrication biotinylation of hydrogel nanovials via gelatin, which were then functionalized with streptavidin, followed by biotinylated capture antibodies. FIG. 11D shows detection of cytokines released by activated cells that were captured within the nanovial, using fluorescent sandwich antibodies.

FIG. 12A-FIG. 12D show presentation of both HER2/EABR and CD54 proteins on purified extracellular vesicles present in transfection supernatant after co-expression of HER2/EABR and CD54. FIG. 12A shows a schematic showing the plasmid construct for HER2 vesicles featuring a G3S spacer (SEQ ID NO: 9) and the EABR vesicle-forming sequence. The plasmid was transfected into HEK293T or Expi293F cells and vesicles were harvested as shown in FIG. 11A. FIG. 12B shows a schematic showing the plasmid construct for HER2+CD54 vesicles. Only HER2 required a vesicle-forming EABR sequence to be appended for both CD54 and HER2 to appear on the harvested vesicles. FIG. 12C shows results of sandwich ELISA of APVs purified by ultracentrifugation from transfected Expi293F cells. Cells were co-transfected with HER2/EABR and CD54, HER2/EABR alone, HER2/EABR and CD54/EABR, or HER2 constructs (2 replicates per dilution). In the top panel anti-HER2 antibodies were used for capture, biotinylated anti-HER2 antibodies were used for detection, and HRP-conjugated streptavidin secondary antibodies were used for visualization. In the bottom panel, anti-CD54 antibodies were used for capture, biotinylated anti-HER2 antibodies were used for detection, and RP-conjugated streptavidin secondary antibodies were used for visualization. FIG. 12D shows immuno-EM image of vesicles purified by ultracentrifugation and size-exclusion chromatography from Expi293F cells co-transfected with HER2/EABR and CD54 constructs. Primary antibodies used were mouse anti-HER2 and rabbit anti-CD54. Secondary antibodies 12 nm gold-conjugated anti-mouse antibodies for visualization of HER2 appeared as black punctae in image and highlighted by arrows with “*”. Secondary 6 nm gold-conjugated anti-rabbit antibodies for visualization of CD54 appeared as smaller black punctae in image and highlighted by arrows without “*”. Scale bar, 200 nm.

FIG. 13A-FIG. 13D show that coating hydrogel nanovials with vesicles co-presenting HER2/EABR and CD54 improved cell loading and CAR-T activation compared to coating with recombinant HER2. FIG. 13A shows immobilization of HER2 eVLP on 35 μm and 50 μm nanovials based on concentration of aHER2 capture antibodies on the nanovials. Maximum capture signal was detected at around 20 μg/mL aHER2 capture antibodies for both 35 μm and 50 μm nanovials. FIG. 13B shows the significantly higher capture rate of CD54/HER2 eVLP on nanovials compared to the HER2-only eVLP. This signal was comparable to the positive control of nanovials conjugated with 20 μg/mL recombinant HER2 (rHER2). FIG. 13C shows aHER2 CAR cell and control untransfected (UTX) T cell loading in nanovials using both HER2 eVLP and CD54/HER2 eVLP. FIG. 13D displays flow data showing improvement in nanovial assay using eVLP.

FIG. 14A-FIG. 14H show that co-expression of SCT pMHCI/EABR and CD274, CD83 or CD80 immunoregulatory proteins allowed for fine-tuned synthesis of known endogenous immunoregulatory extracellular vesicles. FIG. 14A shows a schematic of the assay performed for the top row of FIG. 14C-FIG. 14E. Expression of immunoregulatory membrane proteins CD274, CD83, or CD80 on engineered extracellular vesicles were captured with plated antibody, then co-presented NYESO1/HLA-A*02:01 were detected and quantified with biotinylated anti-NYESO:HLA-A*02:01 and RP-conjugated streptavidin (strep-RP). FIG. 14B shows a schematic of the assay performed for the bottom row of FIG. 14C-FIG. 14E. Identical assay was performed in FIG. 14A and FIG. 14B, except that the capture antibody and detection antibody were swapped. FIG. 14C shows results of sandwich ELISA of APVs purified by ultracentrifugation from transfected Expi293F cells using anti-CD274 for capture, biotinylated anti-NYESO1:HLA-A*02:01 (3M4E5) for detection, and HRP-conjugated streptavidin secondary. Cells were co-transfected with SCT NYESO1/D9/EABR and CD274, SCT NYESO1/D9/EABR and CD274/EABR, SCT NYESO1/D9 and CD274/EABR, or SCT NYESO1/D9 and CD274 constructs (2 replicates per dilution). FIG. 14D shows results of sandwich ELISA of APVs purified by ultracentrifugation from transfected Expi293F cells using anti-CD83 for capture, biotinylated anti-NYESO1:HLA-A*02:01 (3M4E5) for detection, and RP-conjugated streptavidin secondary. Cells were co-transfected with SCT NYESO1/D9/EABR and CD83, SCT NYESO1/D9/EABR and CD83/EABR, SCT NYESO1/D9 and CD83/EABR, or SCT NYESO1/D9 and CD83 constructs (2 replicates per dilution). FIG. 14E shows results of sandwich ELISA of APVs purified by ultracentrifugation from transfected HEK293T cells using anti-CD80 for capture, biotinylated anti-NYESO1:HLA-A*02:01 (3M4E5) for detection, and RP-conjugated streptavidin secondary. Cells were co-transfected with SCT NYESO1/D9/EABR and CD80, SCT NYESO1/D9/EABR and CD80/EABR, SCT NYESO1/D9 and CD80/EABR, or SCT NYESO1/D9 and CD80 constructs (2 replicates per dilution). FIG. 14F shows results of nano flow cytometry (NanoFCM) of APVs purified by ultracentrifugation from transfected HEK293T cells, and co-stained with APC anti-CD80 and Alexa488 anti-NYESO1:HLA-A*02:01 (3M4E5). In the top-left panel of FIG. 14F, vesicles were purified from HEK293T transfected with the MART1/D9/EABR construct were used for gating non-specific signal from irrelevant extracellular vesicles. In the top-right panel of FIG. 14F, vesicles were from HEK293T cells transfected with the NYESO1/D9/EABR construct. In the bottom-left panel of FIG. 14F, vesicles were from HEK293T cells transfected with NYESO1/D9/EABR and CD80 constructs. In the bottom-right panel of FIG. 14F, vesicles were from HEK293T cells transfected with NYESO1/D9/EABR and CD80/EABR constructs. FIG. 14G shows results of NanoFCM size analysis of vesicles purified by ultracentrifugation from HEK293T cells co-transfected with NYESO1/D9/EABR and CD80/EABR constructs. FIG. 14H shows immuno-EM image of vesicles purified by ultracentrifugation and size-exclusion chromatography from Expi293F cells co-transfected with NYESO1/D9/EABR and CD80/EABR constructs. Primary antibodies used were human anti-NYESO1:HLA-A*02:01 (3M4E5) antibody and mouse anti-CD80. Secondary 12 nm gold-conjugated anti-mouse antibody appeared as black punctae in image and highlighted by arrow with “*”. Secondary 6 nm gold-conjugated anti-human antibody appeared as smaller black punctae in image and highlighted by arrow without “*”. Scale bar, 200 nm.

FIG. 15A-FIG. 15I show that coating hydrogel nanovials with synthetic antigen-presenting vesicles co-presenting pMHCI/EABR and CD80 improved cell loading and T cell activation compared to coating with recombinant pMHCI. FIG. 15A-FIG. 15D show flow cytometry gating strategy for T-cell loaded nanovials and secretion detection. FIG. 15A shows that single nanovials were separated from hydrogel/PEG debris and nanovial “doublets,” which were incorrectly formed during microfluidic fabrication. FIG. 15B shows that live cells were stained positively with Calcein AM. FIG. 15C shows that NGFR+ cells were identified as having been successfully transfected with the 1G4 T cell receptor specific for NYESO1/HLA-A*0201. FIG. 15D shows that nanovials passing the previous flow cytometry gates were then assessed for their anti-IFN-7 BV421 signal, which was a readout for T cell activation. FIG. 15E-FIG. 15I show the results of the flow cytometry analysis illustrated in FIG. 15A-FIG. 15D on nanovial cavity surface with different conjugations: NYESO1/MHCI (FIG. 15E), aCD45 antibodies (FIG. 15F), aCD45 antibodies and MART1/MHCI (FIG. 15G), NYESO1 pMHC-eVLPs conjugated to nanovial cavity through α NYESO1/MHCI antibodies (FIG. 15H), and NYESO1 and CD80 pMHC-eVLPs conjugated to nanovial cavity through α NYESO1/MHCI antibodies (FIG. 15I). First panels from top of FIG. 15E-FIG. 15I show comparison of interaction between T cells and nanovial cavity surface with different conjugations. Second panels from top of FIG. 15E-FIG. 15I show relative cell loading rates into nanovials using different binding moieties. Third panels from top of FIG. 15E-FIG. 15I show IFN-7 secretion levels from single T cells on nanovials. Matched pMHCs (protein monomer and eVLP format) triggered T cell activation.

FIG. 16 displays a western blot showing presence of pMHC-EABR particles in exosomal fraction after centrifugation using an anti-HLA-A antibody.

FIG. 17 shows a diagram explaining a proposed mechanism of action for how the SCT pMHC mRNA LNP vaccine would work to down-regulate immune cells against a specific target.

FIG. 18 shows a diagram explaining a proposed mechanism of action for how a SCT pMHC and CD86 mRNA LNP vaccine would work to activate immune cells against a specific target.

FIG. 19 shows a flowchart explaining how the vesicles disclosed herein can be generated and purified.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include compositions. In some embodiments, the composition comprises: a nucleic acid composition comprising one or more polynucleotides encoding components of an antigen presentation complex (APC). In some embodiments, the components of the APC are capable of assembling into an antigen-presenting vesicle (APV) budded from a cell in which the APC components are expressed. In some embodiments, the APV comprises a plurality of APCs. In some embodiments, the APC comprises an APC-assembly domain, one or more antigen presentation polypeptide(s) and a presenting peptide. Disclosed herein include compositions. In some embodiments, the composition comprises: a population of an antigen-presenting vesicles (APVs). In some embodiments, each of the APVs comprises a plurality of antigen presentation complexes (APCs). In some embodiments, the APC comprises an APC-assembly domain, one or more antigen presentation polypeptide(s) and a presenting peptide. The composition can further comprises instructions for use of the composition for: stimulating an immune response in a subject in need thereof; treating or preventing a disease or disorder in a subject in need thereof, and/or treating or preventing a cancer or an autoimmune disorder in a subject in need thereof.

Disclosed herein include kits. In some embodiments, the kit comprises the composition disclosed herein. Disclosed herein include cells. In some embodiments, the cell comprises the nucleic acid composition disclosed herein.

Disclosed herein include methods of stimulating an immune response in a subject in need thereof. In some embodiments, the method comprises: administering to the subject a therapeutically effective amount of the composition or the cell disclosed herein, thereby stimulating an immune response in the subject. Disclosed herein include methods of treating or preventing a disease or disorder in a subject in need thereof. In some embodiments, the method comprises: administering to the subject a therapeutically effective amount of the composition or the cell disclosed herein, thereby treating or preventing the disease or disorder in the subject. In some embodiments, the disease or disorder is a cancer or an autoimmune disorder. Disclosed herein include methods for treating or preventing a cancer or an autoimmune disorder in a subject in need thereof. In some embodiments, the method comprises: administering to the subject a therapeutically effective amount of the composition or the cell disclosed herein, thereby treating or preventing a cancer or the autoimmune disorder in the subject.

Disclosed herein include methods of modulating an immune cell. In some embodiments, the method comprises: contacting the immune cell with the composition disclosed herein. In some embodiments, modulating comprising activating, regulating, stimulating, and/or suppressing. Disclosed herein include method of isolating an immune cell of interest. In some embodiments, the method comprises: contacting a population of cells suspected of comprising of an immune cell of interest with the composition disclosed herein.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the terms “antigen” or “immunogen” are used interchangeably to refer to a substance, typically a protein, which is capable of inducing an immune response in a subject (e.g. a mammal, such as a human). The term also refers to proteins that are immunologically active in the sense that once administered to a subject, either directly or in the form of a nucleotide sequence or vector that encodes the protein, is able to evoke an immune response of the humoral and/or cellular type directed against that protein or a variant thereof.

As used herein, “sequence identity” or “identity” in the context of two nucleic acid or polypeptide sequences makes reference to the nucleotide bases or residues in the two sequences that are the same when aligned for maximum correspondence over a specified comparison window. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; Pearson et al., Meth. Mol. Bio. 24:307-31, 1994; and Altschul et al., J. Mol. Biol. 215:403-10, 1990 (the content of each of these references is incorporated herein in its entirety).

When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule. A functionally equivalent residue of an amino acid used herein typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to one of skill in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

The term “substantially identical” as used herein in the context of two or more sequences refers to a specified percentage of amino acid residues or nucleotides that are identical or functionally equivalent, such as about, at least or at least about 65% identity, optionally, about, at least or at least about 70%, 75%, 80%, 85%, 90%, 95%, or 99% identity over a specified region or over the entire sequence.

As used herein, the term “variant” refers to a polynucleotide or polypeptide having a sequence substantially similar or identical to a reference (e.g., the parent) polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least, or at least about, 50%, 55%, 60%, 65%, 70%, 75%, 80%, 85%, 90%, 91%, 92%, 93%, 94%, 95%, 96%, 97%, 98%, 99%, or more sequence identity to the reference polynucleotide as determined by sequence alignment programs known in the art. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. A variant of a polypeptide can have, for example, at least, or at least about, 60%, 65%, 70%, 75%, 80%, 85%, 90%, 91%, 92%, 93%, 94%, 95%, 96%, 97%, 98%, 99%, or more sequence identity to the reference polypeptide as determined by sequence alignment programs known in the art.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those commonly known and used in the art. Standard techniques can be used for chemical syntheses, chemical analyses, pharmaceutical preparation, formulation, and delivery, and treatment of patients.

The term “construct,” as used herein, refers to a recombinant nucleic acid that has been generated for the purpose of the expression of a specific nucleotide sequence(s), or that is to be used in the construction of other recombinant nucleotide sequences.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

### Assembly of Antigen Presentation Vesicles

All nucleated cells of the human body present intracellular proteins on their surface as digested peptides bound to class I major histocompatibility complexes (MHCI). When a peptide is bound to MHCI, the complex is referred to as peptide:MHCI (pMHCI). The T cell receptors (TCRs) of CD8+ T cells can recognize the presence of infection or cancer by binding pMHCI strongly and specifically, when the peptide presented by MHCI is of pathologic origin. Class II major histocompatibility complex, MHCII, is present on the membrane surface of professional antigen presenting cells, such as macrophages and dendritic cells, which are specialized cells of the immune system that are efficient at presenting pMHCII. In this case, the peptide on MHCII originates from extracellular origins such as a foreign pathogen or virus. When pMHCII binds to its cognate TCR on CD4+ T cells, a focused, adaptive immune response is initiated to eliminate targets presenting the antigenic peptide. In cases of both classes of MHC, the activation of T cells and the initiation of an immune response requires additional co-stimulatory/molecules, such as CD80/CD86 and CD28, to bind along with the pMHC-TCR interaction. Without these co-stimulatory signals, pMHC complexes binding to TCRs can do the opposite: they can cause T cell anergy, apoptosis, or differentiation into a regulatory phenotype that turns down immune responses specifically for that peptide.

In 1996, the release of B cell-derived extracellular vesicles (EVs) with functional pMHC complexes on their surface was reported. Over the next two decades, EVs that were previously labeled as simple cellular “debris” were increasingly recognized for their immunoregulatory potential. Naturally occurring antigen-presenting vesicles (APVs), in particular, have been demonstrated to orchestrate immune responses either directly or with the aid of antigen-presenting cells.

The significance of antigen presenting cells and APVs in regulating immune responses has inspired researchers to try recreating the functionality of the antigen presenting cells synthetically. In 1978, pMHCI was incorporated into phospholipid vesicles to induce cytotoxic T lymphocytes. Research teams have since ventured to engineer more complex versions of these APVs that can direct the immune system to attack a target of interest, deeming them “artificial antigen-presenting cells.” Initial rudimentary artificial antigen-presenting cells were designed by conjugating MHC together as tetramers using biotin and streptavidin, which is still used today to capture and identify T cells with high affinity for a specific pMHC. Later designs for artificial antigen-presenting cells included whole mammalian cells conjugated with immunostimulatory molecules, iron-oxide or polymer nanoparticles conjugated with effector proteins from antigen presenting cells, nanoparticles coated with membranes of antigen presenting cell via extrusion or sonication, or engineered extracellular blebs from dendritic cells. These existing approaches often involve non-specific activation of the adaptive immune system to demonstrate a proof-of-concept, or they require the patient's own cells (i.e. autologous cells) to be harvested and processed before treatment can be administered. Furthermore, while the direct manipulation of dendritic cells through gene therapy or targeted antigen delivery has shown promising results, there are also some limitations of this route. For example, copy numbers of dendritic cell in blood are relatively low. Thus, the generation of sufficient autologous antigen presenting cells to prepare exosomes or artificial antigen presenting cells from dendritic cell lysates remains a barrier to the broad utility of antigen-presenting cells —based therapies. Moreover, better T cell activation from the nanoparticle artificial antigen-presenting cells than from actual dendritic cells can be due in part to the low copy number and activation potential of the autologous dendritic cells and the added benefit afforded by “cross-dressing” nearby cells with the nano-sized artificial antigen-presenting cells.

Significant attention has been given to producing antigen-presenting cells via microfluidics, but existing microfluidic vesicles suffer from short half-lives and are very susceptible to rupture due to osmotic changes and the turbulent flow of the lymphatic and cardiovascular systems. It was demonstrated that iron-oxide nanoparticles coated in pMHCI complexes could restore normoglycemia in non-obese diabetic mice. Since then, the efficacy of his pMHCI and pMHCII nanoparticles was demonstrated for restoring motor function in a murine model of experimental autoimmune encephalitis (a corollary to multiple sclerosis) and collagen-induced arthritis (a corollary to rheumatoid arthritis), in addition to increasing the efficacy of the normoglycemia-restoring nanoparticles in diabetic mice. There are some critical limitations of these iron-oxide nanoparticles. For example, rigid adherence of the pMHC complex to the nanoparticle surface reduces pMHC movement, which has been considered important for allowing signal protein and receptor reorganization in response to T cell contact, i.e. forming an immunological synapse between a T cell TCR cluster and a pMHC bouquet. Rigid spherical structure of the nanoparticle scaffold reduces the potential surface area for ideal pMHC-TCR interactions, as oblong artificial antigen-presenting cells have been demonstrated to more effectively activate T cells than spherical artificial antigen-presenting cells. The biodistribution of pMHC iron-oxide nanoparticles was largely sequestered to the liver and spleen. Particles that more closely resemble endogenous extracellular vesicles may have better biodistribution. Exogenously generated iron-oxide nanoparticles require each patient's HLA type to be sequenced before administration of the therapy. In comparison, an endogenously generated APV could potentially use the patient's own MHC molecules without requiring any patient HLA-typing.

An ideal antigen-presenting vesicle therapeutic, such as the antigen-presenting vesicle (APV) disclosed herein, would avoid the aforementioned limitations. The APV can also have advantages that has not been fully accomplished by existing therapeutic. Targeted immunoregulation of a single immune pathology can be achieved without suppressing any desirable immune responses to viral, bacterial, or parasitic pathogens. the APV can recapitulate existing, physiologically natural pathways for immunoregulation. The APVs can be made/produced in sufficient quantities without difficulty (e.g. therapies relying on the use of antigen-presenting cells may be difficult to manufacture or collect from the patient). To achieve good biodistribution, the APV can be >30 nm in size to avoid excretion in the kidneys and <100 nm in size to avoid the reticuloendothelial system, which sequesters larger particles in the liver and spleen. The APV can allow for additional “tissue targeting” moieties to be added if desirable. Moreover, the APV can also be easily administered for both the patient and healthcare team, well-tolerated by patients and of low-cost.

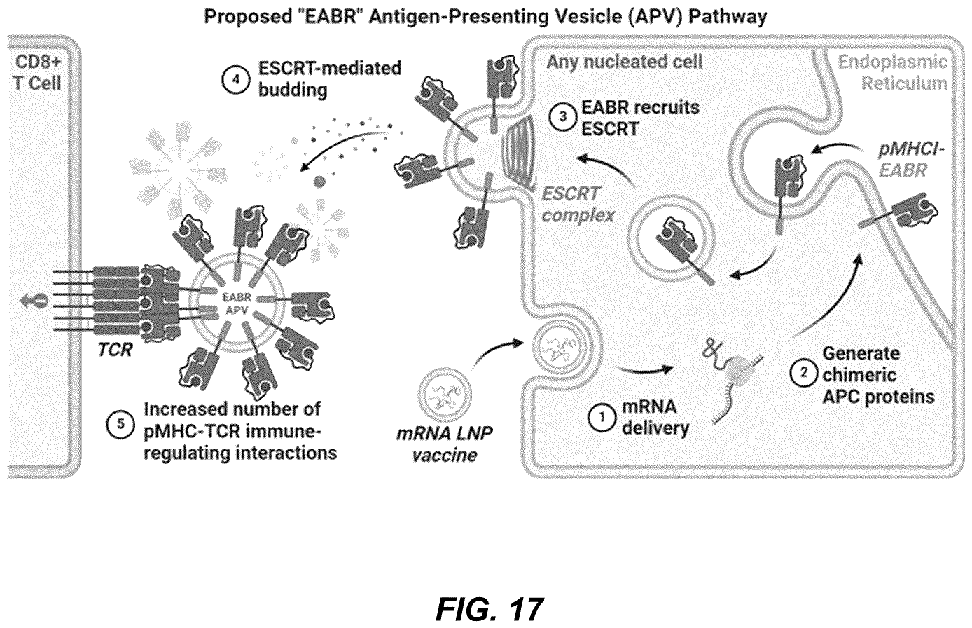

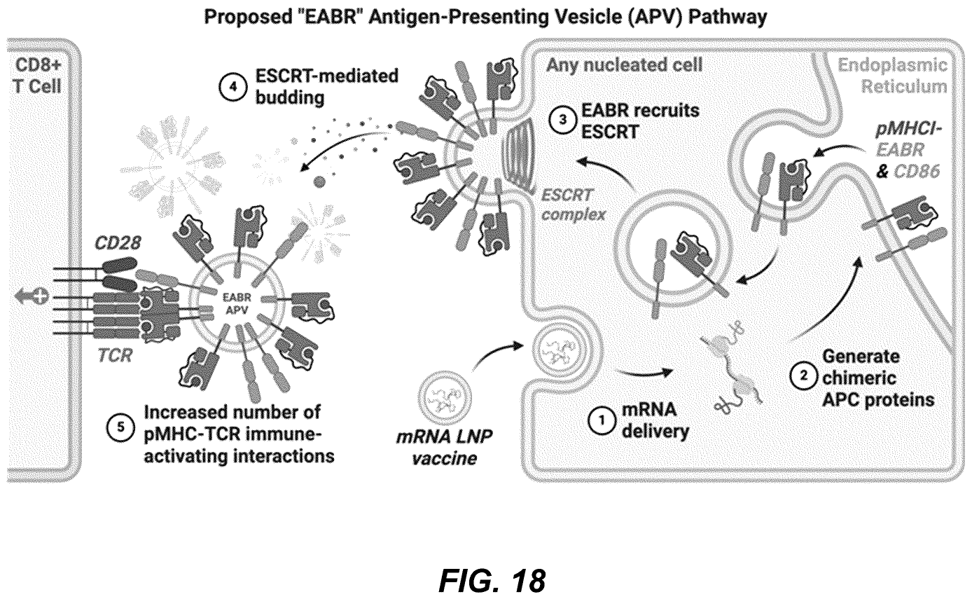

By leveraging recent advances in the creation of enveloped virus like particles (eVLPs) generated in vivo from mRNA vaccination, APVs that can either stimulate an immune response against targets of interest (e.g., cancer and viral targets) or down-regulate an undesirable autoimmune response (e.g. type I diabetes, rheumatoid arthritis, multiple sclerosis) are disclosed herein (FIG. 17 and FIG. 18).

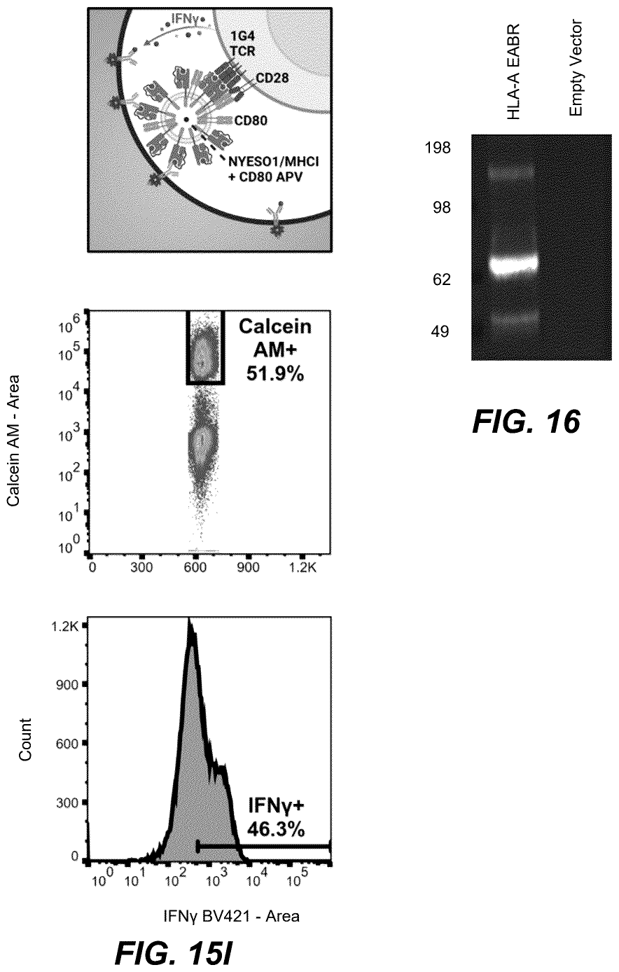

These APVs supported pMHC complexes in lipid membranes that were sturdy and survived paraformaldehyde fixation, disruption by repeated aspiration and centrifugation, and changes in temperature. The pMHC complexes were distributed throughout the enveloped lipid membrane of exosome-like vesicles when observed by negative stain immuno-electron microscopy, and the vesicles were produced in high-copy number as evident by both western blotting and enzyme-linked immunosorbent assay (ELISA) (FIG. 16). Both pMHCI and pMHCII vesicles were made using this technology.

Additional immunoregulatory proteins (e.g., costimulatory molecules CD80/CD86, immunosuppressive proteins such as CD73, cellular adhesion proteins such as LFA-1, anti-phagocytic proteins such as CD47, or tissue-homing proteins such as CCR7) can be added to the membranes of the vesicles in a similar fashion as disclosed herein.

In one embodiment, the APVs disclosed herein can be manufactured using large-scale biomanufacturing, processed externally, optionally stored for an extended period of time, and later injected into patients as therapeutic immunoregulatory vesicles.

In another embodiment, the APVs disclosed herein can be delivered therapeutically in the form of a lipid nanoparticle mRNA vaccine, and the APVs are translated/generated in the patient by the patient's own cells. These APVs can recruit/use the patient's own cellular proteins in addition to new proteins that were generated by an RNA transcript delivered as part of the nanoparticle mRNA vaccine.

In another embodiment, the APVs can be used as a way to activate/regulate/stimulate and/or isolate PBMCs or T cells in an in vitro context—these pMHC vesicles can be generated to search for unconventional major histocompatibility complex (MIC) molecules or rare TCRs in a large cell-based screen, e.g. Dino Di Carlo's nanovial system.

In some embodiments, the APC comprises an amino acid sequence having at least 65%, 70%, 75%, 80%, 85%, 90%, 95%, 98%, or 99% sequence identity to an amino acid sequence selected from SEQ ID NOs: 37-52, 55-56, 59-60, 63-80, 114-119, 122-143, 146-151, and 154-159. Exemplary APCs and/or portions thereof provided herein are listed in Table 1 below.

TABLE 1
Exemplary APCs and portions thereof

| | SEQ |
|---|---|
| Name | ID NO |
| D1 SCT MART1/HLA-A*02:01 | 37-38 |
| D1 SCT MART1/HLA-A*02:01/EABR | 39-40 |
| D9 SCT MART1/HLA-A*02:01 | 41-42 |
| D9 SCT MART1/HLA-A*02:01/EABR | 43-44 |
| D1 SCT NYESO1/HLA-A*02:01 | 45-46 |
| D1 SCT NYESO1/HLA-A*02:01/EABR | 47-48 |
| D9 SCT NYESO1/HLA-A*02:01 | 49-50 |
| D9 SCT NYESO1/HLA-A*02:01/EABR | 51-52 |
| NYESO1/β2M | 55-56 |
| D9 SCD β2M/HLA-A*02:01/EABR | 59-60 |
| D9 HLA-A*02:01 HC/EABR | 63-64 |
| NRP-V7/H-2Kd | 65-66 |
| NRP-V7/H-2Kd/EABR | 67-68 |
| IGRP206-214/H-2Kd | 69-70 |
| IGRP206-214/H-2Kd/EABR | 71-72 |
| NYESO1/A2_D9/H-2Db | 73-74 |
| NYESO1/A2_D9/H-2Db/EABR | 75-76 |
| NYESO1/A2_D9/H-2Kb | 77-78 |
| NYESO1/A2_D9/H-2Kb/EABR | 79-80 |
| Human sequence optimized Hgh/Ea/I-Ab-a | 114-115 |
| Human sequence optimized Hgh/I-Ab-a/EABR | 116-117 |
| Human sequence optimized Hgh/Ea/I-Ab-a/EABR | 118-119 |
| Human sequence optimized Hgh/Ea/I-Ab-b | 122-123 |
| Human sequence optimized Hgh/I-Ab-b/EABR | 124-125 |
| Human sequence optimized Hgh/Ea/I-Ab-b/EABR | 126-127 |
| Human sequence optimized “SCT-T” Hgh/Ea/I-Ab | 128-129 |
| Human sequence optimized “SCT-T” Hgh/Ea/I-Ab/EABR | 130-131 |
| Human sequence optimized Hgh/pMOG/I-Ab-a | 132-133 |
| Human sequence optimized Hgh/pMOG/I-Ab-a/EABR | 134-135 |
| Human sequence optimized Hgh/pMOG/I-Ab-b | 136-137 |
| Human sequence optimized Hgh/pMOG/I-Ab-b/EABR | 138-139 |
| Human sequence optimized “SCT-T” Hgh/pMOG/I-Ab | 140-141 |
| Human sequence optimized “SCT-T” Hgh/pMOG/I-Ab/EABR | 142-143 |
| Murine sequence optimized for mRNA Tx: Ea/I-Ab-a | 146-147 |
| Murine sequence optimized for mRNA Tx: I-Ab-a/EABR | 148-149 |
| Murine sequence optimized for mRNA Tx: Ea/I-Ab-a/EABR | 150-151 |
| Murine sequence optimized for mRNA Tx: Ea/I-Ab-b | 154-155 |
| Murine sequence optimized for mRNA Tx: I-Ab-b/EABR | 156-157 |
| Murine sequence optimized for mRNA Tx: Ea/I-Ab-b/EABR | 158-159 |

### The APC-Assembly Domain

In some embodiments, the APC-assembly domain recruits one or more ESCRT proteins to the cytoplasmic tail of the APC. In some embodiments, the recruitment of ESCRT proteins via the APC-assembly domain induces the self-assembly and budding of APVs. The cytoplasmic portion of the APC can comprise the APC-assembly domain. The cytoplasmic tail of the APC can comprise the APC-assembly domain. In some embodiments, the APC-assembly domain interacts with the ESCRT proteins TSG101, NEDD4, and/or ALIX. The systems, methods, compositions, and kits provided herein can, in some embodiments, be employed in concert with the systems, methods, compositions, and kits described in PCT Application Publication No. WO2022261230A1, the content of which is incorporated herein by reference in its entirety.

The APC-assembly domain can comprise or can be derived from a nonhuman protein, optionally a nonmammalian protein, further optionally a chicken protein, a mouse protein, a lizard protein, a reptile protein, a hamster protein, or a goldfish protein. The APC-assembly domain can comprise or can be derived from the ESCRT and ALIX binding region (EABR) of the human CEP55 protein, optionally residues 170-213. The APC-assembly domain can comprise or can be derived from Syntenin-1, rat Galectin-3 (rGalectin-3), Hrs, and/or CD2AP. The APC-assembly domain can comprise or can be derived from a viral protein, optionally a fragment of a viral protein, further optionally a retroviral protein, herpes simplex viral protein, vaccinia viral protein, hepadnaviral protein, togaviral protein, flaviviral protein, arenaviral protein, coronaviral protein, orthomyxoviral protein, paramyxoviral protein, bunyaviral protein, bornaviral protein, rhabdoviral protein or filoviral protein, optionally a Gag protein, further optionally derived from EIAV, HTLV-1, MLV, or MPMV, optionally EIAV p9 and/or HIV-1 p6. The APC-assembly domain can comprise or can be derived from: an Ebola protein, optionally EBOV VP40. The APC-assembly domain can comprise an amino acid sequence at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to

(SEQ ID NO: 33)
FNSSINNIHEMEIQLKDALEKNQQWLVYDQQREVYVKGLLAKIFEL
EKKTETAAHSLPHSLP.

In some embodiments, the nucleic acid composition does not comprise a polynucleotide encoding SpyTag or lentiviral Gag. In some embodiments, the APC-assembly domain do not comprise SpyTag or lentiviral Gag.

The APC can comprise an endocytosis-preventing motif (EPM) capable of preventing endocytosis of the APC. In some embodiments, the EPM tethers the APC to the cytoskeleton, thereby preventing localization to coated pits and endocytosis; enhances APV assembly, APV production, and/or APV secretion; and/or prevents endocytosis of the APC, thereby extending the time an APV remains at the plasma membrane to interact with ESCRT proteins. In some embodiments, an EPM disclosed herein is employed to generate fusion protein complex with APC, which can, in some embodiments, enhance the activity and/or potency of the APC (e.g., activating T cells), optionally at least about 1.1-fold. There are provided, in some embodiments, an EPM-APC fusion protein complex wherein an EPM is fused with an APC. There are provided, in some embodiments, nucleic acid compositions encoding one or more EPM-APC fusion protein complex. There are provided, in some embodiments, methods of treating a disease or disorder (e.g., a cancer, an autoimmune disease, or an infectious disease) comprising administering an effective amount of said nucleic acid compositions and/or population of APVs to a subject.

The EPM can be capable of: increasing the abundance and/or density of APCs on and/or in the APV by at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) as compared to an APV comprising an APC that does not comprise the EPM; and/or increasing the number of APVs secreted by a cell by at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) as compared to a cell expressing an APC that does not comprise the EPM.

The EPM can comprise or can be derived from a portion of murine low-affinity gamma Fc region receptor II isoform FcRII-B1. The EPM can comprise all or a portion of the cytoplasmic tail of FcRII-B1.

### The Antigen Presentation Polypeptide

Antigen presentation comprises the cellular and biochemical events necessary for the recognition and stimulation of CD4 and CD8 T lymphocytes. Antigen presentation requires the T cell receptor to engage the major histocompatibility complex (MHC) molecules together with the presenting peptide (e.g., a segment of the protein antigen). Naturally, the MHCs with a presenting peptide are displayed on the membrane of antigen-presenting cells. The receptor does not engage the antigen unless the antigen forms part of a biomolecular complex with the MHCs. In some embodiments, the antigen presentation polypeptide(s) is capable of forming a binding site that binds the presenting peptide.

There are two sets of MHC molecules, class I and class II, are required for the recognition of protein antigens. Antigens presented in association with class I MHC molecules are recognized by CD8 T cells, while the antigens in association with class II MHC molecules are recognized by CD4 T cells.

In some embodiments, the MHC can be derived from a mammal. Mammals includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the MHC is derived from a human. Human MHC is also known as Human Leukocyte Antigen (HLA). In some embodiments, the MHC comprises human leukocyte antigen (HLA) and/or mouse MHC. However, in some embodiments, the MHC is not derived from a human. In some embodiments, the MHC is derived from mouse. Mouse MHC is also called H-2.

The MHC Class I proteins (“MHCI” or “MHC-I”) are complex transmembrane glycoproteins composed of two main components: a heavy chain, also known as the α chain, and a smaller non-covalently associated protein called β2-microglobulin. The α chain is highly polymorphic. It can exist in various allelic forms leading to a diverse array of MHC-I molecules. The α chain consists of three domains: α1, α2, and α3. The α1 and α2 domains form a peptide-binding cleft, while the α3 domain anchors the complex to the cell membrane. The β2-microglobulin, which is non-polymorphic, is essential for the proper folding and stability of the MHC-I. In some embodiments, the MHC-I is mouse MHC-I or human MHC-I.

MHC Class II proteins (“MHCII” or “MHC-II”) are primarily expressed on antigen-presenting cells, including dendritic cells, macrophages, and B cells. The MHC-II consists of two transmembrane chains: an α chain and a β chain. Both chains are synthesized in the endoplasmic reticulum and form a heterodimeric complex. The α and β chains each have two domains: α1, α2, 31, and 02. In some embodiments, the MHC-II is mouse MHC-II.

The subclasses of the human HLA-I can be classified into HLA-A, HLA-B, HLA-C, HLA-E, HLA-F, and HLA-G. The sub-classes of human HLA-II can be classified into HLA-DMA, HLA-DMB, HLA-DOA, HLADOB, HLA-DPA1, HLA-DPB1, HLA-DQA1, HLADQB1, HLA-DRA, and HLA-DRB. In some embodiments, the antigen presentation polypeptide(s) are selected from MHC-I α chain, beta-2 microglobulin (β2M), MHC-II α chain, MHC-II β chain, a classical HLA Class I molecule, a non-classical HLA Class I molecule, a classical HLA Class II molecule, a non-classical HLA Class II molecule, HLA-A, HLA-B, HLA-C, HLA-E, HLA-F, HLA-G, HLA-DRA, HLA-DRB1, HLA-DRB3, HLA-DRB4, HLA-DRB5, HLA-DQA1, HLA-DQB1, HLA-DPA1, HLA-DPB1, HLA-DMA, HLA-DMB, HLA-DOA, HLA-DOB, derivatives thereof, homologs thereof, or any combination thereof.

HLA can be further classified based on serological typing and/or allele variations. In some embodiments, the HLA comprises serological typing A*02, A*03, A*11, A*23, A*24, A*26, A*31, A*33, A*34, A*43, A*66, and/or A*74. In some embodiments, the HLA comprises HLA A*11:01, HLA-A*03:01, and/or HLA-A*02:01. In some embodiments, the HLA is HLA-A*02:01. In some embodiments, the HLA class I is selected from an HLA allele selected from HLA-A*01:01, HLA-A*02:01, HLA-A*02:03, HLA-A*02:06, HLA-A*03:01, HLA-A*11:01, HLA-A*23:01, HLA-A*24:02, HLA-A*26:01, HLA-A*30:01, HLA-A*30:02, HLA-A*31:01, HLA-A*32:01, HLA-A*33:01, HLA-A*68:01, HLA-A*68:02, HLA-B*07:02, HLA-B*08:01, HLA-B*15:01, HLA-B*35:01, HLA-B*40:01, HLA-B*44:02, HLA-B*44:03, HLA-B*51:01, HLA-B*53:01, HLA-B*57:01, and HLA-B*58:01. In some embodiments, the HLA class II is selected from an HLA allele selected from HLA-DRB1*01:01, HLA-DRB1*15:01, HLA-DRB1*03:01, HLA-DRB1*04:01, HLA-DRB1*11:01, HLA-DRB1*13:01, HLA-DRB1*07:01, HLA-DRB1*01:01, HLA-DRB1*01:02, HLA-DRB1*04:02, HLA-DRB1*04:05, HLA-DRB1*04:07, HLA-DRB1*04:08, HLA-DRB1*08:04, HLA-DRB1*09:01, HLA-DRB1*10:01, HLA-DRB1*11:02, HLA-DRB1*11:03, HLA-DRB1*11:04, HLA-DRB1*15:02, HLA-DRB1*15:03, HLA-DRB1*16:01, HLA-DRB1*16:02, HLA-DRB3*02:02, HLA-DRB3*03:01, HLA-DRB5*01:01, HLA-DQA1*01:01/DQB1*05:01, HLA-DQA1*05:01/DQB1*02:01, HLA-DQA1*01:02/DQB1*05:02, HLA-DQA1*01:02/DQB1*06:02, HLA-DQA1*03:01/DQB1*03:02, HLA-DQA1*01:02/DQB1*06:04, HLA-DQA1*05:01/DQB1*03:01, HLA-DQA1*02:01/DQB1*02:02, HLA-DQA1*03:01/DQB1*03:01, HLA-DQA1*02:01/DQB1*03:03, HLA-DQA1*03:03/DQB1*03:03, HLA-DPA1*01:03/DPB1*01:01, HLA-DPA1*01:03/DPB1*02:01, HLA-DPA1*01:03/DPB1*03:01, HLA-DPA1*01:03/DPB1*04:01, HLA-DPA1*01:03/DPB1*04:02, HLA-DPA1*01:03/DPB1*05:01, HLA-DPA1*02:01/DPB1*01:01, HLA-DPA1*02:01/DPB1*02:01, HLA-DPA1*02:01/DPB1*03:01, HLA-DPA1*02:01/DPB1*04:01, HLA-DPA1*02:01/DPB1*04:02, HLA-DPA1*02:01/DPB1*05:01, HLA-DPA1*02:01/DPB1*06:01, HLA-DPA1*02:01/DPB1*09:01, HLA-DPA1*02:01/DPB1*11:01, HLA-DPA1*02:01/DPB1*13:01, HLA-DPA1*02:01/DPB1*14:01, HLA-DPA1*02:01/DPB1*15:01, and HLA-DPA1*02:01/DPB1*17:01.

Mouse MHC-I includes the “classical MHC class I” (also called MHC-Ia) that comprises H-2D, H-2K and H-2L subclasses, and the “non-classical MHC class I” (MHC-Ib) that comprises H-2Q, H-2M and H-2T subclasses. Mouse MHC-II includes the “classical MHC class II” (MHC-IIa) that includes H-2A (I-A), H-2E (I-E) and H-2P subclasses, and the “non-classical MHC class II” (MHC-IIb) comprises H-2M and H-20. The mouse MHC can be further classified into haplotypes that designated by a small letter (e.g., a, b, d, k, q, s, etc.). In some embodiments, the mouse MHC comprises H-2Dd, H-2Kd, H-2Ld, H-2Qa, H-2Qb, H-2Ad (I-Ad), H-2Ed (I-Ed), H-2Db, H-2Kb, H-2Ab (I-Ab), H-2Dk, H-2Kk, H-2Ak (I-Ak), H-2Ek (I-Ek), H-2Dp, H-2Kp, H-2Lp, H-2Ap (I-Ap), H-2Ep (I-Ep), H-2Dq, H-2Kq, H-2Lq, H-2Aq (I-Aq), H-2Eq (I-Eq), H-2Db, H-2Kb, H-2Ab (I-Ab), H-2Ds, H-2Dw3, H-2Kj, H-2jA (I-Aj), H-2Ej (I-Ej), H-2Af (I-Af), H-2Ag7 (I-Ag7), H-2Dz, H-2Ku, H-2Lz, H-2Au (I-Au), H-2Eu (I-Eu), H-2Dr, H-2Kr, H-2Lr, H-2Ar (I-Ar), H-2Er (I-Er), H-2Ds, H-2Ks, H-2Ls, H-2As (I-As), H-2Dv, H-2Kv, H-2Lv, H-2Av (I-Av), or H-2Ev (I-Ev). In some embodiments, the mouse MHC is H-2Kd, H-2Db, or I-Ab.

In some embodiments, the antigen presentation polypeptide(s) comprises an amino acid sequence having at least 65%, 70%, 75%, 80%, 85%, 90%, 95%, 98%, or 99% sequence identity to an amino acid sequence selected from SEQ ID NOs: 53-54, 57-58, 61-62, 81-86, 112-113, 120-121, 144-145, and 152-153, as listed in Table 2 below.

TABLE 2
Exemplary antigen presentation polypeptides

| | SEQ |
|---|---|
| Name | ID NO |
| β2M | 53-54 |
| D9 SCD β2M/HLA-A*02:01 | 57-58 |
| D9 HLA-A*02:01 HC | 61-62 |
| H-2Kd | 81 |
| H-2Kd heavy chain | 82 |
| A2_D9/H-2Db | 83 |
| A2_D9/H-2Db heavy chain | 84 |
| A2_D9/H-2Kb | 85 |
| A2_D9/H-2Kb heavy chain | 86 |
| Human sequence optimized Hgh/I-Ab-a | 112-113 |
| Human sequence optimized Hgh/I-Ab-b | 120-121 |
| Murine sequence optimized for mRNA Tx: I-Ab-a | 144-145 |
| Murine sequence optimized for mRNA Tx: I-Ab-b | 152-153 |

In some embodiments, the antigen presentation polypeptide comprises at least one mutation. In some embodiment, the mutation is an addition, a deletion, or a substitution. In some embodiments, the mutation is a point mutation. In some embodiments, the mutation is at positions equivalent to Y84 and/or A139 of HLA-A. In some embodiments, the mutation is Y84A, Y84C, and/or A139C.

### The Linker

The APC can comprise one or more linkers. The one or more linkers can comprise one or more flexible amino acid residues, optionally about 1, 2, 3, 4, 5, 6, 7, 8, 9, 10, 11, 12, 13, 14, 15, 16, 17, 18, 19, 20, 25, 30, 35, 40, 50, or a number or a range between any two of these values, flexible amino acid residues, further optionally the flexible amino acid residues comprise glycine, serine, or a combination thereof. The one or more linkers can be a glycine-serine (GS) linker, optionally 1, 2, 3, 4, 5, 6, 7, 8, 9, 10, 11, 12, 13, 14, 15, 16, 17, 18, 19, 20, 25, 30, 35, 40, 50, or a number or a range between any two of these values, amino acids in length. In some embodiments, the linker comprises no more than 25 amino acids. In some embodiments, the linkers is about 1, 2, 3, 4, 5, 6, 7, 8, 9, 11, 12, 15, 20, 25, amino acids in length.

The one or more linkers can be situated between the APC-assembly domain and the presenting peptide, between the APC-assembly domain and the EPM, between the APC-assembly domain and the antigen presentation polypeptide, between the EPM and the antigen presentation polypeptide, between the presenting peptide and the EPM, and/or between the presenting peptide and the antigen presentation polypeptide.

In some embodiments, the linker is a flexible linker. In some embodiments, the flexible linker is a G/S-rich linker. In some embodiments, the flexible linker is a peptide (G/S)n. In some embodiments, n is a position integer that is smaller than 21. In some embodiments, the linker is, or comprises, one or more of GSGSGSG (SEQ ID NO: 1), GGGGS (SEQ ID NO: 2), GSGSG (SEQ ID NO: 3), GGGG (SEQ ID NO: 4), GGG, GG, GS, GSGS (SEQ ID NO: 8), GGGS (SEQ ID NO: 9), GGS, GTS, GGSGGS (SEQ ID NO: 12), GGG, GGGGGG (SEQ ID NO: 14), GGGGGGGGG (SEQ ID NO: 15), GGGGGGGGGGGG (SEQ ID NO: 16), GGGGGGGGGGGGGGG (SEQ ID NO: 17), GGS, GGSGGS (SEQ ID NO: 19), GGSGGSGGS (SEQ ID NO: 20), GGSGGSGGSGGS (SEQ ID NO: 21), GGSGGSGGSGGSGGS (SEQ ID NO: 22), GSG, GSGGSG (SEQ ID NO: 24), GSGGSGGSG (SEQ ID NO: 25), GSGGSGGSGGSG (SEQ ID NO: 26), GSGGSGGSGGSGGSG (SEQ ID NO: 27), (GGGGS)n (SEQ ID NO: 28), GGGGSGGGGS (SEQ ID NO: 29), GGGGSGGGGSGGGGS (SEQ ID NO: 30), GGGGSGGGGSGGGGSGGGGS (SEQ ID NO: 31), and GGGGSGGGGSGGGGSGGGGSGGGGS (SEQ ID NO: 32). In some embodiments, the linker is, or comprises, GGGGS (SEQ ID NO: 2), and/or GGGS (SEQ ID NO: 9). In some embodiments, the linker is, or comprises, n GGGGS (SEQ ID NO: 2) or GGGS (SEQ ID NO: 9). In some embodiments, n is an integer from 1 to 10, optionally n is 3 or 4. In some embodiments, the linker is, or comprises GGGGSGGGGSGGGGS (SEQ ID NO: 30), and/or GGGGSGGGGSGGGGSGGGGS (SEQ ID NO: 31).

### The Presenting Peptide

The presenting peptides, or a portion thereof, presented on the APVs herein described can be displayed on its surface. Alternatively, the presenting peptide presented on the APVs herein described can be partially encapsulated or embedded such that at least an immunogenic portion of the presenting peptide is exposed and accessible by an immune cell receptor so as to induce an immune response. The presenting peptide, or a portion thereof, can be displayed in and/or on the surface of the APV. The presenting peptide can be about 1 to about 10000 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, or a number or a range between any of these values) amino acids in length. In some embodiments, the presenting peptide is 5-20 amino acids in length.

The presenting peptide can comprise or can be derived from an antigenic protein associated with a disease or disorder, optionally an immunogenic variant and/or an immunogenic fragment of said antigenic protein. The presenting peptide can comprise or be derived from at least a portion of an antigenic protein. The presenting peptide can comprise or can be derived from a conserved portion of said antigenic protein. The presenting peptide can be present on and/or in the APV in its natural membrane-associated conformation. The presenting peptide can comprise or can be derived from at least about 5 percent (e.g., 5%, 6%, 7%, 8%, 9%, 10%, 11%, 12%, 13%, 14%, 1%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) of the full length of said antigenic protein. The presenting peptide can comprise a membrane protein. In some embodiments, the presenting peptide is not configured to be a soluble protein. In some embodiments, the presenting peptide does not comprise one or more mutations configured to enhance its solubility and/or stability. In some embodiments, the presenting peptide is present on and/or in the APV in its natural membrane-associated conformation. In some embodiments, antigenic proteins/peptides provided herein can be intra-cellular, membrane bound or extra-cellular.

Accordingly, the presenting peptides can contain amino acid substitutions relative to the antigenic proteins disclosed herein. Any amino acid substitution is permissible so long as the immunogenic activity of the protein is not significantly altered (e.g., at most 10%, 20%, 30%, 40% or 50% decrease relative to the coronavirus protein antigens disclosed herein) and the variants retain the desired activity. Preferred variants typically contains substitutions with one or more amino acids substituted with their functional equivalents. In some embodiments, the presenting peptide does not comprise a transmembrane domain and/or is a soluble protein, and the APC comprises a transmembrane domain.

In some embodiments, the presenting peptide is derived from one or more of MART-1; NYESO1; neuropilin (NRP); IGRP; CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp10O); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some embodiments, the presenting peptide comprise the nucleotide sequence of any one of SEQ ID NOs: 184-266 or a sequence that is at least 80%, 85%, 90%, 95%, 98%, 99%, or 100% identical to any one of SEQ ID NOs: 184-266. The sequences of exemplary presenting peptides are in Table 3 below.

TABLE 3
Exemplary presenting peptides

| SEQ ID NO | Name | Antigen/Type | Sequence |
|---|---|---|---|
| 184 | 10-mer MART1 | MART1 | ELAGIGILTV |
| 185 | 9-mer NYESO1 | NYESO1 | SLLMWITQV |
| 186 | Ealpha peptide (Ea) | Ealpha | ASFEAQGALANIAVDKA |
| 187 | pMOG peptide | pMOG | GWYRSPFSRVVH |
| 188 | DSG3 78-94 | DSG3 | QATQKITYRISGVGIDQ |
| 189 | DSG3 96-112 | DSG3 | PFGIFVVDKNTGDINIT |
| 190 | DSG3 189-205 | DSG3 | HLNSKIAFKIVSQEPAG |
| 191 | DSG3 205-221 | DSG3 | GTPMFLLSRNTGEVRTL |
| 192 | DSG3 250-266 | DSG3 | QCECNIKVKDVNDNFPM |
| 193 | DSG3 342-358 | DSG3 | SVKLSIAVKNKAEFHQS |
| 194 | DSG3 376-392 | DSG3 | NVREGIAFRPASKTFTV |
| 195 | DSG3 483-499 | DSG3 | RDSTFIVNKTITAEVLA |
| 196 | p33 | DSG3 | LLSRNTGEVRTLTNSLD |
| 197 | ADAMTSL5 57-65 | ADAMTSL5 | VRSRRCLRL |
| 198 | HIP4 | HIP | GQVELGGGGIVEQCC |
| 199 | HIP5 | HIP | GQVELGGGTPIESHQ |
| 200 | HIP6 | HIP | GQVELGGGNAVEVLK |
| 201 | HIP8 | HIP | GQVELGGGSSPETLI |
| 202 | HIP11 | HIP | SLQPLALEAEDLQV |
| 203 | HIP12 | HIP | SLQPLALGIVEQCC |
| 204 | HIP14 | HIP | SLQPLALNAVEVLK |
| 205 | HIP16 | HIP | SLQPLALSSPETLI |
| 206 | insB: 9-23 | B-Chain | SHL VEALYL VCGER |
| 207 | insB: 9-23 R22E | B-Chain | SHL VEAL YL VCGEE |
| 208 | insB: 11-23 | B-Chain | LVEALYLVCGERG |
| 209 | insB: 11-23 R22E | B-Chain | LVEALYLVCGEEG |
| 210 | Cpept-IAPP2 | HIP | GQVELGGGNAVEVLK |
| 211 | Cpept-NeuropeptY | HIP | GQVELGGGSSPETLI |
| 212 | Cpept-InsA | HIP | GQVELGGGGIVEQCC |
| 213 | Cpept-IAPP1 | HIP | GQVELGGGTPIESHQ |
| 214 | InsB-SG1 | HIP | VCGERGFFEEL VARSE |
| 215 | InsB(1)-SG1 | HIP | HL VEAL YLEEL VARSE |
| 216 | InsA-InsB | HIP | ICSLYQLEFVNQHLCG |
| 217 | Cpept/InsA-SG1 | HIP | SLQKRGIVEELVARSE |
| 218 | InsA-SGV | HIP | CSLYQLENSVPHFSDE |
| 219 | Cpept-GRP78 | HIP | QPLALEGSALSSQHQA |
| 220 | B3.1 | C-Peptide | PGAGSLQPLALE |
| 221 | B3.3 | C-Peptide | SLQPLALEGSL |
| 222 | K3.2 | C-Peptide | QPLALEGSL |
| 223 | K4.4 | C-Peptide | QPLALEGSLQ |
| 224 | K6.2 | C-Peptide | GAGSLQPLAL |
| 225 | K9.6 | C-Peptide | QVELGGGPGAG |
| 226 | D1.1 | C-Peptide | AEDLQVGQVE |
| 227 | T6.1 | C-Peptide | SLQPLALEGSLQ |
| 228 | T6.6 | C-Peptide | LALEGSL |
| 229 | H3.7 | C-Peptide | AGSLQPLAL |
| 230 | H6.4 | C-Peptide | SLQPLALEGS |
| 231 | H11.5 | C-Peptide | VELGGGPGAG |
| 232 | E2.5 | C-Peptide | EDLQVGQVELGG |
| 233 | DQ8-1.9 | C-Peptide | VELGGGPGA |
| 234 | DQ8-4.13 | C-Peptide | QVELGGGPGAG |
| 235 | DQ8-5.5 | C-Peptide | QVELGGGPG |
| 236 | DQ8-1.1 | C-Peptide | AGSLQPLALE |
| 237 | DQ8-1.2 | C-Peptide | AGSLQPLAL |
| 238 | DQ8-2.4 | C-Peptide | SLQPLALEGSL |
| 239 | HIP-B | HIP | VGQVELGGGPIVEQCC |
| 240 | HIP-E | HIP | VGQVELGGGPALEGSL |
| 241 | HIP-G | HIP | VELGGGPGAEAEDLQ |
| 242 | HIP-I | HIP | LQPLALEGSLLALEGS |
| 243 | HIP-J | HIP | QPLALEGLQVGQVELG |
| 244 | HIP-L | HIP | EAEDLQVGLALEGSLQ |
| 245 | NRP-V7 | NRP | KYNKANVFL |
| 246 | IGRP 206-214 | IGRP | VYLKTNVFL |
| 247 | IGRP 265-273 | IGRP | VLFGLGFAI |
| 248 | IGRP 211-219 | IGRP | NLFLFLFAV |
| 249 | IGRP 215-223 | IGRP | FLFAVGFYL |
| 250 | IGRP 222-230 | IGRP | YLLLRVLNI |
| 251 | GAD65 1-12 | GAD65 | MASPGSGFWSFG |
| 252 | GAD65 19-30 | GAD65 | DSENPGTARAWC |
| 253 | GAD65 25-36 | GAD65 | TARAWCQVAQKF |
| 254 | GAD65 193-204 | GAD65 | ADWLTSTANTNM |
| 255 | GAD65 211-222 | GAD65 | PVFVLLEYVTLK |
| 256 | GAD65 265-276 | GAD65 | KGMAALPRLIAF |
| 257 | GAD65 349-360 | GAD65 | AVADICKKYKIW |
| 258 | GAD65 367-378 | GAD65 | WGGGLLMSRKHK |
| 259 | GAD65 373-384 | GAD65 | MSRKHKWKLSGV |
| 260 | GAD65 445-456 | GAD65 | QCGRHVDVFKLW |
| 261 | GAD65 499-510 | GAD65 | QCGRHVDVFKLW |
| 262 | GAD65p217 | GAD65 | EYVTLKKMREIIGWPGGSGD |
| 263 | GAD65p290 | GAD65 | AALGIGTDSVILIKCDERGK |
| 264 | GAD65 555-567 557I | GAD65 | NFIRMVISNPAAT |
| 265 | GAD65p121 | GAD65 | YVVKSFDRSTKVIDFHYPNE |
| 266 | GAD65p270 | GAD65 | LPRLIAFTSEHSHFSLKKGA |

### Pathogenic Antigens

The presenting peptide can comprise or can be derived from the full-length surface protein of an infectious agent. The disease or disorder can be an infectious disease or disorder caused by an infectious agent, the presenting peptide can comprise or can be derived from an antigenic protein of said infectious agent, and the antigenic protein of said infectious agent can be a pathogenic antigen. In some embodiments, the pathogenic antigen is selected from: Outer membrane protein A OmpA, biofilim associated protein Blap, transport protein MucK (Acinetobacter baumannii, Acinetobacter infections)); variable surface glycoprotein VSG, microtubule-associated protein MAPP15, trans-sialidase TSA (Trypanosoma brucei, African sleeping sickness (African trypanosomiasis)); HIV β24 antigen, HIV envelope proteins (Gp120, Gp41, Gp160), polyprotein GAG, negative factor protein Nef, trans-activator of transcription Tat (HIV (Human immunodeficiency virus), AIDS (Acquired immunodeficiency syndrome)); galactose-inhibitable adherence protein GIAP, 29 kDa antigen Eh29, Gal/GaINAc lectin, protein CRT, 125 kDa immunodominant antigen, protein M17, adhesin ADH112, protein STIRP (Entamoeba histolytica, Amoebiasis); Major surface proteins 1-5 (MSP1a, MSP1b, MSP2, MSP3, MSP4, MSP5), type IV secreotion system proteins (VirB2, VirB7, VirBi11, VirD4) (Anaplasma genus, Anaplasmosis); protective Antigen PA, edema factor EF, lethal facotor LF, the S-layer homology proteins SLH (Bacillus anthracis, Anthrax); acranolysin, phospholipase D, collagen-binding protein CbpA (Arcanobacterium haemolyticum, Arcanobacterium haemolyticum infection); nucleocapsid protein NP, glycoprotein precursor GPC, glycoprotein GP1, glycoprotein GP2 (Junin virus, Argentine hemorrhagic fever); chitin-protein layer proteins, 14 kDa suarface antigen A14, major sperm protein MSP, MSP polymerization-organizing protein MPOP, MSP fiber protein 2 MFP2, MSP polymerization-activating kinase MPAK, ABA-1-like protein ALB, protein ABA-1, cuticulin CUT-1 (Ascaris lumbricoides, Ascariasis); 41 kDa allergen Asp v13, allergen Asp f3, major conidial surface protein rodlet A, protease Pep1p, GPI-anchored protein Gellp, GPI-anchored protein Crap (Aspergillus genus, Aspergillosis); family VP26 protein, VP29 protein (Astroviridae, Astrovirus infection); Rhoptry-associated protein 1 RAP-1, merozoite surface antigens MSA-1, MSA-2 (α1, α2, c), 12D3, 1105, 21134, P29, variant erythrocyte surface antigen VESA1, Apical Membrane Antigen 1 AMA-1 (Babesia genus, Babesiosis); hemolysin, enterotoxin C, PXO1-51, glycolate oxidase, ABC-transporter, penicillin-bingdn protein, zinc transporter family protein, pseudouridine synthase Rsu, plasmid replication protein RepX, oligoendopeptidase F, prophage membrane protein, protein HemK, flagellar antigen H, 28.5-kDa cell surface antigen (Bacillus cereus, Bacillus cereus infection); large T antigen LT, small T antigen, capsid protein VP1, capsid protein VP2 (BK virus, BK virus infection); 29 kDa-protein, caspase-3-like antigens, glycoproteins (Blastocystis hominis, Blastocystis hominis infection); yeast surface adhesin WI-1 (Blastomyces dermatitidis, Blastomycosis); nucleoprotein N, polymerase L, matrix protein Z, glycoprotein GP (Machupo virus, Bolivian hemorrhagic fever); outer surface protein A OspA, outer surface protein OspB, outer surface protein OspC, decorin binding protein A DbpA, decorin binding protein B DbpB, flagellar filament 41 kDa core protein Fla, basic membrane protein A precursor BmpA (Immunodominant antigen P39), outer surface 22 kDa lipoprotein precursor (antigen IPLA7), variable surface lipoprotein vlsE (Borrelia genus, Borrelia infection); Botulinum neurotoxins BoNT/A1, BoNT/A2, BoNT/A3, BoNT/B, BoNT/C, BoNT/D, BoNT/E, BoNT/F, BoNT/G, recombinant botulinum toxin F He domain FHc (Clostridium botulinum, Botulism (and Infant botulism)); nucleocapsid, glycoprotein precursor (Sabia virus, Brazilian hemorrhagic fever); copper/Zinc superoxide dismutase SodC, bacterioferritin Bfr, 50S ribosomal protein RplL, OmpA-like transmembrane domain-containing protein Omp31, immunogenic 39-kDa protein M5 P39, zinc ABC transporter periplasmic zinc-bnding protein znuA, periplasmic immunogenic protein Bp26, 30S ribosomal protein S12 RpsL, glyceraldehyde-3-phosphate dehydrogenase Gap, 25 kDa outer-membrane immunogenic protein precursor Omp25, invasion protein B laIB, trigger factor Tig, molecular chaperone DnaK, putative peptidyl-prolyl cis-trans isomerase SurA, lipoprotein Omp19, outer membrane protein MotY Omp16, conserved outer membrane protein D15, malate dehydrogenase Mdh, component of the Type-IV secretion system (TOSS) VirJ, lipoprotein of unknown function BAB1_0187 (Brucella genus, Brucellosis); members of the ABC transporter family (LoIC, OppA, and PotF), putative lipoprotein releasing system transmembrane protein LoIC/E, flagellin FliC, Burkholderia intracellular motility A BimA, bacterial Elongation factor-Tu EF-Tu, 17 kDa OmpA-like protein, boaA coding protein, boaB coding protein (Burkholderia cepacia and other Burkholderia species, Burkholderia infection); mycolyl-transferase Ag85A, heat-shock protein Hsp65, protein TB10.4, 19 kDa antigen, protein PstS3, heat-shock protein Hsp70 (Mycobacterium ulcerans, Buruli ulcer); norovirus major and minor viral capsid proteins VP1 and VP2, genome polyprotein, Sapoviurus capsid protein VP1, protein Vp3, geome polyprotein (Caliciviridae family, Calicivirus infection (Norovirus and Sapovirus)); major outer membrane protein PorA, flagellin FlaA, surface antigen CjaA, fibronectin binding protein CadF, aspartate/glutamate-binding ABC transporter protein PeblA, protein FspA1, protein FspA2 (Campylobacter genus, Campylobacteriosis); glycolytic enzyme enolase, secreted aspartyl proteinases SAP1-10, glycophosphatidylinositol (GPI)-linked cell wall protein, protein Hyrl, complement receptor 3-related protein CR3-RP, adhesin Als3p, heat shock protein 90 kDa hsp90, cell surface hydrophobicity protein CSH (usually Candida albicans and other Candida species, Candidiasis); 17-kDa antigen, protein P26, trimeric autotransporter adhesins TAAs, Bartonella adhesin A BadA, variably expressed outer-membrane proteins Vomps, protein Pap3, protein HbpA, envelope-associated protease HtrA, protein OMP89, protein GroEL, protein LaIB, protein OMP43, dihydrolipoamide succinyltransferase SucB (Bartonella henselae, Cat-scratch disease); amastigote surface protein-2, amastigote-specific surface protein SSP4, cruzipain, trans-sialidase TS, trypomastigote surface glycoprotein TSA-1, complement regulatory protein CRP-10, protein G4, protein G2, paraxonemal rod protein PAR2, paraflagellar rod component Part, mucin-Associated Surface Proteins MPSP (Trypanosoma cruzi, Chagas Disease (American trypanosomiasis)); envelope glycoproteins (gB, gC, gE, gH, gI, gK, gL) (Varicella zoster virus (VZV), Chickenpox); major outer membrane protein MOMP, probable outer membrane protein PMPC, outer membrane complex protein B OmcB, heat shock proteins Hsp60 HSP10, protein IncA, proteins from the type III secretion system, ribonucleotide reductase small chain protein NrdB, plasmid protein Pgp3, chlamydial outer protein N CopN, antigen CT521, antigen CT425, antigen CT043, antigen TC0052, antigen TC0189, antigen TC0582, antigen TC0660, antigen TC0726, antigen TC0816, antigen TC0828 (Chlamydia trachomatis, Chlamydia); low calcium response protein E LCrE, chlamydial outer protein N CopN, serine/threonine-protein kinase PknD, acyl-carrier-protein S-malonyltransferase FabD, single-stranded DNA-binding protein Ssb, major outer membrane protein MOMP, outer membrane protein 2 Omp2, polymorphic membrane protein family (Pmp1, Pmp2, Pmp3, Pmp4, Pmp5, Pmp6, Pmp7, Pmp8, Pmp9, Pmp10, Pmp11, Pmp12, Pmp13, Pmp14, Pmp15, Pmp16, Pmp17, Pmp18, Pmp19, Pmp20, Pmp21) (Chlamydophila pneumoniae, Chlamydophila pneumoniae infection); cholera toxin B CTB, toxin coregulated pilin A TcpA, toxin coregulated pilin TcpF, toxin co-regulated pilus biosynthesis ptrotein F TcpF, cholera enterotoxin subunit A, cholera enterotoxin subunit B, Heat-stable enterotoxin ST, mannose-sensitive hemagglutinin MSHA, outer membrane protein U Porin ompU, Poring B protein, polymorphic membrane protein-D (Vibrio cholerae, Cholera); propionyl-CoA carboxylase PCC, 14-3-3 protein, prohibitin, cysteine proteases, glutathione transferases, gelsolin, cathepsin L proteinase CatL, Tegumental Protein 20.8 kDa TP20.8, tegumental protein 31.8 kDa TP31.8, lysophosphatidic acid phosphatase LPAP, (Clonorchis sinensis, Clonorchiasis); surface layer proteins SLPs, glutamate dehydrogenase antigen GDH, toxin A, toxin B, cysteine protease Cwp84, cysteine protease Cwp13, cysteine protease Cwp19, Cell Wall Protein CwpV, flagellar protein FliC, flagellar protein FliD (Clostridium difficile, Clostridium difficile infection); rhinoviruses: capsid proteins VP1, VP2, VP3, VP4; coronaviruses: sprike proteins S, envelope proteins E, membrane proteins M, nucleocapsid proteins N (usually rhinoviruses and coronaviruses, Common cold (Acute viral rhinopharyngitis; Acute coryza)); prion protein Prp (CID prion, Creutzfeldt-Jakob disease (CJD)); envelope protein Gc, envelope protein Gn, nucleocapsid proteins (Crimean-Congo hemorrhagic fever virus, Crimean-Congo hemorrhagic fever (CCHF)); virulence-associated DEAD-box RNA helicase VAD1, galactoxylomannan-protein GaIXM, glucuronoxylomannan GXM, mannoprotein MP (Cryptococcus neoformans, Cryptococcosis); acidic ribosomal protein P2 CpP2, mucin antigens Mucd, Muc2, Muc3 Muc4, Muc5, Muc6, Muc7, surface adherence protein CP20, surface adherence protein CP23, surface protein CP12, surface protein CP21, surface protein CP40, surface protein CP60, surface protein CP15, surface-associated glycopeptides gp40, surface-associated glycopeptides gp15, oocyst wall protein AB, profilin PRF, apyrase (Cryptosporidium genus, Cryptosporidiosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, adult-specific secreted factor Xa serine protease inhibitor anticoagulant AP, cathepsin D-like aspartic protease ARR-1 (usually Ancylostoma braziliense; multiple other parasites, Cutaneous larva migrans (CLM)); cathepsin L-like proteases, 53/25-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia solium, Cysticercosis); pp65 antigen, membrane protein pp15, capsid-proximal tegument protein pp150, protein M45, DNA polymerase UL54, helicase UL105, glycoprotein gM, glycoprotein gN, glcoprotein H, glycoprotein B gB, protein UL83, protein UL94, protein UL99 (Cytomegalovirus (CMV), Cytomegalovirus infection); capsid protein C, premembrane protein prM, membrane protein M, envelope protein E (domain I, domain II, domain II), protein NS1, protein NS2A, protein NS2B, protein NS3, protein NS4A, protein 2K, protein NS4B, protein NS5 (Dengue viruses (DEN-1, DEN-2, DEN-3 and DEN-4)-Flaviviruses, Dengue fever); 39 kDa protein (Dientamoeba fragilis, Dientamoebiasis); diphtheria toxin precursor Tox, diphteria toxin DT, pilin-specific sortase SrtA, shaft pilin protein SpaA, tip pilin protein SpaC, minor pilin protein SpaB, surface-associated protein DIP1281 (Corynebacterium diphtheriae, Diphtheria); glycoprotein GP, nucleoprotein NP, minor matrix protein VP24, major matrix protein VP40, transcription activator VP30, polymerase cofactor VP35, RNA polymerase L (Ebolavirus (EBOV), Ebola hemorrhagic fever); prion protein (vCJD prion, Variant Creutzfeldt-Jakob disease (vCJD, nvCJD)); UvrABC system protein B, protein Flp1, protein Flp2, protein Flp3, protein TadA, hemoglobin receptor HgbA, outer membrane protein TdhA, protein CpsRA, regulator CpxR, protein SapA, 18 kDa antigen, outer membrane protein NcaA, protein LspA, protein LspA1, protein LspA2, protein LspB, outer membrane component DsrA, lectin DItA, lipoprotein Hip, major outer membrane protein OMP, outer membrane protein OmpA2 (Haemophilus ducreyi, Chancroid); aspartyl protease 1 Pep1, phospholipase B PLB, alpha-mannosidase 1 AMN1, glucanosyltransferase GEL1, urease URE, peroxisomal matrix protein Pmp1, proline-rich antigen Pra, humal T-cell relative protein TcrP (Coccidioides immitis and Coccidioides posadasii, Coccidioidomycosis); allergen Tri r 2, heat shock protein 60 Hsp60, fungal actin Act, antigen Tri r2, antigen Tri r4, antigen Tri t1, protein IV, glycerol-3-phosphate dehydrogenase Gpd1, osmosensor HwSholA, osmosensor HwSho1B, histidine kinase HwHhk7B, allergen Mala s 1, allergen Mala s 11, thioredoxin Trx Mala s 13, allergen Mala f, allergen Mala s (usually Trichophyton spp, Epidermophyton spp., Malassezia spp., Hortaea werneckii, Dermatophytosis); protein EG95, protein EG10, protein EG18, protein EgA31, protein EM18, antigen EPC1, antigen B, antigen 5, protein P29, protein 14-3-3, 8-kDa protein, myophilin, heat shock protein 20 HSP20, glycoprotein GP-89, fatty acid binding protein FAPB (Echinococcus genus, Echinococcosis); major surface protein 2 MSP2, major surface protein 4 MSP4, MSP variant SGV1, MSP variant SGV2, outer membrane protein OMP, outer membrande protein 19 OMP-19, major antigenic protein MAP1, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia genus, Ehrlichiosis); secreted antigen SagA, sagA-like proteins SalA and SaIB, collagen adhesin Scm, surface proteins Fmsl (EbpA(fm), Fms5 (EbpB(fm), Fms9 (EpbC(fm) and Fms10, protein EbpC(fm), 96 kDa immunoprotective glycoprotein G1 (Enterococcus genus, Enterococcus infection); genome polyprotein, polymerase 3D, viral capsid protein VP1, viral capsid protein VP2, viral capsid protein VP3, viral capsid protein VP4, protease 2A, protease 3C (Enterovirus genus, Enterovirus infection); outer membrane proteins OM, 60 kDa outer membrane protein, cell surface antigen OmpA, cell surface antigen OmpB (sca5), 134 kDa outer membrane protein, 31 kDa outer membrane protein, 29.5 kDa outer membrane protein, cell surface protein SCA4, cell surface protein Adr1 (RP827), cell surface protein Adr2 (RP828), cell surface protein SCAl, Invasion protein invA, cell division protein fts, secretion proteins sec Ofamily, virulence proteins virB, tlyA, tlyC, parvulin-like protein Plp, preprotein translocase SecA, 120-kDa surface protein antigen SPA, 138 kD complex antigen, major 100-kD protein (protein I), intracytoplasmic protein D, protective surface protein antigen SPA (Rickettsia prowazekii, Epidemic typhus); Epstein-Barr nuclear antigens (EBNA-1, EBNA-2, EBNA-3A, EBNA-3B, EBNA-3C, EBNA-leader protein (EBNA-LP)), latent membrane proteins (LMP-1, LMP-2A, LMP-2B), early antigen EBV-EA, membrane antigen EBV-MA, viral capsid antigen EBV-VCA, alkaline nuclease EBV-AN, glycoprotein glycoprotein gp350, glycoprotein gp110, glycoprotein gp42, glycoprotein gHgL, glycoprotein gB (Epstein-Barr Virus (EBV), Epstein-Barr Virus Infectious Mononucleosis); cpasid protein VP2, capsid protein VP1, major protein NS1 (Parvovirus B19, Erythema infectiosum (Fifth disease)); pp65 antigen, glycoprotein 105, major capsid protein, envelope glycoprotein H, protein U51 (Human herpesvirus 6 (HHV-6) and Human herpesvirus 7 (HHV-7), Exanthem subitum); thioredoxin-glutathione reductase TGR, cathepsins L1 and L2, Kunitz-type protein KTM, leucine aminopeptidase LAP, cysteine proteinase Fast, saposin-like protein-2 SAP-2, thioredoxin peroxidases TPx, Prx-1, Prx-2, cathepsin I cysteine proteinase CL3, protease cathepsin L CL1, phosphoglycerate kinase PGK, 27-kDa secretory protein, 60 kDa protein HSP35alpha, glutathione transferase GST, 28.5 kDa tegumental antigen 28.5 kDa TA, cathepsin B3 protease CatB3, Type I cystatin stefin-1, cathepsin L5, cathepsin Llg and cathepsin B, fatty acid binding protein FABP, leucine aminopeptidases LAP (Fasciola hepatica and Fasciola gigantica, Fasciolosis); prion protein (FFI prion, Fatal familial insomnia (FFI)); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, Thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, secreted larval acidic proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, Cox-2 (Filarioidea superfamily, Filariasis); phospholipase C PLC, heat-labile enterotoxin B, Iota toxin component Ib, protein CPE1281 pyruvate ferredoxin oxidoreductase, elongation factor G EF-G, perfringolysin 0 Pfo, glyceraldehyde-3-phosphate dehydrogenase GapC, Fructose-bisphosphate aldolase Alf2, Clostridium perfringens enterotoxin CPE, alpha toxin AT, alpha toxoid ATd, epsilon-toxoid ETd, protein HP, large cytotoxin TpeL, endo-beta-N-acetylglucosaminidase Naglu, phosphoglyceromutase Pgm (Clostridium perfringens, Food poisoning by Clostridium perfringens); leukotoxin IktA, adhesion FadA, outer membrane protein RadD, high-molecular weight arginine-binding protein (Fusobacterium genus, Fusobacterium infection); phospholipase C PLC, heat-labile enterotoxin B, Iota toxin component Ib, protein CPE1281, pyruvate ferredoxin oxidoreductase, elongation factor G EF-G, perfringolysin 0 Pfo, glyceraldehyde-3-phosphate dehydrogenase GapC, fructose-bisphosphate aldolase Alf2, Clostridium perfringens enterotoxin CPE, alpha toxin AT, alpha toxoid ATd, epsilon-toxoid ETd, protein HP, large cytotoxin TpeL, endo-beta-N-acetylglucosaminidase Naglu, phosphoglyceromutase Pgm (usually Clostridium perfringens; other Clostridium species, Gas gangrene (Clostridial myonecrosis)); lipase A, lipase B, peroxidase Deci (Geotrichum candidum, Geotrichosis); prion protein (GSS prion, Gerstmann-Straussler-Scheinker syndrome (GSS)); cyst wall proteins CWP1, CWP2, CWP3, variant surface protein VSP, VSP1, VSP2, VSP3, VSP4, VSP5, VSP6, 56 kDa antigen, pyruvate ferredoxin oxidoreductase PFOR, alcohol dehydrogenase E ADHE, alpha-giardin, alpha8-giardin, alpha1-guiardin, beta-giardin, cystein proteases, glutathione-S-transferase GST, arginine deiminase ADI, fructose-1,6-bisphosphat aldolase FBA, Giardia trophozoite antigens GTA (GTA1, GTA2), ornithine carboxyl transferase OCT, striated fiber-asseblin-like protein SALP, uridine phosphoryl-like protein UPL, alpha-tubulin, beta-tubulin (Giardia intestinalis, Giardiasis); members of the ABC transporter family (LoIC, OppA, and PotF), putative lipoprotein releasing system transmembrane protein LoIC/E, flagellin FliC, Burkholderia intracellular motility A BimA, bacterial Elongation factor-Tu EF-Tu, 17 kDa OmpA-like protein, boaA coding protein (Burkholderia mallei, Glanders); cyclophilin CyP, 24 kDa third-stage larvae protien GS24, excretion-secretion products ESPs (40, 80, 120 and 208 kDa) (Gnathostoma spinigerum and Gnathostoma hispidum, Gnathostomiasis); pilin proteins, minor pilin-associated subunit pilC, major pilin subunit and variants pilE, pilS, phase variation protein porA, Porin B PorB, protein TraD, Neisserial outer membrane antigen H.8, 70 kDa antigen, major outer membrane protein PI, outer membrane proteins PIA and PIB, W antigen, surface protein A NspA, transferrin binding protein TbpA, transferrin binding protein TbpB PBP2, mtrR coding protein, ponA coding protein, membrane permease FbpBC, FbpABC protein system, LbpAB proteins, outer membrane protein Opa, outer membrane transporter FetA, iron-repressed regulator MpeR (Neisseria gonorrhoeae, Gonorrhea); outer membrane protein A OmpA, outer membrane protein C OmpC, outer membrane protein K17 OmpK17 (Klebsiella granulomatis, Granuloma inguinale (Donovanosis)); fibronectin-binding protein Sfb, fibronectin/fibrinogen-binding protein FBP54, fibronectin-binding protein FbaA, M protein type 1 Emml, M protein type 6 Emm6, immunoglobulin-binding protein 35 Sib35, Surface protein R28 Spr28, superoxide dismutase SOD, C5a peptidase ScpA, antigen I/II AgI/II, adhesin AspA, G-related alpha2-macroglobulin-binding protein GRAB, surface fibrillar protein M5 (Streptococcus pyogenes, Group A streptococcal infection); C protein R antigen, arginine deiminase proteins, adhesin BibA, 105 kDA protein BPS, surface antigens c, surface antigens R, surface antigens X, trypsin-resistant protein R1, trypsin-resistant protein R3, trypsin-resistant protein R4, surface immunogenic protein Sip, surface protein Rib, Leucine-rich repeats protein LrrG, serine-rich repeat protein Srr-2, C protein alpha-antigen Bca, Beta antigen Bag, surface antigen Epsilon, alpha-like protein ALP1, alpha-like protein ALP5 surface antigen delta, alpha-like protein ALP2, alpha-like protein ALP3, alpha-like protein ALP4, Cbeta protein Bac (Streptococcus agalactiae, Group B streptococcal infection); transferrin-binding protein 2 Tbp2, phosphatase P4, outer membrane protein P6, peptidoglycan-associated lipoprotein Pal, protein D, protein E, adherence and penetration protein Hap, outer membrane protein 26 Omp26, outer membrane protein P5 (Fimbrin), outer membrane protein D15, outer membrane protein OmpP2, 5′-nucleotidase NucA, outer membrane protein P1, outer membrane protein P2, outer membrane lipoprotein Pcp, Lipoprotein E, outer membrane protein P4, fuculokinase FucK, [Cu,Zn]-superoxide dismutase SodC, protease HtrA, protein 0145, alpha-galactosylceramide (Haemophilus influenzae, Haemophilus influenzae infection); polymerase 3D, viral capsid protein VP1, viral capsid protein VP2, viral capsid protein VP3, viral capsid protein VP4, protease 2A, protease 3C (Enteroviruses, mainly Coxsackie A virus and Enterovirus 71 (EV71), Hand, foot and mouth disease (HFMD)); RNA polymerase L, protein L, glycoprotein Gn, glycoprotein Gc, nucleocapsid protein 5, envelope glycoprotein G1, nucleoprotein NP, protein N, polyprotein M (Sin Nombre virus, Hantavirus, Hantavirus Pulmonary Syndrome (UPS)); heat shock protein HspA, heat shock protein HspB, citrate synthase GItA, protein UreB, heat shock protein Hsp60, neutrophil-activating protein NAP, catalase KatA, vacuolating cytotoxin VacA, urease alpha UreA, urease beta Ureb, protein Cpn10, protein groES, heat shock protein Hsp10, protein MopB, cytotoxicity-associated 10 kDa protein CAG, 36 kDa antigen, beta-lactamase HcpA, Beta-lactamase HcpB (Helicobacter pylori, Helicobacter pylori infection); integral membrane proteins, aggregation-prone proteins, 0-antigen, toxin-antigens Stx2B, toxin-antigen StxlB, adhesion-antigen fragment Int28, protein EspA, protein EspB, Intimin, protein Tir, protein IntC300, protein Eae (Escherichia coli O157:H7, O111 and O104:H4, Hemolytic-uremic syndrome (HUS)); RNA polymerase L, protein L, glycoprotein Gn, glycoprotein Gc, nucleocapsid protein 5, envelope glycoprotein G1, nucleoprotein NP, protein N, polyprotein M (Bunyaviridae family, Hemorrhagic fever with renal syndrome (HFRS)); glycoprotein G, matrix protein M, nucleoprotein N, fusion protein F, polymerase L, protein W, proteinC, phosphoprotein p, non-structural protein V (Henipavirus (Hendra virus Nipah virus), Henipavirus infections); polyprotein, glycoproten Gp2, hepatitis A surface antigen HBAg, protein 2A, virus protein VP1, virus protein VP2, virus protein VP3, virus protein VP4, protein PlB, protein P2A, protein P3AB, protein P3D (Hepatitis A Virus, Hepatitis A); hepatitis B surface antigen HBsAg, Hepatitis B core antigen HbcAg, polymerase, protein Hbx, preS2 middle surface protein, surface protein L, large S protein, virus protein VP1, virus protein VP2, virus protein VP3, virus protein VP4 (Hepatitis B Virus (HBV), Hepatitis B); envelope glycoprotein E1 gp32 gp35 envelope glycoprotein E2 NS1 gp68 gp70, capsid protein C core protein Core, polyprotein, virus protein VP1, virus protein VP2, virus protein VP3, virus protein VP4, antigen G, protein NS3, protein NSSA, (Hepatitis C Virus, Hepatitis C); virus protein VP1, virus protein VP2, virus protein VP3, virus protein VP4, large hepaptitis delta antigen, small hepaptitis delta antigen (Hepatitis D Virus, Hepatitis D); virus protein VP1, virus protein VP2, virus protein VP3, virus protein VP4, capsid protein E2 (Hepatitis E Virus, Hepatitis E); glycoprotein L UL1, uracil-DNA glycosylase UL2, protein UL3, protein UL4, DNA replication protein UL5, portal protein UL6, virion maturation protein UL7, DNA helicase UL8, replication origin-binding protein UL9, glycoprotein M UL10, protein UL 11, alkaline exonuclease UL12, serine-threonine protein kinase UL13, tegument protein UL14, terminase UL15, tegument protein UL16, protein UL17, capsid protein VP23 UL18, major capsid protein VP5 UL19, membrane protein UL20, tegument protein UL21, Glycoprotein H (UL22), Thymidine Kinase UL23, protein UL24, protein UL25, capsid protein P40 (UL26, VP24, VP22A), glycoprotein B (UL27), ICP18.5 protein (UL28), major DNA-binding protein ICP8 (UL29), DNA polymerase UL30, nuclear matrix protein UL31, envelope glycoprotein UL32, protein UL33, inner nuclear membrane protein UL34, capsid protein VP26 (UL35), large tegument protein UL36, capsid assembly protein UL37, VP19C protein (UL38), ribonucleotide reductase (Large subunit) UL39, ribonucleotide reductase (Small subunit) UL40, tegument protein/virion host shutoff VHS protein (UL41), DNA polymerase processivity factor UL42, membrane protein UL43, glycoprotein C (UL44), membrane protein UL45, tegument proteins VP11/12 (UL46), tegument protein VP13/14 (UL47), virion maturation protein VP16 (UL48, Alpha-TIF), envelope protein UL49, dUTP diphosphatase UL50, tegument protein UL51, DNA helicase/primase complex protein UL52, glycoprotein K (UL53), transcriptional regulation protein 1E63 (ICP27, UL54), protein UL55, protein UL56, viral replication protein ICP22 (1E68, US1), protein U52, serine/threonine-protein kinase U53, glycoprotein G (U54), glycoprotein J (U55), glycoprotein D (U56), glycoprotein I (U57), glycoprotein E (U58), tegument protein U59, capsid/tegument protein US10, Vmw21 protein (US 11), ICP47 protein (IE12, US12), major transcriptional activator ICP4 (1E175, RS1), E3 ubiquitin ligase ICPO (IE110), latency-related protein 1 LRP1, latency-related protein 2 LRP2, neurovirulence factor RL1 (ICP34.5), latency-associated transcript LAT (Herpes simplex virus 1 and 2 (HSV-1 and HSV-2), Herpes simplex); heat shock protein Hsp60, cell surface protein H1C, dipeptidyl peptidase type IV DppIV, M antigen, 70 kDa protein, 17 kDa histone-like protein (Histoplasma capsulatum, Histoplasmosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, surface-associated antigen SAA-2, adult-specific secreted factor Xa, serine protease inhibitor anticoagulant AP, cathepsin D-like aspartic protease ARR-1, 5-transferase GST, aspartic protease APR-1, acetylcholinesterase AChE (Ancylostoma duodena le and Necator americanus, Hookworm infection); protein NS1, protein NP1, protein VP1, protein VP2, protein VP3 (Human bocavirus (HBoV), Human bocavirus infection); major surface protein 2 MSP2, major surface protein 4 MSP4, MSP variant SGV1, MSP variant SGV2, outer membrane protein OMP, outer membrande protein 19 OMP-19, major antigenic protein MAP1, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia ewingii, Human ewingii ehrlichiosis); major surface proteins 1-5 (MSP1a, MSP1b, MSP2, MSP3, MSP4, MSP5), type IV secreotion system proteins VirB2, VirB7, VirB11, VirD4 (Anaplasma phagocytophilum, Human granulocytic anaplasmosis (HGA)); protein NS1, small hydrophobic protein N52, SH protein, fusion protein F, glycoprotein G, matrix protein M, matrix protein M2-1, matrix protein M2-2, phosphoprotein P, nucleoprotein N, polymerase L (Human metapneumovirus (hMPV), Human metapneumovirus infection); major surface protein 2 MSP2, major surface protein 4 MSP4, MSP variant SGV1, MSP variant SGV2, outer membrane protein OMP, outer membrande protein 19 OMP-19, major antigenic protein MAP1, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia chaffeensis, Human monocytic ehrlichiosis); replication protein E1, regulatory protein E2, protein E3, protein E4, protein ES, protein E6, protein E7, protein E8, major capsid protein L1, minor capsid protein L2 (Human papillomavirus (HPV), Human papillomavirus (HPV) infection); fusion protein F, hemagglutinin-neuramidase HN, glycoprotein G, matrix protein M, phosphoprotein P, nucleoprotein N, polymerase L (Human parainfluenza viruses (HPIV), Human parainfluenza virus infection); Hemagglutinin (HA), Neuraminidase (NA), Nucleoprotein (NP), M1 protein, M2 protein, NS1 protein, NS2 protein (NEP protein: nuclear export protein), PA protein, PB1 protein (polymerase basic 1 protein), PB1-F2 protein and PB2 protein (Orthomyxoviridae family, Influenza virus (flu)); genome polyprotein, protein E, protein M, capsid protein C (Japanese encephalitis virus, Japanese encephalitis); RTX toxin, type IV pili, major pilus subunit PilA, regulatory transcription factors PilS and PilR, protein sigma54, outer membrane proteins (Kingella kingae, Kingella kingae infection); prion protein (Kuru prion, Kuru); nucleoprotein N, polymerase L, matrix protein Z, glycoprotein GP (Lassa virus, Lassa fever); peptidoglycan-associated lipoprotein PAL, 60 kDa chaperonin Cpn60 (groEL, HspB), type IV pilin PilE, outer membrane protein MIP, major outer membrane protein MompS, zinc metalloproteinase MSP (Legionella pneumophila, Legionellosis (Legionnaires' disease, Pontiac fever)); P4 nuclease, protein WD, ribonucleotide reductase M2, surface membrane glycoprotein Pg46, cysteine proteinase CP, glucose-regulated protein 78 GRP-78, stage-specific S antigen-like protein A2, ATPase F1, beta-tubulin, heat shock protein 70 Hsp70, KMP-11, glycoprotein GP63, protein BT1, nucleoside hydrolase NH, cell surface protein B1, ribosomal protein P1-like protein P1, sterol 24-c-methyltransferase SMT, LACK protein, histone H1, SPB1 protein, thiol specific antioxidant TSA, protein antigen STIl, signal peptidase SP, histone H2B, suface antigen PSA-2, cystein proteinase b Cpb (Leishmania genus, Leishmaniasis); major membrane protein I, serine-rich antigen-45 kDa, 10 kDa caperonin GroES, HSP kDa antigen, amino-oxononanoate synthase AONS, protein recombinase A RecA, Acetyl-/propionyl-coenzyme A carboxylase alpha, alanine racemase, 60 kDa chaperonin 2, ESAT-6-like protein EcxB (L-ESAT-6), protein Lsr2, protein ML0276, Heparin-binding hemagglutinin HBHA, heat-shock protein 65 Hsp65, mycP1 or ML0041 coding protein htrA2 or ML0176 coding protein htrA4 or ML2659 coding protein, gcp or ML0379 coding protein, clpC or ML0235 coding protein (Mycobacterium leprae and Mycobacterium lepromatosis, Leprosy); outer membrane protein LipL32, membrane protein LIC10258, membrane protein LP30, membrane protein LIC12238, Ompa-like protein Lsa66, surface protein LigA, surface protein LigB, major outer membrane protein OmpL1, outer membrane protein LipL41, protein LigAni, surface protein LcpA, adhesion protein LipL53, outer membrane protein UpL32, surface protein Lsa63, flagellin FlaB1, membran lipoprotein LipL21, membrane protein pL40, leptospiral surface adhesin Lsa27, outer membrane protein OmpL36, outer membrane protein OmpL37, outer membrane protein OmpL47, outer membrane protein OmpL54, acyltransferase LpxA (Leptospira genus, Leptospirosis); listeriolysin 0 precursor Hly (LLO), invasion-associated protein lap (P60), Listeriolysin regulatory protein PrfA, Zinc metalloproteinase Mpl, Phosphatidylinositol-specific phospholipase C PLC (PIcA, PlcB), 0-acetyltransferase Oat, ABC-transporter permease Im.G_1771, adhesion protein LAP, LAP receptor Hsp60, adhesin LapB, haemolysin listeriolysin 0 LLO, protein ActA, Internalin A InIA, protein InIB (Listeria monocytogenes, Listeriosis); outer surface protein A OspA, outer surface protein OspB, outer surface protein OspC, decorin binding protein A DbpA, decorin binding protein B DbpB, flagellar filament 41 kDa core protein Fla, basic membrane protein A BmpA (Immunodominant antigen P39), outer surface 22 kDa lipoprotein precursor (antigen IPLA7), variable surface lipoprotein vlsE (usually Borrelia burgdorferi and other Borrelia species, Lyme disease (Lyme borreliosis)); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, Secreted Larval Acidic Proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, protein Cox-2 (Wuchereria bancrofti and Brugia malayi, Lymphatic filariasis (Elephantiasis)); glycoprotein GP, matrix protein polymerase L, nucleoprotein N (Lymphocytic choriomeningitis virus (LCMV), Lymphocytic choriomeningitis); thrombospondin-related anonymous protein TRAP, SSP2 Sporozoite surface protein 2, apical membrane antigen 1 AMAl, rhoptry membrane antigen RMA1, acidic basic repeat antigen ABRA, cell-traversal protein PF, protein Pvs25, merozoite surface protein 1 MSP-1, merozoite surface protein 2 MSP-2, ring-infected erythrocyte surface antigen RESALiver stage antigen 3 LSA-3, protein Eba-175, serine repeat antigen 5 SERA-5, circumsporozoite protein CS, merozoite surface protein 3 MSP3, merozoite surface protein 8 MSP5, enolase PF10, hepatocyte erythrocyte protein 17 kDa HEP17, erythrocyte membrane protein 1 EMP1, protein Kbetamerozoite surface protein 4/5 MSP 4/5, heat shock protein Hsp90, glutamate-rich protein GLURP, merozoite surface protein 4 MSP-4, protein STARP, circumsporozoite protein-related antigen precursor CRA (Plasmodium genus, Malaria); nucleoprotein N, membrane-associated protein VP24, minor nucleoprotein VP30, polymerase cofactor VP35, polymerase L, matrix protein VP40, envelope glycoprotein GP (Marburg virus, Marburg hemorrhagic fever (MHF)); protein C, matrix protein M, phosphoprotein P, non-structural protein V, hemagglutinin glycoprotein H, polymerase L, nucleoprotein N, fusion protein F (Measles virus, Measles); members of the ABC transporter family (LoIC, OppA, and PotF), putative lipoprotein releasing system transmembrane protein LoIC/E, flagellin FliC, Burkholderia intracellular motility A BimA, bacterial Elongation factor-Tu EF-Tu, 17 kDa OmpA-like protein, boaA coding protein, boaB coding protein (Burkholderia pseudomallei, Melioidosis (Whitmore's disease)); pilin proteins, minor pilin-associated subunit pilC, major pilin subunit and variants pilE, pilS, phase variation protein porA, Porin B PorB, protein TraD, Neisserial outer membrane antigen H.8, 70 kDa antigen, major outer membrane protein PI, outer membrane proteins PIA and PIB, W antigen, surface protein A NspA, transferrin binding protein TbpA, transferrin binding protein TbpB PBP2, mtrR coding protein, ponA coding protein, membrane permease FbpBC, FbpABC protein system, LbpAB proteins, outer membrane protein Opa, outer membrane transporter FetA, iron-repressed regulator MpeR, factor H-binding protein flbp, adhesin NadA, protein NhbA, repressor FarR (Neisseria meningitidis, Meningococcal disease); 66 kDa protein, 22 kDa protein (usually Metagonimus yokagawai, Metagonimiasis); polar tube proteins (34, 75, and 170 kDa in Glugea, 35, 55 and 150 kDa in Encephalitozoon), kinesin-related protein, RNA polymerase II largest subunit, similar of integral membrane protein YIPA, anti-silencing protein 1, heat shock transcription factor HSF, protein kinase, thymidine kinase, NOP-2 like nucleolar protein (Microsporidia phylum, Microsporidiosis); CASP8 and FADD-like apoptosis regulator, Glutathione peroxidase GPX1, RNA helicase NPH-II NPH2, Poly(A) polymerase catalytic subunit PAPL, Major envelope protein P43K, early transcription factor 70 kDa subunit VETFS, early transcription factor 82 kDa subunit VETFL, metalloendopeptidase G1-type, nucleoside triphosphatase I NPH1, replication protein A28-like MC134L, RNA polymease 7 kDa subunit RPO7 (Molluscum contagiosum virus (MCV), Molluscum contagiosum (MC)); matrix protein M, phosphoprotein P/V, small hydrophobic protein SH, nucleoprotein N, protein V, fusion glycoprotein hemagglutinin-neuraminidase HN, RNA polymerase L (Mumps virus, Mumps); Outer membrane proteins OM, cell surface antigen OmpA, cell surface antigen OmpB (sca5), cell surface protein SCA4, cell surface protein SCAl, intracytoplasmic protein D, crystalline surface layer protein SLP, protective surface protein antigen SPA (Rickettsia typhi, Murine typhus (Endemic typhus)); adhesin P1, adhesion P30, protein p116, protein P40, cytoskeletal protein HMW1, cytoskeletal protein HMW2, cytoskeletal protein HMW3, MPN152 coding protein, MPN426 coding protein, MPN456 coding protein, MPN-500coding protein (Mycoplasma pneumoniae, Mycoplasma pneumonia); NocA, Iron dependent regulatory protein, VapA, VapD, VapF, VapG, caseinolytic protease, filament tip-associated 43-kDa protein, protein P24, protein P61, 15-kDa protein, 56-kDa protein (usually Nocardia asteroides and other Nocardia species, Nocardiosis); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, Thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, Secreted Larval Acidic Proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, Cox-2 (Onchocerca volvulus, Onchocerciasis (River blindness)); 43 kDa secreted glycoprotein, glycoprotein gp0, glycoprotein gp75, antigen Pb27, antigen Pb40, heat shock protein Hsp65, heat shock protein Hsp70, heat shock protein Hsp90, protein P10, triosephosphate isomerase TPI, N-acetyl-glucosamine-binding lectin Paracoccin, 28 kDa protein Pb28 (Paracoccidioides brasiliensis, Paracoccidioidomycosis (South American blastomycosis)); 28-kDa cruzipain-like cystein protease Pw28CCP (usually Paragonimus westermani and other Paragonimus species, Paragonimiasis); outer membrane protein OmpH, outer membrane protein Omp28, protein PM1539, protein PM0355, protein PM1417, repair protein MutL, protein BcbC, prtein PM0305, formate dehydrogenase-N, protein PM0698, protein PM1422, DNA gyrase, lipoprotein PIpE, adhesive protein Cp39, heme aquisition system receptor HasR, 39 kDa capsular protein, iron-regulated OMP IROMP, outer membrane protein OmpA87, fimbrial protein Ptf, fimbrial subunit protein PtfA, transferrin binding protein Tbpl, esterase enzyme MesA, Pasteurella multocida toxin PMT, adhesive protein Cp39 (Pasteurella genus, Pasteurellosis); “filamentous hemagglutinin FhaB, adenylate cyclase CyaA, pertussis toxin subunit 4 precursor PtxD, pertactin precursor Prn, toxin subunit 1 PtxA, protein Cpn60, protein brkA, pertussis toxin subunit 2 precursor PtxB, pertussis toxin subunit 3 precursor PtxC, pertussis toxin subunit 5 precursor PtxE, pertactin Pm, protein Fim2, protein Fim3;” (Bordetella pertussis, Pertussis (Whooping cough)); “F1 capsule antigen, virulence-associated V antigen, secreted effector protein LcrV, V antigen, outer membrane protease Pla, secreted effector protein YopD, putative secreted protein-tyrosine phosphatase YopH, needle complex major subunit YscF, protein kinase YopO, putative autotransporter protein YapF, inner membrane ABC-transporter YbtQ (Irp7), putative sugar binding protein YP00612, heat shock protein 90 HtpG, putative sulfatase protein YdeN, outer-membrane lipoprotein carrier protein LoIA, secretion chaperone YerA, putative lipoprotein YP00420, hemolysin activator protein HpmB, pesticin/yersiniabactin outer membrane receptor Psn, secreted effector protein YopE, secreted effector protein YopF, secreted effector protein YopK, outer membrane protein YopN outer membrane protein YopM, Coagulase/fibrinolysin precursor Pla;” (Yersinia pestis, Plague); protein PhpA, surface adhesin PsaA, pneumolysin Ply, ATP-dependent protease CIp, lipoate-protein ligase LpIA, cell wall surface anchored protein psrP, sortase SrtA, glutamyl-tRNA synthetase GItX, choline binding protein A CbpA, pneumococcal surface protein A PspA, pneumococcal surface protein C PspC, 6-phosphogluconate dehydrogenase Gnd, iron-binding protein PiaA, Murein hydrolase LytB, proteon LytC, protease A1 (Streptococcus pneumoniae, Pneumococcal infection); major surface protein B, kexin-like protease KEX1, protein A12, 55 kDa antigen P55, major surface glycoprotein Msg (Pneumocystis jirovecii, Pneumocystis pneumonia (PCP)); genome polyprotein, polymerase 3D, viral capsid protein VP1, viral capsid protein VP2, viral capsid protein VP3, viral capsid protein VP4, protease 2A, protease 3C (Poliovirus, Poliomyelitis); protein Nfal, exendin-3, secretory lipase, cathepsin B-like protease, cysteine protease, cathepsin, peroxiredoxin, protein CrylAc (usually Naegleria fowleri, Primary amoebic meningoencephalitis (PAM)); agnoprotein, large T antigen, small T antigen, major capsid protein VP1, minor capsid protein Vp2 (JC virus, Progressive multifocal leukoencephalopathy); low calcium response protein E LCrE, chlamydial outer protein N CopN, serine/threonine-protein kinase PknD, acyl-carrier-protein S-ma lonyltransferase FabD, single-stranded DNA-binding protein Ssb, major outer membrane protein MOMP, outer membrane protein 2 Omp2, polymorphic membrane protein family (Pmp1, Pmp2, Pmp3, Pmp4, Pmp5, Pmp6, Pmp7, Pmp8, Pmp9, Pmp10, Pmp11, Pmp12, Pmp13, Pmp14, Pmp15, Pmp16, Pmp17, Pmp18, Pmp19, Pmp20, Pmp21) (Chlamydophila psittaci, Psittacosis); outer membrane protein P1, heat shock protein B HspB, peptide ABC transporter, GTP-binding protein, protein IcmB, ribonuclease R, phosphatas SixA, protein DsbD, outer membrane protein ToIC, DNA-binding protein PhoB, ATPase DotB, heat shock protein B HspB, membrane protein Coml, 28 kDa protein, DNA-3-methyladenine glycosidase I, pouter membrane protein OmpH, outer membrane protein AdaA, glycine cleavage system T-protein (Coxiella burnetii, Q fever); nucleoprotein N, large structural protein L, phophoprotein P, matrix protein M, glycoprotein G (Rabies virus, Rabies); fusionprotein F, nucleoprotein N, matrix protein M, matrix protein M2-1, matrix protein M2-2, phophoprotein P, small hydrophobic protein SH, major surface glycoprotein G, polymerase L, non-structural protein 1 NS1, non-structural protein 2 NS2 (Respiratory syncytial virus (RSV), Respiratory syncytial virus infection); genome polyprotein, polymerase 3D, viral capsid protein VP1, viral capsid protein VP2, viral capsid protein VP3, viral capsid protein VP4, protease 2A, protease 3C (Rhinovirus, Rhinovirus infection); outer membrane proteins OM, cell surface antigen OmpA, cell surface antigen OmpB (sca5), cell surface protein SCA4, cell surface protein SCAl, protein PS120, intracytoplasmic protein D, protective surface protein antigen SPA (Rickettsia genus, Rickettsial infection); outer membrane proteins OM, cell surface antigen OmpA, cell surface antigen OmpB (sca5), cell surface protein SCA4, cell surface protein SCAl, intracytoplasmic protein D (Rickettsia akari, Rickettsialpox); envelope glycoprotein GP, polymerase L, nucleoprotein N, non-structural protein NSS (Rift Valley fever virus, Rift Valley fever (RVF)); outer membrane proteins OM, cell surface antigen OmpA, cell surface antigen OmpB (sca5), cell surface protein SCA4, cell surface protein SCAl, intracytoplasmic protein D (Rickettsia rickettsii, Rocky mountain spotted fever (RMSF)); non-structural protein 6 N56, non-structural protein 2 N52, intermediate capsid protein VP6, inner capsid protein VP2, non-structural protein 3 NS3, RNA-directed RNA polymerase L, protein VP3, non-structural protein 1 NS1, non-structural protein 5 N55, outer capsid glycoprotein VP7, non-structural glycoprotein 4 N54, outer capsid protein VP4; (Rotavirus, Rotavirus infection); polyprotein P200, glycoprotein E1, glycoprotein E2, protein N52, capsid protein C (Rubella virus, Rubella); chaperonin GroEL (MopA), inositol phosphate phosphatase SopB, heat shock protein HslU, chaperone protein DnaJ, protein TviB, protein IroN, flagellin FliC, invasion protein SipC, glycoprotein gp43, outer membrane protein LamB, outer membrane protein PagC, outer membrane protein ToIC, outer membrane protein NmpC, outer membrane protein FadL, transport protein SadA, transferase WgaP, effector proteins SifA, SteC, SseL, SseJ and SseF (Salmonella genus, Salmonellosis); “protein 14, non-structural protein NS7b, non-structural protein NS8a, protein 9b, protein 3a, nucleoprotein N, non-structural protein NS3b, non-structural protein N56, protein 7a, non-structural protein NS8b, membrane protein M, envelope small membrane protein EsM, replicase polyprotein 1a, spike glycoprotein S, replicase polyprotein lab; SARS coronavirus, SARS (Severe Acute Respiratory Syndrome)); serin protease, Atypical Sarcoptes Antigen 1 ASAI, glutathione 5-transferases GST, cystein protease, serine protease, apolipoprotein (Sarcoptes scabiei, Scabies); glutathione 5-transferases GST, paramyosin, hemoglbinase SM32, major egg antigen, 14 kDa fatty acid-binding protein Sm14, major larval surface antigen P37, 22.6 kDa tegumental antigen, calpain CANP, triphospate isomerase Tim, surface protein 9B, outer capsid protein VP2, 23 kDa integral membrane protein Sm23, Cu/Zn-superoxide dismutase, glycoprotein Gp, myosin (Schistosoma genus, Schistosomiasis (Bilharziosis)); 60 kDa chaperonin, 56 kDa type-specific antigen, pyruvate phosphate dikinase, 4-hydroxybenzoate octaprenyltransferase (Orientia tsutsugamushi, Scrub typhus); dehydrogenase GuaB, invasion protein Spa32, invasin IpaA, invasin IpaB, invasin IpaC, invasin IpaD, invasin IpaH, invasin IpaJ (Shigella genus, Shigellosis (Bacillary dysentery)); protein P53, virion protein US10 homolog, transcriptional regulator 1E63, transcriptional transactivator 1E62, protease P33, alpha trans-inducing factor 74 kDa protein, deoxyuridine 5′-triphosphate nucleotidohydrolase, transcriptional transactivator 1E4, membrane protein UL43 homolog, nuclear phosphoprotein UL3 homolog, nuclear protein UL4 homolog, replication origin-binding protein, membrane protein 2, phosphoprotein 32, protein 57, DNA polymerase processivity factor, portal protein 54, DNA primase, tegument protein UL14 homolog, tegument protein UL21 homolog, tegument protein UL55 homolog, tripartite terminase subunit UL33 homolog, tripartite terminase subunit UL15 homolog, capsid-binding protein 44, virion-packaging protein 43 (Varicella zoster virus (VZV), Shingles (Herpes zoster)); truncated 3-beta hydroxy-5-ene steroid dehydrogenase homolog, virion membrane protein A13, protein A19, protein A31, truncated protein A35 homolog, protein A37.5 homolog, protein A47, protein A49, protein A51, semaphorin-like protein A43, serine proteinase inhibitor 1, serine proteinase inhibitor 2, serine proteinase inhibitor 3, protein A6, protein B15, protein C1, protein C5, protein C6, protein F7, protein F8, protein F9, protein F11, protein F14, protein F15, protein F16 (Variola major or Variola minor, Smallpox (Variola)); adhesin/glycoprotein gp70, proteases (Sporothrix schenckii, Sporotrichosis); heme-iron binding protein IsdB, collagen adhesin Cna, clumping factor A CIfA, protein MecA, fibronectin-binding protein A FnbA, enterotoxin type A EntA, enterotoxin type B EntB, enterotoxin type C EntC1, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcus genus, Staphylococcal food poisoning); heme-iron binding protein IsdB, collagen adhesin Cna, clumping factor A CIfA, protein MecA, fibronectin-binding protein A FnbA, enterotoxin type A EntA, enterotoxin type B EntB, enterotoxin type C EntC1, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcusgenus e.g. aureus, Staphylococcal infection); antigen Ss-IR, antigen NIE, strongylastacin, Na+-K+ ATPase Sseat-6, tropomysin SsTmy-1, protein LEC-5, 41 kDa aantigen P5, 41-kDa larval protein, 31-kDa larval protein, 28-kDa larval protein (Strongyloides stercoralis, Strongyloidiasis); glycerophosphodiester phosphodiesterase GlpQ (Gpd), outer membrane protein TmpB, protein Tp92, antigen TpF1, repeat protein Tpr, repeat protein F TprF, repeat protein G TprG, repeat protein I Tprl, repeat protein J TprJ, repeat protein KTprK, treponemal membrane protein A TmpA, lipoprotein, 15 kDa Tpp15, 47 kDa membrane antigen, miniferritin TpF1, adhesin Tp0751, lipoprotein TP0136, protein TpN17, protein TpN47, outer membrane protein TP0136, outer membrane protein TP0155, outer membrane protein TP0326, outer membrane protein TP0483, outer membrane protein TP0956 (Treponema pallidum, Syphilis); Cathepsin L-like proteases, 53/25-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia genus, Taeniasis); tetanus toxin TetX, tetanus toxin C TTC, 140 kDa S layer protein, flavoprotein beta-subunit CT3, phospholipase (lecithinase), phosphocarrier protein HPr (Clostridium tetani, Tetanus (Lockjaw)); genome polyprotein, protein E, protein M, capsid protein C (Tick-borne encephalitis virus (TBEV), Tick-borne encephalitis); 58-kDa antigen, 68-kDa antigens, Toxocara larvae excretory-secretory antigen TES, 32-kDa glycoprotein, glycoprotein TES-70, glycoprotein GP31, excretory-secretory antigen TcES-57, perienteric fluid antigen Pe, soluble extract antigens Ex, excretory/secretory larval antigens ES, antigen TES-120, polyprotein allergen TBA-1, cathepsin L-like cysteine protease c-cpl-1, 26-kDa protein (Toxocara canis or Toxocara cati, Toxocariasis (Ocular Larva Migrans (OLM) and Visceral Larva Migrans (VLM))); microneme proteins (MIC1, MIC2, MIC3, MIC4, MIC5, MICE, MIC7, MICE), rhoptry protein Rop2, rhoptry proteins (Rop1, Rop2, Rop3, Rop4, Rop5, Rop6, Rop7, Rop16, Rjop17), protein SRi, surface antigen P22, major antigen β24, major surface antigen p30, dense granule proteins (GRA1, GRA2, GRA3, GRA4, GRA5, GRA6, GRA7, GRAB, GRA9, GRA10), 28 kDa antigen, surface antigen SAG1, SAG2 related antigen, nucleoside-triphosphatase 1, nucleoside-triphosphatase 2, protein Stt3, HesB-like domain-containing protein, rhomboid-like protease 5, toxomepsin 1 (Toxoplasma gondii, Toxoplasmosis); 43 kDa secreted glycoprotein, 53 kDa secreted glycoprotein, paramyosin, antigen Ts21, antigen Ts87, antigen p46000, TSL-1 antigens, caveolin-1 CAV-1, 49 kDa newborn larva antigen, prosaposin homologue, serine protease, serine proteinase inhibitor, 45-kDa glycoprotein Gp45 (Trichinella spiralis, Trichinellosis); Myb-like transcriptional factors (Myb1, Myb2, Myb3), adhesion protein AP23, adhesion protein AP33, adhesin protein AP33-3, adhesins AP51, adhesin AP65, adhesion protein AP65-1, alpha-actinin, kinesin-associated protein, teneurin, 62 kDa proteinase, subtilisin-like serine protease SUB1, cysteine proteinase gene 3 CP3, alpha-enolase Enol, cysteine proteinase CP30, heat shock proteins (Hsp70, Hsp60) immunogenic protein P270, (Trichomonas vaginalis, Trichomoniasis); beta-tubulin, 47-kDa protein, secretory leucocyte-like proteinase-1 SLP-1, 50-kDa protein TT50, 17 kDa antigen, 43/47 kDa protein (Trichuris trichiura, Trichuriasis (Whipworm infection)); protein ESAT-6 (EsxA), 10 kDa filtrate antigen EsxB, secreted antigen 85-B FBPB, fibronectin-binding protein A FbpA (Ag85A), serine protease PepA, PPE family protein PPE18, fibronectin-binding protein D FbpD, immunogenic protein MPT64, secreted protein MPT51, catalase-peroxidase-peroxynitritase T KATG, periplasmic phosphate-binding lipoprotein PSTS3 (PBP-3, Phos-1), iron-regulated heparin binding hemagglutinin Hbha, PPE family protein PPE14, PPE family protein PPE68, protein Mtb72F, protein Apa, immunogenic protein MPT63, periplasmic phosphate-binding lipoprotein PSTS1 (PBP-1), molecular chaperone DnaK, cell surface lipoprotein Mpt83, lipoprotein P23, phosphate transport system permease protein pstA, 14 kDa antigen, fibronectin-binding protein C FbpCl, Alanine dehydrogenase TB43, Glutamine synthetase 1, ESX-1 protein, protein CFP10, TB10.4 protein, protein MPT83, protein MTB12, protein MTBE, Rpf-like proteins, protein MTB32, protein MTB39, crystallin, heat-shock protein HSP65, protein PST-S (usually Mycobacterium tuberculosis, Tuberculosis); outer membrane protein FobA, outer membrane protein FobB, intracellular growth locus IgIC1, intracellular growth locus IgIC2, aminotransferase Wbtl, chaperonin GroEL, 17 kDa major membrane protein TUL4, lipoprotein LpnA, chitinase family 18 protein, isocitrate dehydrogenase, Nif3 family protein, type IV pili glycosylation protein, outer membrane protein toIC, FAD binding family protein, type IV pilin multimeric outer membrane protein, two component sensor protein KdpD, chaperone protein DnaK, protein TolQ (Francisella tularensis, Tularemia); “MB antigen, urease, protein GyrA, protein GyrB, protein ParC, protein ParE, lipid associated membrane proteins LAMP, thymidine kinase TK, phospholipase PL-A1, phospholipase PL-A2, phospholipase PL-C, surface-expressed 96-kDa antigen;” (Ureaplasma urealyticum, Ureaplasma urealyticum infection); non-structural polyprotein, structural polyprotein, capsid protein CP, protein E1, protein E2, protein E3, protease Pb, protease P2, protease P3 (Venezuelan equine encephalitis virus, Venezuelan equine encephalitis); glycoprotein GP, matrix protein Z, polymerase L, nucleoprotein N (Guanarito virus, Venezuelan hemorrhagic fever); polyprotein, protein E, protein M, capsid protein C, protease NS3, protein NS1, protein NS2A, protein AS2B, brotein NS4A, protein NS4B, protein NS5 (West Nile virus, West Nile Fever); cpasid protein CP, protein E1, protein E2, protein E3, protease P2 (Western equine encephalitis virus, Western equine encephalitis); genome polyprotein, protein E, protein M, capsid protein C, protease NS3, protein NS1, protein NS2A, protein AS2B, protein NS4A, protein NS4B, protein NS5 (Yellow fever virus, Yellow fever); putative Yop targeting protein YobB, effector protein YopD, effector protein YopE, protein YopH, effector protein YopJ, protein translocation protein YopK, effector protein YopT, protein YpkA, flagellar biosyntheses protein FIhA, peptidase M48, potassium efflux system KefA, transcriptional regulatoer RovA, adhesin Ifp, translocator portein LcrV, protein PcrV, invasin Inv, outer membrane protein OmpF-like porin, adhesin YadA, protein kinase C, phospholipase C1, protein PsaA, mannosyltransferase-like protein WbyK, protein YscU, antigen YPMa (Yersinia pseudotuberculosis, Yersinia pseudotuberculosis infection); and effector protein YopB, 60 kDa chaperonin, protein WbcP, tyrosin-protein phosphatase YopH, protein YopQ, enterotoxin, Galactoside permease, reductaase NrdE, protein YasN, Invasin Inv, adhesin YadA, outer membrane porin F OmpF, protein UspAl, protein EibA, protein Hia, cell surface protein Ail, chaperone SycD, protein LcrD, protein LcrG, protein LcrV, protein SycE, protein YopE, regulator protein TyeA, protein YopM, protein YopN, protein YopO, protein YopT, protein YopD, protease CIpP, protein MyfA, protein FilA, and protein PsaA (Yersinia enterocolitica, Yersiniosis).

The infectious agent can be a bacterium, a fungus, a virus, or a protist. The infectious agent can be a coronavirus (CoV) (e.g., an alphacoronavirus, a betacoronavirus, a gammacoronavirus, or a deltacoronavirus). The infectious agent can be selected from Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CID prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157:H7, O111 and O104:H4, Fasciola hepatica and Fasciola gigantica, FFI prion, Filarioidea superfamily, Filoviruses, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

The presenting peptide can comprise or can be derived from the full-length surface protein of a coronavirus. The disease or disorder can be an infectious disease or disorder caused by a coronavirus, and the presenting peptide can comprise or can be derived from an antigenic protein of a coronavirus. The term “coronavirus” as used herein refers to a virus in the family Coronaviridae, which is in turn classified within the order Nidovirales. The coronaviruses are large, enveloped, positive-stranded RNA viruses. The coronaviruses have the largest genomes of the RNA viruses known in the art and replicate by a unique mechanism that results in a high frequency of recombination. The coronaviruses include antigenic groups I, II, and III. Nonlimiting examples of coronaviruses include SARS coronavirus (e.g., SARS-CoV and SARS-CoV-2), MERS coronavirus, transmissible gastroenteritis virus (TGEV), human respiratory coronavirus, porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus, as well as chimeras thereof. Additional information related to coronavirus including classification, virion structure, genome structure, genetics and pathology is described, for example, in KV Holmes, Encyclopedia of Virology, 1999: 291-298, the content of which is incorporated herein by reference.

In some embodiments, a coronavirus described herein is in the genus of Alpha-coronavirus and the coronavirus antigens can be of or derived from any species or strains in the genus of Alpha-coronavirus. In some embodiments, a coronavirus described herein is in the genus of Beta-coronavirus and the coronavirus antigens can be of or derived from any species or strains in the genus of Beta-coronavirus. Member viruses in the genus of Alpha-coronavirus and Beta-coronavirus are enveloped, positive-strand RNA viruses that can infect mammals.

A coronavirus described herein can be of any subgenus of Alpha-coronavirus genus, including but not limited to Colacovirus (e.g. Bat coronavirus CDPHE15), Decacovirus (e.g. Bat coronavirus HKU10 and Rhinolophus ferrumequinum alphacoronavirus HuB-2013), Duvinacovirus (Human coronavirus 229E), Luchacovirus (e.g. Lucheng Rn rat coronavirus), Minacovirus (e.g. Mink coronavirus 1), Minunacovirus (e.g. Miniopterus bat coronavirus 1 and Miniopterus bat coronavirus HKU8), Myotacovirus (e.g. Myotis ricketti alphacoronavirus Sax-2011), Nyctacovirus (e.g. Nyctalus velutinus alphacoronavirus SC-2013 and Pipistrellus kuhlii coronavirus 3398), Pedacovirus (e.g. Porcine epidemic diarrhea virus and Scotophilus bat coronavirus 512), Rhinacovirus (e.g. Rhinolophus bat coronavirus HKU2), Setracovirus (e.g. Human coronavirus NL63 and NL63-related bat coronavirus strain BtKYNL63-9b), Soracovirus (e.g. Sorex araneus coronavirus T14), Sunacovirus (e.g. Suncus murinus coronavirus X74), and Tegacovirus (e.g. Alphacoronavirus 1).

Within the genus Beta-coronavirus, five subgenera or lineages have been recognized, including Embecovirus (lineage A), Sarbecovirus (lineage B), Merbecovirus (lineage C), Nobecovirus (lineage D), and Hibecovirus. Accordingly, in some embodiments, a coronavirus described herein can be any strain or species in any of the subgenera or lineages of Beta-coronavirus.

For example, a coronavirus antigen can be of or derived from any species or strains in the subgenus of Embecovirus, including but not limited to Beta-coronavirus 1 (e.g. Bovine coronavirus and human coronavirus OC43), China Rattus coronavirus HKU24, Human coronavirus HKUJ, Murine coronavirus (e.g. mouse heptatitis virus), and Myodes coronavirus 2JL14. The coronavirus antigen can be of or derived from any species or strains in the subgenus of Sarbecovirus, including but not limited to SARS-CoV, SARS-CoV2, 16B0133, Bat SARS CoV Rfl, Bat coronavirus HKU3 (BtCoV HKU3), LYRal 1, Bat SARS-CoV/Rp3, Bat SL-CoV YNLF_31C, Bat SL-CoV YNLF_34C, SHC014-CoV, WIV1, WIV16, Civet SARS-CoV, Re-o319, SL-ZXC21, SL-ZC45, Pangolin SARSr-COV-GX, Pangolin SARSr-COV-GD, RshSTT182, RshSTT200, RacCS203, RmYNO2, RpYN06, RaTG13, Bat CoV BtKY72, and Bat CoV BM48-31. The coronavirus antigen can be of any species or strains in the subgenus of Merbecovirus, including but not limited to Hedgehog coronavirus 1, MERS-CoV, Pipistrellus bat coronavirus HKU5, and Tylonycteris bat coronavirus HKU4. The coronavirus antigen can be of any species or strains in the subgenus of Nobecovirus, including but not limited to Eidolon bat coronavirus C704, Rousettus bat coronavirus GCCDC1, and Rousettus bat coronavirus HKU9. The coronavirus antigen can be of any species or strains in the subgenus of Hibecovirus, including but not limited to Bat Hp-betacoronavirus Zhejiang 2013.

The coronaviruses described herein can be, for example, phylogenetically clustered in functionally distinct clades. For example, the coronaviruses of lineage B Beta-coronavirus (Sarbecovirus) can be clustered into clade 1, clade 2, clade 1/2, or clade 3 using the nucleotide sequences of nonstructural protein gene ORF 1a and ORFlb (see, for example, Hu et al., PLoS Pathog 13(11): e1006698). Accordingly, the coronavirus antigens can be of or derived from any species or strain in any one of these clades. For example, the coronavirus antigens can be of any species or strain in clade 1, including but not limited to SARS-CoV, WIV1, LYRal 1, Rs7327, Rs4231, Rs4084, and SHC014. The coronavirus antigens can be of any species or strain in clade 2, including but not limited to As6526, Yunnan 2011, Shaanxi 2011, 279-2005, Rs4237, Rs4081, Rp3, Rs4247, HKU3-8, HKU3-13, GX2013, Longquan-140,YN2013, Rf4092, ZXC21, ZC45, JL2012, HuB2013, Rfl, HeB2013, and 273-2005. The coronavirus antigens can be of any species or strain in clade 1/2, including but not limited to SARS-CoV2. The coronavirus antigens can be of any species or strain in clade 3, including but not limited to BM48-31. The coronavirus antigen described herein can be of a coronavirus, for example, SARS, SARS-2, WIV1, SHC014, Rfl, RmYN02, pangl7, RaTG13, and Rs4081.

As exemplified herein, SARS virus (e.g., SARS-CoV and SARS-CoV-2) is an enveloped coronavirus carrying a single-stranded positive-sense RNA genome (~30 kb), belonging to the genus Betacoronavirus from the Coronaviridae family. The virus RNA encodes four structural proteins including spike (S), envelope (E), membrane (M), and nucleocapsid (N) proteins, 16 non-structural proteins, and nine accessory proteins. The S glycoprotein contains an ectodomain that can be processed into S1 and S2 subunits, a transmembrane domain, and an intracellular domain. Both SARS-CoV and SARS-CoV-2 bind the human ACE2 via the receptor binding domain within the S1 subunit to facilitate entry into host cells, followed by membrane fusion mediated by the S2 subunit.

A coronavirus antigen of a coronavirus herein described can be any of a variety of coronavirus proteins capable of inducing an immune response against a coronavirus. Suitable coronavirus antigens are those that can elicit a protective immune response, such as producing broadly neutralizing antibodies. For example, the coronavirus antigen can comprise a coronavirus spike (S) protein, spike receptor binding domain (RBD), S1 subunit, S2 subunit, spike full ectodomain proteins, papain-like proteases, 3CL proteases, nucleocapsid proteins, envelope proteins, membrane proteins, or any of the structural, non-structural or accessory proteins that form a coronavirus.

In some embodiments, a coronavirus antigen used herein comprises a spike (S) protein or a portion thereof. An S protein is one of four major structural proteins covering the surface of each virion. The S protein, comprising a S1 subunit and a S2 subunit, is a highly glycosylated, type I transmembrane protein capable of binding to a host-cell receptor and mediates viral entry. The S protein comprises a domain referred to as the RBD that mediates the interaction with the host-cell receptor to enter the host cell after one or more RBDs adopts an “up” position to bind the host receptor. It is believed that after binding the receptor, a nearby host protease cleaves the spike, which releases the spike fusion peptide, facilitating virus entry. Known host receptors for coronaviruses (e.g., Beta-coronaviruses) include antiotensin-converting enzyme 2 (ACE2), dipeptidyl peptidase-4 (DPP4) or sialic acids. For example, the RBDs of human coronaviruses SARS-CoV-2, SARS-CoV, HCoV-NL63, and related animal coronaviruses (WIV1 and SCH014) use ACE2 as their host receptor, while MERS-CoV uses DPP4 as its host receptor.

The coronavirus antigen used herein can, for example, comprise a coronavirus nucleocapsid protein (N protein) or a portion thereof. The N protein is a multifunctional RNA-binding protein required for viral RNA transcription, replication, and packaging. The N protein consists of three domains, an N-terminal RNA-binding domain, a central intrinsically disordered region, followed by a C-terminal dimerization domain. The RNA-binding domain contains multiple positively charged binding surfaces that form charged interactions with RNA promoting its helical arrangement. The coronavirus antigen used herein can comprise any of these N protein domains or a portion thereof.

In some embodiments, the coronavirus antigen used herein comprises a coronavirus membrane protein (M protein) or a portion thereof. The M protein is the most abundant structural protein and defines the shape of the viral envelope. The M protein is regarded as the central organizer of the viral assembly, interacting with other major coronaviral structural proteins.

In some embodiments, the coronavirus antigen used herein comprises a coronavirus envelope protein (E protein) or a portion thereof. The E protein is a small membrane protein and minor component of the virus particles. Without being bound to any theory, it is believed that the E protein plays roles in virion assembly and morphogenesis, alteration of the membrane of host cells and virus-host cell interaction.

In some embodiments, the coronavirus antigen used herein comprises a coronavirus hemagglutinin-esterase protein (HE protein) or a portion thereof. The HE protein, which is another envelope protein, mediates reversible attachment to 0-acetylated sialic acids by acting both as lectins and receptor-destroying enzymes.

In some embodiments, the coronavirus antigen used herein comprises a coronavirus papain-like protease or a portion thereof. The coronavirus papain-like protease is one of several nonstructural proteins, and is responsible for processing of viral proteins into functional, mature subunits during maturation. For example, the coronavirus papain-like protease can cleave a site at the amino-terminal end of the viral replicase region. In addition to its role in viral protein maturation, papain-like protease exhibits both a deubiquitinating and deISG15ylating activity. In vivo, this protease antagonizes innate immunity by acting on IFN beta and NF-kappa B signaling pathways.

In some embodiments, the coronavirus antigen used herein comprises a coronavirus 3CL protease or a portion thereof. The 3CL protease is another main protease in addition to the papain-like protease and is required for processing of viral polypeptides into distinct, functional proteins. In some embodiments, the 3CL protease is a SARS-CoV-2 3CL Protease, which is a C30-type cysteine protease located within the non-structural proteins 3 (NS3) region of the viral polypeptide. Analysis of the Coronavirus genome reveals at least 11 sites of cleavage for the 3CL protease, many containing the amino acid sequence LQ[S/A/G].

The coronavirus antigen disclosed herein can, in some embodiments, comprise a S protein or a portion thereof, a N protein or a portion thereof, a HE protein or a portion thereof, a papain-like protease or a portion thereof, a coronavirus 3CL protease or a portion thereof, a M protein or a portion thereof, or a combination thereof.

In some embodiments, the coronavirus antigen can be an immunogenic portion of a coronavirus protein herein described. It will be appreciated by those skilled in the art that an immunogenic portion of a coronavirus antigen can be fragments of the S protein (e.g., spike protein RBD), N protein, HE protein, papain-like protease, 3CL protease, or M protein capable of eliciting an immune response against one or more coronaviruses. The immunogenic portion can comprise about, at least or at least about, at most or at most about, 50, 60, 70, 80, 90, 100, 110, 120, 130, 140, 150, 160, 170, 180, 190, 200, 210, 220, 230, 240, 250, or a number or a range between any two of these values, contiguous amino acid residues from the coronavirus proteins. In some embodiments, the immunogenic portion comprises a S protein RBD or a portion thereof. The portion of the S protein RBD can comprise the receptor binding motif of the S protein RBD.

One or more of the plurality of CoV antigens can be of CoVs in the genus of Alpha-CoV and/or Beta-CoV, and optionally each of the plurality of CoV antigens are of CoVs in the genus of Beta-CoV. The plurality of CoV antigens can be of CoVs in the subgenus of Sarbecovirus. The first CoV and the second CoV can be in the genus of Beta-CoV, optionally in the subgenus of Sarbecovirus. The plurality of CoV antigens can be of CoVs selected from: SARS-CoV, SARS-CoV-2, WIV1, SHC014, Rfl, RmYNO2, pangl7, RaTG13, Rs4081, LYRal1, HKU3, Yunnan2011, BtKY72, BM48-31, WIV16, Khosta-1, and Khosta-2. The first CoV, the second CoV, or both can be selected from: SARS-CoV, SARS-CoV-2, WIV1, SHC014, Rfl, RmYNO2, pangl7, RaTG13, Rs4081, LYRa11, HKU3, Yunnan2011, BtKY72, BM48-31, WIV16, Khosta-1, and Khosta-2. The CoV can be selected from a species or subspecies of SARS-CoV, SARS-CoV-1, SARS-CoV-2, MERS-CoV, SL-CoV-WIVI, HKU4, HKU5, HCoV-OC43, HCoV-HKUl, HKU9, HKU3, HKU8, HKU24, NL63, SHC014, 229E and/or SARS-CoV-2 variants B.1.351, B.1.1.7, P.1, B.1.617.2, B.1.1.529, BA.1, BA.1.1, BA.2, BA.3, BA.4, BA.5 and other descendent lineages.

The CoV can be a species or subspecies of Embecovirus, Sarbecovirus, Merbecovirus, Nobevovirus, Hibecovirus, SARSr-CoV, MERS-CoV, or any combination thereof. The CoV can be selected from a beta-CoV from the sarbe-, embeco-, merbeco-, and/or nobecovirus lineages. The CoV can be selected from a sarbecovirus strain, optionally, SARS, LYRa11, Rfl, Rs4081, BtKY72, and/or BM48-31. The CoV can be selected from a merbecovirus strain, optionally HKU4, HKU5, HKU25, BtCoV-Vs-CoV1, MERS-related NL13845, MERS-related NL140422. The CoV can be selected from an embecovirus strain, optionally HKU1, Rat CoV Parker, PHEV, Equine CoV, Rodent CoV, Longquan Rat CoV.

The plurality of presenting peptide can be of a same protein type or corresponding proteins. Presenting peptide of a same protein type may or may not have identical amino acid sequences, but generally share some sequence homology. For example, the coronavirus S proteins of different coronaviruses are of a same protein type or corresponding proteins. As another example, envelope proteins from different coronaviruses are considered the same protein type or corresponding proteins. In some embodiments, proteins of different coronavirus taxonomic groups having the same function are considered the same protein type or corresponding proteins.

Alternatively, in some embodiments the plurality of presenting peptide can derive from antigens of different protein types. Antigens of different protein types typically have different functions. For example, the plurality of presenting peptide can derive from coronavirus S proteins or portions thereof as well as other coronavirus proteins such as a coronavirus N protein or a portion thereof, a coronavirus HE protein or a portion thereof, a coronavirus papain-like protease or a portion thereof, a coronavirus 3CL protease or a portion thereof, and/or a coronavirus M protein or a portion thereof.

The same APV can comprise the presenting peptides derived from two or more strains of the same family, same genus, and/or same species, of infectious agent. The plurality of disparate presenting peptide can have a sequence identity of about, at least, or at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, with one another.

The plurality of disparate (e.g., heterologous) presenting peptide can comprise at least m pathogenic antigens of an mth infectious agent, wherein m is an integer greater than 2 (e.g., 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or a number or a range between any two of these values), and wherein each mth pathogenic antigen is different from one another (e.g., heterologous). In some embodiments, m is an integer greater than 50. The plurality of disparate presenting peptide can comprise two or more of a 1st pathogenic antigen (PA) of a 1st infectious agent (IA), a 2nd PA of a 2nd IA, a 3rd PA of a 3rd IA, a 4th PA of a 4th IA, a 5th PA of a 5th IA, a 6th PA of a 6th IA, a 7th PA of a 7th IA, a 8th PA of a 8th IA, a 9th PA of a 9th IA, a 10th PA of a 10th IA, a 11th PA of a 11th IA, a 12th PA of a 12th IA, a 13th PA of a 13th IA, a 14th PA of a 14th IA, a 15th PA of a 15th IA, a 16th PA of a 16th IA, a 17th PA of a 17th IA, a 18th PA of a 18th IA, a 19th PA of a 19th IA, a 20th PA of a 20th IA, a 21st PA of a 21st IA, a 22nd PA of a 22nd IA, a 23rd PA of a 23rd IA, a 24th PA of a 24th IA, a 25th PA of a 25th IA, a 26th PA of a 26th IA, a 27th PA of a 27th IA, a 28th PA of a 28th IA, a 29th PA of a 29th IA, a 30th PA ofa 30th IA, a 31st PA ofa 31st IA, a 32nd PA ofa 32nd IA, a 33rd PA ofa 33rd IA, a 34th PA of a 34th IA, a 35th PA of a 35th IA, a 36th PA of a 36th IA, a 37th PA of a 37th IA, a 38th PA of a 38th IA, a 39th PA of a 39th IA, a 40th PA of a 40th IA, a 41st PA of a 41st IA, a 42nd PA of a 42nd IA, a 43rd PA of a 43rd IA, a 44th PA of a 44th IA, a 45th PA of a 45th IA, a 46th PA of a 46th IA, a 47th PA of a 47th IA, a 48th PA of a 48th IA, a 49th PA of a 49th IA, and a 50th PA of a 50th IA. The 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, 24th, 25th, 26th, 27th, 28th, 29th, 30th, 31st, 32nd, 33rd, 34th, 35th, 36th, 37th, 38th, 39th, 40th, 41st, 42nd, 43rd, 44th, 45th, 46th, 47th, 48th, 49th, and 50th pathogenic antigens can be the same or different from one another. The 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, 24th, 25th, 26th, 27th, 28th, 29th, 30th, 31st, 32nd, 33rd, 34th, 35th, 36th, 37th, 38th, 39th, 40th, 41st, 42nd, 43rd, 44th, 45th, 46th, 47th, 48th, 49th, and 50th infectious agents can be the same or different from one another. The plurality of disparate (e.g., heterologous) presenting peptide can comprise a plurality of CoV antigens, a plurality of influenza antigens, and/or a plurality of HIV antigens.

The compositions provided herein can induce broadly protective immune responses by eliciting broadly neutralizing antibodies and cytokines, and activating T cells with broad specificities. As an example, the broadly neutralizing antibodies, cytokine production or T cell activation can neutralize or target coronaviruses from a taxonomic group that is not only the same as but also differs from the taxonomic groups of the coronaviruses from which the coronavirus antigens used to elicit the antibodies, the cytokine production and T cell activation are derived. Broadly neutralizing response, cytokine production or T cell activation can also be referred to as heterologous response. In some embodiments, the compositions herein described can elicit broadly neutralizing antibodies, cytokine production or T cell activation that neutralize or target one or more infectious agents from a subfamily, genus, subgenus, species, and/or strain that differ from the subfamily, genus, subgenus, species, and/or strain of the infectious agents from which presenting peptide are derived to produce the APCs provided herein.

### Tumor-Associated Antigens, Autoimmune Antigens, and Allergenic Antigens

The disease or disorder can be a disease associated with expression of a tumor-associated antigen, and the antigenic protein can be a tumor-associated antigen. A tumor-associated antigen can be a tumor-specific antigen. The tumor-associated antigen can be a neoantigen identified in a subject. In some embodiments, the tumor-associated antigen is selected from: 1A01_HLA-A/m (UniProtKB: P30443); 1A02 (UniProtKB: P01892); 5T4 (UniProtKB: Q13641); ACRBP (UniProtKB: Q8NEB7); AFP (UniProtKB: P02771); AKAP4 (UniProtKB: Q5JQC9); alpha-actinin-_4/m (UniProtKB: B4DSXO); alpha-actinin-_4/m (UniProtKB: B4E337); alpha-actinin-_4/m (UniProtKB: 043707); alpha-methylacyl-coenzyme_A_racemase (UniProtKB: AOA024RE16); alpha-methylacyl-coenzyme_A_racemase (UniProtKB: A8KAC3); ANDR (UniProtKB: P10275); ART-4 (UniProtKB: Q9ULX3); ARTC1/m (UniProtKB: P52961); AURKB (UniProtKB: Q96GD4); B2MG (UniProtKB: P61769); B3GN5 (UniProtKB: Q9BYGO); B4GN1 (UniProtKB: Q00973); B7H4 (UniProtKB: Q7Z7D3); BAGE-1 (UniProtKB: Q13072); BASI (UniProtKB: P35613); BCL-2 (UniProtKB: A9QXG9); ber/abl (UniProtKB: A9UEZ4); ber/abl (UniProtKB: A9UEZ7); ber/abl (UniProtKB: A9UEZ8); ber/abl (UniProtKB: A9UEZ9); ber/abl (UniProtKB: A9UF00); ber/abl (UniProtKB: A9UF01); ber/abl (UniProtKB: A9UF03); ber/abl (UniProtKB: A9UF04); ber/abl (UniProtKB: A9UF05); ber/abl (UniProtKB: A9UF06); ber/abl (UniProtKB: A9UF08); beta-catenin/m (UniProtKB: P35222); beta-catenin/m (UniProtKB: Q8WYA6); BING-4 (UniProtKB: 015213); BIRC7 (UniProtKB: Q96CA5); BRCA1/m (UniProtKB: A0A024R1V0); BRCA1/m (UniProtKB: A0A024R1V7); BRCA1/m (UniProtKB: A0A024R1Z8); BRCA1/m (UniProtKB: A0A068BFX7); BRCA1/m (UniProtKB: C6YB45); BRCA1/m (UniProtKB: C6YB47); BRCA1/m (UniProtKB: G3XAC3); BY55 (UniProtKB: 095971); calreticulin (UniProtKB: B4DHR1); calreticulin (UniProtKB: B4E2Y9); calreticulin (UniProtKB: P27797); calreticulin (UniProtKB: Q96L12); CAMEL (UniProtKB: 095987); CASP-8/m (UniProtKB: Q14790); CASPA (UniProtKB: Q92851-4); cathepsin_B (UniProtKB: A0A024R374); cathepsin_B (UniProtKB: P07858); cathepsin_L (UniProtKB: A0A024R276); cathepsin_L (UniProtKB: P07711); cathepsin_L (UniProtKB: Q9HBQ7); CD1A (UniProtKB: P06126); CD1B (UniProtKB: P29016); CD1C (UniProtKB: P29017); CD1D (UniProtKB: P15813); CD1E (UniProtKB: P15812); CD20 (UniProtKB: P11836); CD22 (UniProtKB: 060926); CD22 (UniProtKB: P20273); CD22 (UniProtKB: QOEAF5); CD276 (UniProtKB: Q5ZPR3); CD33 (UniProtKB: B4DF51); CD33 (UniProtKB: P20138); CD33 (UniProtKB: Q546G0); CD3E (UniProtKB: P07766); CD3Z (UniProtKB: P20963); CD44_Isoform_1 (UniProtKB: P16070); CD44_Isoform_6 (UniProtKB: P16070-6); CD4 (UniProtKB: P01730); CD52 (UniProtKB: P31358); CD52 (UniProtKB: Q6IBDO); CD52 (UniProtKB: V9HWN9); CD55 (UniProtKB: B1AP15); CD55 (UniProtKB: D3DT85); CD55 (UniProtKB: D3DT86); CD55 (UniProtKB: P08174); CD56 (UniProtKB: P13591); CD80 (UniProtKB: AONOP2); CD80 (UniProtKB: P33681); CD86 (UniProtKB: P42081); CD8A (UniProtKB: P01732); CDCl27/m (UniProtKB: G5EA36); CDCl27/m (UniProtKB: P30260); CDE30 (UniProtKB: P28908); CDK4/m (UniProtKB: AOA024RBB6); CDK4/m (UniProtKB: P11802); CDK4/m (UniProtKB: Q6LC83); CDK4/m (UniProtKB: Q96BE9); CDKN2A/m (UniProtKB: D1LYX3); CDKN2A/m (UniProtKB: G3XAG3); CDKN2A/m (UniProtKB: K7PML8); CDKN2A/m (UniProtKB: L8E941); CDKN2A/m (UniProtKB: Q8N726); CEA (RefSeq: NP_004354); CEAM6 (UniProtKB: P40199); CH3L2 (UniProtKB: Q15782); CLCA2 (UniProtKB: Q9UQC9); CML28 (UniProtKB: Q9NQT4); CML66 (UniProtKB: Q96RS6); COA-1/m (UniProtKB: Q5T124); coactosin-like_protein (UniProtKB: Q14019); collagen_XXIII (UniProtKB: L8EAS4); collagen_XXIII (UniProtKB: Q86Y22); COX-2 (UniProtKB: Q6ZYK7); CP1B1 (UniProtKB: Q16678); CSAG2 (UniProtKB: Q9Y5P2-2); CSAG2 (UniProtKB: Q9Y5P2); CT45A1 (UniProtKB: Q5HYN5); CT55 (UniProtKB: Q8WUE5); CT-_9/BRD6 (UniProtKB: Q58F21); CTAG2_Isoform_LAGE-1A (UniProtKB: 075638-2); CTAG2_Isoform_LAGE-1B (UniProtKB: 075638); CTCFL (UniProtKB: Q8NI51); Cten (UniProtKB: Q8IZW8); cyclin_B1 (UniProtKB: P14635); cyclin_D1 (UniProtKB: P24385); cyp-B (UniProtKB: P23284); DAM-10 (UniProtKB: P43366); DEP1A (UniProtKB: Q5TB30); E7 (UniProtKB: P03129); E7 (UniProtKB: P06788); E7 (UniProtKB: P17387); E7 (UniProtKB: P06429); E7 (UniProtKB: P27230); E7 (UniProtKB: P24837); E7 (UniProtKB: P21736); E7 (UniProtKB: P26558); E7 (UniProtKB: P36831); E7 (UniProtKB: P36833); E7 (UniProtKB: Q9QCZ1); E7 (UniProtKB: Q81965); E7 (UniProtKB: Q80956); EF1A2 (UniProtKB: Q05639); EFTUD2/m (UniProtKB: Q15029); EGFR (UniProtKB: A0A0B4J1Y5); EGFR (UniProtKB: E7BSVO); EGFR (UniProtKB: LOR6G1); EGFR (UniProtKB: P00533-2); EGFR (UniProtKB: P00533); EGFR (UniProtKB: Q147T7); EGFR (UniProtKB: Q504U8); EGFR (UniProtKB: Q8NDU8); EGLN3 (UniProtKB: Q9H6Z9); ELF2/m (UniProtKB: B7Z720); EMMPRIN (UniProtKB: Q54A51); EpCam (UniProtKB: P16422); EphA2 (UniProtKB: P29317); EphA3 (UniProtKB: P29320); EphA3 (UniProtKB: Q6P4R6); ErbB3 (UniProtKB: B3KWG5); ErbB3 (UniProtKB: B4DGQ7); ERBB4 (UniProtKB: Q15303); ERG (UniProtKB: P11308); ETV6 (UniProtKB: P41212); EWS (UniProtKB: Q01844); EZH2 (UniProtKB: F2YMM1); EZH2 (UniProtKB: G3XAL2); EZH2 (UniProtKB: LOR855); EZH2 (UniProtKB: Q15910); EZH2 (UniProtKB: S4S3R8); FABP7 (UniProtKB: 015540); FCGR3A_Version_1 (UniProtKB: P08637); FCGR3A_Version_2 (CCDS: CCDS1232.1); FGFS (UniProtKB: P12034); FGFS (UniProtKB: Q60518); FGFR2 (UniProtKB: P21802); fibronectin (UniProtKB: A0A024R5I6); fibronectin (UniProtKB: AOA024RB01); fibronectin (UniProtKB: AOA024RDT9); fibronectin (UniProtKB: AOA024RDV5); fibronectin (UniProtKB: A6NH44); fibronectin (UniProtKB: A8K6A5); fibronectin (UniProtKB: B2R627); fibronectin (UniProtKB: B3KXM5); fibronectin (UniProtKB: B4DIC5); fibronectin (UniProtKB: B4DN21); fibronectin (UniProtKB: B4DS98); fibronectin (UniProtKB: B4DTH2); fibronectin (UniProtKB: B4DTK1); fibronectin (UniProtKB: B4DU16); fibronectin (UniProtKB: B7Z3W5); fibronectin (UniProtKB: B7Z939); fibronectin (UniProtKB: G5E9X3); fibronectin (UniProtKB: Q9H382); FOS (UniProtKB: P01100); FOXP3 (UniProtKB: Q9BZS1); FUT1 (UniProtKB: P19526); G250 (UniProtKB: Q16790); GAGE-1 (Genbank: AAA82744); GAGE-2 (UniProtKB: Q6NT46); GAGE-3 (UniProtKB: Q13067); GAGE-4 (UniProtKB: Q13068); GAGE-5 (UniProtKB: Q13069); GAGE-6 (UniProtKB: Q13070); GAGE7b (UniProtKB: 076087); GAGE-8_(GAGE-2D) (UniProtKB: Q9UEU5); GASR (UniProtKB: P32239); GnT-V (UniProtKB: Q09328); GPC3 (UniProtKB: I6QTG3); GPC3 (UniProtKB: P51654); GPC3 (UniProtKB: Q8IYG2); GPNMB/m (UniProtKB: AOA024RA55); GPNMB/m (UniProtKB: Q14956); GPNMB/m (UniProtKB: Q8IXJ5); GPNMB/m (UniProtKB: Q96F58); GRM3 (UniProtKB: Q14832); HAGE (UniProtKB: Q9NXZ2); hepsin (UniProtKB: B2ZDQ2); hepsin (UniProtKB: P05981); Her2/neu (UniProtKB: B4DTR1); Her2/neu (UniProtKB: L8E8G2); Her2/neu (UniProtKB: P04626); Her2/neu (UniProtKB: Q9UK79); HLA-A2/m (UniProtKB: Q95387); HLA-A2/m (UniProtKB: Q9MYF8); homeobox_NKX3.1 (UniProtKB: Q99801); HOM-TES-85 (UniProtKB: B2RBQ6); HOM-TES-85 (UniProtKB: Q9P127); HPG1 (Pubmed: 12543784); HS71A (UniProtKB: PODMV8); HS71B (UniProtKB: PODMV9); HST-2 (UniProtKB: P10767); hTERT (UniProtKB: 094807); iCE (UniProtKB: 000748); IF2B3 (UniProtKB: 000425); IL10 (UniProtKB: P22301); IL-13Ra2 (UniProtKB: Q14627); IL2-RA (UniProtKB: P01589); IL2-RB (UniProtKB: P14784); IL2-RG (UniProtKB: P31785); IL-5 (UniProtKB: P05113); IMP3 (UniProtKB: Q9NV31); ITA5 (UniProtKB: P08648); ITB1 (UniProtKB: P05556); ITB6 (UniProtKB: P18564); kallikrein-2 (UniProtKB: A0A024R4J4); kallikrein-2 (UniProtKB: A0A024R4N3); kallikrein-2 (UniProtKB: BOAZU9); kallikrein-2 (UniProtKB: B4DU77); kallikrein-2 (UniProtKB: P20151); kallikrein-2 (UniProtKB: Q6T774); kallikrein-2 (UniProtKB: Q6T775); kallikrein-4 (UniProtKB: AOAOC4DFQ5); kallikrein-4 (UniProtKB: Q5BQAO); kallikrein-4 (UniProtKB: Q96PTO); kallikrein-4 (UniProtKB: Q96PT1); kallikrein-4 (UniProtKB: Q9Y5K2); KI20A (UniProtKB: 095235); KIAA0205 (UniProtKB: Q92604); KIF2C (UniProtKB: Q99661); KK-LC-1 (UniProtKB: Q5H943); LDLR (UniProtKB: P01130); LGMN (UniProtKB: Q99538); LIRB2 (UniProtKB: Q8N423); LY6K (UniProtKB: Q17RY6); MAGAS (UniProtKB: P43359); MAGA8 (UniProtKB: P43361); MAGAB (UniProtKB: P43364); MAGE-A10 (UniProtKB: AOA024RC14); MAGE-A12 (UniProtKB: P43365); MAGE-A1 (UniProtKB: P43355); MAGE-A2 (UniProtKB: P43356); MAGE-A3 (UniProtKB: P43357); MAGE-A4 (UniProtKB: AOA024RC12); MAGE-A4 (UniProtKB: P43358); MAGE-A4 (UniProtKB: Q1RN33); MAGE-A6 (UniProtKB: A8K072); MAGE-A6 (UniProtKB: P43360); MAGE-A6 (UniProtKB: Q6FHI5); MAGE-A9 (UniProtKB: P43362); MAGE-B10 (UniProtKB: Q96LZ2); MAGE-B16 (UniProtKB: A2A368); MAGE-B17 (UniProtKB: A8MXT2); MAGE-_B1 (UniProtKB: Q96TG1); MAGE-B2 (UniProtKB: 015479); MAGE-B3 (UniProtKB: 015480); MAGE-B4 (UniProtKB: 015481); MAGE-B5 (UniProtKB: Q9BZ81); MAGE-B6 (UniProtKB: Q8N7X4); MAGE-C1 (UniProtKB: 060732); MAGE-C2 (UniProtKB: Q9UBF1); MAGE-C3 (UniProtKB: Q8TD91); MAGE-D1 (UniProtKB: Q9Y5V3); MAGE-D2 (UniProtKB: Q9UNF1); MAGE-D4 (UniProtKB: Q96JG8); MAGE-_E1 (UniProtKB: Q6IAI7); MAGE-E1_(MAGE1) (UniProtKB: Q9HCI5); MAGE-E2 (UniProtKB: Q8TD90); MAGE-F1 (UniProtKB: Q9HAY2); MAGE-H1 (UniProtKB: Q9H213); MAGEL2 (UniProtKB: Q9U355); mammaglobin_A (UniProtKB: Q13296); mammaglobin_A (UniProtKB: Q6NX70); MART-1/melan-A (UniProtKB: Q16655); MART-2 (UniProtKB: Q5VTY9); MC1_R (UniProtKB: Q01726); MC1_R (UniProtKB: Q1JUL4); MC1_R (UniProtKB: Q1JJUL6); MC1_R (UniProtKB: Q1JUL8); MCi_R (UniProtKB: Q1JUL9); MC1_R (UniProtKB: Q1JTJUMO); MC1_R (UniProtKB: Q1JJUM2); MCi_R (UniProtKB: Q1JUM3); MC1_R (UniProtKB: Q1JUM4); MC1_R (UniProtKB: Q1JUM5); MC1_R (UniProtKB: Q6UR92); MC1_R (UniProtKB: Q6UR94); MCi_R (UniProtKB: Q6UR95); MC1_R (UniProtKB: Q6UR96); MCiR (UniProtKB: Q6UR97); MC1_R (UniProtKB: Q6UR98); MCiR (UniProtKB: Q6UR99); MC1_R (UniProtKB: Q6URAO); MC1_R (UniProtKB: Q86YW1); MC1_R (UniProtKB: V9Q5S2); MCi_R (UniProtKB: V9Q671); MC1_R (UniProtKB: V9Q783); MC1_R (UniProtKB: V9Q7F1); MCi_R (UniProtKB: V9Q8N1); MC1_R (UniProtKB: V9Q977); MC1_R (UniProtKB: V9Q9P5); MC1_R (UniProtKB: V9Q9R8); MC1_R (UniProtKB: V9QAEO); MC1_R (UniProtKB: V9QAR2); MC1_R (UniProtKB: V9QAW3); MC1_R (UniProtKB: V9QB02); MC1_R (UniProtKB: V9QB58); MC1_R (UniProtKB: V9QBY6); MC1_R (UniProtKB: V9QC17); MC1_R (UniProtKB: V9QC66); MC1_R (UniProtKB: V9QCQ4); MC1_R (UniProtKB: V9QDF4); MC1_R (UniProtKB: V9QDN7); MC1_R (UniProtKB: V9QDQ6); M-CSF (UniProtKB: P09603); mesothelin (UniProtKB: Q13421); MITF (UniProtKB: 075030-8); MITF (UniProtKB: 075030-9); MITF (UniProtKB: 075030); MMP1_1 (UniProtKB: B3KQS8); MMP7 (UniProtKB: P09237); MUC-1 (Genbank: AAA60019); MUM-1/m (RefSeq: NP_116242); MUM-2/m (UniProtKB: Q9Y5R8); MYCN (UniProtKB: P04198); MYO1A (UniProtKB: Q9UBC5); MYO1B (UniProtKB: 043795); MYO1C (UniProtKB: 000159); MYO1D (UniProtKB: 094832); MYO1E (UniProtKB: Q12965); MYO1F (UniProtKB: 000160); MYO1G (UniProtKB: BOI1T2); MYO1H (RefSeq: NP_001094891); NA17 (UniProtKB: Q3V5L5); NA88-A (Pubmed: 10790436); Neo-PAP (UniProtKB: Q9BWT3); NFYC/m (UniProtKB: Q13952); NGEP (UniProtKB: Q6IWH7); NPM (UniProtKB: P06748); NRCAM (UniProtKB: Q92823); NSE (UniProtKB: P09104); NUF2 (UniProtKB: Q9BZD4); NY-ESO-1 (UniProtKB: P78358); OA1 (UniProtKB: P51810); OGT (UniProtKB: 015294); OS-9 (UniProtKB: B4DH11); OS-9 (UniProtKB: B4E321); OS-9 (UniProtKB: B7Z8E7); OS-9 (UniProtKB: Q13438); osteocalcin (UniProtKB: P02818); osteopontin (UniProtKB: AOA024RDE2); osteopontin (UniProtKB: AOA024RDE6); osteopontin (UniProtKB: AOA024RDJO); osteopontin (UniProtKB: B7Z351); osteopontin (UniProtKB: F2YQ21); osteopontin (UniProtKB: P10451); p53 (UniProtKB: P04637); PAGE-4 (UniProtKB: 060829); PAI-1 (UniProtKB: P05121); PAI-2 (UniProtKB: P05120); PAP (UniProtKB: Q06141); PAP (UniProtKB: Q53S56); PATE (UniProtKB: Q8WXA2); PAX3 (UniProtKB: P23760); PAXS (UniProtKB: Q02548); PD1L1 (UniProtKB: Q9NZQ7); PDCD1 (UniProtKB: Q15116); PDEF (UniProtKB: 095238); PECA1 (UniProtKB: P16284); PGCB (UniProtKB: Q96GW7); PGFRB (UniProtKB: P09619); Pim-1_-Kinase (UniProtKB: A0A024RD25); Pin-1 (UniProtKB: 015428); Pin-1 (UniProtKB: Q13526); Pin-1 (UniProtKB: Q49AR7); PLAC1 (UniProtKB: Q9HBJO); PMEL (UniProtKB: P40967); PML (UniProtKB: P29590); POTEF (UniProtKB: A5A3E0); POTE (UniProtKB: Q86YR6); PRAME (UniProtKB: AOA024R1E6); PRAME (UniProtKB: P78395); PRDX5/m (UniProtKB: P30044); PRM2 (UniProtKB: P04554); prostein (UniProtKB: Q96JT2); proteinase-3 (UniProtKB: D6CHE9); proteinase-3 (UniProtKB: P24158); PSA (UniProtKB: P55786); PSB9 (UniProtKB: P28065); PSCA (UniProtKB: D3DWI6); PSCA (UniProtKB: 043653); PSGR (UniProtKB: Q9H255); PSM (UniProtKB: Q04609); PTPRC (RefSeq: NP_002829); RAB8A (UniProtKB: P61006); RAGE-1 (UniProtKB: Q9UQ07); RARA (UniProtKB: P10276); RASH (UniProtKB: P01112); RASK (UniProtKB: P01116); RASN (UniProtKB: P01111); RGSS (UniProtKB: 015539); RHANM/CD168 (UniProtKB: 075330); RHOC (UniProtKB: P08134); RSSA (UniProtKB: P08865); RU1 (UniProtKB: Q9UHJ3); RU2 (UniProtKB: Q9UHGO); RUNX1 (UniProtKB: Q01196); S-100 (UniProtKB: V9HW39); SAGE (UniProtKB: Q9NXZ1); SART-_1 (UniProtKB: 043290); SART-2 (UniProtKB: Q9UL01); SART-3 (UniProtKB: Q15020); SEPR (UniProtKB: Q12884); SERPINBS (UniProtKB: P36952); SIA7F (UniProtKB: Q969X2); SIA8A (UniProtKB: Q92185); SIAT9 (UniProtKB: Q9UNP4); SIRT2/m (UniProtKB: AOA024ROG8); SIRT2/m (UniProtKB: Q8IXJ6); SOX10 (UniProtKB: P56693); SP17 (UniProtKB: Q15506); SPNXA (UniProtKB: Q9NS26); SPXN3 (UniProtKB: Q5MJO9); SSX-1 (UniProtKB: Q16384); SSX-2 (UniProtKB: Q16385); SSX3 (UniProtKB: Q99909); SSX-4 (UniProtKB: 060224); ST1A1 (UniProtKB: P50225); STAG2 (UniProtKB: Q8N3U4-2); STAMP-1 (UniProtKB: Q8NFT2); STEAP-1 (UniProtKB: A0A024RA63); STEAP-1 (UniProtKB: Q9UHE8); Survivin-2B (UniProtKB: 015392-2); survivin (UniProtKB: 015392); SYCP1 (UniProtKB: A0A024R0I2); SYCP1 (UniProtKB: B7ZLS9); SYCP1 (UniProtKB: Q15431); SYCP1 (UniProtKB: Q3MHC4); SYT-SSX-1 (UniProtKB: A4PIV7); SYT-SSX-1 (UniProtKB: A4PIV8); SYT-SSX-2 (UniProtKB: A4PIV9); SYT-SSX-2 (UniProtKB: A4PIWO); TARP (UniProtKB: QOVGM3); TCRg (UniProtKB: A2JGV3); TF2AA (UniProtKB: P52655); TGFB1 (UniProtKB: P01137); TGFR2 (UniProtKB: P37173); TGM-4 (UniProtKB: B2R7D1); TIE2 (UniProtKB: Q02763); TKIL1 (UniProtKB: P51854); TPI/m (UniProtKB: P60174); TRGV11 (UniProtKB: Q99601); TRGV9 (UniProtKB: A4D1X2); TRGV9 (UniProtKB: Q99603); TRGV9 (UniProtKB: Q99604); TRPC1 (UniProtKB: P48995); TRP-p8 (UniProtKB: Q7Z2W7); TSG10 (UniProtKB: Q9BZW7); TSPY1 (UniProtKB: Q01534); TVC_(TRGV3) (Genbank: M13231.1); TX101 (UniProtKB: Q9BYl4-2); tyrosinase (UniProtKB: AOA024DBG7); tyrosinase (UniProtKB: L8B082); tyrosinase (UniProtKB: L8B086); tyrosinase (UniProtKB: L8B0B9); tyrosinase (UniProtKB: 075767); tyrosinase (UniProtKB: P14679); tyrosinase (UniProtKB: U3M8NO); tyrosinase (UniProtKB: U3M9D5); tyrosinase (UniProtKB: U3M9J2); TYRP1 (UniProtKB: P17643); TYRP2 (UniProtKB: P40126); UPA (UniProtKB: Q96NZ9); VEGFR1 (UniProtKB: B5A924); WT1 (UniProtKB: AOAOH5AUYO); WT1 (UniProtKB: P19544); WT1 (UniProtKB: Q06250); and XAGE1 (UniProtKB: Q9HD64).

The disease or disorder can be an autoimmune disease or disorder, and the antigenic protein can be an autoimmune antigen. The autoimmune antigen can be an autoimmune antigen listed in Table 3. Autoimmune antigens include, but are not limited to, one or more of myelin basic protein (MBP), proteolipid protein (PLP), and myelin oligodendrocyte glycoprotein (MOG), in each case associated with multiple sclerosis (MS); CD44, preproinsulin, proinsulin, insulin, glutamic acid decaroxylase (GAD65), tyrosine phosphatase-like insulinoma antigen 2 (IA2), zinc transporter ((ZnT8), and heat shock protein 60 (HSP60), in each case associated with diabetes Typ I; interphotoreceptor retinoid-binding protein (TRBP) associated with autoimmune uveitis; acetylcholine receptor AchR, and insulin-like growth factor-1 receptor (IGF-1R), in each case associated with Myasthenia gravis; M-protein from beta-hemolytic streptocci (pseudo-autoantigen) associated with Rheumatic Fever, or any combination thereof. Autoimmune antigens (antigens associated with autoimmune disease or autoantigens) can be selected from autoantigens associated with autoimmune diseases selected from Addison disease (autoimmune adrenalitis, Morbus Addison), alopecia areata, Addison's anemia (Morbus Biermer), autoimmune hemolytic anemia (AIHA), autoimmune hemolytic anemia (AIHA) of the cold type (cold hemagglutinine disease, cold autoimmune hemolytic anemia (AIHA) (cold agglutinin disease), (CHAD)), autoimmune hemolytic anemia (AIHA) of the warm type (warm AIHA, warm autoimmune haemolytic anemia (AIHA)), autoimmune hemolytic Donath-Landsteiner anemia (paroxysmal cold hemoglobinuria), antiphospholipid syndrome (APS), atherosclerosis, autoimmune arthritis, arteriitis temporalis, Takayasu arteriitis (Takayasu's disease, aortic arch disease), temporal arteriitis/giant cell arteriitis, autoimmune chronic gastritis, autoimmune infertility, autoimmune inner ear disease (AIED), Basedow's disease (Morbus Basedow), Bechterew's disease (Morbus Bechterew, ankylosing spondylitis, spondylitis ankylosans), Behcet's syndrome (Morbus Behcet), bowel disease including autoimmune inflammatory bowel disease (including colitis ulcerosa (Morbus Crohn, Crohn's disease), cardiomyopathy, particularly autoimmune cardiomyopathy, idiopathic dilated cardiomyopathy (DCM), celiac sprue dermatitis (gluten mediated enteropathia), chronic fatigue immune dysfunction syndrome (CFIDS), chronic inflammatory demyelinating polyneuropathy (CIDP), chronic polyarthritis, Churg-Strauss syndrome, cicatricial pemphigoid, Cogan syndrome, CREST syndrome (syndrom with Calcinosis cutis, Raynaud phenomenon, motility disorders of the esophagus, sklerodaktylia and teleangiectasia), Crohn's disease (Morbus Crohn, colitis ulcerosa), dermatitis herpetiformis during, dermatologic autoimmune diseases, dermatomyositis, Diabetes, Diabetes mellitus Type 1 (type I diabetes, insuline dependent Diabetes mellitus), Diabetes mellitus Type 2 (type II diabetes), essential mixed cryoglobulinemia, essential mixed cryoglobulinemia, fibromyalgia, fibromyositis, Goodpasture syndrome (anti-GBM mediated glomerulonephritis), graft versus host disease, Guillain-Barre syndrome (GBM, Polyradikuloneuritis), haematologic autoimmune diseases, Hashimoto thyroiditis, hemophilia, acquired hemophilia, hepatitis, autoimmune hepatitis, particularly autoimmune forms of chronic hepatitis, idiopathic pulmonary fibrosis (IPF), idiopathic thrombocytopenic purpura, Immuno-thrombocytopenic purpura (Morbus Werlhof; ITP), IgA nephropathy, infertility, autoimmune infertility, juvenile rheumatoid arthritis (Morbus Still, Still syndrome), Lambert-Eaton syndrome, lichen planus, lichen sclerosus, lupus erythematosus, systemic lupus erythematosus (SLE), lupus erythematosus (discoid form), Lyme arthritis (Lyme disease, borrelia arthritis), Meniere's disease (Morbus Meniere); mixed connective tissue disease (MCTD) multiple sclerosis (MS, encephalomyelitis disseminate, Charcot's disease), Myasthenia gravis (myasthenia, MG), myosits, polymyositis, neural autoimmune diseases, neurodermitis, pemphigus vulgaris, bullous pemphigoid, scar forming pemphigoid; polyarteriitis nodosa (periarteiitis nodosa), polychondritis (panchondritis), polyglandular (autoimmune) syndrome (PGA syndrome, Schmidt's syndrome), Polymyalgia rheumatica, primary agammaglobulinemia, primary biliary cirrhosis PBC, primary autoimmune cholangitis), progressive systemic sclerosis (PSS), Psoriasis, Psoriasis vulgaris, Raynaud's phenomena, Reiter's syndrome (Morbus Reiter, urethral conjunctive synovial syndrome)), rheumatoid arthritis (RA, chronic polyarthritis, rheumatic disease of the joints, rheumatic fever), sarcoidosis (Morbus Boeck, Besnier-Boeck-Schaumann disease), stiff-man syndrome, Sclerodermia, Scleroderma, Sjögren's syndrome, sympathetic ophtalmia; Transient gluten intolerance, transplanted organ rejection, uveitis, autoimmune uveiitis, Vasculitis, Vitiligo, (leucoderma, piebold skin), and Wegner's disease (Morbus Wegner, Wegner's granulomatosis).

The disease or disorder can be an allergic disease or disorder, and the antigenic protein can be an allergenic antigen. In some embodiments, the allergenic antigen is selected from: Acarus spp (Aca s 1, Aca s 10, Aca s 10.0101, Aca s 13, Aca s 13.0101, Aca s 2, Aca s 3, Aca s 7, Aca s 8), Acanthocybium spp (Aca so 1), Acanthocheilonema spp (Aca v 3, Aca v 3.0101), Acetes spp (Ace ja 1), Actinidia spp (Act a 1, Act c 1, Act c 10, Act c 10.0101, Act c 2, Act c 4, Act c 5, Act c 5.0101, Act c 8, Act c 8.0101, Act c Chitinase, Act d 1, Act d 1.0101, Act d 10, Act d 10.0101, Act d 10.0201, Act d 11, Act d 11.0101, Act d 2, Act d 2.0101, Act d 3, Act d 3.0101, Act d 3.02, Act d 4, Act d 4.0101, Act d 5, Act d 5.0101, Act d 6, Act d 6.0101, Act d 7, Act d 7.0101, Act d 8, Act d 8.0101, Act d 9, Act d 9.0101, Act d Chitinase, Act e 1, Act e 5), Acyrthosiphon spp (Acy pi 7, Acy pi 7.0101, Acy pi 7.0102), Adenia spp (Ade v RIP), Aedes spp (Aed a 1, Aed a 1.0101, Aed a 2, Aed a 2.0101, Aed a 3, Aed a 3.0101, Aed a 4, Aed a 7, Aed a 7.0101, Aed a 7.0102, Aed a 7.0103, Aed a 7.0104, Aed a 7.0105, Aed a 7.0106, Aed a 7.0107, Aed a 7.0108, Aed a 7.0109, Aed a 7.0110, Aed a 7.0111, Aed al 1, Aed al 3, Aed al 37 kD, Aed v 37 kD, Aed v 63 kD), Aegilops spp (Aeg ta 28, Aeg ta alpha_Gliadin, Aeg um 28, Aeg un 28), Aethaloperca spp (Aet ro 1), Agropyron spp (Agr c 7), Agrostis spp (Agr ca 1, Agr ca 5, Agr g 1, Agr g 4, Agr s 5), Agrobacterium spp (Agr sp CP4 EPSPS), Ailuropoda spp (Ail me Phosvitin, Ail me TCTP), Aix spp (Aix ga 1, Aix sp 1), Aleuroglyphus spp (Ale o 1, Ale o 10, Ale o 10.0101, Ale o 10.0102, Ale o 13, Ale o 14, Ale o 2, Ale o 20, Ale o 3, Ale o 5, Ale o 7, Ale o 8, Ale o 9), Allium spp (All a 3, All a Alliin lyase, All c 3, All c 30 kD, All c 4, All c Alliin lyase, All p Alliin lyase, All s Alliin lyase), Alnus spp (Aln g 1, Aln g 1.0101, Aln g 1/Bet v 1/Cor a 1 TPC7, Aln g 1/Bet v 1/Cor a 1 TPC9, Aln g 2, Aln g 4, Aln g 4.0101), Alopochen spp (Alo ae 1), Alopecurus spp (Alo p 1, Alo p 5), Alternaria spp (Alt a 1, Alt a 1.0101, Alt a 1.0102, Alt a 10, Alt a 10.0101, Alt a 12, Alt a 12.0101, Alt a 13, Alt a 13.0101, Alt a 2, Alt a 3, Alt a 3.0101, Alt a 4, Alt a 4.0101, Alt a 5, Alt a 5.0101, Alt a 6, Alt a 6.0101, Alt a 7, Alt a 7.0101, Alt a 70 kD, Alt a 8, Alt a 8.0101, Alt a 9, Alt a MnSOD, Alt a NTF2, Alt a TCTP, Alt ar 1, Alt arg 1, Alt b 1, Alt bl 1, Alt br 1, Alt c 1, Alt ca 1, Alt ce 1, Alt ch 1, Alt ci 1, Alt co 1, Alt cr 1, Alt ct 1, Alt cu 1, Alt cy 1, Alt d 1, Alt du 1, Alt e 1, Alt et 1, Alt eu 1, Alt ga 1, Alt gr 1, Alt j 1, Alt l 1, Alt lo 1, Alt m 1, Alt me 1, Alt mi 1, Alt mo 1, Alto 1, Alt p 1, Alt ph 1, Alt po 1, Alt ps 1, Alt r 1, Alt s 1, Alt se 1, Alt sm 1, Alt so 1, Alt su 1, Alt t 1, Alt to 1, Alt to 1), Amaranthus spp (Ama r 2, Ama r 2.0101, Ama v 2, Ama v 2.0101, Ama v 2.0201), Ambrosia spp (Amb a 1, Amb a 1.0101, Amb a 1.0201, Amb a 1.0202, Amb a 1.0301, Amb a 1.0302, Amb a 1.0303, Amb a 1.0304, Amb a 1.0305, Amb a 1.0401, Amb a 1.0402, Amb a 1.0501, Amb a 1.0502, Amb a 10, Amb a 10.0101, Amb a 3, Amb a 3.0101, Amb a 4, Amb a 4.0101, Amb a 5, Amb a 5.0101, Amb a 6, Amb a 6.0101, Amb a 7, Amb a 7.0101, Amb a 8, Amb a 8.0101, Amb a 8.0102, Amb a 9, Amb a 9.0101, Amb a 9.0102, Amb a CPI, Amb p 1, Amb p 5, Amb p 5.0101, Amb p 5.0201, Amb t 5, Amb t 5.0101, Amb t 8), Ammothea spp (Amm h 7, Amm h 7.0101), Anadara spp (Ana br 1), Ananas spp (Ana c 1, Ana c 1.0101, Ana c 2, Ana c 2.0101, Ana c 2.0101 (MUXF3)), Anas spp (Ana ca 1), Anarhichas spp (Ana I 1), Anacardium spp (Ana o 1, Ana o 1.0101, Ana o 1.0102, Ana o 2, Ana o 2.0101, Ana o 3, Ana o 3.0101), Anas spp (Ana p 1, Ana p 2, Ana p 3), Anguilla spp (Ang a 1, Ang j 1), Anisakis spp (Ani s 1, Ani s 1.0101, Ani s 10, Ani s 10.0101, Ani s 11, Ani s 11.0101, Ani s 12, Ani s 12.0101, Ani s 2, Ani s 2.0101, Ani s 24 kD, Ani s 3, Ani s 3.0101, Ani s 4, Ani s 4.0101, Ani s 5, Ani s 5.0101, Ani s 6, Ani s 6.0101, Ani s 7, Ani s 7.0101, Ani s 8, Ani s 8.0101, Ani s 9, Ani s 9.0101, Ani s CCOS3, Ani s Cytochrome B, Ani s FBPP, Ani s NADHDS4L, Ani s NARaS, Ani s PEPB, Ani s Troponin), Annona spp (Ann c Chitinase), Anopheles spp (Ano da 17, Ano da 17.0101, Ano da 27, Ano da 27.0101, Ano da 7, Ano da 7.0101, Ano g 7, Ano g 7.0101), Anser spp (Ans a 1, Ans a 2, Ans a 3, Ans in 1), Anthoxanthum spp (Ant o 1, Ant o 1.0101, Ant o 12, Ant o 13, Ant o 2, Ant o 4, Ant o 5, Ant o 6, Ant o 7), Apis spp (Api c 1, Api c 1.0101, Api c 10, Api c 2, Api c 4, Api d 1, Api d 1.0101, Api d 4, Api fl 4), Apium spp (Api g 1, Api g 1.0101, Api g 1.0201, Api g 2, Api g 2.0101, Api g 3, Api g 3.0101, Api g 4, Api g 4.0101, Api g 5, Api g 5.0101, Api g 6, Api g 6.0101), Apis spp (Api m 1, Api m 1.0101, Api m 10, Api m 10.0101, Api m 11, Api m 11.0101, Api m 11.0201, Api m 13 kD, Api m 2, Api m 2.0101, Api m 3, Api m 3.0101, Api m 4, Api m 4.0101, Api m 5, Api m 5.0101, Api m 6, Api m 6.0101, Api m 7, Api m 7.0101, Api m 8, Api m 8.0101, Api m 9, Api m 9.0101, Api m A1-A2, Api m A1-A2-A3, Api m Apalbumin 1, Api m Apalbumin 2, Api me 1, Api me 4), Arachis spp (Ara d 2, Ara d 6, Ara f 3, Ara f 4, Ara h 1, Ara h 1.0101, Ara h 10, Ara h 10.0101, Ara h 10.0102, Ara h 11, Ara h 11.0101, Ara h 2, Ara h 2.0101, Ara h 2.0102, Ara h 2.0201, Ara h 2.0202, Ara h 3, Ara h 3.0101, Ara h 4, Ara h 4.0101, Ara h 5, Ara h 5.0101, Ara h 6, Ara h 6.0101, Ara h 7, Ara h 7.0101, Ara h 7.0201, Ara h 7.0202, Ara h 8, Ara h 8.0101, Ara h 8.0201, Ara h 9, Ara h 9.0101, Ara h 9.0201, Ara h Agglutinin, Ara h Oleosin 18 kD, Ara i 2, Ara i 6), Arabidopsis spp (Ara t 3, Ara t 8, Ara t GLP), Archosargus spp (Arc pr 1), Archaeopotamobius spp (Arc s 8, Arc s 8.0101), Aequipecten spp (Arg i 1), Argas spp (Arg r 1, Arg r 1.0101), Ariopsis spp (Ari fe 1), Armoracia spp (Arm r HRP), Arrhenatherum spp (Arr e 1, Arr e 5), Artemisia spp (Art a 1, Art ap 1), Artemia spp (Art fr 1, Art fr 1.0101, Art fr 5, Art fr 5.0101), Arthrobacter spp (Art gl CO), Achorion spp (Art gy 7), Artocarpus spp (Art h 17 kD, Art h 4), Arthrospira spp (Art pl beta_Phycocyanin), Artemisia spp (Art v 1, Art v 1.0101, Art v 1.0102, Art v 1.0103, Art v 1.0104, Art v 1.0105, Art v 1.0106, Art v 1.0107, Art v 2, Art v 2.0101, Art v 3, Art v 3.0101, Art v 3.0201, Art v 3.0202, Art v 3.0301, Art v 4, Art v 4.0101, Art v 4.0201, Art v 47 kD, Art v 5, Art v 5.0101, Art v 6, Art v 6.0101, Art v 60 kD), Arthroderma spp (Art va 4), Ascaris spp (Asc 13, Asc 1 3.0101, Asc 1 3.0102, Asc 1 34 kD, Asc s 1, Asc s 1.0101, Asc s 3, Asc s 3.0101, Asc s GST), Aspergillus spp (Asp aw Glucoamylase, Asp c 22, Asp f 1, Asp f 1.0101, Asp f 10, Asp f 10.0101, Asp f 11, Asp f 11.0101, Asp f 12, Asp f 12.0101, Asp f 13, Asp f 13.0101, Asp f 15, Asp f 15.0101, Asp f 16, Asp f 16.0101, Asp f 17, Asp f 17.0101, Asp f 18, Asp f 18.0101, Asp f 2, Asp f 2.0101, Asp f 22, Asp f 22.0101, Asp f 23, Asp f 23.0101, Asp f 27, Asp f 27.0101, Asp f 28, Asp f 28.0101, Asp f 29, Asp f 29.0101, Asp f 3, Asp f 3.0101, Asp f 34, Asp f 34.0101, Asp f 4, Asp f 4.0101, Asp f 5, Asp f 5.0101, Asp f 56 kD, Asp f 6, Asp f 6.0101, Asp f 7, Asp f 7.0101, Asp f 8, Asp f 8.0101, Asp f 9, Asp f 9.0101, Asp f AfCalAp, Asp f AT_V, Asp f Catalase, Asp f Chitosanase, Asp f CP, Asp f DPPV, Asp f FDH, Asp f gamma_Actin, Asp f Glucosidase, Asp f GPI, Asp f GST, Asp f GT, Asp f IAO, Asp f IPMI, Asp f LPL1, Asp f LPL3, Asp f Mannosidase, Asp f MDH, Asp f PL, Asp f PUP, Asp f RPS3, Asp f SXR, Asp fl 13, Asp fl 13.0101, Asp fl 18, Asp fl 2, Asp fl 21, Asp fl 3, Asp fl 4, Asp fl 7, Asp fl 8, Asp fl 9, Asp me Sea prose, Asp n 14, Asp n 14.0101, Asp n 18, Asp n 18.0101, Asp n 25, Asp n 25.0101, Asp n 30, Asp n Glucoamylase, Asp n Hemicellulase, Asp n Pectinase, Asp o 13, Asp o 13.0101, Asp o 21, Asp o 21.0101, Asp o 3, Asp o 4, Asp o 7, Asp o 8, Asp o Lactase, Asp o Lipase, Asp oc 13, Asp r 1, Asp sa AP, Asp sp Glucoamylase, Asp sp Glucoseoxidase, Asp sp PL, Asp sp PME, Asp sy 13, Asp v 13, Asp v 13.0101, Asp v Catalase A, Asp v Enolase, Asp v GAPDH, Asp v MDH, Asp v SXR), Asparagus spp (Aspa o 1, Aspa o 1.01, Aspa o 1.02, Aspa o 17 kD, Aspa o 4), Aspergillus spp (Aspe ni 2, Aspe ni 3, Aspe ni 4, Aspe ni 7, Aspe ni 8, Aspe ni 9), Avena spp (Ave s 1, Ave s 12, Ave s 13, Ave s 2, Ave s 4, Ave s 5, Ave s 7), Babylonia spp (Bab ja 1), Bacillus spp (Bac al Subtilisin, Bac cl Subtilisin, Bac I Subtilisin, Bac Ii aA, Bac Ii Subtilisin), Bactrocera spp (Bac o1 27, Bac o1 27.0101), Bacillus spp (Bac sp aAl, Bac sp aA3, Bac sp Decarboxylase, Bac st amyM, Bac su Subtilisin, Bac t CrylAb, Bac t CrylFa, Bac t Cry3Bbl, Bac t Cry9c), Bagre spp (Bag ma 1), Balistes spp (Bal ca 1), Balanus spp (Bal r 1, Bal r 1.0101), Beauveria spp (Bea b AId, Bea b Enol, Bea b f2, Bea b Hex), Bertholletia spp (Ber e 1, Ber e 1.0101, Ber e 2, Ber e 2.0101), Beryx spp (Ber sp 1), Betula spp (Bet ab 1, Bet al 1, Bet ch 1, Bet co 1, Bet da 1, Bet gr 1, Bet hu 1, Bet le 1, Bet me 1, Bet n 1, Bet p 1, Bet pa 1, Bet po 1, Bet pu 1, Bet pu 2, Bet pu 4, Bet pu 6, Bet pu 7, Bet sc 1, Bet ut 1, Bet v 1, Bet v 1 B1-131-131, Bet v 1 fv Mal 4×, Bet v 1.0101, Bet v 1.0102, Bet v 1.0103, Bet v 1.0201, Bet v 1.0301, Bet v 1.0401, Bet v 1.0402, Bet v 1.0501, Bet v 1.0601, Bet v 1.0602, Bet v 1.0701, Bet v 1.0801, Bet v 1.0901, Bet v 1.1001, Bet v 1.1101, Bet v 1.1201, Bet v 1.1301, Bet v 1.1401, Bet v 1.1402, Bet v 1.1501, Bet v 1.1502, Bet v 1.1601, Bet v 1.1701, Bet v 1.1801, Bet v 1.1901, Bet v 1.2001, Bet v 1.2101, Bet v 1.2201, Bet v 1.2301, Bet v 1.2401, Bet v 1.2501, Bet v 1.2601, Bet v 1.2701, Bet v 1.2801, Bet v 1.2901, Bet v 1.3001, Bet v 1.3101, Bet v 2, Bet v 2.0101, Bet v 3, Bet v 3.0101, Bet v 4, Bet v 4.0101, Bet v 6, Bet v 6.0101, Bet v 6.0102, Bet v 7, Bet v 7.0101, Bet v 8, Bet v Glucanase), Beta spp (Beta v 1, Beta v 1.0101, Beta v 2, Beta v 2.0101), Blattella spp (Bla g 1, Bla g 1.0101, Bla g 1.0102, Bla g 1.0103, Bla g 1.0201, Bla g 1.0202, Bla g 2, Bla g 2.0101, Bla g 2.0201, Bla g 36 kD, Bla g 4, Bla g 4.0101, Bla g 4.0201, Bla g 5, Bla g 5.0101, Bla g 5.0201, Bla g 6, Bla g 6.0101, Bla g 6.0201, Bla g 6.0301, Bla g 7, Bla g 7.0101, Bla g 8, Bla g 8.0101, Bla g 9, Bla g Enolase, Bla g GSTD1, Bla g RACK1, Bla g TPI, Bla g Trypsin, Bla g Vitellogenin), Blatta spp (Bla o 1, Bla o 7), Blomia spp (Blo t 1, Blo t 1.0101, Blo t 1.0201, Blo t 10, Blo t 10.0101, Blo t 10.0102, Blo t 11, Blo t 11.0101, Blo t 12, Blo t 12.0101, Blo t 12.0102, Blo t 13, Blo t 13.0101, Blo t 14, Blo t 15, Blo t 18, Blo t 19, Blo t 19.0101, Blo t 2, Blo t 2.0101, Blo t 2.0102, Blo t 2.0103, Blo t 20, Blo t 21, Blo t 21.0101, Blo t 3, Blo t 3.0101, Blo t 4, Blo t 4.0101, Blo t 5, Blo t 5.0101, Blo t 6, Blo t 6.0101, Blo t 7, Blo t 8, Blo t 9, Blo t HSP70), Bombus spp (Bom ar 4, Bom by 4, Bom p 1, Bom p 1.0101, Bom p 2, Bom p 3, Bom p 4, Bom p 4.0101, Bom t 1, Bom t 1.0101, Bom t 4, Bom t 4.0101), Bombyx spp (Bomb m 1, Bomb m 1.0101, Bomb m 7, Bomb m 7.0101, Bomb m 7.0102, Bomb m 7.0103, Bomb m 7.0104, Bomb m 7.0105, Bomb m 7.0106), Boophilus spp (Boo m 1, Boo m 7, Boo m 7.0101), Bos spp (Bos d 2, Bos d 2.0101, Bos d 2.0102, Bos d 2.0103, Bos d 3, Bos d 3.0101, Bos d 4, Bos d 4.0101, Bos d 5, Bos d 5.0101, Bos d 5.0102, Bos d 6, Bos d 6 (MDA), Bos d 6.0101, Bos d 7, Bos d 7.0101, Bos d 8, Bos d 8 alphaSi, Bos d 8 alphaS2, Bos d 8 beta, Bos d 8 kappa, Bos d alpha2I, Bos d alpha2L.0101, Bos d Chymosin, Bos d Fibrin, Bos d Gelatin, Bos d HG, Bos d Insulin, Bos d Lactoferrin, Bos d Lactoperoxidase, Bos d Myoglobin, Bos d OBP, Bos d OSCP, Bos d Phosvitin, Bos d PLA2, Bos d PRVB, Bos d Thrombin, Bos d TI, Bos gr ALA, Bos gr Myoglobin), Bothrops spp (Bot as 1, Bot at 1), Bouteloua spp (Bou g 1), Biting spp (Boy ov 1), Brama spp (Bra du 1), Brassica spp (Bra j 1, Bra j 1.0101, Bra n 1, Bra n 1.0101, Bra n 4, Bra n 7, Bra n 8, Bra n PG, Bra ni 8, Bra o 3, Bra o 3.0101, Bra r 1, Bra r 1.0101, Bra r 2, Bra r 2.0101, Bra r 3, Bra r 4, Bra r 7), Bromus spp (Bro a 1, Bro a 4), Brosme spp (Bro br 1), Bromus spp (Bro i 1, Bro i 5, Bro i 7), Brugia spp (Bru m 3, Bru m 3.0101, Bru m Bm33), Bubalus spp (Bub b ALA, Bub b BLG, Bub b Casein, Bub b Casein alphaS1, Bub b Casein alphaS2, Bub b Casein beta, Bub b Casein kappa), Caenorhabditis spp (Cae b 3, Cae b 3.0101, Cae br 3, Cae br 3.0101, Cae e 3, Cae e 3.0101, Cae e 3.0102, Cae re 13, Cae re 13.0101), Cajanus spp (Caj c 1), Caligus spp (Cal cl 1, Cal cl 1.0101, Cal cl 1.0102), Calamus spp (Cal le 1), Callinectes spp (Cal s 2), Camelus spp (Cam d ALA, Cam d Casein, Cam d Casein alphaS1, Cam d Casein alphaS2, Cam d Casein beta, Cam d Casein kappa), Camponotus spp (Cam fl 7, Cam fl 7.0101), Canis spp (Can f 1, Can f 1.0101, Can f 2, Can f 2.0101, Can f 3, Can f 3.0101, Can f 4, Can f 4.0101, Can f 5, Can f 5.0101, Can f 6, Can f 6.0101, Can f Feld1-like, Can f Homs2-like, Can f Phosvitin, Can f TCTP), Canthidermis spp (Can ma 1), Cancer spp (Can mg 2, Can p 1), Cannabis spp (Can s 3), Candida spp (Cand a 1, Cand a 1.0101, Cand a 3, Cand a 3.0101, Cand a CAAP, Cand a CyP, Cand a Enolase, Cand a FPA, Cand a MnSOD, Cand a PGK, Cand b 2, Cand b 2.0101, Cand b FDH, Cand r Lipase), Capsicum spp (Cap a 1, Cap a 1.0101, Cap a 17 kD, Cap a 2, Cap a 2.0101, Cap a 30 kD, Cap a Glucanase, Cap ch 17 kD), Caprella spp (Cap e 1), Capra spp (Cap h ALA, Cap h BLG, Cap h Casein, Cap h Casein alphaS1, Cap h Casein alphaS2, Cap h Casein beta, Cap h Casein kappa, Cap h GSA), Capitulum spp (Cap m 1), Carassius spp (Car au 1), Carpinus spp (Car b 1, Car b 1.0101, Car b 1.0102, Car b 1.0103, Car b 1.0104, Car b 1.0105, Car b 1.0106, Car b 1.0107, Car b 1.0108, Car b 1.0109, Car b 1.0110, Car b 1.0111, Car b 1.0112, Car b 1.0113, Car b 1.0201, Car b 1.0301, Car b 1.0302, Car b 2, Car b 4), Caranx spp (Car cr 1), Carya spp (Car i 1, Car i 1.0101, Car i 2, Car i 4, Car i 4.0101), Carcinus spp (Car ma 2), Caryota spp (Car mi 2), Carica spp (Car p 1, Car p Chitinase, Car p Chymopapain, Car p Endoproteinase), Castanea spp (Cas c 24 kD, Cas s 1, Cas s 1.0101, Cas s 1.0102, Cas s 1.0103, Cas s 2, Cas s 5, Cas s 5.0101, Cas s 8, Cas s 8.0101, Cas s 9, Cas s 9.0101), Catharanthus spp (Cat r 1, Cat r 1.0101, Cat r 17 kD, Cat r 2), Caulolatilus spp (Cau ch 1), Cavia spp (Cav p 1, Cav p 1.0101, Cav p 2, Cav p 2.0101, Cav p 3, Cav p 3.0101, Cav p Gelatin, Cav p GSA), Centropristis spp (Cen s 1), Cephalopholis spp (Cep so 1), Charybdis spp (Cha f 1, Cha f 1.0101), Chaetodipterus spp (Cha fa 1), Chamaecyparis spp (Cha o 1, Cha o 1.0101, Cha o 2, Cha o 2.0101), Chenopodium spp (Che a 1, Che a 1.0101, Che a 2, Che a 2.0101, Che a 3, Che a 3.0101), Chironomus spp (Chi k 1, Chi k 10, Chi k 10.0101), Chinchilla spp (Chi I 21 kD_a, Chi I 21 kD_b), Chionoecetes spp (Chi o 1, Chi o 1.0101, Chi o 2, Chi o 4, Chi o 6, Chi o alpha_Actin, Chi o SERCA), Chironomus spp (Chi t 1, Chi t 1.0101, Chi t 1.0201, Chi t 2, Chi t 2.0101, Chi t 2.0102, Chi t 3, Chi t 3.0101, Chi t 4, Chi t 4.0101, Chi t 5, Chi t 5.0101, Chi t 6, Chi t 6.0101, Chi t 6.0201, Chi t 7, Chi t 7.0101, Chi t 8, Chi t 8.0101, Chi t 9, Chi t 9.0101), Chlamys spp (Chl n 1), Chloephaga spp (Chl pi 1), Chortoglyphus spp (Cho a 10), Chrysomela spp (Chr tr 7, Chr tr 7.0101), Cicer spp (Cic a 2S Albumin, Cic a Albumin), Cichorium spp (Cic i 1), Cimex spp (Cim I Nitrophorin), Citrus spp (Cit 11, Cit I 3, Cit I 3.0101), Citrullus spp (Cit 1a 2, Cit 1a MDH, Cit 1a TPI), Citrus spp (Cit r 3, Cit r 3.0101, Cit s 1, Cit s 1.0101, Cit s 2, Cit s 2.0101, Cit s 3, Cit s 3.0101, Cit s 3.0102, Cit s IFR), Cladosporium spp (Cla c 14, Cla c 14.0101, Cla c 9, Cla c 9.0101, Cla h 1, Cla h 10, Cla h 10.0101, Cla h 12, Cla h 12.0101, Cla h 2, Cla h 2.0101, Cla h 42 kD, Cla h 5, Cla h 5.0101, Cla h 6, Cla h 6.0101, Cla h 7, Cla h 7.0101, Cla h 8, Cla h 8 CSP, Cla h 8.0101, Cla h 9, Cla h 9.0101, Cla h abH, Cla h GST, Cla h HChl, Cla h HSP70, Cla h NTF2, Cla h TCTP), Clostridium spp (Clo hi Collagenase, Clo t Toxoid), Clupea spp (Clu h 1, Clu h 1.0101, Clu h 1.0201, Clu h 1.0301), Cocos spp (Coc n 2, Coc n 4, Coc n 5), Coccidioides spp (Coc po 8), Coffea spp (Cof a 1, Cof a 1.0101), Columba spp (Col I PSA), Coprinus spp (Cop c 1, Cop c 1.0101, Cop c 2, Cop c 2.0101, Cop c 3, Cop c 3.0101, Cop c 4, Cop c 5, Cop c 5.0101, Cop c 6, Cop c 7, Cop c 7.0101), Corylus spp (Cor a 1, Cor a 1.0101, Cor a 1.0102, Cor a 1.0103, Cor a 1.0104, Cor a 1.0201, Cor a 1.0301, Cor a 1.0401, Cor a 1.0402, Cor a 1.0403, Cor a 1.0404, Cor a 10, Cor a 10.0101, Cor a 11, Cor a 11.0101, Cor a 12, Cor a 12.0101, Cor a 13, Cor a 13.0101, Cor a 14, Cor a 14.0101, Cor a 2, Cor a 2.0101, Cor a 2.0102, Cor a 8, Cor a 8.0101, Cor a 9, Cor a 9.0101), Corynebacterium spp (Cor d Toxoid), Corylus spp (Cor he 1), Coryphaena spp (Cor hi 1), Coriandrum spp (Cor s 1, Cor s 11 kD, Cor s 2), Cotoneaster spp (Cot 13), Crangon spp (Cra c 1, Cra c 1.0101, Cra c 2, Cra c 2.0101, Cra c 4, Cra c 4.0101, Cra c 5, Cra c 5.0101, Cra c 6, Cra c 6.0101, Cra c 8, Cra c 8.0101), Crassostrea spp (Cra g 1), Cricetus spp (Cri c HSA), Crivellia spp (Cri pa 1), Crocus spp (Cro s 1, Cro s 1.0101, Cro s 2, Cro s 2.0101, Cro s 3, Cro s 3.01, Cro s 3.02), Cryptomeria spp (Cry j 1, Cry j 1.0101, Cry j 1.0102, Cry j 1.0103, Cry j 2, Cry j 2.0101, Cry j 2.0102, Cry j 3, Cry j 3.1, Cry j 3.2, Cry j 3.3, Cry j 3.4, Cry j 3.5, Cry j 3.6, Cry j 3.7, Cry j 3.8, Cry j 4, Cry j AP, Cry j Chitinase, Cry j CPA9, Cry j IFR, Cry j LTP, Cry j P1-P2), Cryphonectria spp (Cry p AP), Ctenocephalides spp (Cte f 1, Cte f 1.0101, Cte f 2, Cte f 2.0101, Cte f 3, Cte f 3.0101), Ctenopharyngodon spp (Cte id 1), Cucumis spp (Cuc m 1, Cuc m 1.0101, Cuc m 2, Cuc m 2.0101, Cuc m 3, Cuc m 3.0101, Cuc m Lec17, Cuc m MDH), Cucurbita spp (Cuc ma 18 kD, Cuc ma 2, Cuc p 2, Cuc p AscO), Cucumis spp (Cuc s 2), Culicoides spp (Cul n 1, Cul n 10, Cul n 11, Cul n 2, Cul n 3, Cul n 4, Cul n 5, Cul n 6, Cul n 7, Cul n 8, Cul n 9, Cul n HSP70), Culex spp (Cul q 28 kD, Cul q 35 kD, Cul q 7, Cul q 7.0101, Cul q 7.0102), Culicoides spp (Cul so 1), Cuminum spp (Cum c 1, Cum c 2), Cupressus spp (Cup a 1, Cup a 1.0101, Cup a 1.02, Cup a 2, Cup a 3, Cup a 4, Cup a 4.0101, Cups 1, Cups 1.0101, Cups 1.0102, Cup s 1.0103, Cup s 1.0104, Cup s 1.0105, Cup s 3, Cup s 3.0101, Cup s 3.0102, Cup s 3.0103, Cup s 8), Cochliobolus spp (Cur 11, Cur 11.0101, Cur 12, Cur I 2.0101, Cur I 3, Cur I 3.0101, Cur I 4, Cur I 4.0101, Cur I ADH, Cur I GST, Cur I MnSOD, Cur I Oryzin, Cur I Trx, Cur I ZPS1), Cyanochen spp (Cya cy 1), Cynoscion spp (Cyn ar 1), Cynosurus spp (Cyn er 1, Cyn er 5), Cynodon spp (Cyn d 1, Cyn d 1.0101, Cyn d 1.0102, Cyn d 1.0103, Cyn d 1.0104, Cyn d 1.0105, Cyn d 1.0106, Cyn d 1.0107, Cyn d 1.0201, Cyn d 1.0202, Cyn d 1.0203, Cyn d 1.0204, Cyn d 10, Cyn d 11, Cyn d 12, Cyn d 12.0101, Cyn d 13, Cyn d 15, Cyn d 15.0101, Cyn d 2, Cyn d 22, Cyn d 22.0101, Cyn d 23, Cyn d 23.0101, Cyn d 24, Cyn d 24.0101, Cyn d 4, Cyn d 5, Cyn d 6, Cyn d 7, Cyn d 7.0101), Cynoscion spp (Cyn ne 1), Cynomys spp (Cyn sp Lipocalin), Cyprinus spp (Cyp c 1, Cyp c 1.01, Cyp c 1.02), Daboia spp (Dab ru 1), Dactylis spp (Dac g 1, Dac g 1.01, Dac g 1.0101, Dac g 1.02, Dac g 12, Dac g 13, Dac g 2, Dac g 2.0101, Dac g 3, Dac g 3.0101, Dac g 4, Dac g 4.0101, Dac g 5, Dac g 5.0101, Dac g 7), Dama spp (Dam d CSA), Danio spp (Dan re 1, Dan re 2, Dan re alpha2l, Dan re CK), Dasyatis spp (Das ak 1, Das am 1, Das sa 1), Daucus spp (Dau c 1, Dau c 1.0101, Dau c 1.0102, Dau c 1.0103, Dau c 1.0104, Dau c 1.0105, Dau c 1.0201, Dau c 1.0301, Dau c 3, Dau c 4, Dau c 4.0101, Dau c CyP), Decapterus spp (Dec ru 1), Dendronephthya spp (Den n 1, Den n 1.0101), Dermatophagoides spp (Der f 1, Der f 1.0101, Der f 1.0102, Der f 1.0103, Der f 1.0104, Der f 1.0105, Der f 1.0106, Der f 1.0107, Der f 1.0108, Der f 1.0109, Der f 1.0110, Der f 10, Der f 10.0101, Der f 10.0102, Der f 11, Der f 11.0101, Der f 13, Der f 13.0101, Der f 14, Der f 14.0101, Der f 15, Der f 15.0101, Der f 16, Der f 16.0101, Der f 17, Der f 17.0101, Der f 18, Der f 18.0101, Der f 2, Der f 2.0101, Der f 2.0102, Der f 2.0103, Der f 2.0104, Der f 2.0105, Der f 2.0106, Der f 2.0107, Der f 2.0108, Der f 2.0109, Der f 2.0110, Der f 2.0111, Der f 2.0112, Der f 2.0113, Der f 2.0114, Der f 2.0115, Der f 2.0116, Der f 2.0117, Der f 20, Der f 21, Der f 22, Der f 22.0101, Der f 3, Der f 3.0101, Der f 4, Der f 5, Der f 6, Der f 6.0101, Der f 7, Der f 7.0101, Der f 8, Der f 9, Der f HSP70), Dermanyssus spp (Der g 10, Der g 10.0101), Dermatophagoides spp (Der m 1, Der m 1.0101, Der p 1, Der p 1.0101, Der p 1.0102, Der p 1.0103, Der p 1.0104, Der p 1.0105, Der p 1.0106, Der p 1.0107, Der p 1.0108, Der p 1.0109, Der p 1.0110, Der p 1.0111, Der p 1.0112, Der p 1.0113, Der p 1.0114, Der p 1.0115, Der p 1.0116, Der p 1.0117, Der p 1.0118, Der p 1.0119, Der p 1.0120, Der p 1.0121, Der p 1.0122, Der p 1.0123, Der p 1.0124, Der p 10, Der p 10.0101, Der p 10.0102, Der p 10.0103, Der p 11, Der p 11.0101, Der p 13, Der p 14, Der p 14.0101, Der p 15, Der p 18, Der p 2, Der p 2.0101, Der p 2.0102, Der p 2.0103, Der p 2.0104, Der p 2.0105, Der p 2.0106, Der p 2.0107, Der p 2.0108, Der p 2.0109, Der p 2.0110, Der p 2.0111, Der p 2.0112, Der p 2.0113, Der p 2.0114, Der p 2.0115, Der p 20, Der p 20.0101, Der p 21, Der p 21.0101, Der p 23, Der p 23.0101, Der p 3, Der p 3.0101, Der p 4, Der p 4.0101, Der p 5, Der p 5.0101, Der p 5.0102, Der p 6, Der p 6.0101, Der p 7, Der p 7.0101, Der p 8, Der p 8.0101, Der p 9, Der p 9.0101, Der p 9.0102, Der p P1-P2, Der p P2-P1, Der s 1, Der s 2, Der s 3), Dianthus spp (Dia c RIP), Dicranopteris spp (Dic I 2S Albumin), Diospyros spp (Dio k 17 kD, Dio k 4, Dio k IFR), Dioscorea spp (Dio p TSP), Diplodus spp (Dip ho 1), Distichlis spp (Dis s 1, Dis s 7), Ditrema spp (Dit to 1), Dolichovespula spp (Dol a 1, Dol a 2, Dol a 5, Dol a 5.0101), Dolichos spp (Dol b Agglutinin), Dolichovespula spp (Dol m 1, Dol m 1.0101, Dol m 1.02, Dol m 2, Dol m 2.0101, Dol m 5, Dol m 5.0101, Dol m 5.02), Drosophila spp (Dro an 7, Dro an 7.0101, Dro er 7, Dro er 7.0101, Dro er 7.0102, Dro gr 7, Dro gr 7.0101, Dro gr 7.0102, Dro m 7, Dro m 7.0101, Dro m 7.0102, Dro m 7.0103, Dro m 7.0104, Dro m 7.0105, Dro m 7.0106, Dro m 7.0107, Dro m 7.0108, Dro m 7.0109, Dro m 7.0110, Dro m 7.0111, Dro m 7.0112, Dro m 7.0113, Dro m 9, Dro m MnSOD, Dro mo 7, Dro mo 7.0101, Dro pp 7, Dro pp 7.0101, Dro se 7, Dro se 7.0101, Dro si 7, Dro si 7.0101, Dro si 7.0102, Dro vi 7, Dro vi 7.0101, Dro wi 7, Dro wi 7.0101, Dro y 7, Dro y 7.0101, Dro y 7.0102, Dro y 7.0103), Echium spp (Ech p Cytochrome C), Elaeis spp (Ela g 2, Ela g Bd31 kD), Elops spp (Elo sa 1), Embellisia spp (Emb a 1, Emb i 1, Emb nz 1, Emb t 1), Engraulis spp (Eng e 1), Enteroctopus spp (Ent d 1), Epinephelus spp (Epi bl 1, Epi co 1, Epi fl 1, Epi me 1, Epi mo 1), Epicoccum spp (Epi p 1, Epi p 1.0101, Epi p 12 kD, Epi p GST), Epinephelus spp (Epi po 1, Epi un 1), Equisetum spp (Equ a 17 kD), Equus spp (Equ as 4, Equ as DSA, Equ bu 4, Equ c 1, Equ c 1.0101, Equ c 2, Equ c 2.0101, Equ c 2.0102, Equ c 3, Equ c 3.0101, Equ c 4, Equ c 4.0101, Equ c 5, Equ c 5.0101, Equ c ALA, Equ c BLG, Equ c Casein, Equ c Casein beta, Equ c Casein kappa, Equ c PRVB, Equ he 4, Equ z ZSA), Erimacrus spp (En i 1, En i 1.0101, Eri i 1.0102), Eriocheir spp (Eri s 1, Eri s 1.0101, En s 2), Erwinia spp (Erw ch Asparaginase), Escherichia spp (Esc c Asparaginase, Esc c beta GAL), Esox spp (Eso 11), Euphausia spp (Eup p 1, Eup p 1.0101), Euphasia spp (Eup s 1, Eup s 1.0101), Euroglyphus spp (Eur m 1, Eur m 1.0101, Eur m 1.0102, Eur m 1.0103, Eur m 10, Eur m 14, Eur m 14.0101, Eur m 2, Eur m 2.0101, Eur m 2.0102, Eur m 3, Eur m 3.0101, Eur m 4, Eur m 4.0101), Evynnis spp (Evy j 1), Fagopyrum spp (Fag e 1, Fag e 1.0101, Fag e 10 kD, Fag e 19 kD, Fag e 2, Fag e 2.0101, Fag e TI), Fagus spp (Fag s 1, Fag s 1.0101, Fag s 2, Fag s 4), Fagopyrum spp (Fag t 1, Fag t 10 kD, Fag t 2, Fag t 2.0101), Felis spp (Fel d 1, Fel d 1.0101, Fel d 2, Fel d 2.0101, Fel d 3, Fel d 3.0101, Fel d 4, Fel d 4.0101, Fel d 5, Fel d 5.0101, Fel d 6, Fel d 6.0101, Fel d 7, Fel d 7.0101, Fel d 8, Fel d 8.0101, Fel d IgG), Fenneropenaeus spp (Fen c 1, Fen c 2, Fen me 1, Fen me 1.0101), Festuca spp (Fes e 1, Fes e 13, Fes e 4, Fes e 5, Fes e 7, Fes p 1, Fes p 13, Fes p 4, Fes p 4.0101, Fes p 5, Fes r 1, Fes r 5), Ficus spp (Fic c 17 kD, Fic c 4, Fic c Ficin), Foeniculum spp (Foe v 1, Foe v 2), Forsythia spp (For s 1), Forcipomyia spp (Fort 1, Fort 1.0101, Fort 2, Fort 2.0101, Fort 7, Fort FPA, Fort Myosin, Fort TPI), Fragaria spp (Fra a 1, Fra a 1.0101, Fra a 3, Fra a 3.0101, Fra a 3.0102, Fra a 3.0201, Fra a 3.0202, Fra a 3.0203, Fra a 3.0204, Fra a 3.0301, Fra a 4, Fra a 4.0101, Fra c 1), Fraxinus spp (Fra e 1, Fra e 1.0101, Fra e 1.0102, Fra e 1.0201, Fra e 12, Fra e 2, Fra e 3, Fra e 9), Fragaria spp (Fra v 1), Fusarium spp (Fus c 1, Fus c 1.0101, Fus c 2, Fus c 2.0101, Fus c 3, Fus s 1, Fus s 45 kD, Fus sp Lipase), Gadus spp (Gad c 1, Gad c 1.0101, Gad c APDH, Gad m 1, Gad m 1.0101, Gad m 1.0102, Gad m 1.0201, Gad m 1.0202, Gad m 45 kD, Gad m Gelatin, Gad ma 1), Gallus spp (Gal d 1, Gal d 1.0101, Gal d 2, Gal d 2.0101, Gal d 3, Gal d 3.0101, Gal d 4, Gal d 4.0101, Gal d 5, Gal d 5.0101, Gal d 6, Gal d 6.0101, Gal d Apo I, Gal d Apo VI, Gal d GPI, Gal d HG, Gal d IgY, Gal d L-PGDS, Gal d Ovomucin, Gal d Phosvitin, Gal d PRVB, Gal 1a 4), Galleria spp (Gal m 18 kD, Gal m 24 kD), Gallus spp (Gal so 4), Gammarus spp (Gam s TM), Gelonium spp (Gel m RIP), Geothelphusa spp (Geo de 1), Glossina spp (Glo m 5, Glo m 5.0101, Glo m 7, Glo m 7.0101, Glo m 7.0102, Glo m 7.0103), Glycine spp (Gly a Bd30K, Gly ar Bd30K, Gly ca Bd30K, Gly el Bd30K, Gly cu Bd30K, Gly cy Bd30K), Glycyphagus spp (Gly d 10, Gly d 10.0101, Gly d 13, Gly d 2, Gly d 2.0101, Gly d 2.0201, Gly d 2.03, Gly d 2/Lep d 2 L1, Gly d 2/Lep d 2 L2, Gly d 2/Lep d 2 L3, Gly d 2/Lep d 2 L4, Gly d 2/Lep d 2 R1, Gly d 2/Lep d 2 R2, Gly d 2/Lep d 2 R3, Gly d 2/Lep d 2 R4, Gly d 2/Lep d 2 R5, Gly d 20, Gly d 3, Gly d 5, Gly d 5.01, Gly d 5.02, Gly d 7, Gly d 8), Glycine spp (Gly f Bd30K, Gly I Bd30K, Gly m 1, Gly m 1.0101, Gly m 1.0102, Gly m 2, Gly m 2.0101, Gly m 2S Albumin, Gly m 3, Gly m 3.0101, Gly m 3.0102, Gly m 39 kD, Gly m 4, Gly m 4.0101, Gly m 5, Gly m 5.0101, Gly m 5.0201, Gly m 5.0301, Gly m 5.0302, Gly m 50 kD, Gly m 6, Gly m 6.0101, Gly m 6.0201, Gly m 6.0301, Gly m 6.0401, Gly m 6.0501, Gly m 68 kD, Gly m Agglutinin, Gly m Bd28K, Gly m Bd30K, Gly m Bd60K, Gly m CPI, Gly m EAP, Gly m TI, Gly mi Bd30K, Gly s Bd30K, Gly t Bd30K, Gly to Bd30K), Gossypium spp (Gos h Vicilin), Haemophilus spp (Hae in P6), Haemaphysalis spp (Hae 17, Hae 1 7.0101, Hae q 7, Hae q 7.0101), Haliotis spp (Hal a 1, Hal d 1, Hal di 1, Hal di PM, Hal m 1, Hal m 1.0101, Hal r 1, Hal r 49 kD, Hal ru 1), Harmonia spp (Har a 1, Har a 1.0101, Har a 2, Har a 2.0101), Harpegnathos spp (Har sa 7, Har sa 7.0101, Har sa 7.0102), Helianthus spp (Hel a 1, Hel a 1.0101, Hel a 2, Hel a 2.0101, Hel a 2S Albumin, Hel a 3, Hel a 3.0101, Hel a 4), Helix spp (Hel ap 1, Hel as 1, Hel as 1.0101), Heligmosomoides spp (Hel p 3, Hel p 3.0101), Helianthus spp (Hel to 1), Hemanthias spp (Hem le 1), Hemifusus spp (Hem t 1), Heterodera spp (Het g 3, Het g 3.0101), Hevea spp (Hey b 1, Hey b 1.0101, Hey b 10, Hey b 10.0101, Hey b 10.0102, Hey b 10.0103, Hey b 11, Hey b 11.0101, Hey b 11.0102, Hey b 12, Hey b 12.0101, Hey b 13, Hey b 13.0101, Hey b 14, Hey b 14.0101, Hey b 2, Hey b 2.0101, Hey b 3, Hey b 3.0101, Hey b 4, Hey b 4.0101, Hey b 5, Hey b 5.0101, Hey b 6, Hey b 6.01, Hey b 6.02, Hey b 6.0202, Hey b 6.03, Hey b 7, Hey b 7.01, Hey b 7.02, Hey b 7.D2, Hey b 7.S2, Hey b 8, Hey b 8.0101, Hey b 8.0102, Hey b 8.0201, Hey b 8.0202, Hey b 8.0203, Hey b 8.0204, Hey b 9, Hey b 9.0101, Hey b Citrate binding Protein, Hey b GAPDH, Hey b HSP80, Hey b IFR, Hey b Proteasome subunit, Hey b Rotamase, Hey b SPI, Hey b Trx, Hey b UDPGP), Hexagrammos spp (Hex of 1), Hippoglossus spp (Hip h 1), Hippoglossoides spp (Hip pl 1), Hippoglossus spp (Hip st 1), Hirudo spp (Hir me Hirudin), Holcus spp (Hol 1 1, Hol 1 1.0101, Hol 1 1.0102, Hol 1 2, Hol 1 4, Hol 1 5, Hol 1 5.0101, Hol 1 5.0201), Holocnemus spp (Hol p^{l} 9, Hol pl Hemocyanin), Homarus spp (Hom a 1, Hom a 1.0101, Hom a 1.0102, Hom a 1.0103, Hom a 3, Hom a 3.0101, Hom a 4, Hom a 6, Hom a 6.0101, Hom g 1, Hom g 2), Homo spp (Hom s 1, Hom s 1.0101, Hom s 2, Hom s 2.0101, Hom s 3, Hom s 3.0101, Hom s 4, Hom s 4.0101, Hom s 5, Hom s 5.0101, Hom s AAT, Hom s ACTH, Hom s Adalimumab, Hom s ALA, Hom s alpha Actin, Hom s alpha-Galactosidase, Hom s APDH, Hom s Arylsulfatase B, Hom s Casein, Hom s CyP A, Hom s CyP B, Hom s CyP C, Hom s DSF70, Hom s DSG3, Hom s eIF6, Hom s Etanercept, Hom s Factor IX, Hom s Factor VII, Hom s Factor VIII, Hom s G-CSF, Hom s Glucocerebrosidase, Hom s Glucosidase, Hom s HLA-DR-alpha, Hom s HSA, Hom s Iduronidase, Hom s Idursulfase, Hom s IgA, Hom s Insulin, Hom s Lactoferrin, Hom s Laminin gamma 2, Hom s MnSOD, Hom s Oxytocin, Hom s P2, Hom s Phosvitin, Hom s Profilin, Hom s PSA, Hom s RP1, Hom s TCTP, Hom s TL, Hom s TPA, Hom s TPO, Hom s Transaldolase, Hom s Trx, Hom s Tubulin-alpha, Hom s/Mus m Basiliximab, Hom s/Mus m Cetuximab, Hom s/Mus m Cetuximab (Gal-Gal), Hom s/Mus m Infliximab, Hom s/Mus m Natalizumab, Hom s/Mus m Omalizumab, Hom s/Mus m Palivizumab, Hom s/Mus m Rituximab, Hom s/Mus m Tocilizumab, Hom s/Mus m Trastuzumab), Hoplostethus spp (Hop a 1), Hordeum spp (Hor v 1, Hor v 12, Hor v 12.0101, Hor v 13, Hor v 14, Hor v 15, Hor v 15.0101, Hor v 16, Hor v 16.0101, Hor v 17, Hor v 17.0101, Hor v 18 kD, Hor v 2, Hor v 21, Hor v 21.0101, Hor v 28, Hor v 33, Hor v 4, Hor v 5, Hor v 5.0101, Hor v BDAI, Hor v BTI), Humicola spp (Hum in Cellulase), Humulus spp (Hum j 1, Hum j 1.0101, Hum j 10 kD, Hum j 2), Huso spp (Hus h 1), Hylocereus spp (Hyl un LTP), Hymenocephalus spp (Hym st 1), Hyperoglyphe spp (Hyp by 1), Hypophthalmichthys spp (Hyp mo 1), Hypophthalmichthy spp (Hyp no 1), Ictalurus spp (Ict fu 1, Ict p 1), Imperata spp (Imp c 4, Imp c 5, Imp c VIIIel), Ixodes spp (Ixo r 2, Ixo sc 7, Ixo se 7.0101), Jasus spp (Jas 1a 1, Jas 1a 1.0101, Jas 1a 1.0102), Juglans spp (Jug ca 1, Jug ca 2, Jug ci 1, Jug ci 2, Jug n 1, Jug n 1.0101, Jug n 2, Jug n 2.0101, Jug r 1, Jug r 1.0101, Jug r 2, Jug r 2.0101, Jug r 3, Jug r 3.0101, Jug r 4, Jug r 4.0101, Jug r 5), Juniperus spp (Jun a 1, Jun a 1.0101, Jun a 1.0102, Jun a 2, Jun a 2.0101, Jun a 3, Jun a 3.0101, Jun c 1, Jun o 1, Jun o 4, Jun o 4.0101, Jun r 3, Jun r 3.1, Jun r 3.2, Jun v 1, Jun v 1.0101, Jun v 1.0102, Jun v 3, Jun v 3.0101, Jun v 3.0102, Jun v 4), Katsuwonus spp (Kat p 1), Kyphosus spp (Kyp se 1), Lachnolaimus spp (Lac ma 1), Lachesis spp (Lac mu 1), Lactuca spp (Lac s 1, Lac s 1.0101), Lagocephalus spp (Lag 1a 1), Larus spp (Lar a 1, Lar a 2, Lar a 3), Larimichthys spp (Lar po 1), Lates spp (Lat c 1), Lateolabrax spp (Lat ja 1), Lathyrus spp (Lat oc Agglutinin), Leiostomus spp (Lei xa 1), Lens spp (Len c 1, Len c 1.0101, Len c 1.0102, Len c 1.0103, Len c 2, Len c 2.0101, Len c 3, Len c 3.0101, Len c Agglutinin), Leopardus spp (Leo p 1), Lepidoglyphus spp (Lep d 10, Lep d 10.0101, Lep d 12, Lep d 13, Lep d 13.0101, Lep d 2, Lep d 2.0101, Lep d 2.0102, Lep d 2.0201, Lep d 2.0202, Lep d 3, Lep d 39 kD, Lep d 5, Lep d 5.0101, Lep d 5.0102, Lep d 5.0103, Lep d 7, Lep d 7.0101, Lep d 8, Lep d alpha Tubulin), Lepomis spp (Lep gi 1), Leptomelanosoma spp (Lep i 1), Lepomis spp (Lep ma 1), Lepisma spp (Lep s 1, Lep s 1.0101, Lep s 1.0102), Lepeophtheirus spp (Lep sa 1, Lep sa 1.0101, Lep sa 1.0102, Lep sa 1.0103), Leptailurus spp (Lep se 1), Lepidorhombus spp (Lep w 1, Lep w 1.0101), Lethocerus spp (Let in 7, Let in 7.0101, Let in 7.0102), Leuciscus spp (Leu ce 1), Lewia spp (Lew in 1), Ligustrum spp (Lig v 1, Lig v 1.0101, Lig v 1.0102, Lig v 2), Lilium spp (Lil 12, Lil I PG), Limanda spp (Lim fe 1), Limnonectes spp (Lim m 1), Limulus spp (Lim p 1, Lim p 1.0101, Lim p 2, Lim p LPA), Liposcelis spp (Lip b 1, Lip b 1.0101), Litchi spp (Lit c 1, Lit c 1.0101, Lit c IFR, Lit c TPI), Lithobates spp (Lit ca 1), Litopenaeus spp (Lit se 1, Lit v 1, Lit v 1.0101, Lit v 2, Lit v 2.0101, Lit v 3, Lit v 3.0101, Lit v 4, Lit v 4.0101), Filiaria spp (Loa lo 3, Loa lo 3.0101), Lobotes spp (Lob su 1), Locusta spp (Loc m 7, Loc m 7.0101), Loligo spp (Lol b 1, Lol e 1), Lolium spp (Lol m 2, Lol m 5, Lol p 1, Lol p 1.0101, Lol p 1.0102, Lol p 1.0103, Lol p 10, Lol p 11, Lol p 11.0101, Lol p 12, Lol p 13, Lol p 2, Lol p 2.0101, Lol p 3, Lol p 3.0101, Lol p 4, Lol p 4.0101, Lol p 5, Lol p 5.0101, Lol p 5.0102, Lol p 7, Lol p CyP, Lol p FT, Lol p Legumin), Lonomia spp (Lon o 7, Lon o 7.0101), Lophodytes spp (Lop cu 1), Lophonetta spp (Lop sp 1), Lupinus spp (Lup a 1, Lup a alpha_Conglutin, Lup a delta_Conglutin, Lup a gamma_Conglutin, Lup an 1, Lup an 1.0101, Lup an alpha_Conglutin, Lup an delta_Conglutin, Lup an gamma_Conglutin, Lup 117 kD), Lutjanus spp (Lut a 1, Lut c 1, Lut cy 1, Lut gr 1, Lut gu 1, Lut jo 1), Lutraria spp (Lut p 1), Lutjanus spp (Lut pu 1, Lut sy 1), Lycopersicon spp (Lyc e 1, Lyc e 1.0101, Lyc e 11 S Globulin, Lyc e 2, Lyc e 2.0101, Lyc e 2.0102, Lyc e 3, Lyc e 3.0101, Lyc e 4, Lyc e 4.0101, Lyc e ARP60S, Lyc e Chitinase, Lyc e Glucanase, Lyc e Peroxidase, Lyc e PG, Lyc e PME, Lyc e PR23, Lyc e Vicilin), Maconellicoccus spp (Mac h 7, Mac h 7.0101), Macruronus spp (Mac ma 1, Mac n 1), Macdura spp (Mac po 17 kD), Macrobrachium spp (Mac ro 1, Mac ro 1.0101, Mac ro Hemocyanin), Macropus spp (Marr s Gelatin), Malus spp (Mal d 1, Mal d 1.0101, Mal d 1.0102, Mal d 1.0103, Mal d 1.0104, Mal d 1.0105, Mal d 1.0106, Mal d 1.0107, Mal d 1.0108, Mal d 1.0109, Mal d 1.0201, Mal d 1.0202, Mal d 1.0203, Mal d 1.0204, Mal d 1.0205, Mal d 1.0206, Mal d 1.0207, Mal d 1.0208, Mal d 1.0301, Mal d 1.0302, Mal d 1.0303, Mal d 1.0304, Mal d 1.0401, Mal d 1.0402, Mal d 1.0403, Mal d 2, Mal d 2.0101, Mal d 3, Mal d 3.0101, Mal d 3.0102, Mal d 3.0201, Mal d 3.0202, Mal d 3.0203, Mal d 4, Mal d 4.0101, Mal d 4.0102, Mal d 4.0201, Mal d 4.0202, Mal d 4.0301, Mal d 4.0302), Malpighia spp (Mal g 4, Mal g Hevein), Malus spp (Mal p 1), Malassezia spp (Mala f 2, Mala f 2.0101, Mala f 3, Mala f 3.0101, Mala f 4, Mala f 4.0101, Mala g 10, Mala s 1, Mala s 1.0101, Mala s 10, Mala s 10.0101, Mala s 11, Mala s 11.0101, Mala s 12, Mala s 12.0101, Mala s 13, Mala s 13.0101, Mala s 5, Mala s 5.0101, Mala s 6, Mala s 6.0101, Mala s 7, Mala s 7.0101, Mala s 8, Mala s 8.0101, Mala s 9, Mala s 9.0101), Manihot spp (Man e 5, Man e 5.0101, Man e FPA, Man e GAPDH), Mangifera spp (Man i 1, Man i 14 kD, Man i 2, Man i 3, Man i 3.01, Man i 3.02, Man i Chitinase), Marsupenaeus spp (Mar j 1, Mar j 1.0101, Mar j 2, Mar j 4), Matricaria spp (Mat c 17 kD), Mecopoda spp (Mec e 7), Megalobrama spp (Meg am 2, Meg am CK), Megathura spp (Meg c Hemocyanin), Megalops spp (Meg sp 1), Melanogrammus spp (Mel a 1), Meleagris spp (Mel g 1, Mel g 2, Mel g 3, Mel g PRVB, Mel g TSA), Melicertus spp (Mel 11), Menticirrhus spp (Men am 1), Mercurialis spp (Mer a 1, Mer a 1.0101), Merluccius spp (Mer ap 1, Mer au 1, Mer bi 1, Mer ca 1, Mer ga 1, Mer hu 1), Merlangius spp (Mer me 1), Merluccius spp (Mer mr 1, Mer pa 1, Mer po 1, Mer pr 1, Mer se 1), Meriones spp (Mer un 23 kD), Metarhizium spp (Met a 30), Metapenaeopsis spp (Met ba 1), Metapenaeus spp (Mete 1, Mete 1.0101, Met e 2), Metasequoia spp (Met gl 2), Metapenaeus spp (Met j 1, Met j 2), Metanephrops spp (Met ja 1), Metapenaeopsis spp (Met 1a 1), Metanephrops spp (Met t 2), Micromesistius spp (Mic po 1), Micropogonias spp (Mic un 1), Mimachlamys spp (Mim n 1), Momordica spp (Mom c RIP), Morus spp (Mor a 17 kD, Mor a 4), Morone spp (Mor am 1), Morus spp (Mor n 3, Mor n 3.0101), Morone spp (Mor sa 1, Mor sc 1), Mugil spp (Mug c 1), Muraenolepis spp (Mur mi 1), Musa spp (Mus a 1, Mus a 1.0101, Mus a 2, Mus a 2.0101, Mus a 3, Mus a 3.0101, Mus a 4, Mus a 4.0101, Mus a 5, Mus a 5.0101, Mus a 5.0102), Mus spp (Mus m 1,Musm 1.0101, Mus m 1.0102, Mus m 2, Mus m Gelatin, Mus m IgG, Mus m MSA, Mus m Muromonab, Mus m Phosvitin), Mustela spp (Mus p 17 kD), Musa spp (Mus xp 1, Mus xp 2, Mus xp 5), Mycteroperca spp (Myc bo 1, Myc mi 1, Myc ph 1), Myceiophthora spp (Myc sp Laccase), Myrmecia spp (Myr p 1, Myr p 1.0101, Myr p 2, Myr p 2.0101, Myr p 2.0102, Myr p 3, Myr p 3.0101), Mytilus spp (Myt e 1, Myt g 1, Myt g PM), Myzus spp (Myz p 7, Myz p 7.0101), Nemorhedus spp (Nae go Hya), Necator spp (Nec a Calreticulin), Nemipterus spp (Nem vi 1), Neosartorya spp (Neo fi 1, Neo fi 22), Neochen spp (Neo ju 1), Neoscona spp (Neo n 7, Neo n 7.0101), Nephelium spp (Nep I GAPDH), Nephrops spp (Nep n 1, Nep n DF9), Neptunea spp (Nep po 1, Nep po 1.0101), Nicotiana spp (Nic t 8, Nic t Osmotin, Nic t Villin), Nimbya spp (Nim c 1, Nim s 1), Nippostrongylus spp (Nip b Agl), Nycticebus spp (Nyc c 1), Octopus spp (Oct f 1, Oct 11, Oct v 1, Oct v 1.0101, Oct v PM), Ocyurus spp (Ocy ch 1), Olea spp (Ole e 1, Ole e 1.0101, Ole e 1.0102, Ole e 1.0103, Ole e 1.0104, Ole e 1.0105, Ole e 1.0106, Ole e 1.0107, Ole e 10, Ole e 10.0101, Ole e 11, Ole e 11.0101, Ole e 11.0102, Ole e 12, Ole e 13, Ole e 2, Ole e 2.0101, Ole e 3, Ole e 3.0101, Ole e 36 kD, Ole e 4, Ole e 4.0101, Ole e 5, Ole e 5.0101, Ole e 6, Ole e 6.0101, Ole e 7, Ole e 7.0101, Ole e 8, Ole e 8.0101, Ole e 9, Ole e 9.0101), Ommastrephes spp (Omm b 1, Omm b 1.0101), Oncorhynchus spp (One ke 1, One ke 18 kD, One ke alpha2I, One ke Vitellogenin, One m 1, One m 1.0101, One m 1.0201, One m alpha2I, One m Protamine, One m Vitellogenin, One ma 1, One ma FPA, One ma FSA, One ma TPI, One n 1), Onchocerca spp (One o 3, One o 3.0101), Oncorhynchus spp (One is 1), Onchocerca spp (One v 3, One v 3.0101), Oratosquilla spp (Ora o 1, Ora o 1.0101), Oreochromis spp (Ore a 1, Ore mo 1, Ore mo 2, Ore mo FPA, Ore mo SCAF7145, Ore ni 1, Ore ni 18 kD, Ore ni 45 kD), Ornithonyssus spp (Orn sy 10, Orn sy 10.0101, Orn sy 10.0102), Oryctolagus spp (Ory c 1, Ory c 1.0101, Ory c 2, Ory c Casein, Ory c Phosvitin, Ory c RSA), Oryza spp (Ory s 1, Ory s 1.0101, Ory s 11, Ory s 12, Ory s 12.0101, Ory s 13, Ory s 14, Ory s 17 kD, Ory s 19 kD, Ory s 2, Ory s 23, Ory s 3, Ory s 7, Ory s akTI, Ory s GLP52, Ory s GLP63, Ory s Glyoxalase I, Ory s NRA), Ostrya spp (Ost c 1, Ost c 1.0101), Ovis spp (Ovi a ALA, Ovi a BLG, Ovi a Casein, Ovi a Casein alphaSl, Ovi a Casein alphaS2, Ovi a Casein beta, Ovi a Casein kappa, Ovi a Phosvitin, Ovi a SSA), Pachycondyla spp (Pac c 3), Pagrus spp (Pag m 1, Pag pa 1), Pampus spp (Pam ar 1, Pam c 1), Pandalus spp (Pan b 1, Pan b 1.0101), Pangasius spp (Pan bo 1), Pandalus spp (Pan e 1, Pan e 1.0101, Pan e 4), Panulirus spp (Pan h 1, Pan hy 1), Pangasius spp (Pan hy 18 kD, Pan hy 45 kD), Panulirus spp (Pan j 1), Panthera spp (Pan 11, Pan o 1, Pan p 1), Panulirus spp (Pan s 1, Pan s 1.0101), Panthera spp (Pan t 1), Pan spp (Pan tr TCTP), Papaver spp (Pap s 17 kD, Pap s 2, Pap s 34 kD), Papilio spp (Pap xu 7, Pap xu 7.0101, Pap xu 7.0102), Paralichthys spp (Par a 1), Parasilurus spp (Par as 1, Par c 1), Paralithodes spp (Par c 1.0101, Par c 1.0102, Par f 1), Parthenium spp (Par h 1), Parietaria spp (Par j 1, Par j 1.0101, Par j 1.0102, Par j 1.0103, Par j 1.0201, Par j 2, Par j 2.0101, Par j 2.0102, Par j 3, Par j 3.0101, Par j 3.0102, Par j 4, Par j 4.0101, Par j J1-J2), Paralichthys spp (Par le 1), Parietaria spp (Par m 1, Par o 1, Par o 1.0101), Paralichthys spp (Par of 1, Par of alpha2I), Parahucho spp (Par pe Vitellogenin), Passiflora spp (Pas e Chitinase, Pas e Hevein), Paspalum spp (Pas n 1, Pas n 1.0101, Pas n 13), Patinopecten spp (Pat y 1), Pediculus spp (Ped h 7, Ped h 7.0101), Penaeus spp (Pen a 1, Pen a 1.0101, Pen a 1.0102, Pen a 1.0102 (103-117), Pen a 1.0102 (109-123), Pen a 1.0102 (1-15), Pen a 1.0102 (115-129), Pen a 1.0102 (121-135), Pen a 1.0102 (127-141), Pen a 1.0102 (13-27), Pen a 1.0102 (133-147), Pen a 1.0102 (139-153), Pen a 1.0102 (145-159)), Farfantepenaeus spp (Pen a 1.0102 (151-165)), Penaeus spp (Pen a 1.0102 (157-171), Pen a 1.0102 (163-177), Pen a 1.0102 (169-183), Pen a 1.0102 (175-189), Pen a 1.0102 (181-195), Pen a 1.0102 (187-201), Pen a 1.0102 (193-207), Pen a 1.0102 (19-33), Pen a 1.0102 (199-213), Pen a 1.0102 (205-219), Pen a 1.0102 (211-225), Pen a 1.0102 (217-231), Pen a 1.0102 (223-237), Pen a 1.0102 (229-243)), Farfantepenaeus spp (Pen a 1.0102 (235-249)), Penaeus spp (Pen a 1.0102 (241-255), Pen a 1.0102 (247-261), Pen a 1.0102 (253-267), Pen a 1.0102 (25-39), Pen a 1.0102 (259-273), Pen a 1.0102 (265-279), Pen a 1.0102 (270-284), Pen a 1.0102 (31-45), Pen a 1.0102 (37-51), Pen a 1.0102 (43-57), Pen a 1.0102 (49-63)), Farfantepenaeus spp (Pen a 1.0102 (55-69)), Penaeus spp (Pen a 1.0102 (61-75), Pen a 1.0102 (67-81), Pen a 1.0102 (7-21), Pen a 1.0102 (73-87), Pen a 1.0102 (79-93), Pen a 1.0102 (85-99), Pen a 1.0102 (91-105), Pen a 1.0102 (97-111), Pen a 1.0103), Penicillium spp (Pen b 13, Pen b 13.0101, Pen b 26, Pen b 26.0101, Pen c 1, Pen c 13, Pen c 13.0101, Pen c 18, Pen c 19, Pen c 19.0101, Pen c 2, Pen c 22, Pen c 22.0101, Pen c 24, Pen c 24.0101, Pen c 3, Pen c 3.0101, Pen c 30, Pen c 30.0101, Pen c 32, Pen c 32.0101, Pen c MnSOD, Pen ch 13, Pen ch 13.0101, Pen ch 18, Pen ch 18.0101, Pen ch 20, Pen ch 20.0101, Pen ch 31, Pen ch 31.0101, Pen ch 33, Pen ch 33.0101, Pen ch 35, Pen ch 35.0101, Pen ch MnSOD), Penaeus spp (Pen i 1, Pen i 1.0101, Pen m 1, Pen m 1.0101, Pen m 1.0102, Pen m 2, Pen m 2.0101, Pen m 3, Pen m 3.0101, Pen m 4, Pen m 4.0101, Pen m 6, Pen m 6.0101), Penicillium spp (Pen o 18, Pen o 18.0101), Penaeus spp (Pena o 1, Pena o 1.0101), Periplaneta spp (Per a 1, Per a 1.0101, Per a 1.0102, Per a 1.0103, Per a 1.0104, Per a 1.0105, Per a 1.0201, Per a 10, Per a 10.0101, Per a 2, Per a 3, Per a 3.0101, Per a 3.0201, Per a 3.0202, Per a 3.0203, Per a 4, Per a 5, Per a 6, Per a 6.0101, Per a 7, Per a 7.0101, Per a 7.0102, Per a 7.0103, Per a 9, Per a 9.0101, Per a Cathepsin, Per a FABP, Per a Trypsin, Per f 1, Per f 7, Per f 7.0101), Perna spp (Per v 1), Persea spp (Pers a 1, Pers a 1.0101, Pers a 4), Petroselinum spp (Pet c 1, Pet c 2, Pet c 3), Phalaris spp (Pha a 1, Pha a 1.0101, Pha a 5, Pha a 5.0101, Pha a 5.02, Pha a 5.03, Pha a 5.04), Phaseolus spp (Pha v 3, Pha v 3.0101, Pha v 3.0201, Pha v aAI, Pha v aAI.0101, Pha v Chitinase, Pha v PHA, Pha v Phaseolin), Phleum spp (Phl p 1, Phl p 1.0101, Phl p 1.0102, Phl p 11, Phl p 11.0101, Phl p 12, Phl p 12.0101, Phl p 12.0102, Phl p 12.0103, Phl p 13, Phl p 13.0101, Phl p 2, Phl p 2.0101, Phl p 3, Phl p 3.0101, Phl p 3.0102, Phl p 4, Phl p 4.0101, Phl p 4.0102, Phl p 4.0201, Phl p 4.0202, Phl p 4.0203, Phl p 4.0204, Phl p 5, Phl p 5.0101, Phl p 5.0102, Phl p 5.0103, Phl p 5.0104, Phl p 5.0105, Phl p 5.0106, Phl p 5.0107, Phl p 5.0108, Phl p 5.0109, Phl p 5.0201, Phl p 5.0202, Phl p 5.0203, Phl p 5.0204, Phl p 5.0205, Phl p 5.0206, Phl p 5.0207, Phl p 6, Phl p 6.0101, Phl p 6.0102, Phl p 7, Phl p 7.0101, Phl p P1-P2-P5-P6, Phl p P2-P6, Phl p P5-P1, Phl p P6-P2), Phoenix spp (Pho d 2, Pho d 2.0101, Pho d 40 kD, Pho d 90 kD), Phodopus spp (Pho s 21 kD), Phoma spp (Pho t 1), Phragmites spp (Phr a 1, Phr a 12, Phr a 13, Phr a 4, Phr a 5), Phytolacca spp (Phy a RIP), Pimpinella spp (Pim a 1, Pim a 2), Pinna spp (Pin a 1), Piper spp (Pip n 14 kD, Pip n 28 kD), Pisum spp (Pis s 1, Pis s 1.0101, Pis s 1.0102, Pis s 2, Pis s 2.0101, Pis s 5, Pis s Agglutinin, Pis s Albumin), Pistacia spp (Pis v 1, Pis v 1.0101, Pis v 2, Pis v 2.0101, Pis v 2.0201, Pis v 3, Pis v 3.0101, Pis v 4, Pis v 4.0101, Pis v 5, Pis v 5.0101), Platanus spp (Pla a 1, Pla a 1.0101, Pla a 2, Pla a 2.0101, Pla a 3, Pla a 3.0101, Pla a 8), Platichthys spp (Pla f 1), Plantago spp (Pla 11, Pla 11.0101, Pla I 1.0102, Pla 11.0103, Pla I Cytochrome C), Platanus spp (Pla oc 1, Pla or 1, Pla or 1.0101, Pla or 2, Pla or 2.0101, Pla or 3, Pla or 3.0101, Pla or 4, Pla or CyP, Pla r 1), Plectropomus spp (Ple ar 1), Pleospora spp (Ple h 1), Plectropomus spp (Ple le 1), Plodia spp (Plo i 1, Plo i 1.0101, Plo i 2, Plo i 2.0101), Poa spp (Poa p 1, Poa p 1.0101, Poa p 10, Poa p 12, Poa p 13, Poa p 2, Poa p 4, Poa p 5, Poa p 5.0101, Poa p 6, Poa p 7), Polistes spp (Pol a 1, Pol a 1.0101, Pol a 2, Pol a 2.0101, Pol a 5, Pol a 5.0101, Pol d 1, Pol d 1.0101, Pol d 1.0102, Pol d 1.0103, Pol d 1.0104, Pol d 4, Pol d 4.0101, Pol d 5, Pol d 5.0101, Pol e 1, Pol e 1.0101, Pol e 2, Pol e 4, Pol e 4.0101, Pol e 5, Pol e 5.0101, Pol f 5, Pol f 5.0101, Pol g 1, Pol g 1.0101, Pol g 2, Pol g 4, Pol g 5, Pol g 5.0101, Pol he MLT, Pol m 5, Pol m 5.0101), Polypedilum spp (Pol n 1), Pollicipes spp (Pol po 1), Pollachius spp (Pol vi 1), Polybia spp (Poly p 1, Poly p 1.0101, Poly p 2, Poly p 5, Poly s 5, Poly s 5.0101), Pomatomus spp (Porn sa 1), Pongo spp (Pon ab HSA), Pontastacus spp (Pon I 4, Pon I 4.0101, Pon I 7, Pon I 7.0101), Portunus spp (Por s 1, Por s 1.0101, Por s 1.0102, Por tr 1, Por tr 1.0101), Protortonia spp (Pro ca 38 kD), Procumbarus spp (Pro cl 1, Pro cl 1.0101, Pro cl 21 kD), Prosopis spp (Pro j 20 kD), Prunus spp (Pru ar 1, Pru ar 1.0101, Pru ar 3, Pru ar 3.0101, Pru av 1, Pru av 1.0101, Pru av 1.0201, Pru av 1.0202, Pru av 1.0203, Pru av 2, Pru av 2.0101, Pru av 3, Pru av 3.0101, Pru av 4, Pru av 4.0101, Pru c 1, Pru d 1, Pru d 2, Pru d 3, Pru d 3.0101, Pru d 4, Pru du 1, Pru du 2, Pru du 2S Albumin, Pru du 3, Pru du 3.0101, Pru du 4, Pru du 4.0101, Pru du 4.0102, Pru du 5, Pru du 5.0101, Pru du 6, Pru du 6.0101, Pru du 6.0201, Pru du Conglutin, Pru p 1, Pru p 1.0101, Pru p 2, Pru p 2.0101, Pru p 2.0201, Pru p 2.0301, Pru p 3, Pru p 3.0101, Pru p 3.0102, Pru p 4, Pru p 4.0101, Pru p 4.0201, Pru sa 3), Psilocybe spp (Psi c 1, Psi c 1.0101, Psi c 2, Psi c 2.0101), Psoroptes spp (Pso o 1, Pso o 10, Pso o 10.0101, Pso o 11, Pso o 13, Pso o 14, Pso o 2, Pso o 21, Pso o 3, Pso o 5, Pso o 7), Puma spp (Pum c 1), Punica spp (Pun g 3), Pyrus spp (Pyr c 1, Pyr c 1.0101, Pyr c 3, Pyr c 3.0101, Pyr c 4, Pyr c 4.0101, Pyr c 5, Pyr c 5.0101, Pyr py 2), Quercus spp (Que a 1, Que a 1.0101, Que a 1.0201, Que a 1.0301, Que a 1.0401, Que a 2, Que a 4), Rachycentron spp (Rac ca 1), Rana spp (Ran e 1, Ran e 1.0101, Ran e 2, Ran e 2.0101), Ranina spp (Ran ra 1), Rangifer spp (Ran t BLG), Rattus spp (Rat n 1, Rat n 1.0101, Rat n Casein, Rat n Gelatin, Rat n IgG, Rat n Phosvitin, Rat n RSA, Rat n Transferrin), Rhizomucor spp (Rhi m AP), Rhizopus spp (Rhi nv Lipase, Rhi o Lipase), Rhomboplites spp (Rho au 1), Rhodotorula spp (Rho m 1, Rho m 1.0101, Rho m 2, Rho m 2.0101), Ricinus spp (Ric c 1, Ric c 1.0101, Ric c 2, Ric c 3, Ric c 8, Ric c RIP), Rivulus spp (Riv ma 1), Robinia spp (Rob p 2, Rob p 4, Rob p Glucanase), Rosa spp (Ros r 3), Roystonea spp (Roy e 2), Rubus spp (Rub i 1, Rub i 1.0101, Rub i 3, Rub i 3.0101, Rub i Chitinase, Rub i CyP), Saccharomyces spp (Sac c Carboxypeptidase Y, Sac c CyP, Sac c Enolase, Sac c Glucosidase, Sac c Invertase, Sac c MnSOD, Sac c P2, Sac c Profilin), Salvelinus spp (Sal f 1), Salsola spp (Sal k 1, Sal k 1.0101, Sal k 1.0201, Sal k 1.0301, Sal k 1.0302, Sal k 2, Sal k 2.0101, Sal k 3, Sal k 3.0101, Sal k 4, Sal k 4.0101, Sal k 4.0201, Sal k 5, Sal k 5.0101), Salvelinus spp (Sal le Vitellogenin), Salmo spp (Sal s 1, Sal s 1.0101, Sal s 1.0201, Sal s 2, Sal s 2.0101, Sal s Gelatin), Sambucus spp (Sam n 1), Sander spp (San lu 1), Saponaria spp (Sap o RIP), Sardinops spp (Sar m 1), Sarkidiornis spp (Sar ml 1), Sardina spp (Sar p 1), Sarcoptes spp (Sar s 1, Sar s 14, Sar s 3, Sar s GST, Sar s PM), Sardinops spp (Sar sa 1, Sar sa 1.0101), Schistosoma spp (Sch j GST, Sch j PM, Sch j Sj22, Sch j Sj67, Sch ma Sm20, Sch ma Sm21, Sch ma Sm22, Sch ma Sm31), Sciaenops spp (Sci oc 1), Scomber spp (Sco a 1), Scombermorus spp (Sco ca 1), Scomberomorus spp (Sco g 1), Scomber spp (Sco j 1, Sco ma 1, Sco s 1), Scolopendra spp (Sco y 7, Sco y 7.0101), Scylla spp (Scy o 1, Scy o 1.0101, Scy o 2, Scy pa 1, Scy pa 2, Scy s 1, Scy s 1.0101, Scy s 2), Sebastes spp (Seb fa 1, Seb in 1, Seb m 1, Seb m 1.0101, Seb m 1.0201), Secale spp (Sec c 1, Sec c 12, Sec c 13, Sec c 2, Sec c 20, Sec c 20.0101, Sec c 20.0201, Sec c 28, Sec c 3, Sec c 4, Sec c 4.0101, Sec c 4.0201, Sec c 5, Sec c 5.0101, Sec c akTI, Sec c akTI.0101), Senecio spp (Sen j MDH, Sen j PL), Sepia spp (Sep e 1, Sep e 1.0101), Sepioteuthis spp (Sep 11, Sep I 1.0101), Sepia spp (Sep m 1), Seriola spp (Ser d 1, Ser 1a 1), Sergestes spp (Ser lu 1), Seriola spp (Ser q 1, Ser ri 1), Sesamum spp (Ses i 1, Ses i 1.0101, Ses i 2, Ses i 2.0101, Ses i 3, Ses i 3.0101, Ses i 4, Ses i 4.0101, Ses i 5, Ses i 5.0101, Ses i 6, Ses i 6.0101, Ses i 7, Ses i 7.0101, Ses i 8), Shigella spp (Shi bo GST, Shi dy GST), Simulia spp (Sim vi 1, Sim vi 2, Sim vi 3, Sim vi 4, Sim vi 70 kD), Sinapis spp (Sin a 1, Sin a 1.0101, Sin a 1.0104, Sin a 1.0105, Sin a 1.0106, Sin a 1.0107, Sin a 1.0108, Sin a 2, Sin a 2.0101, Sin a 3, Sin a 3.0101, Sin a 4, Sin a 4.0101), Sinonovacula spp (Sin c 1, Sin c 1.0101), Solenopsis spp (Sol g 2, Sol g 2.0101, Sol g 3, Sol g 3.0101, Sol g 4, Sol g 4.0101, Sol g 4.0201, Sol i 1, Sol i 1.0101, Sol i 2, Sol i 2.0101, Sol i 3, Sol i 3.0101, Sol i 4, Sol i 4.0101), Solenocera spp (Sol me 1), Solenopsis spp (Sol r 1, Sol r 2, Sol r 2.0101, Sol r 3, Sol r 3.0101, Sol s 2, Sol s 2.0101, Sol s 3, Sol s 3.0101, Sol s 4), Solea spp (Sol so 1, Sol so TPI), Solanum spp (Sola t 1, Sola t 1.0101, Sola t 2, Sola t 2.0101, Sola t 3, Sola t 3.0101, Sola t 3.0102, Sola t 4, Sola t 4.0101, Sola t 8, Sola t Glucanase), Sorghum spp (Sor b 1, Sor h 1, Sor h 1.0101, Sor h 12, Sor h 7), Sparus spp (Spa a 1), Sphyrna spp (Sph ti 1), Spirulina spp (Spi mx beta_Phycocyanin), Spinacia spp (Spi o 2, Spi o RuBisCO), Squilla spp (Squ ac 1, Squ ac 1.0101, Squ o 1, Squ o 1.0101), Staphylococcus spp (Sta a FBP, Sta a SEA, Sta a SEB, Sta a SEC, Sta a SED, Sta a SEE, Sta a TSST), Stachybotrys spp (Sta c 3, Sta c 3.0101, Sta c Cellulase, Sta c Hemolysin, Sta c SchS34, Sta c Stachyrase A), Stemphylium spp (Ste b 1, Ste c 1, Ste v 1), Stolephorus spp (Sto i 1), Struthio spp (Str c 1, Str c 2, Str c 3), Streptococcus spp (Str dy Streptokinase), Streptomyces spp (Str g Pronase), Streptococcus spp (Str pn PspC), Strongylocentrotus spp (Str pu 18 kD, Str pu Vitellogenin), Streptococcus spp (Str py SPEA, Str py SPEC, Str py Streptokinase), Strongyloides spp (Str st 45 kD), Streptomyces spp (Str v PAT), Styela spp (Sty p 1), Suidasia spp (Sui m 1, Sui m 13, Sui m 2, Sui m 3, Sui m 5, Sui m 5.01, Sui m 5.02, Sui m 5.03, Sui m 6, Sui m 7, Sui m 8, Sui m 9), Sus spp (Sus s ACTH, Sus s ALA, Sus s Amylase, Sus s BLG, Sus s Casein, Sus s Casein alphaS1, Sus s Casein alphaS2, Sus s Casein beta, Sus s Casein kappa, Sus s Gelatin, Sus s HG, Sus s Insulin, Sus s Lipase, Sus s Pepsin, Sus s Phosvitin, Sus s PRVB, Sus s PSA, Sus s TCTP), Syntelopodeuma spp (Syn y 7, Syn y 7.0101), Syringa spp (Syr v 1, Syr v 1.0101, Syr v 1.0102, Syr v 1.0103, Syr v 2, Syr v 3, Syr v 3.0101), Tabanus spp (Tab y 1, Tab y 1.0101, Tab y 2, Tab y 2.0101, Tab y 5, Tab y 5.0101), Tadorna spp (Tad ra 1), Talaromyces spp (Tal st 22, Tal st 3, Tal st 8), Taraxacum spp (Tar o 18 kD), Taxodium spp (Tax d 2), Tegenaria spp (Teg d Hemocyanin), Teladorsagia spp (Tel ci 3), Thaumetopoea spp (Tha p 1, Tha p 1.0101, Tha p 2, Tha p 2.0101), Theragra spp (The c 1), Thermomyces spp (The I Lipase, The sp Lipase, The sp Xylanase), Thunnus spp (Thu a 1, Thu a 1.0101, Thu a Collagen, Thu al 1, Thu at 1, Thu o 1, Thu o Collagen), Thuja spp (Thu oc 3, Thu p 1), Thunnus spp (Thu t 1, Thu to 1), Thyrsites spp (Thy at 1), Thyrophygus spp (Thy y 7, Thy y 7.0101), Todarodes spp (Tod p 1, Tod p 1.0101, Tod p 1.0102), Toxoptera spp (Tox c 7, Tox c 7.0101), Toxocara spp (Tox ca TES120, Tox ca TES26, Tox ca TES30), Toxoplasma spp (Tox g HSP70), Trachypenaeus spp (Tra c 1), Trachinotus spp (Tra ca 1), Trachurus spp (Tra j 1, Tra j Gelatin, Tra tr Gelatin), Triticum spp (Tri a 1, Tri a 10 kD, Tri a 12, Tri a 12.0101, Tri a 12.0102, Tri a 12.0103, Tri a 12.0104, Tri a 13, Tri a 14, Tri a 14.0101, Tri a 14.0201, Tri a 15, Tri a 15.0101, Tri a 18, Tri a 18.0101, Tri a 19, Tri a 19.0101, Tri a 2, Tri a 21, Tri a 21.0101, Tri a 23kd, Tri a 25, Tri a 25.0101, Tri a 26, Tri a 26.0101, Tri a 27, Tri a 27.0101, Tri a 28, Tri a 28.0101, Tri a 29, Tri a 29.0101, Tri a 29.0201, Tri a 3, Tri a 30, Tri a 30.0101, Tri a 31, Tri a 31.0101, Tri a 32, Tri a 32.0101, Tri a 33, Tri a 33.0101, Tri a 34, Tri a 34.0101, Tri a 35, Tri a 35.0101, Tri a 36, Tri a 36.0101, Tri a 37, Tri a 37.0101, Tri a 4, Tri a 4.0101, Tri a 4.0201, Tri a 5, Tri a 7, Tri a aA_SI, Tri a alpha_Gliadin, Tri a bA, Tri a Bd36K, Tri a beta_Gliadin, Tri a Chitinase, Tri a CM16, Tri a DH, Tri a Endochitinase, Tri a gamma_Gliadin, Tri a Germin, Tri a Gliadin, Tri a GST, Tri a LMW Glu, Tri a LMW-GS B16, Tri a LMW-GS P42, Tri a LMW-GS P73, Tri a LTP2, Tri a omega2_Gliadin, Tri a Peroxidase, Tri a Peroxidase 1, Tri a SPI, Tri a TLP, Tri a Tritin, Tri a XI), Tritirachium spp (Tri al Proteinase K), Tribolium spp (Tri ca 17, Tri ca 17.0101, Tri ca 7, Tri ca 7.0101), Trichostrongylus spp (Tri co 3, Tri co 3.0101), Trichophyton spp (Tri eq 4), Trigonella spp (Tri fg 1, Tri fg 2, Tri fg 3, Tri fg 4), Trichosanthes spp (Tri k RIP), Trichiurus spp (Tri le 1), Triticum spp (Tri m Peroxidase), Trichophyton spp (Tri me 2, Tri me 4), Trisetum spp (Tri p 1, Tri p 5), Trichinella spp (Tri ps 3, Tri ps 3.0101), Trichophyton spp (Tri r 2, Tri r 2.0101, Tri r 4, Tri r 4.0101), Trichoderma spp (Tri rs Cellulase), Triticum spp (Tri s 14), Trichophyton spp (Tri sc 2, Tri sc 4, Tri so 2), Trichinella spp (Tri sp 3, Tri sp 3.0101, Tri sp 3.0102, Tri sp 3.0103, Tri sp 3.0104, Tri sp 3.0105, Tri sp 3.0106), Trichophyton spp (Tri t 1, Tri t 1.0101, Tri t 4, Tri t 4.0101), Triticum spp (Tri td 14, Tri td akTI), Trichoderma spp (Tri v Cellulase), Trichophyton spp (Tri ye 4), Triatoma spp (Tria p 1, Tria p 1.0101), Triplochiton spp (Trip s 1), Turbo spp (Tur c 1, Tur c PM), Tyrophagus spp (Tyr p 1, Tyr p 10, Tyr p 10.0101, Tyr p 10.0102, Tyr p 13, Tyr p 13.0101, Tyr p 2, Tyr p 2.0101, Tyr p 24, Tyr p 24.0101, Tyr p 3, Tyr p 3.0101, Tyr p 4, Tyr p 5, Tyr p 5.01, Tyr p 5.02, Tyr p 5.03, Tyr p 7, Tyr p alpha Tubulin), Ulocladium spp (Ulo a 1, Ulo at 1, Ulo b 1, Ulo c 1, Ulo co 1, Ulo cu 1, Ulo mu 1, Ulo ob 1, Ulo se 1, Ulo su 1, Ulo to 1), Uncia spp (Unc u 1), Urophycis spp (Uro to 1), Vaccinium spp (Vac m 3), Varroa spp (Var j 13 kD), Venerupis spp (Ven ph 1, Ven ph 1.0101), Vespula spp (Ves f 1, Ves f 2, Ves f 5, Ves f 5.0101, Ves g 1, Ves g 2, Ves g 5, Ves g 5.0101, Ves m 1, Ves m 1.0101, Ves m 2, Ves m 2.0101, Ves m 5, Ves m 5.0101, Ves m MLT, Ves p 1, Ves p 2, Ves p 5, Ves p 5.0101, Ves s 1, Ves s 1.0101, Ves s 2, Ves s 5, Ves s 5.0101, Ves v 1, Ves v 1.0101, Ves v 2, Ves v 2.0101, Ves v 2.0201, Ves v 3, Ves v 3.0101, Ves v 5, Ves v 5.0101, Ves v 5-Pol a 5, Ves vi 5, Ves vi 5.0101), Vespa spp (Vesp c 1, Vesp c 1.0101, Vesp c 2, Vesp c 5, Vesp c 5.0101, Vesp c 5.0102, Vesp m 1, Vesp m 1.0101, Vesp m 5, Vesp m 5.0101, Vesp ma 1, Vesp ma 2, Vesp ma 5, Vesp ma MLT, Vesp v MLT), Vigna spp (Vig r 1, Vig r 1.0101, Vig r 17 kD, Vig r 5, Vig r 8S Globulin, Vig r Albumin, Vig r beta-Conglycinin), Vitis spp (Vit v 1, Vit v 1.0101, Vit v 4, Vit v 5, Vit v Glucanase, Vit v TLP), Xiphias spp (Xip g 1, Xip g 1.0101, Xip g 25 kD), Zea spp (Zea m 1, Zea m 1.0101, Zea m 11, Zea m 12, Zea m 12.0101, Zea m 12.0102, Zea m 12.0103, Zea m 12.0104, Zea m 12.0105, Zea m 13, Zea m 14, Zea m 14.0101, Zea m 14.0102, Zea m 2, Zea m 20S, Zea m 22, Zea m 25, Zea m 25.0101, Zea m 27 kD Zein, Zea m 3, Zea m 4, Zea m 5, Zea m 50 kD Zein, Zea m 7, Zea m Chitinase, Zea m G1, Zea m G2, Zea m PAO, Zea m Zml3), Zeus spp (Zeu fa 1), Ziziphus spp (Ziz m 1, Ziz m 1.0101), Zoarces spp (Zoa a ISP III), and Zygophyllum spp (Zyg f 2).

### Heterologous Antigens

The compositions herein described can comprise a plurality of presenting peptides derived from heterologous antigens (e.g, a plurality of disparate presenting peptide). In some embodiments, “heterologous antigens” are antigens that are of different origins, such as derived from pathogens of different taxonomic groups such as different strains, species, subgenera, genera, subfamilies or families and/or from antigenically divergent pathogens (e.g., variants thereof). Classification of viruses into various taxonomic groups is well understood by those skilled in the art. Each of the disparate presenting peptide of the plurality of disparate presenting peptide can differ with respect to each other.

At least two of the APCs of the plurality of APCs can be different from each other with respect to the presenting peptide, and wherein the population of APVs thereby display a plurality of disparate presenting peptide. The population of APVs can comprise one or more homotypic APVs, wherein the plurality of APCs of a homotypic APV can be the same as each other with respect to the presenting peptide, and wherein a homotypic APV thereby does not display a plurality of disparate presenting peptide. The population of APVs can comprise one or more heterotypic APVs, wherein at least two of the APCs of a heterotypic APV can be different from each other with respect to the presenting peptide, and wherein a heterotypic APV thereby displays a plurality of disparate presenting peptide. The population of APVs can comprise a mixture of two or more homotypic APVs that differ from each other with respect to the presenting peptide of the plurality of APCs present in said two or more homotypic APVs, and wherein the population of APVs thereby displays a plurality of disparate presenting peptide. The population of APVs can comprise a mixture of two or more heterotypic APVs that differ from each other with respect to the presenting peptide of the plurality of APCs of said two or more heterotypic APVs. The population of APVs can comprise a mixture of heterotypic APVs and homotypic APVs. Heterotypic APVs can be capable of eliciting heterologous antibody responses against an additional antigen, and wherein said heterotypic APVs do not display presenting peptide derived from said additional antigen.

In some embodiments, the plurality of disparate presenting peptide comprises: between about 2 and about 500 (e.g., 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or a number or a range between any two of these values) presenting peptides that differ from each other; presenting peptide of a same protein type; and/or presenting peptide of different protein types.

The plurality of presenting peptide can be of a same protein type or corresponding proteins. Presenting peptide of a same protein type may or may not have identical amino acid sequences, but generally share some sequence homology. For example, the coronavirus S proteins of different coronaviruses are of a same protein type or corresponding proteins. As another example, envelope proteins from different coronaviruses are considered the same protein type or corresponding proteins. In some embodiments, proteins of different coronavirus taxonomic groups having the same function are considered the same protein type or corresponding proteins.

Alternatively, in some embodiments the plurality of presenting peptide can derive from antigens of different protein types. Antigens of different protein types typically have different functions. For example, the plurality of presenting peptide can derive from coronavirus S proteins or portions thereof as well as other coronavirus proteins such as a coronavirus N protein or a portion thereof, a coronavirus HE protein or a portion thereof, a coronavirus papain-like protease or a portion thereof, a coronavirus 3CL protease or a portion thereof, and/or a coronavirus M protein or a portion thereof.

The number of the first presenting peptides (e.g., derived from the first antigen molecules) and the number of the second presenting peptides (e.g., derived from the second antigen molecules) can be in a ratio from 1:100 to 100:1 (e.g., 1:1, 1:1.1, 1:1.2, 1:1.3, 1:1.4, 1:1.5, 1:1.6, 1:1.7, 1:1.8, 1:1.9, 1:2, 1:2.5, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100 to 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any of these values). The plurality of cancer-related antigens can comprise three, four, five, size seven, or eight cancer-related antigens, each of the cancer-related antigens different from one another.

### Supplemental Polypeptides

In some embodiments, the supplemental polypeptides comprise co-stimulatory molecules. The co-stimulatory molecules cannot activate T cells independently, but are critical to the functional naïve T cell response, the nature of which depends upon the outcome of integration of these stimulatory or inhibitory signals. For example, in the absence of CD28 ligation, stimulation of naïve T cells induces T cell anergy in vitro, precluding an effective T cell response and promoting tolerance.

In some embodiments, the APVs comprise one or more supplemental polypeptide(s) (e.g., immunosuppressive polypeptide(s), immunostimulatory polypeptide(s), costimulatory polypeptide(s), cell adhesion polypeptide(s), anti-phagocytic polypeptide(s), tissue-homing polypeptide(s), or any combination thereof). The one or more supplemental polypeptide(s) can comprise cell-surface-molecule binding polypeptide(s), optionally antibody polypeptide(s), antibody-like polypeptide(s) and/or receptor ligand(s). In some embodiments, the use of APV-displayed antibodies/nanobodies/receptor ligands can result in targeting of the APVs to specific cell types (e.g., as a way to control tropism).

In some embodiments, the supplemental polypeptide molecules are selected from CD3ε, CD3ζ, CD4, CD5, CD8α, CD9, CD16, CD22, CD27, CD28, CD33, CD3γ, CD45, CD64, CD80, CD86, CD 134, CD137, CD152, CD 154, PD1, CARD11, CD2, CD7, CD27, CD28, CD30, CD40, CD54 (ICAM), CD3, CD3γ, CD3δ, CD25, CD39, GITR, LRRC32, Neuropilin-1, CCR6, CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, ZAP70, CD73, LFA-1, CD47, CCR7, derivatives thereof, or any combination thereof.

CD80 (Cluster of differentiation 80, B7-1) is a 254 residue type 1 transmembrane protein, consisting of an extracellular IgV-like domain that binds to it's functional partners (e.g., PD-L1, CTLA-4 (cytotoxic T lymphocyte-associated protein 4) and CD28 (Cluster of differentiation 28)), followed by a membrane proximal IgC-like domain. CD80 plays a key regulatory role within the immune synapse. CD80 can form heterodimers with PD-L1 on the same cell membrane (in cis). The formation of CD80/PD-L1 heterodimers inhibits PD-L1 binding to PD-1, as both CD80 and PD-1 share overlapping binding sites on PD-L1. PD-L1 shows widespread expression in tumor cells, whereas CD80 is expressed primarily on antigen-presenting cells, including dendritic cells, and is upregulated after these cells are activated. Therefore, CD80 can play a key role in antitumor immune response.

The CD83 molecule has been identified to be expressed on numerous activated immune cells, including B and T lymphocytes, monocytes, dendritic cells, microglia, and neutrophils. CD83 has two isoforms, the membrane-bound and soluble isoforms. Several studies revealed that CD83 plays a critical role in controlling and resolving immune responses. Moreover, CD83 is an essential factor during the differentiation of T and B lymphocytes, and the development and maintenance of tolerance. Orthologs of CD83 have been detected in more than 50 vertebrates including fish, reptiles, birds, and mammals. Both, murine (muCD83) and human CD83 (hCD83) are composed of an extracellular V-type Ig-like domain, a transmembrane domain, and a cytoplasmic tail.

PD-L1, also known as CD274 or B7H1, stands for programmed cell death ligand 1. It is a type I transmembrane protein that is thought to repress immune responses by binding to its receptor (PD-1), thus inhibiting T-cell activation, proliferation, and cytokine production. It contains V-like and C-like immunoglobulin domains. PD-L1 expression is regulated by various cytokines, such as TNF-α or LPS. CD274 is a checkpoint ligand, which is expressed by various immune cells and antigen presenting cells, but also overexpressed in different cancer cells. 3 The latter results in a reduced elimination of tumour cells by CD8+ cytotoxic T lymphocytes (CTL) thereby promoting tumour progression. Increased expression of this protein in certain types of cancers, e.g., renal cell carcinoma or colon cancer, correlates with poor prognosis.

In some embodiments, the supplemental polypeptide(s) is: (i) associated with an APC-assembly domain; and/or (ii) not associated with an APC-assembly domain. In some embodiments, the nucleic acid composition comprises one or more second polynucleotide(s) encoding the one or more supplemental polypeptide(s).

### Antigen Presentation Complex (APC)

In some embodiments, the APC comprises or consists of components of the APC. In some embodiments, the APC comprises all or a portion of a major histocompatibility complex (MHC), a MHC-like protein (e.g., a MHC memetic, a MHC mimetic), or a derivative thereof. In some embodiments, the APC comprises all or a portion of MHC class I (MHC-I) or MHC class II (MHC-II).

In some embodiments, the APC comprises a single fusion protein. In some embodiments, the single fusion protein comprises one or more linker(s), the presenting peptide, the antigen presentation polypeptide(s), and the APC-assembly domain. In some embodiments, the antigen presentation polypeptide(s) is covalently connected to the APC-assembly domain and the presenting peptide. In some embodiments, the antigen presentation polypeptide(s) is situated between the APC-assembly domain and the presenting peptide. In some embodiments, at least one of the antigen presentation polypeptide(s) is situated between the APC-assembly domain and the presenting peptide. In some embodiments, the antigen presentation polypeptide(s) comprise two or more antigen presentation polypeptides with the presenting peptide situated between said two or more antigen presentation polypeptides. In some embodiments, the antigen presentation polypeptide(s) comprise MHC-I α chain and beta-2 microglobulin (β2M). In some embodiments, the components of the APC are connected in the order from N-terminus to C-terminus: the presenting peptide, beta-2 microglobulin (β2M), MHC-I α chain and the APC-assembly domain; or the APC-assembly domain, MHC-I α chain, beta-2 microglobulin (β2M) and the presenting peptide. In some embodiments, the antigen presentation polypeptide(s) comprise MHC-II α chain and MHC-II β chain. In some embodiments, the components of the APC are connected in the order from N-terminus to C-terminus: the APC-assembly domain, MHC-II β chain, the presenting peptide and the MHC-II α chain; the APC-assembly domain, MHC-II α chain, the presenting peptide and the MHC-II β chain; the MHC-II α chain, the presenting peptide, MHC-II β chain and the APC-assembly domain; or the MHC-II β chain, the presenting peptide, MHC-II α chain and the APC-assembly domain.

In some embodiments, the APC comprises two or more separate polypeptides. In some embodiments, the APC comprises two separate polypeptides. In some embodiments, at least two of the two or more separate polypeptides comprise an APC-assembly domain. In some embodiments, at least one of the two or more separate polypeptides is a fusion protein. In some embodiments, the fusion protein comprises one or more linker(s), the antigen presentation polypeptide(s), and the APC-assembly domain. In some embodiments, the fusion protein comprises (i) a linker, a first antigen presentation polypeptide, and the APC-assembly domain; or (ii) a linker, a second antigen presentation polypeptide, and the presenting peptide. In some embodiments, the first antigen presentation polypeptide is covalently connected to the APC-assembly domain, and the second antigen presentation polypeptide is covalently connected to the presenting peptide, optionally via a peptide bond. In some embodiments, the first antigen presentation polypeptide is capable of binding to the second antigen presentation polypeptide. In some embodiments, the first antigen presentation polypeptide comprises MHC-I α chain and the second antigen presentation polypeptide comprises beta-2 microglobulin (β2M). In some embodiments, the first antigen presentation polypeptide comprises beta-2 microglobulin (β2M) and the second antigen presentation polypeptide comprises MHC-I α chain. In some embodiments, the first antigen presentation polypeptide comprises MHC-II α chain and the second antigen presentation polypeptide comprises MHC-II β chain. In some embodiments, the first antigen presentation polypeptide comprises MHC-II β chain and the second antigen presentation polypeptide comprises MHC-II α chain. In some embodiments, MHC-I α chain is capable of forming a binding site that binds the presenting peptide. In some embodiments, MHC-II α chain and the MHC-II β chain are capable of forming a binding site that binds the presenting peptide.

In some embodiments, at least two components of the APC are derived from different species. In some embodiments, the antigen presentation polypeptides comprises peptides derived from at at least two species. In some embodiments, the antigen presentation polypeptides comprises a peptide derived from human and a peptide derived from mouse. In some embodiments, the antigen presentation polypeptides comprises at least one peptide derived from at at least two species. In some embodiments, the antigen presentation polypeptides comprises a peptide comprising a portion derived from human and a portion derived from mouse.

### Antigen Presenting Vesicles (APV)

In some embodiments, the APVs are derived from expression of the nucleic acid composition disclosed herein in a cell. In some embodiments, the cell is: a cell of a subject; an in vivo cell, an ex vivo cell, or an in situ cell; and/or an adherent cell or a suspension cell. In some embodiments, the cell: is not an immune cell; or is an immune cell. In some embodiments, the APVs comprise a lipid bilayer. In some embodiments, the APVs comprise a lipid bilayer derived from the cell from which the APVs was budded.

The APVs can comprise at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more of the presenting peptide and/or can be at least as immunogenic (e.g., 1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) as compared to the presenting peptide or complex of presenting peptide and antigen presentation polypeptide that are not assembled into vesicles.

The APVs can comprise at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more of the presenting peptide, an at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) higher density of the presenting peptide, and/or can be at least as immunogenic (e.g., 1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values), as compared to the presenting peptide or complex of presenting peptide and antigen presentation polypeptide that are not assembled into vesicles.

The APVs can have one or more dimensions of a eukaryotic virus. In some embodiments, less than about 1%, 2%, 3%, 4%, 5%, 6%, 7%, 8%, 9%, or 10%, of the APVs of the population of APVs have a particle size smaller than about 10 nm. In some embodiments, less than about 1%, 2%, 3%, 4%, 5%, 6%, 7%, 8%, 9%, or 10%, of the APVs of the population of APVs have a particle size exceeding about 80 nm. In some embodiments, the average diameter of the APVs of the population of APVs range from about 5 nm to about 80 nm, from about 15 nm to about 50 nm, or from about 20 nm to about 40 nm. The average diameter of the APVs of the population of APVs can be about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, or about 50 nm, optionally the average is the mean, median or mode, optionally the mean is the arithmetic mean, geometric mean, and/or harmonic mean.

In some embodiments, the APVs have a minimum diameter of about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, or about 50 nm. In some embodiments, the APVs have a maximum diameter of about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, about 50 nm, about 52 nm, about 54 nm, about 56 nm, about 58 nm, about 60 nm, about 62 nm, about 64 nm, about 66 nm, about 68 nm, about 70 nm, about 72 nm, about 74 nm, about 76 nm, about 78 nm, or about 80 nm. In some embodiments, the APVs: have a minimum diameter of about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, or about 50 nm; and/or have a maximum diameter of about 250 nm, about 252 nm, about 254 nm, about 256 nm, about 258 nm, about 260 nm, about 262 nm, about 264 nm, about 266 nm, about 268 nm, about 270 nm, about 272 nm, about 274 nm, about 276 nm, about 278 nm, or about 280 nm.

In some embodiments, storage of the APVs at 4° C. for at least three months reduces immunogenicity less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%. The composition can be stable for at least about 2 weeks, about 1 month, about 2 months, about 3 months, about 4 months, about 5 months, about 6 months, or about 1 year, after storage as a liquid at a temperature of about 4° C. At least about at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, of the APVs can be immunogenic at least 1 month after storage as a liquid at a temperature of about 5° C.

In some embodiments, the population of APVs comprise: one or more homotypic APVs. In some embodiments, the plurality of APCs of a homotypic APV are the same as each other with respect to the presenting peptide, and a homotypic APV thereby does not display a plurality of disparate presenting peptides. In some embodiments, all the homotypic APVs in the population of APVs are the same with respect to the presenting peptide. In some embodiments, the plurality of APCs of a homotypic APV are the same as each other with respect to the antigen presentation polypeptide. In some embodiments, all the homotypic APVs in the population of APVs are the same with respect to the antigen presentation polypeptide.

In some embodiments, the population of APVs comprise: a mixture of two or more homotypic APVs that differ from each other with respect to the presenting peptide of the plurality of APCs present in said two or more homotypic APVs, and the population of APVs thereby displays a plurality of disparate presenting peptides. In some embodiments, the population of APVs comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 homotypic APVs that differ from each other with respect to the presenting peptide. In some embodiments, at least one homotypic APV in the population of APVs comprises the antigen presentation polypeptide or a portion thereof derived from human and at least one homotypic APV in the population of APVs comprises the antigen presentation polypeptide or a portion thereof derived from a non-human source (e.g., from mouse). In some embodiments, the population of APVs comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 homotypic APVs that differ from each other with respect to the antigen presentation polypeptide. In some embodiments, the antigen presentation polypeptides of the population of homotypic APVs are derived from at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 different species. In some embodiments, at least one homotypic APV in the population of APVs comprises MHC-I α nd at least one homotypic APV in the population of APVs comprises MHC-II.

In some embodiments, the population of APVs comprise: one or more heterotypic APVs. In some embodiments, at least two of the APCs of a heterotypic APV are different from each other with respect to the presenting peptide, and a heterotypic APV thereby displays a plurality of disparate presenting peptides. In some embodiments, all the heterotypic APVs in the population of APVs are the same with respect to the presenting peptide. In some embodiments, at least two of the APCs of a heterotypic APV are different from each other with respect to the antigen presentation polypeptides. In some embodiment, the antigen presentation polypeptides of each of the APVs are from different species or comprise a mixture of MHC-I and MHC-II

In some embodiments, the population of APVs comprise: a mixture of two or more heterotypic APVs that differ from each other with respect to the presenting peptide of the plurality of APCs of said two or more heterotypic APVs. In some embodiments, the population of APVs comprise: a mixture of two or more heterotypic APVs that differ from each other with respect to the antigen presentation polypeptides of the plurality of APCs of said two or more heterotypic APVs.

In some embodiments, the population of APVs comprise: a mixture of one or more homotypic APVs and one or more heterotypic APVs with respect to the presenting peptide of the plurality of APCs of said two or more heterotypic APVs, and the population of APVs thereby displays a plurality of disparate presenting peptides. In some embodiments, the population of APVs comprise: a mixture of one or more homotypic APVs and one or more heterotypic APVs with respect to the antigen presentation polypeptides of the plurality of APCs of said two or more heterotypic APVs.

### Vectors and Carriers

The nucleic acid composition (e.g., a plurality of polynucleotides encoding components of the APCs) can be complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition (e.g., a plurality of polynucleotides encoding components of the APCs) is, comprises, or further comprises, one or more vectors. At least one of the one or more vectors can be a viral vector, a plasmid, a transposable element, a naked DNA vector, a lipid nanoparticle (LNP), or any combination thereof. The viral vector can be an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. The transposable element can be piggybac transposon or sleeping beauty transposon.

The polynucleotide(s) encoding components of the APC(s) can be comprised in the one or more vectors. The polynucleotide(s) encoding components of the APC(s) can be comprised in the same vector and/or different vectors. The polynucleotide(s) encoding components of the APC(s) can be situated on the same nucleic acid and/or different nucleic acids.

In some embodiments, the one or more polynucleotides comprise the nucleotide sequence of any one of SEQ TD NOs: 90-111 and 160-183 or a sequence that is at least 80%, 85%, 90%, 95%, 98%, 99%, or 100% identical to any one of SEQ ID NOs: 90-111 and 160-183. Exemplary sequences of the one or more polynucleotides encoding components of the APCs are listed in Table 4 below.

TABLE 4
Exemplary polynucleotides

| SEQ | |
|---|---|
| ID NO | Name |
| 90 | D1 SCT MART1/HLA-A*02:01 |
| 91 | D1 SCT MART1/HLA-A*02:01/EABR |
| 92 | D9 SCT MART1/HLA-A*02:01 |
| 93 | D9 SCT MART1/HLA-A*02:01/EABR |
| 94 | D1 SCT NYESO1/HLA-A*02:01 |
| 95 | D1 SCT NYESO1/HLA-A*02:01/EABR |
| 96 | D9 SCT NYESO1/HLA-A*02:01 |
| 97 | D9 SCT NYESO1/HLA-A*02:01/EABR |
| 98 | β2M |
| 99 | NYESO1/β2M |
| 100 | D9 SCD β2M/HLA-A*02:01 |
| 101 | D9 SCD β2M/HLA-A*02:01/EABR |
| 102 | D9 HLA-A*02:01 HC |
| 103 | D9 HLA-A*02:01 HC/EABR |
| 104 | NRP-V7/H-2Kd |
| 105 | NRP-V7/H-2Kd/EABR |
| 106 | IGRP206-214/H-2Kd |
| 107 | IGRP206-214/H-2Kd/EABR |
| 108 | NYESO1/A2_D9/H-2Db |
| 109 | NYESO1/A2_D9/H-2Db/EABR |
| 110 | NYESO1/A2_D9/H-2Kb |
| 111 | NYESO1/A2_D9/H-2Kb/EABR |
| 160 | Human sequence optimized Hgh/I-Ab-a |
| 161 | Human sequence optimized Hgh/Ea/I-Ab-a |
| 162 | Human sequence optimized Hgh/I-Ab-a/EABR |
| 163 | Human sequence optimized Hgh/Ea/I-Ab-a/EABR |
| 164 | Human sequence optimized Hgh/I-Ab-b |
| 165 | Human sequence optimized Hgh/Ea/I-Ab-b |
| 166 | Human sequence optimized Hgh/I-Ab-b/EABR |
| 167 | Human sequence optimized Hgh/Ea/I-Ab-b/EABR |
| 168 | Human sequence optimized “SCT-T” Hgh/Ea/I-Ab |
| 169 | Human sequence optimized “SCT-T” Hgh/Ea/I-Ab/EABR |
| 170 | Human sequence optimized Hgh/pMOG/I-Ab-a |
| 171 | Human sequence optimized Hgh/pMOG/I-Ab-a/EABR |
| 172 | Human sequence optimized Hgh/pMOG/I-Ab-b |
| 173 | Human sequence optimized Hgh/pMOG/I-Ab-b/EABR |
| 174 | Human sequence optimized “SCT-T” Hgh/pMOG/I-Ab |
| 175 | Human sequence optimized “SCT-T” Hgh/pMOG/I-Ab/EABR |
| 176 | Murine sequence optimized for mRNA Tx: I-Ab-a |
| 177 | Murine sequence optimized for mRNA Tx: Ea/I-Ab-a |
| 178 | Murine sequence optimized for mRNA Tx: I-Ab-a/EABR |
| 179 | Murine sequence optimized for mRNA Tx: Ea/I-Ab-a/EABR |
| 180 | Murine sequence optimized for mRNA Tx: I-Ab-b |
| 181 | Murine sequence optimized for mRNA Tx: Ea/I-Ab-b |
| 182 | Murine sequence optimized for mRNA Tx: I-Ab-b/EABR |
| 183 | Murine sequence optimized for mRNA Tx: Ea/I-Ab-b/EABR |

The sequence identity between an APC or component(s) of an APC provided herein (e.g., APC-assembly domain, antigen presentation polypeptide(s), presenting peptide, linker(s), supplemental polypeptide(s)) and the sequence of all or a portion (comprising said component(s)) of any one of SEQ ID NOS: 1-86, 112-159, and 184-266 can be, or be about, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values. Provided herein are nucleic acids (e.g., encoding an APC or component(s) of an APC disclosed herein) that comprise at least about 5 consecutive nucleotides (e.g., about 5 nt, 10 nt, 15 nt, 20 nt, 25 nt, 30 nt, 35 nt, 40 nt, 45 nt, 50 nt, 60 nt, 70 nt, 80 nt, 90 nt, 100 nt, 110 nt, 120 nt, 128 nt, 130 nt, 140 nt, 150 nt, 160 nt, 170 nt, 180 nt, 190 nt, 200 nt, 210 nt, 220 nt, 230 nt, 240 nt, 250 nt, 260 nt, 270 nt, 280 nt, 290 nt, 300 nt, 310 nt, 320 nt, 330 nt, 340 nt, 350 nt, 360 nt, 370 nt, 380 nt, 390 nt, 400 nt, 410 nt, 420 nt, 430 nt, 440 nt, 450 nt, 460 nt, 470 nt, 480 nt, 490 nt, 500 nt, 510 nt, 520 nt, 530 nt, 540 nt, 550 nt, 560 nt, 570 nt, 580 nt, 590 nt, 600 nt, 610 nt, 620 nt, 630 nt, 640 nt, 650 nt, 660 nt, 670 nt, 680 nt, 690 nt, 700 nt, 710 nt, 720 nt, 730 nt, 740 nt, 750 nt, 760 nt, 770 nt, 780 nt, 790 nt, 800 nt, 810 nt, 820 nt, 830 nt, 840 nt, 850 nt, 860 nt, 870 nt, 880 nt, 890 nt, 900 nt, 910 nt, 920 nt, 930 nt, 940 nt, 950 nt, 960 nt, 970 nt, 980 nt, 990 nt, 1000 nt, 1500 nt, 2000 nt, 5000 nt, 10000 nt, 50000 nt, or a number or a range between any two of these values) of a sequence described by SEQ ID NOS: 87-111 and 160-183 (or a complement thereof). Also provided herein are polypeptides (e.g., polypeptides comprising one or more APC components) that comprise at least about 5 consecutive amino acids (e.g., about 5 aa, 10 aa, 15 aa, 20 aa, 25 aa, 30 aa, 35 aa, 40 aa, 45 aa, 50 aa, 60 aa, 70 aa, 80 aa, 90 aa, 100 aa, 110 aa, 120 aa, 128 aa, 130 aa, 140 aa, 150 aa, 160 aa, 170 aa, 180 aa, 190 aa, 200 aa, 210 aa, 220 aa, 230 aa, 240 aa, 250 aa, 260 aa, 270 aa, 280 aa, 290 aa, 300 aa, 310 aa, 320 aa, 330 aa, 340 aa, 350 aa, 360 aa, 370 aa, 380 aa, 390 aa, 400 aa, 410 aa, 420 aa, 430 aa, 440 aa, 450 aa, 460 aa, 470 aa, 480 aa, 490 aa, 500 aa, 510 aa, 520 aa, 530 aa, 540 aa, 550 aa, 560 aa, 570 aa, 580 aa, 590 aa, 600 aa, 610 aa, 620 aa, 630 aa, 640 aa, 650 aa, 660 aa, 670 aa, 680 aa, 690 aa, 700 aa, 710 aa, 720 aa, 730 aa, 740 aa, 750 aa, 760 aa, 770 aa, 780 aa, 790 aa, 800 aa, 810 aa, 820 aa, 830 aa, 840 aa, 850 aa, 860 aa, 870 aa, 880 aa, 890 aa, 900 aa, 910 aa, 920 aa, 930 aa, 940 aa, 950 aa, 960 aa, 970 aa, 980 aa, 990 aa, 1000 aa, 1500 aa, 2000 aa, 5000 aa, 10000 aa, or a number or a range between any two of these values) of any one of SEQ ID NOS: 1-86, 112-159, and 184-266. The APC can comprise a single antigen presentation polypeptide. The APC can comprise 2, 3, 4, 5, 6, 7, or more, antigen presentation polypeptides. Each antigen presentation polypeptide can comprise one or more components of a major histocompatibility complex (MHC) or a derivative thereof (e.g. MHC class I (MHC-I) or MHC class II (MHC-II)). For example, an antigen presentation polypeptide can comprise β2M, or a portion thereof. An antigen presentation polypeptide can be a fusion protein, and can comprise more than one component of an MHC. For example, an antigen presentation polypeptide can comprise the N-terminus of the HLA-A*02:01 alpha chain extended with a linker connecting to the C-terminus of β2M. In some embodiments, such as, for example, those constructs labeled “Murine sequence optimized for mRNA Tx”, constructs feature nucleotide sequences that include a T7 RNA polymerase site, a 5′ UTR, a 3′ UTR, and a poly-A tail, and can encompass the full mRNA sequence to be transfected into cell cultures. In some embodiments, the corresponding amino acid sequence is for the protein translated from that mRNA sequence. In some embodiments, the amino acid sequences for the murine sequences and human sequences will differ, e.g., employing a human signal sequence (Hgh) for transfecting human cell lines and employing the natural murine MHCII (or I-Ab) signal sequences when transfecting murine NMuMG cells with mRNA. In some embodiments provided herein, the signal sequence of the APC or component thereof comprises an alternative signal sequence relative to the one employed in the indicated SEQ ID NO.

The one or more vectors can be a DNA vaccine. The DNA vaccine can be a plasmid-based DNA vaccine, a minicircle-based DNA vaccine, a bacmid-based DNA vaccine, a minigene-based DNA vaccine, a ministring DNA (linear covalently closed DNA vector) vaccine, a closed-ended linear duplex DNA (CELiD or ceDNA) vaccine, a Doggybone™ DNA vaccine, a dumbbell shaped DNA vaccine, or a minimalistic immunological-defined gene expression (MIDGE)-vector DNA vaccine. In some embodiments, the DNA vaccine elicits at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) higher immune responses against an the presenting peptide or an antigen from which the presenting peptide derives as compared to a DNA vaccine that encodes the presenting peptide but not the APC-assembly domain.

The polynucleotide(s) encoding components of the APCs can be operably linked to one or more promoters capable of inducing transcription of said polynucleotide(s). The promoter can comprise a ubiquitous promoter, an inducible promoter, a tissue-specific promoter and/or a lineage-specific promoter. The ubiquitous promoter can be selected from a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF 1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human R-actin (HBA) promoter, chicken R-actin (CBA) promoter, a CAG promoter, a CASI promoter, a CBH promoter, or any combination thereof.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The polynucleotide(s) encoding components of the APC(s) can be operably linked to a tandem gene expression element (e.g., an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof). The polynucleotide(s) encoding components of the APC(s) can comprise a transcript stabilization element (e.g., woodchuck hepatitis post-translational regulatory element (WPRE), bovine growth hormone polyadenylation (bGH-polyA) signal sequence, human growth hormone polyadenylation (hGH-polyA) signal sequence, or any combination thereof).

The nucleic acid composition can be or can comprise mRNA. The composition (e.g., nucleic acid composition) can be an mRNA vaccine. The mRNA can be formulated in a lipid nanoparticle (LNP). The term “lipid nanoparticle”, also referred to as LNP, refers to a particle having at least one dimension on the order of nanometers (e.g., 1-1,000 nm) which includes one or more lipids. In some embodiments, such lipid nanoparticles comprise a cationic lipid and one or more excipient selected from neutral lipids, charged lipids, steroids and polymer conjugated lipids (e.g., a pegylated lipid). In some embodiments, the mRNA, or a portion thereof, is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response. In some embodiments, the mRNA or a portion thereof is associated with the lipid nanoparticles. An LNP may comprise any lipid capable of forming a particle to which the one or more nucleic acid molecules are attached, or in which the one or more nucleic acid molecules are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

The LNP can comprise one or more of an ionizable cationic lipid, a non-cationic lipid (e.g., a neutral lipid), a sterol, and a PEG-modified lipid. The LNP can comprise 0.5-15 mol % PEG-modified lipid, 5-25 mol % non-cationic lipid, 25-55 mol % sterol, and 20-60 mol % ionizable cationic lipid. The LNP can comprise 40-55 mol % ionizable cationic lipid, 5-15 mol % neutral lipid, 35-45 mol % sterol, and 1-5 mol % PEG-modified lipid. In some embodiments, the RNA (e.g., mRNA) of the disclosure is formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the disclosure can be generated using components, compositions, and methods as are generally known in the art, sec for example PCT/US2016/052352; PCT/US2016/068300; PCT/US2017/037551; PCT/US2015/027400; PCT/US2016/047406; PCT/US2016/000129; PCT/US2016/014280; PCT/US2016/014280; PCT/US2017/038426; PCT/US2014/027077; PCT/US2014/055394; PCT/US2016/052117; PCT/US2012/069610; PCT/US2017/027492; PCT/US2016/059575 and PCT/US2016/069491 all of which are incorporated by reference herein in their entirety.

In some embodiments, the LNP comprises: 47 mol % ionizable cationic lipid, 11.5 mol % neutral lipid, 38.5 mol % sterol, and 3.0 mol % PEG-modified lipid; 48 mol % ionizable cationic lipid, 11 mol % neutral lipid, 38.5 mol % sterol, and 2.5 mol % PEG-modified lipid; 49 mol % ionizable cationic lipid, 10.5 mol % neutral lipid, 38.5 mol % sterol, and 2.0 mol % PEG-modified lipid; 50 mol % ionizable cationic lipid, 10 mol % neutral lipid, 38.5 mol % sterol, and 1.5 mol % PEG-modified lipid; or 51 mol % ionizable cationic lipid, 9.5 mol % neutral lipid, 38.5 mol % sterol, and 1.0 mol % PEG-modified lipid.

The ionizable cationic lipid can be heptadecan-9-yl 8 ((2 hydroxyethyl)(6 oxo 6-(undecyloxy)hexyl)amino)octanoate. The neutral lipid can be 1,2 distearoyl sn glycero-3 phosphocholine (DSPC). The sterol can be cholesterol. The PEG-modified lipid can be 1-monomethoxypolyethyleneglycol-2,3-dimyristylglycerol with polyethylene glycol of average molecular weight 2000 (PEG2000 DMG).

The wt/wt ratio of lipid to mRNA can be from about 1:100 to about 100:1 (e.g., 1:1, 1:1.1, 1:1.2, 1:1.3, 1:1.4, 1:1.5, 1:1.6, 1:1.7, 1:1.8, 1:1.9, 1:2, 1:2.5, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100 to 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any of these values).

The LNP can comprise a cationic lipid. The cationic lipid is can be cationisable, i.e. it becomes protonated as the pH is lowered below the pKa of the ionizable group of the lipid, but is progressively more neutral at higher pH values. When positively charged, the lipid is then able to associate with negatively charged nucleic acids. In some embodiments, the cationic lipid comprises a zwitterionic lipid that assumes a positive charge on pH decrease. The LNP may comprise any lipid capable of forming a particle to which the one or more nucleic acid molecules are attached, or in which the one or more nucleic acid molecules are encapsulated. In some embodiments, the LNP may comprise any further cationic or cationisable lipid, i.e. any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3dioleoyloxy)propyl)-N,N,N-trimethylammonium chloride (DOTAP); 3-(N—(N′,N′dimethylaminoethane)-carbamoyl)cholesterol (DC-Chol), N-(1-(2,3-dioleoyloxy)propyl)N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2dimyristyloxyprop-3-yl)-N,N-dimethyl-N-hydroxyethyl ammonium bromide (DMRIE).

Additionally, a number of commercial preparations of cationic lipids are available which can be used in embodiments provided herein. These include, for example, LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-lcarboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In some embodiments, the neutral lipid is 1,2-distearoyl-sn-glycero-3phosphocholine (DSPC).

In some embodiments, the cationic lipid is an amino lipid. Suitable amino lipids useful include those described in WO2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.C1), 1,2-dilinoleoyl-3-trimethylaminopropane chloride salt (DLin-TAP.C1), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In some embodiments, a non-cationic lipid comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is DMG-PEG, PEG-c-DOMG (also referred to as PEG-DOMG), PEG-DSG and/or PEG-DPG. In some embodiments, a sterol comprises cholesterol, fecosterol, sitosterol, ergosterol, campesterol, stigmasterol, brassicasterol, tomatidine, ursolic acid, alpha-tocopherol, and mixtures thereof.

In some embodiments, the nucleic acid composition comprising an mRNA sequence is a modified mRNA sequence. In this context, a modification as defined herein can lead to a stabilization of the mRNA sequence provided herein. In some embodiments, there is thus provided a stabilized mRNA sequence comprising at least one coding region as defined herein (e.g. polynucleotide(s) encoding components of the APC(s)). In some embodiments, the nucleic acid composition comprising an mRNA sequence may thus be provided as a “stabilized mRNA sequence”, that is to say as an mRNA that is essentially resistant to in vivo degradation (e.g. by an exo- or endo-nuclease). Such stabilization can be effected, for example, by a modified phosphate backbone of an mRNA provided herein. A backbone modification can be a modification in which phosphates of the backbone of the nucleotides contained in the mRNA are chemically modified. Nucleotides that can be used in this connection contain e.g. a phosphorothioate-modified phosphate backbone, such as at least one of the phosphate oxygens contained in the phosphate backbone being replaced by a sulfur atom. Stabilized mRNAs may further include, for example: non-ionic phosphate analogues, such as, for example, alkyl and aryl phosphonates, in which the charged phosphonate oxygen is replaced by an alkyl or aryl group, or phosphodiesters and alkylphosphotriesters, in which the charged oxygen residue is present in alkylated form. Such backbone modifications typically include, without implying any limitation, modifications from the group consisting of methylphosphonates, phosphoramidates and phosphorothioates (e.g. cytidine-5′-O-(1-thiophosphate)). The term “mRNA modification” as used herein may refer to chemical modifications comprising backbone modifications as well as sugar modifications or base modifications. In this context, a modified mRNA (sequence) as defined herein may contain nucleotide analogues/modifications, e.g. backbone modifications, sugar modifications or base modifications. A backbone modification can be a modification, in which phosphates of the backbone of the nucleotides contained in an mRNA compound comprising an mRNA sequence as defined herein are chemically modified. A sugar modification can be a chemical modification of the sugar of the nucleotides of the mRNA compound comprising an mRNA sequence as defined herein. Furthermore, a base modification can be a chemical modification of the base moiety of the nucleotides of the mRNA compound comprising an mRNA sequence. In this context, nucleotide analogues or modifications can be selected from nucleotide analogues, which are applicable for transcription and/or translation.

The mRNA provided herein can comprise a 5′ untranslated region (UTR), a 3′ UTR, and/or a cap (e.g., a CAP analogue). A modified mRNA sequence as defined herein, can be modified by the addition of a so-called “5′-CAP structure”, which can stabilize the mRNA as described herein. A 5′-CAP is an entity, typically a modified nucleotide entity, which generally “caps” the 5′-end of a mature mRNA. A 5′-CAP may typically be formed by a modified nucleotide, particularly by a derivative of a guanine nucleotide. In some embodiments, the 5′-CAP is linked to the 5′-terminus via a 5′-5′-triphosphate linkage. A 5′-CAP may be methylated, e.g. m7GpppN, wherein N is the terminal 5′ nucleotide of the nucleic acid carrying the 5′-CAP, typically the 5′-end of an mRNA. m7GpppN is the 5′-CAP structure, which naturally occurs in mRNA transcribed by polymerase II and is therefore in some embodiments is not considered as modification comprised in a modified mRNA in this context. Accordingly, a modified mRNA sequence may comprise a m7GpppN as 5′-cap, but additionally the modified mRNA sequence typically comprises at least one further modification as defined herein. A CAP analogue refers to a non-polymerizable di-nucleotide that has CAP functionality in that it facilitates translation or localization, and/or prevents degradation of the RNA molecule when incorporated at the 5′-end of the RNA molecule. Non-polymerizable means that the CAP analogue will be incorporated only at the 5′-terminus because it does not have a 5′ triphosphate and therefore cannot be extended in the 3′-direction by a template-dependent RNA polymerase. CAP analogues include, but are not limited to, a chemical structure selected from m7GpppG, m7GpppA, m7GpppC; unmethylated CAP analogues (e.g., GpppG); dimethylated CAP analogue (e.g., m2,7GpppG), trimethylated CAP analogue (e.g., m2,2,7GpppG), dimethylated symmetrical CAP analogues (e.g., m7Gpppm7G), or anti reverse CAP analogues (e.g., ARCA; m7,2′OmeGpppG, m7,2′dGpppG, m7,3′OmeGpppG, m7,3′dGpppG and their tetraphosphate derivatives) (Stepinski et al., 2001. RNA 7(10):1486-95). Further CAP analogues have been described previously (U.S. Pat. No. 7,074,596, WO2008/016473, WO2008/157688, WO2009/149253, WO2011/015347, and WO2013/059475).

The mRNA can comprise one or more modified nucleotides selected from pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine. The mRNA can comprise a modified nucleotide in place of one or more uridines. The modified nucleoside can be selected from pseudouridine (ψ), N 1-methyl-pseudouridine (m 1ψ), and 5-methyl-uridine (m5U). In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB 2017/051367 all of which are incorporated by reference herein.

### Pharmaceutical Compositions and Therapeutic Applications

### Pharmaceutical Compositions

Also provided herein include a pharmaceutical composition comprising compositions (e.g., a nucleic acid composition, a population of APVs) as herein described. Pharmaceutical compositions can comprise the compositions provided herein (e.g., a nucleic acid composition, a population of APVs) in combination with one or more compatible and pharmaceutically acceptable carriers. For example, a pharmaceutical composition can be or can comprise an mRNA vaccine, a DNA vaccine, and/or a population of APVs as described herein. A pharmaceutical composition can elicit a prophylactic (e.g., to prevent or delay the onset of a disease, or to prevent the manifestation of clinical or subclinical symptoms thereof) or therapeutic (e.g., suppression or alleviation of symptoms) immune response in a subject. The APVs themselves (e.g., produced ex vivo in patient cells, donor cells or standard human mammalian cell lines) can be employed as a therapeutic as described herein.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In some embodiments, pharmaceutically acceptable carrier comprise a pharmaceutical acceptable salt. As used herein, a “pharmaceutical acceptable salt” includes a salt of an acid form of one of the components of the compositions herein described. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids.

The pharmaceutical composition can further comprise appropriate adjuvants. Adjuvant refers to any immunomodulating substance capable of being combined with the protein antigens herein described to enhance, improve or otherwise modulate an immune response in a subject, such as, for example, immunostimulatory peptides, oligonucleotide CpG motifs, immunostimulatory carbohydrates and polysaccharides, and immunostimulatory protein or peptide molecules (e.g. cytokines, chemokines, flagellin, and derivatives thereof), Freund's adjuvant, sapanin (e.g., Matrix M1), lecithin, aluminum hydroxide, monophosphoryl lipid A, interleukin-12, STING agonist, Advax, and ASOi1, STING agonist (e.g., bis-(3′,5′)-cyclic dimeric guanosine monophosphate (c-di-GMP or cdGMP)). In some embodiments, the nucleic acid composition comprises one or more polynucleotides encoding immunostimulatory agents. In some embodiments, the population of APVs comprises one or more immunostimulatory agents. The one or more immunostimulatory agents can comprise toll-like receptor (TLR) agonists, cytokine receptor agonists, CD40 agonists, Fc receptor agonists, CpG-containing nucleic acids, complement receptor agonists, or any combination thereof). The TLR agonist can be a TLR-1 agonist, TLR-2 agonist, TLR-3 agonist, TLR-4 agonist, TLR-5 agonist, TLR-6 agonist, TLR-7 agonist, TLR-8 agonist, TLR-9 agonist, and/or TLR-10 agonist. The Fc receptor agonist can be a Fc-gamma receptor agonist. In some embodiments, the complement receptor agonist binds to CD21 or CD35. In some embodiments, the complement receptor agonist induces endogenous complement opsonization of the APV. The cytokine receptor agonist can be a cytokine. The cytokine receptor agonist can be a small molecule, antibody, fusion protein, or aptamer.

In some embodiments herein described, the pharmaceutical composition comprises one or more adjuvant selected from aluminum salt-based adjuvants (e.g., aluminum hydroxide, alhydrogel), emulsion adjuvants (e.g., AddaVax™, MF59®, ASO3, Freund's adjuvant, Montanide ISA51), and toll-like receptor agonists (e.g., CpG, Poly I:C, glucopyranosyl lipid A (GLA), flagellin, and resiquimod (R848)). As a skilled person will understand, both MF58® and AddaVax™ are squalene-based oil-in-water nano-emulsion.

The pharmaceutical composition can be formulated for a variety of modes of administration. Techniques for formulation and administration can be found, for example, in “Remington's Pharmaceutical Sciences”, 18^{th} ed., 1990, Mack Publishing Co., Easton, Pa. In some embodiments, the vaccine compositions of the present disclosure may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension: (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the hydrogel composition. The pharmaceutical compositions can comprise one or more pharmaceutically-acceptable carriers.

Formulations useful in the methods of the present disclosure include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a pharmaceutically acceptable carrier to produce a single dosage form will vary depending upon the host being treated, the particular mode of administration. The amount of active ingredient, which can be combined with a pharmaceutically acceptable carrier to produce a single dosage form will generally be that amount of the composition (e.g., a nucleic acid composition, a population of APVs) which produces a therapeutic effect or an immune response. Generally, out of one hundred percent, this amount will range from about 1% to about 99% of active ingredient, optionally from about 5% to about 70%, optionally from about 10% to about 30%.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like.

In solid dosage forms for oral administration (capsules, tablets, pills, dragees, powders, granules and the like), the composition (e.g., a nucleic acid composition, a population of APVs) is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

The pharmaceutical composition can be formulated for parenteral administration by injection, e.g. by bolus injection or continuous infusion. Formulations for injection can be presented in a unit dosage form, e.g. in ampoules or in multi-dose containers, with an optionally added preservative. The pharmaceutical compositions can further be formulated as suspensions, solutions or emulsions in oily or aqueous vehicles, and may contain other agents including suspending, stabilizing and/or dispersing agents.

### Therapeutic Applications

The pharmaceutical compositions disclosed herein (e.g., a nucleic acid composition, a population of APVs) can be employed in a variety of therapeutic or prophylactic applications to modulate an immune response in a subject in need, and/or to treat or prevent a disease or disorder in a subject in need (e.g., a disease or disorder caused by an infectious agent, a cancer or an autoimmune disease or disorder).

There are provided, in some embodiments, methods of modulating an immune response in a subject in need thereof. In some embodiments, the method comprises: administering to the subject a pharmaceutically effective amount of a composition disclosed herein (e.g., a nucleic acid composition, a population of APVs), thereby modulating an immune response in the subject. In some embodiments, modulating an immune response comprises stimulating immune responses. In some embodiments, modulating an immune response comprises suppressing immune responses. In some embodiments, the composition is administered in an effective amount to: elicit a robust CD4 and/or CD8 T cell response against the presenting peptide in the subject; and/or elicit a cytokine release against the presenting peptide in the subject.

In some embodiment, the immune responses are specific to the presenting peptide. In some embodiments, the presence of the antigen presentation polypeptide(s) does not induce an immune response in a subject. In some embodiments, the subject is a subject from which the cell producing the APC was derived. In some embodiments, the presence of the antigen presentation polypeptide(s) induces an immune response in a subject. In some embodiments, the APV and/or the APC is capable of inducing immunogenic responses against the presenting peptide in a subject without the administration of exogenous antigen presenting cells and/or exogenous antigen presenting components other than the APV and/or the APC.

There are provided, in some embodiments, methods of treating or preventing a disease or disorder in a subject in need thereof. In some embodiments, the method comprises: administering to the subject a pharmaceutically effective amount of a composition disclosed herein (e.g., a nucleic acid composition, a population of APVs), thereby treating or preventing the disease or disorder in the subject. The disease or disorder can be a disease or disorder caused by an infectious agent, a cancer or an autoimmune disease or disorder.

There are provided, in some embodiments, methods for treating or preventing a cancer in a subject in need thereof. In some embodiments, the method comprises: administering to the subject a pharmaceutically effective amount of a composition disclosed herein (e.g., a nucleic acid composition, a population of APVs), thereby treating or preventing the cancer in the subject.

There are provided, in some embodiments, methods for treating or preventing an autoimmune disease or disorder in a subject in need thereof. In some embodiments, the method comprises: administering to the subject a pharmaceutically effective amount of a composition disclosed herein (e.g., a nucleic acid composition, a population of APVs), thereby treating or preventing the autoimmune disease or disorder in the subject.

As used herein, the term “treatment” or “treat” refers to an intervention made in response to a disease, disorder or physiological condition (e.g., a cancer or an autoimmune disease or disorder) manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

The term “condition” as used herein indicates a physical status of the body of an individual (as a whole or as one or more of its parts), that does not conform to a standard physical status associated with a state of complete physical, mental and social well-being for the individual. Conditions herein described include but are not limited disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms.

The terms “subject”, “subject in need”, and “individual” as used herein refer to an animal and in particular higher animals and in particular vertebrates such as mammals and more particularly human beings. In some embodiments, the subject or individual has been exposed to an infectious agent. The term “exposed” indicates the subject has come in contact with a person or an animal that is known to be infected with an infectious agent. In some embodiments, a subject in need can be a healthy subject exposed to or at risk of being exposed to an infectious agent. In some embodiments, subjects in need include those already suffering from the disease or disorder caused by an infection of the infectious agent or those diagnosed with an infection. In some embodiments, the subject or individual is at risk of having a cancer or an autoimmune disease or disorder. In some embodiments, the subject or individual has developed symptoms of a cancer or an autoimmune disease or disorder. In some embodiments, the subject or individual is diagnosed of a cancer or an autoimmune disease or disorder.

Accordingly, the pharmaceutical composition can be administered in advance of any symptom, for example, in advance of an infection or a cancer. The pharmaceutical composition can also be administered at or after the onset of a symptom of disease or disorder, for example, after development of a symptom of the disease or disorder or after diagnosis of the disease or disorder.

The phrase “therapeutically effective amount” as used herein means that amount of a composition disclosed herein (e.g., a vaccine composition) which is effective for producing some desired therapeutic effect and/or generating a desired response, such as reduce or eliminate a sign or symptom of a condition or disease, such as pneumonia, at a reasonable benefit/risk ratio. The therapeutically effective amount also varies depending on the structure and presenting peptide of the APC(s), the route of administration utilized, and the specific diseases or disorders to be treated as will be understood to a person skilled in the art. For example, if a given clinical treatment is considered effective when there is at least a 20% reduction in a measurable parameter associated with a disease or disorder, a therapeutically effective amount of the composition for the treatment of that disease or disorder is the amount necessary to achieve at least a 20% reduction in that measurable parameter.

A therapeutically effective amount of the pharmaceutical composition herein described can be estimated from data obtained from cell culture assays and further determined from data obtained in animal studies, followed up by human clinical trials. For example, toxicity and therapeutic efficacy of the pharmaceutical compositions described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred.

In some embodiments, the determination of a therapeutically effective amount of the vaccine composition can be measured by measuring the titer of antibodies produced against an agent or the cytokines produced. Methods of determining antibody titers, cytokine production and methods of performing virus neutralization arrays are known to those skilled in the art as well as exemplified in the example section of the present disclosure (see, Examples).

Immunogenic levels of the APC and/or APV can be produced in serum of the subject at about 1 hour to about 6 months (e.g., 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, or a number or a range between any of these values) post administration of the composition (e.g., pharmaceutical composition).

A neutralizing antibody titer of about 50 to about 100000 half-maximal inhibitory dilutions (ID50s values) can be produced in the serum of the subject at about 1 hour to about 6 months (e.g., 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, or a number or a range between any of these values) post administration of the composition (e.g., pharmaceutical composition).

A cytokine release can be induced in the serum of the subject at about 1 hour to about 6 months (e.g., 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, or a number or a range between any of these values) post administration of the composition (e.g., pharmaceutical composition). The cytokines can be interferon-gamma (IFN-7), tumor necrosis factor-alpha (TNF-α), and one or a plurality of interleukins (e.g., IL-1, IL-2, IL-3, IL-4, IL-6, IL-8, IL-10, IL-12, IL-13, IL-16, IL-18 and IL-23).

T cells can be activated in the subject at about 1 hour to about 6 months (e.g., 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, or a number or a range between any of these values) post administration of the composition (e.g., pharmaceutical composition). In some embodiments, T cells are induced to change from one type of T cell to another type of T cell. In some embodiments, T cells are induced to change to the regulatory T cell type, in the subject at about 1 hour to about 6 months (e.g., 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, or a number or a range between any of these values) post administration of the composition (e.g., pharmaceutical composition).

In some embodiments, the composition (e.g., pharmaceutical composition) elicits an at least 10% (e.g., 10%, 20%, 30%, 40%, 50%, 60%, 70%, 80%, 90%, or a number or a range between any of these values) higher T cell activation and/or cytokine release as compared to an approach comprising administration of a soluble version of the presenting peptide; or the composition elicits an at least 10% (e.g., 10%, 20%, 30%, 40%, 50%, 60%, 70%, 80%, 90%, or a number or a range between any of these values) lower T cell activation and/or cytokine release as compared to an approach comprising administration of a soluble version of the presenting peptide.

In some embodiments, the composition (e.g., pharmaceutical composition) elicits at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) less off-target immune responses against undesired epitopes as compared to a non-enveloped nanoparticle-based composition. Said undesired epitopes can comprise the nanoparticle scaffold of said non-enveloped nanoparticle-based composition.

In some embodiments, the composition (e.g., pharmaceutical composition) elicits an at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) higher neutralizing antibody titer as compared to an approach comprising administration of (i) a soluble version of the presenting peptide, and/or (ii) an mRNA vaccine encoding the presenting peptide and not encoding the APC-assembly domain. In some embodiments, the composition (e.g., pharmaceutical composition) elicits an at least as high neutralizing antibody titer (e.g., 1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) as compared to an approach comprising administration of a protein-based nanoparticle presenting the presenting peptide. In some embodiments, administration of the composition (e.g., pharmaceutical composition) elicits protective and long-lasting immunity against the antigen from which the presenting peptide derives and variants thereof.

In some embodiments, an at least as low dose of the composition (e.g., pharmaceutical composition) can be needed to generate a comparable immune response as compared to an approach comprising administration of a protein-based nanoparticle presenting the presenting peptide. In some embodiments, an at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) lower dose of the composition (e.g., pharmaceutical composition) can be needed to generate a comparable immune response as compared to an approach comprising administration of (i) a soluble version of the presenting peptide, and/or (ii) an mRNA vaccine encoding the presenting peptide and not encoding the APC-assembly domain.

In some embodiments, an at least as low dose of the composition (e.g., pharmaceutical composition) can be needed to generate a comparable immunosuppression as compared to an approach comprising administration of a protein-based nanoparticle presenting the presenting peptide. In some embodiments, an at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) lower dose of the composition (e.g., pharmaceutical composition) can be needed to generate a comparable immune response as compared to an approach comprising administration of (i) a soluble version of the presenting peptide, and/or (ii) an mRNA vaccine encoding the presenting peptide and not encoding the APC-assembly domain.

In some embodiments, following administration of a first dose or second dose of the composition (e.g., pharmaceutical composition), the composition induces: (i) an at least as potent (e.g., 1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) serum neutralizing titers against the antigen from which the presenting peptide derives or variants thereof as compared to an approach comprising administration of a protein-based nanoparticle presenting the presenting peptide, optionally the composition comprises a population of APVs; (ii) an at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more potent serum neutralizing titers against the antigen from which the presenting peptide derives or variants thereof as compared to an approach comprising administration of a soluble version of the presenting peptide, optionally the composition comprises a population of APVs; and/or (iii) an at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more potent serum neutralizing titers against the antigen from which the presenting peptide derives or variants thereof as compared to an approach comprising administration of an mRNA vaccine encoding the presenting peptide and not encoding the APC-assembly domain, optionally the composition comprises an mRNA vaccine encoding an APC comprising the presenting peptide.

In some embodiments, following administration of a first dose or second dose of the composition (e.g., pharmaceutical composition), the composition induces: (i) an at least as potent (e.g., 1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) T cell activation and/or cytokine release against the presenting peptide or variants thereof as compared to an approach comprising administration of a protein-based nanoparticle presenting the presenting peptide, optionally the composition comprises a population of APVs; (ii) an at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more potent T cell activation and/or cytokine release against the presenting peptide or variants thereof as compared to an approach comprising administration of a soluble version of the presenting peptide, optionally the composition comprises a population of APVs; and/or (iii) an at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more potent T cell activation and/or cytokine release against the presenting peptide or variants thereof as compared to an approach comprising administration of an mRNA vaccine encoding the presenting peptide and not encoding the APC-assembly domain, optionally the composition comprises an mRNA vaccine encoding an APC comprising the presenting peptide.

In some embodiments, following administration of a first dose or second dose of the composition (e.g., pharmaceutical composition), the composition induces: (i) an at least as potent (e.g., 1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) immunosuppression against the presenting peptide or variants thereof as compared to an approach comprising administration of a protein-based nanoparticle presenting the presenting peptide, optionally the composition comprises a population of APVs; (ii) an at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more potent immunosuppression against the presenting peptide or variants thereof as compared to an approach comprising administration of a soluble version of the presenting peptide, optionally the composition comprises a population of APVs; and/or (iii) an at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more potent immunosuppression against the presenting peptide or variants thereof as compared to an approach comprising administration of an mRNA vaccine encoding the presenting peptide and not encoding the APC-assembly domain, optionally the composition comprises an mRNA vaccine encoding an APC comprising the presenting peptide.

In some embodiments, potency of serum neutralizing titers can be measured by geometric means for serum half-maximal inhibitory dilutions (ID50s values) against the infectious agent or variants thereof, optionally about 1 day to about 6 months (e.g., 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, or a number or a range between any of these values) after administration of a first dose or a second dose of the composition (e.g., pharmaceutical composition).

In some embodiments, the subject: is a human subject; is a newborn or infant of an age of not more than 3 years, of not more than 2 years, of not more than 1.5 years, of not more than 1 year (12 months), of not more than 9 months, 6 months or 3 months, or is between 6 months and 2 years; is immunocompromised, has a pulmonary disease, and/or is 65 years of age or older; has a chronic pulmonary disease, optionally chronic obstructive pulmonary disease (COPD) or asthma; has a cancer; has an autoimmune disease or disorder, optionally type I diabetes; and/or has an underlying comorbid condition, optionally selected from heart disease, diabetes, and lung disease.

The composition (e.g., pharmaceutical composition) can be administered in an effective amount to: (i) induce a robust antibody response against the presenting peptide in the subject, optionally a robust antibody response comprises a neutralizing antibody response, further optionally a robust antibody response comprises Fc domain effector functions that recruit immune cells to infected cells, optionally said immune cells are macrophages, neutrophils, and/or natural killer cells, further optionally said recruitment induces antibody-dependent cellular cytotoxicity (ADCC) and/or antibody-dependent cellular phagocytosis (ADCP); (ii) elicit a robust CD4 and/or CD8 T cell response against the presenting peptide in the subject; and/or (iii) elicit a balanced Th1/Th2 response against the presenting peptide in the subject.

The method can comprise administering to the subject at least two doses (e.g., 2, 3, 4, 5, 6, 7, 8, 9, 10, 11, 12, 13, 14, 15, 16, 17, 18, 19, 20, or a number or a range between any of these values) of the composition (e.g., pharmaceutical composition). The second dose of the composition (e.g., pharmaceutical composition) can be administered to the subject at least 1 day (e.g., 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, or a number or a range between any of these values) after a first dose of the composition (e.g., pharmaceutical composition) is administered to the subject.

The pharmaceutical compositions herein described can be administered using techniques well known to those skilled in the art, such as injection, inhalation or insulation or by oral, parenteral or rectal administration. The vaccine composition can be administered by means including, but not limited to, traditional syringes and needleless injection devices. Suitable routes of administration include, but are not limited to, parenteral delivery, such as intramuscular, intradermal, subcutaneous, intramedullary injections, as well as, intrathecal, direct intraventricular, intravenous, intraperitoneal, intranasal, or intraocular injections. For injection, the vaccine composition herein described can be formulated in aqueous solutions, optionally in physiologically compatible buffers such as Hanks' solution, Ringer's solution, or physiological saline buffer. In some embodiments, administering can comprise aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. The composition can be administered intramuscularly (e.g., into a deltoid region of an arm). In some embodiments, the composition is: (i) co-administered with an adjuvant; or (ii) not co-administered with an adjuvant.

In some embodiments, the compositions provided herein can be administered to a subject systematically. The wording “systemic administration” as used herein indicates any route of administration by which a vaccine composition is brought in contact with the body of the individual, so that the resulting composition location in the body is systemic (i.e. non limited to a specific tissue, organ or other body part where the vaccine is administered). Systemic administration includes enteral and parenteral administration. Enteral administration is a systemic route of administration where the substance is given via the digestive tract, and includes but is not limited to oral administration, administration by gastric feeding tube, administration by duodenal feeding tube, gastrostomy, enteral nutrition, and rectal administration. Parenteral administration is a systemic route of administration where the substance is given by route other than the digestive tract and includes but is not limited to intravenous administration, intra-arterial administration, intramuscular administration, subcutaneous administration, intradermal, administration, intraperitoneal administration, and intravesical infusion.

The pharmaceutical composition herein disclosed can be administered to a subject using a prime/boost protocol. In such protocol, a first pharmaceutical composition is administered to the subject (prime) and then after a period of time, a second pharmaceutical composition can be administered to the subject (boost). Administration of the second composition (boost composition) can occur days, weeks or months after administration of the first composition (prime composition). For example, the boost composition can be administered about three days, 1 week, 2 weeks, 3 weeks, 4 weeks, 5 weeks, 6 weeks, 7 weeks, 8 weeks, 12 weeks, 16 weeks, 20 weeks, 24 weeks, or 28 weeks, or a number or a range between any two of these values, after the prime composition is administered. In some embodiments, the boost composition can be administered about 4 weeks after administration of the prime composition.

Therefore, the pharmaceutical composition can be administered to the subject in need two or more times. For example, the methods herein described can comprise administering to the subject a first pharmaceutical composition, and after a period of time, administering to the subject a second pharmaceutical composition. The prime pharmaceutical composition and the boost pharmaceutical composition can be, but need not be, the same composition. In some embodiments, the prime vaccine composition and the boost vaccine composition can contain same or different APC(s).

The vaccine compositions provided herein can be used to protect a subject against disease or disorder caused by heterologous antigens (e.g., infectious agents different from those that the presenting peptides(s) are derived from). For example, a pharmaceutical composition made using coronavirus antigens of a first coronavirus and a second coronavirus is capable of protecting an individual against infection by not only the first and second coronaviruses (i.e., the matched strains), but also coronaviruses from different taxonomic groups (i.e., mismatched strains or coronavirus strains different from the first and second coronaviruses).

The disease or disorder can be a blood disease, an immune disease, a neurological disease or disorder, a cancer, an infectious disease, a genetic disease, a disorder caused by aberrant mtDNA, a metabolic disease, a disorder caused by aberrant cell cycle, a disorder caused by aberrant angiogenesis, a solid tumor, a disorder cause by aberrant DNA damage repair, or any combination thereof. In some embodiments, the disease or disorder is a solid tumor.

The disease or disorder can be an infectious disease selected from an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1, 2, 3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru (EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection.

The disease can be associated with expression of a tumor-associated antigen (e.g., a proliferative disease, a precancerous condition, a cancer, and a non-cancer related indication associated with expression of the tumor antigen). The cancer can be selected from colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. The cancer can be a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

The disease can be an autoimmune disease or disorder. The autoimmune disease or disorder can be selected from arthritis (rheumatoid arthritis, juvenile rheumatoid arthritis, osteoarthritis, psoriatic arthritis, and ankylosing spondylitis), psoriasis, dermatitis including atopic dermatitis; chronic idiopathic urticaria, including chronic autoimmune urticaria, polymyositis/dermatomyositis, toxic epidermal necrolysis, systemic scleroderma and sclerosis, responses associated with inflammatory bowel disease (IBD) (Crohn's disease, ulcerative colitis), and IBD with co-segregate of pyoderma gangrenosum, erythema nodosum, primary sclerosing cholangitis, and/or episcleritis), respiratory distress syndrome, including adult respiratory distress syndrome (ARDS), meningitis, IgE-mediated diseases such as anaphylaxis and allergic rhinitis, encephalitis such as Rasmussen's encephalitis, uveitis, colitis such as microscopic colitis and collagenous colitis, glomerulonephritis (GN) such as membranous GN, idiopathic membranous GN, membranous proliferative GN (MPGN), including Type I and Type II, and rapidly progressive GN, allergic conditions, eczema, asthma, conditions involving infiltration of T cells and chronic inflammatory responses, atherosclerosis, autoimmune myocarditis, leukocyte adhesion deficiency, systemic lupus erythematosus (SLE) such as cutaneous SLE, lupus (including nephritis, cerebritis, pediatric, non-renal, discoid, alopecia), juvenile onset diabetes, multiple sclerosis (MS) such as spino-optical MS, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including Large Vessel vasculitis (including Polymyalgia Rheumatica and Giant Cell (Takayasu's) Arteritis), Medium Vessel vasculitis (including Kawasaki's Disease and Polyarteritis Nodosa), CNS vasculitis, and ANCA-associated vasculitis, such as Churg-Strauss vasculitis or syndrome (CSS), aplastic anemia, Coombs positive anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome, myasthenia gravis, antigen-antibody complex mediated diseases, anti-glomerular basement membrane disease, anti-phospholipid antibody syndrome, allergic neuritis, Bechet disease, Castleman's syndrome, Goodpasture's Syndrome, Lambert-Eaton Myasthenic Syndrome, Reynaud's syndrome, Sjörgen's syndrome, Stevens-Johnson syndrome, solid organ transplant rejection (including pretreatment for high panel reactive antibody titers, IgA deposit in tissues, and rejection arising from renal transplantation, liver transplantation, intestinal transplantation, cardiac transplantation, etc.), graft versus host disease (GVHD), pemphigoid bullous, pemphigus (including vulgaris, foliaceus, and pemphigus mucus-membrane pemphigoid), autoimmune polyendocrinopathies, Reiter's disease, stiff-man syndrome, immune complex nephritis, IgM polyneuropathies or IgM mediated neuropathy, idiopathic thrombocytopenic purpura (ITP), thrombotic throbocytopenic purpura (TTP), thrombocytopenia (as developed by myocardial infarction patients, for example), including autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM), including pediatric IDDM, and Sheehan's syndrome; autoimmune hepatitis, Lymphoid interstitial pneumonitis (HIV), bronchiolitis obliterans (non-transplant) vs NSIP, Guillain-Barre Syndrome, Berger's Disease (IgA nephropathy), primary biliary cirrhosis, celiac sprue (gluten enteropathy), refractory sprue with co-segregate dermatitis herpetiformis, cryoglobulinemia, amylotrophic lateral sclerosis (ALS; Lou Gehrig's disease), coronary artery disease, autoimmune inner ear disease (AIED), autoimmune hearing loss, opsoclonus myoclonus syndrome (OMS), polychondritis such as refractory polychondritis, pulmonary alveolar proteinosis, amyloidosis, giant cell hepatitis, scleritis, monoclonal gammopathy of uncertain/unknown significance (MGUS), peripheral neuropathy, paraneoplastic syndrome, channelopathies such as epilepsy, migraine, arrhythmia, muscular disorders, deafness, blindness, periodic paralysis, and channelopathies of the CNS; autism, inflammatory myopathy, and focal segmental glomerulosclerosis (FSGS).

Disclosed herein include methods of modulating an immune cell. In some embodiments, modulating comprising activating, regulating, stimulating, and/or suppressing. Disclosed herein include method of isolating an immune cell of interest. In some embodiments, the immune cell comprises a B cell, a plasmablast, a plasma cell, a T cell, or an NK cell. In some embodiments, the method comprises contacting the immune cell with the composition disclosed herein. In some embodiments, the contacting step is performed in vitro, in vivo or in situ.

In some embodiments, the contacting step is performed in a compartment loaded with the composition disclosed herein. In some embodiments, the contacting step is performed in a cavity of a particle. In some embodiments, the particles have a longest dimensional length of around 100 μm or less. In some embodiments, the particles have a minimum dimensional length of at least 10 μm. In some embodiments, the particles have a dimension of 10 μm, 12 μm, 14 μm, 16 μm, 18 μm, 20 μm, 22 μm, 24 μm, 26 μm, 28 μm, 30 μm, 32 μm, 34 μm, 36 μm, 38 μm, 40 μm, 42 μm, 44 μm, 46 μm, 48 μm, 50 μm, 52 μm, 54 μm, 56 μm, 58 μm, 60 μm, 62 μm, 64 μm, 66 μm, 68 μm, 70 μm, 72 μm, 74 μm, 76 μm, 78 μm, 80 μm, 82 μm, 84 μm, 86 μm, 88 μm, 90 μm, 92 μm, 94 μm, 96 μm, 98 μm, or 100 μm. The particles can have a number of shapes including: crescent shaped, bowl shaped, moon shaped, capsule shaped, concentric sphere shaped.

In some embodiments, the particle includes cavity. In some embodiments, the cavity has a longest dimensional length of around 100 μm or less. In some embodiments, the cavity has a minimum dimensional length of at least 10 μm. In some embodiments, the cavity has a dimension of 10 μm, 12 μm, 14 μm, 16 μm, 18 μm, 20 μm, 22 μm, 24 μm, 26 μm, 28 μm, 30 μm, 32 μm, 34 μm, 36 μm, 38 μm, 40 μm, 42 μm, 44 μm, 46 μm, 48 μm, 50 μm, 52 μm, 54 μm, 56 μm, 58 μm, 60 μm, 62 μm, 64 μm, 66 μm, 68 μm, 70 μm, 72 μm, 74 μm, 76 μm, 78 μm, 80 μm, 82 μm, 84 μm, 86 μm, 88 μm, 90 μm, 92 μm, 94 μm, 96 μm, 98 μm, or 100 μm. In some embodiments, the cavity has a dimension that fits a single cell in the cavity. In some embodiments, the cavity has a dimension that fits a plurality of cells in the cavity. In some embodiments, the cavity is dimensioned to hold a sub-nanoliter volume of fluid.

The cavity can have one or more (e.g., 1, 2, 3, 4, 5, 6, 7, 8, 9, or 10) openings that opens to the external environment of the particle. The cavity can have a single opening that opens to the external environment of the particle. The opening of the cavity is dimensioned to allow cells to enter the cavity. In some embodiments, the particle is a nanovial. The systems, methods, compositions, and kits provided herein can, in some embodiments, be employed in concert with the systems, methods, compositions, and kits described in PCT Application Publication Nos. WO2022240655A1, WO2024155631A1, and WO2022240656A1, the contents of which are incorporated herein by reference in their entireties.

In some embodiments, the particles hold cell(s) within the cavity. In some embodiments, the cell(s) is immune cells such as B cell, a plasmablast, a plasma cell, a T cell, or an NK cell. In some embodiments, the cell(s) is located within the volume of fluid within the cavity. In some embodiments, the cell(s) adheres or becomes adherent to a surface of the particle within the cavity. In some embodiments, the cell(s) adheres or becomes adherent to a surface of the particle within the cavity through the composition (e.g., the APV) disclosed herein and/or a binding moiety. In some embodiments, the binding moiety comprises an antibody or fragment thereof capable of specifically binding to one or more selected from CD274, CD83, CD80, CD54, MHC, the presenting peptide, the APC-assembly domain, and a fragment thereof. In some embodiments, the APVs disclosed herein are disposed on the surface of the cavity through a binding moiety. In some embodiments, the binding moiety comprises an antibody or fragment thereof capable of specifically binding the APV. In some embodiments, the cell(s) binds to the APV, which, in turn, is disposed to a surface of the particle within the cavity through a binding moiety.

In some embodiments, one or more capture agent(s) are disposed on the surface of the cavity. In some embodiments, the capture agent(s) comprise an antibody or fragment thereof capable of specifically binding to a cytokine. In some embodiments, the cytokine(s) is released from the cell(s) held in the particles within the cavity. In some embodiments, the capture agent(s) comprise an antibody or fragment thereof capable of specifically binding to one or more selected from IL-2, TNF-α, IFN-γ, and fragments thereof.

In some embodiments, the particles are formed from biocompatible materials or polymers. In some embodiments, the particle is composed of hydrogel. In one embodiment, the particles are formed from polyethylene glycol (PEG).

In some embodiments, activation of the immune cell is at least about 1.1-fold (e.g., 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, or a number or a range between any of these values) higher as compared to a method of activating the immune cell using the antigen presentation polypeptide and the presenting peptide without forming the APV. In some embodiments, level of cytokine secretion is at least about 1.1-fold (e.g., 1.7-fold, 1.8-fold, 1.9-fold, 2-fold, 2.1-fold, 2.2-fold, 2.3-fold, 2.4-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, or a number or a range between any of these values) higher as compared to a method of activating the immune cell using the antigen presentation polypeptide and the presenting peptide without forming the APV. In some embodiments, the cytokine is IFN-γ. In some embodiments, the presence of the supplemental polypeptide(s) further increases the activation of the immune cell and/or the level of cytokine secretion. In some embodiments, the presence of the supplemental polypeptide(s) increases the activation of the immune cell and/or the level of cytokine secretion by at least 10%, 20%, 30%, 40%, 50%, 60%, 70%, 80% or 90%. The method can further comprise isolating immune cell(s) of interest.

### Kits

The compositions disclosed herein (e.g., a nucleic acid composition, a population of APVs) can be provided as components of a kit.

Kits can include compositions disclosed herein (e.g., a nucleic acid composition, a population of APVs) as well components for making such compositions. As such, kits can include, for example, primers, nucleic acid molecules, expression vectors, nucleic acid constructs encoding protein antigens and/or particle-forming subunits described herein, cells, buffers, substrates, reagents, administration means (e.g., syringes), and instructions for using any of said components. It should be appreciated that a kit may comprise more than one container comprising any of the aforementioned, or related, components. For example, certain parts of the kit may require refrigeration, whereas other parts can be stored at room temperature. Thus, as used herein, a kit can comprise components sold in separate containers by one or more entity, with the intention that the components contained therein be used together.

The composition (e.g., a nucleic acid composition, a population of APVs, a vaccine composition) can comprise Tris buffer, sucrose, and/or sodium acetate. The composition can comprise an adjuvant (e.g., aluminum hydroxide, alhydrogel, AddaVax, MF59, ASO3, Freund's adjuvant, Montanide ISA51, CpG, Poly I:C, glucopyranosyl lipid A, flagellin, resiquimod, or any combination thereof). The composition can be a lyophilized composition. In some embodiments, the lyophilized composition has a water content of less than about 10%. The nucleic acid composition and the LNP-forming components can be in separate vials. The composition can be a pharmaceutical composition, and the pharmaceutical composition can comprise one or more pharmaceutically acceptable carriers, diluents and/or excipients. The composition can comprise instructions for use of the composition for: modulating an immune response in a subject in need thereof, treating or preventing a disease or disorder associated with an antigen (e.g., disease or disorder caused by an infectious agent, or by a cancer-related antigen or an autoimmune disease) in a subject in need thereof, and/or treating or preventing a cancer, infection or autoimmune disease in a subject in need thereof.

In some embodiments, the composition is formulated or is to be formulated: as a liquid, a solid, or a combination thereof; for injection; for intramuscular administration, intranasal administration, transdermal administration, aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection; and/or as particles (e.g., iron oxide particles, liposomes, micelles, polymer complexes, cationic peptide nanoemulsions, virus-like particles (VLPs), lipid nanoparticles (LNP) and/or lipoplex (LPX) particles).

### EXAMPLES

Some aspects of the embodiments discussed above are disclosed in further detail in the following examples, which are not in any way intended to limit the scope of the present disclosure.

### Materials and Methods

The following experimental materials and methods were used for Example 1 described below.

### Bacteria

E. coli DH5 Alpha cells (Zymo Research) used for expression plasmid productions were cultured in LB broth (Sigma-Aldrich) with shaking at 225 rpm at 37° C. Plasmids were purified for transfection using Zymogen miniprep kits (Zymo Research) or maxiprep kits (Macherey-Nagel).

### Cell Lines

HEK293T cells plated in 6-well plates (Falcon) were cultured in Dulbecco's modified Eagle's medium (DMEM, Gibco) supplemented with 10% heat-inactivated fetal bovine serum (FBS, R&D Systems) and 1 U/mL penicillin-streptomycin (Gibco) at 37° C. and 5% CO₂. Transfections were carried out with FuGENE transfection reagent (Promega) diluted in Opti-MEM (Gibco). Expi293F cells (Gibco) for APV expression and antibody expression were maintained at 37° C. and 8% CO₂ in Expi293 expression medium (Gibco). Transfections were carried out with an Expi293 Expression System Kit (Gibco). Falcon tubes sealed with AirOtops (VWR) or culture flasks (Corning) containing Expi293F cells were maintained under shaking at 470 rpm for 10-30 mL transfections and 130 rpm for transfections larger than 30 mL. All cell lines were derived from female donors and were not specially authenticated.

### Sequences

HLA-A2-peptide (SLLMWITQV) Complex (3M4E5) Human Monoclonal Antibody heavy chain comprises a nucleotide sequence of SEQ ID NO: 87 and an amino acid sequence of SEQ ID NO: 34. HLA-A2-peptide (SLLMWITQV) Complex (3M4E5) Human Monoclonal Antibody light chain comprises a nucleotide sequence of SEQ ID NO: 88 and an amino acid sequence of SEQ ID NO: 35. p3BNC-SARS-CoV-2 S-2P-DCT-EABR (a.k.a. SARSCoV2-S/EPM/EABR) comprises a nucleotide sequence of SEQ ID NO: 89 and an amino acid sequence of SEQ ID NO: 36.

### T Cells

T cells were isolated from human donor whole blood samples by negative selection using the RosetteSep Human T Cell Enrichment Cocktail Kit (STEMCELL Technologies). Isolated T cells were seeded in a fresh complete ImmunoCult™-XF T cell expansion medium (STEMCELL Technologies) at 1×10⁶ cells/mL with 2 pL/mL ImmunoCult™ human CD3/CD28 T cell activator. T cells were activated for 3 days and expanded for up to 12 days by changing into fresh expansion medium every 2-3 days. All cells were cultured in incubators at 37° C. and 5% CO₂.

Design of EABR constructs

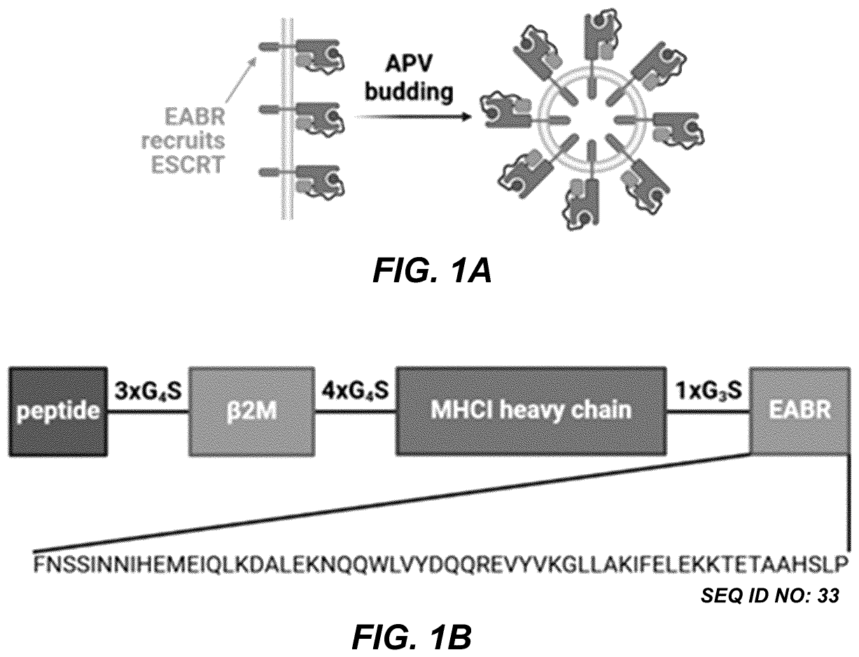

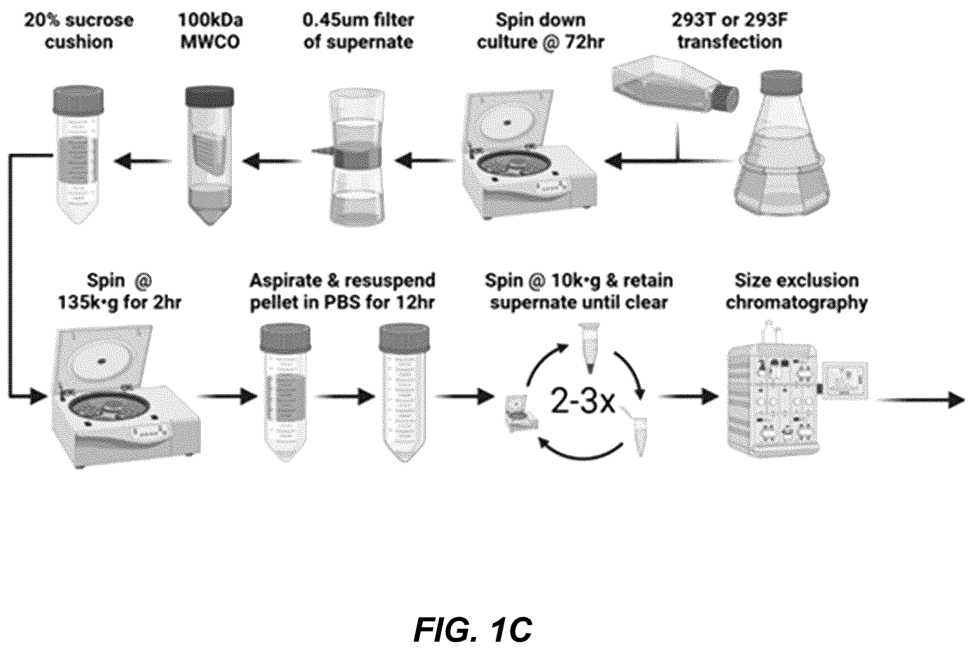

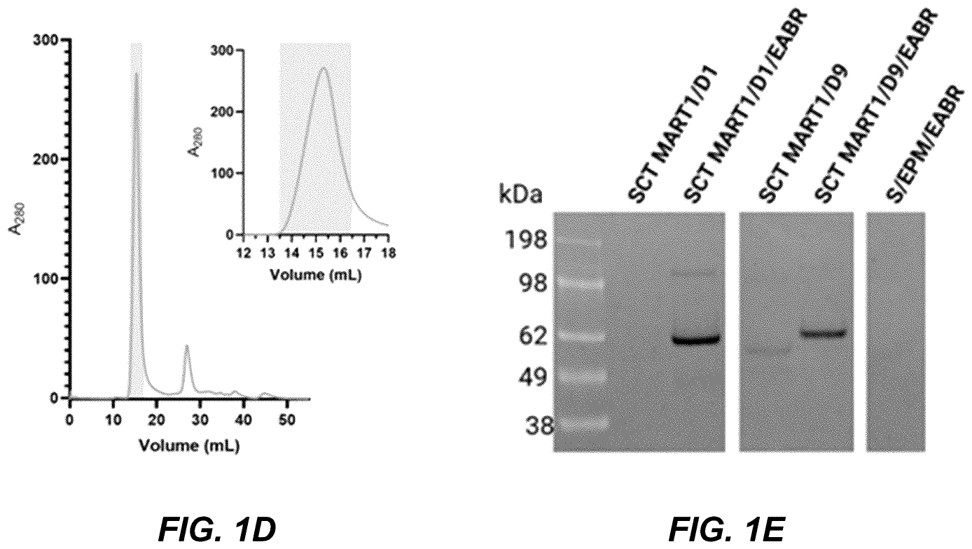

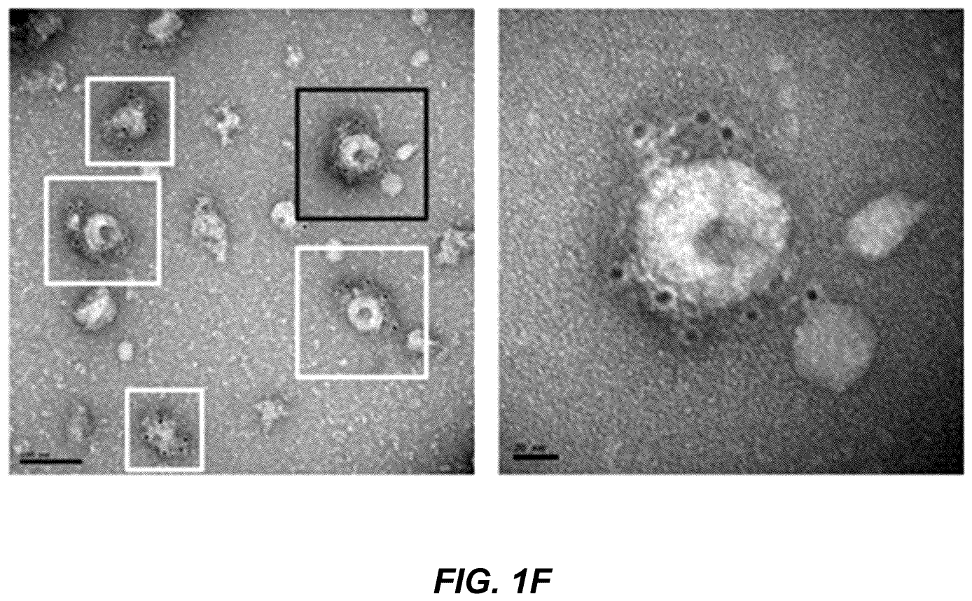

The EABR domain (residues 160-217) of the human CEP55 protein was fused to the C-terminus of the SCT MART1/HLA-A*02:01 alpha chain separated by a (Gly)₃Ser (GS) linker (GGGS) (SEQ ID NO: 9) to generate “D1” and “D9” single-chain heterotrimer pMHCI constructs inserted in the p3BNC expression plasmid. To generate the “D1” SCT construct, the N-terminus of the HLA-A*02:01 alpha chain was extended with a 4×(G₄S) linker (GGGGSGGGGSGGGGSGGGGS) (SEQ ID NO: 31) connecting to the C-terminus of β2M, which was itself extended at its N-terminus by a 3×(G4S) linker (GGGGSGGGGSGGGGS) (SEQ ID NO: 30) connecting to either the 10-mer MART1 (ELAGIGILTV) (SEQ ID NO: 184) or 9-mer NYESO1 (SLLMWITQV) (SEQ ID NO: 185) cancer-related peptides to make a single-chain heterotrimer (SCT) (FIG. 1B). To generate the SCT “D9” construct, the “D1” SCT construct was edited with Y84C and A139C substitutions.

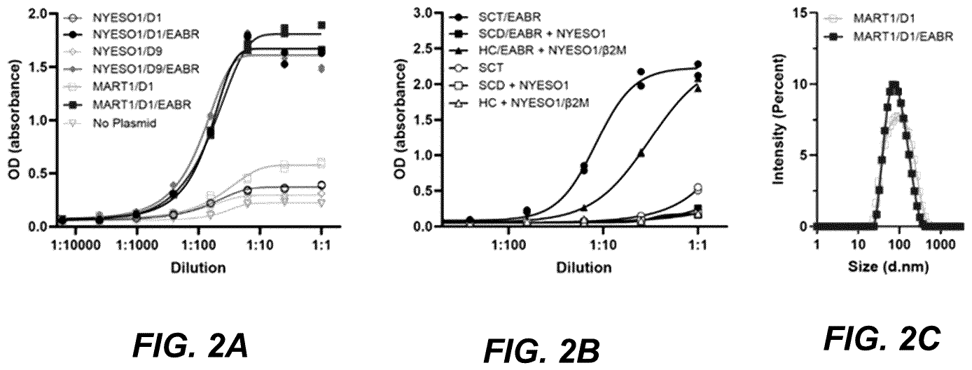

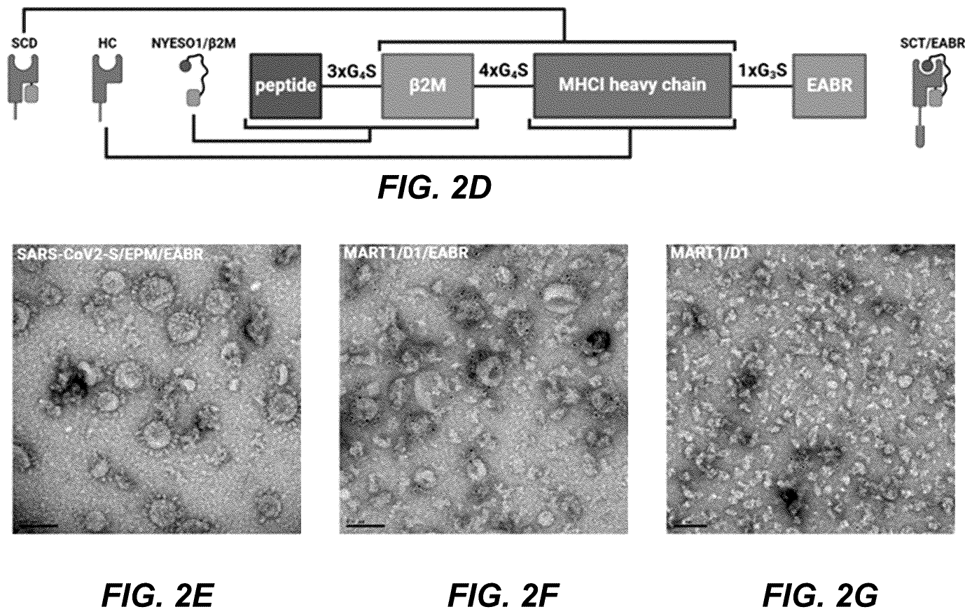

The “single-chain dimer” (SCD) construct removed both the peptide and the 3×(G4S) linker (SEQ ID NO: 30) between the peptide and β2M of the “D9” SCT construct. The “heavy chain” (HC) construct removed the peptide, the 3×(G4S) linker (SEQ ID NO: 30) between the peptide and β2M, β2M, and the 4×(G4S) (SEQ ID NO: 31) linker between β2M and the MHCI heavy chain of the “D9” SCT construct (FIG. 2D). Both the SCD and HC constructs were inserted in p3BNC expression plasmids.

Murine SCT MHCI H-2Kd was constructed similar to the human D1 SCT HLA-A*02:01 design, with either an NRP-V7 or IGRP206-214 presenting peptide fused to a 3×(Gly₄Ser) linker (SEQ ID NO: 30), murine β2M, a 4×(Gly₄Ser) linker (SEQ ID NO: 31), and the H-2Kd heavy chain, i.e., NRP-V7/H-2Kd and IGRP206-214/H-2Kd, respectively. The chimeric human/mouse MHCI HLA-A2/H-2Db and HLA-A2/H-2Kb were designed according to the D9 SCT design, with an NYESO1 presenting peptide fused to a 3×(Gly₄Ser) linker (SEQ ID NO: 30), human β2M, a 4×(Gly₄Ser) linker (SEQ ID NO: 31), and a chimeric MHCI alpha chain composed of the extracellular α1 and α2 domains of human Y84A, A139C HLA-A*02:01 heavy chain and the α3, transmembrane and cytoplasmic domains of murine H-2Kb or H-2Db heavy chain, i.e., NYESO1/A2_D9/H-2Db and NYESO1/A2_D9/H-2Kb, respectively. Murine and chimeric human/murine SCT pMHCI constructs were inserted into the p3BNC expression plasmid.

### Production of EABR APVs

EABR APVs were generated by one of two means: 1) transfecting Expi293F cells (Gibco) cultured in Expi293F expression media (Gibco) on an orbital shaker at 37° C. and 8% CO₂ with plasmid DNA pre-filtered through 0.22 μm Spin-X filters (Corning); 2) transfecting HEK293T cells cultured in Dulbecco's modified Eagle's medium (DMEM, Gibco) supplemented with 10% heat-inactivated fetal bovine serum (FBS, Sigma-Aldrich) and 1 U/ml penicillin-streptomycin (Gibco) at 37° C. and 5% CO₂. 72 hours post-transfection, cells were centrifuged at 1000×g for 10 min, supernatants were passed through a 0.45 μm filter (Corning) with Luer-Lok syringes (BD) or a 0.45 μm vacuum filter (Thermo Fisher Scientific), and concentrated using Amicon Ultra-15 centrifugal filters with 100 kDa molecular weight cut-off (Millipore). APVs were purified by ultracentrifugation at 50,000 rpm (135,000×g) for 2 hours at 4° C. using a TLA100.3 rotor and an Optima™ TLX ultracentrifuge (Beckman Coulter) on a 20% w/v sucrose cushion in polycarbonate centrifuge tubes (Beckman Coulter). Supernatants and the sucrose cushion were removed, and pellets were re-suspended in 200 pL sterile pH 7.4 PBS at 4° C. overnight. To remove residual cell debris, re-suspended samples were transferred to microcentrifuge tubes and centrifuged at 10,000×g for 10 min; clarified supernatants were collected for subsequent ELISA, DLS, and Western blot assays. For immuno-EM grid preparations, quantitative Western blots, and ELISPOT assays, APVs were further purified by SEC using a Superose 6 Increase 10/300 column (Cytiva) equilibrated with pH 7.4 PBS. Peak fractions in the void volume corresponding to pMHCI APVs were combined and concentrated to 250-500 pL in Amicon Ultra-4 centrifugal filters with 100 kDa molecular weight cut-off (Millipore) or Nanosep with 100K Omega centrifugal filters (Pall). Samples were stored at 4° C. and imaged directly after purification.

### Transduction of TCRs in PBMC

Engineering of candidate TCRs in PBMC was performed according to previous publication.

### Antibody Expression

Anti-HLA-A2-peptide (SLLMWITQV (SEQ ID NO: 185)) Complex (3M4E5) antibody was made by expressing the heavy chain and light chain of the 3M4E5 antibody at a 1.5:1 plasmid ratio in Expi293F cells. Supernatant from the transiently-transfected Expi293F cells (Gibco) was separated from the cells by pelleting at 3500 g for 15 minutes and filtering the clarified supernatant through a 0.2 μm syringe filter (VWR) or 0.45 μm vacuum filter unit (Thermo Fisher Scientific). The filtered supernatant was further purified using Fc-affinity chromatography (HiTrap MabSelect, Cytiva) and SEC (HiLoad 16/600 Superdex 200 column, Cytiva). Peak fractions corresponding to purified antibody proteins were pooled, concentrated, and stored at 4° C. Biotinylated antibodies for ELISAs were generated and purified using an EZ-Link biotinylation kit (Thermo Fisher Scientific). The antibody generated using the above method was compared to a commercially available 3M4E5 antibody (RHM03002-100; ProteoGenix) with ELISA and showed identical performance (data not shown).

### Western Blot Analysis

The presence of HLA-A, CD81, and CD63 on purified APVs was detected by Western blot analysis. Samples were diluted in NuPage SDS-PAGE loading buffer (Thermo Fisher Scientific) under reducing conditions, separated on Bolt™ Bis-Tris Plus Mini Protein Gels, 4-12% (Thermo Fisher Scientific), and transferred to nitrocellulose membranes in the form of an iBlot2 NC Mini Stack (Thermo Fisher Scientific) using the iBlot2 Gel Transfer Device (Thermo Fisher Scientific). Nitrocellulose membranes from the stack were transferred to water for 1-5 minutes, then transferred to Flex FD solution (Thermo Fisher Scientific), before placing on an iBind card (Thermo Fisher Scientific) for staining with antibodies per the iBlot2 protocol. The following antibodies were used for detecting pMHCI: rabbit anti-HLA-A protein (PA5-29911; Thermo Fisher Scientific) at 1:1,000, mouse anti-CD81 (10630D; Thermo Fisher Scientific) at 1:100, mouse anti-CD81 (MA5-13548; Thermo Fisher Scientific) at 1:100, mouse anti-CD63 (10628D; Thermo Fisher Scientific) at 1:500, rabbit anti-CD63 (25682-1-AP; Thermo Fisher Scientific) at 1:500, Alex488-conjugated goat anti-rabbit IgG (A32731; Thermo Fisher Scientific) at 1:1,000, and Alex488-conjugated goat anti-mouse IgG (A55058; Thermo Fisher Scientific) at 1:1,000. Protein was compared to a SeeBlue Plus2 Prestained protein ladder (Thermo Fisher Scientific). For T cell studies, the amount of MHCI on APVs was determined by quantitative Western blot analysis. Various dilutions of SEC-purified pMHCI APV samples and known amounts of soluble, biotinylated pMHCI protein (NIH Tetramer Core Facility at Emory) were separated by SDS-PAGE and transferred to nitrocellulose membranes as described above. Band intensities of the pMHCI standards and pMHCI APV sample dilutions were measured using ImageJ to determine MHCI concentrations.

Immuno-EM of pMHCI/EABR APVs

SEC-purified MART1/MHCI/EABR APVs were prepared on grids for negative stain transmission electron microscopy at room temperature. Formvar/Carbon 300 Mesh grids (Electron Microscopy Sciences) were first glow discharged. 20 μL of purified sample was pipetted onto paraffin, glow discharged formvar/carbon 300 mesh grids were placed on the droplet of sample for 5 minutes, then gently wicked away by dabbing the edge of the grid on filter paper. The grids were then placed on a 20 pL droplet of 1% paraformaldehyde (Electron Microscopy Sciences) in pH 7.4 PBS for 10 minutes, and subsequently wicked away. Grids were washed three times by placing the grids on droplets of pH 7.4 PBS for 5 minutes, and wicking away the PBS with filter paper after each wash. Grids were then blocked by placing the grids on droplets of 5% FBS diluted in pH 7.4 PBS for one hour. Blocking solution was wicked away from the grids, and the grids were then placed on droplets of rabbit anti-HLA-A antibody (PA5-29911; Thermo Fisher Scientific) diluted 1:100 dilution in 5% sucrose, 5% FBS pH 7.4 PBS for 2 hours; to prevent drying of the primary antibody solution, the grids were placed in a humidified chamber made from wet paper towels folded under a pyrex glass dish. Grids were again washed three times by placing the grids on droplets of pH 7.4 PBS for 10 minutes, and wicking away the PBS with filter paper after each wash. The grids were then placed on droplets of 6 nm colloidal gold goat anti-rabbit antibody (PA5-29911; Thermo Fisher Scientific) diluted 1:20 dilution in 5% sucrose, 5% FBS pH 7.4 PBS for 1 hour. Grids were then washed three times, ten minutes per wash with pH 7.4 PBS, wicking away the PBS after the ten minute wash each time. Finally, the grid was placed on a droplet of 1.5% uranyl formate (Electron Microscopy Sciences), prepared fresh within 1 week of use, for 1 minute. The excess uranyl formate was wicked away and the grid was left to air dry for 30 minutes before storing at room temperature away from light. Imaging of the grids was performed on a FEI Tecnai T12 Transmission Electron Microscope within two weeks after the grids were prepared.

### Indirect ELISA

96-well plates (Corning) were coated with APV samples diluted 1:10 or 1:4 or 1:1, 4-fold serially diluted in sterile 0.1 M NaHCO₃pH 9.6, and sealed with TempPlate sealing foil (USA Scientific) overnight at 4° C. Plates were emptied and blocked with TBS-T/3% BSA (Sigma-Aldrich) for at least 30 minutes. Plates were again emptied, and 5 μg/mL of primary antibody diluted in TBS-T/3% BSA was added to the plates. The following were used as primary antibodies for detecting pMHCI: rabbit anti-HLA-A antibody (PA5-29911; Thermo Fisher Scientific), mouse anti-HLA-A2-peptide (SLLMWITQV (SEQ ID NO: 185)) Complex (3M4E5) antibody (RHM03002-100; ProteoGenix), mouse anti-H-2Kd antibody (14-5998-82; Thermo Fisher Scientific).

After a 2-hour incubation at room temperature, plates were emptied, washed three times with TBS-T, and 5 μg/mL of HRP-conjugated secondary antibody diluted in TBS-T/3% BSA was added to the plates for 30 minutes at room temperature. The following were used as secondary antibodies: HRP-conjugated goat anti-rabbit IgG (4058-05; Southern Biotech) at 1:2,000, HRP-conjugated goat anti-mouse IgG (1013-05; Southern Biotech) at 1:2,000.

After washing three times with TBS-T, plates were developed using 1-Step™ Ultra TMB-ELISA Substrate Solution (Thermo Fisher Scientific) and absorbance was measured at 450 nm. Standard 4-parameter sigmoidal binding curves were calculated using GraphPad Prism 10.3.1 without any further editing.

### Sandwich ELISA

96-well plates (Corning) were coated with 5 μg/mL of mouse anti-HLA-A2-peptide (SLLMWITQV (SEQ ID NO: 185)) Complex (3M4E5) antibody (RHM03002-100; ProteoGenix) or in-house-made anti-HLA-A2-peptide (SLLMWITQV (SEQ ID NO: 185)) Complex (3M4E5) antibody diluted in sterile 0.1 M NaHCO₃pH 9.6, and sealed with TempPlate sealing foil (USA Scientific) overnight at 4° C. Plates were emptied and blocked with TBS-T/3% BSA for at least 30 minutes. Plates were again emptied, and APV samples diluted 1:10 or 1:4, 4-fold serially diluted in TBS-T/3% BSA were added to the plates. After a 2-hour incubation at room temperature, plates were emptied, and 5 μg/mL of biotinylated detection antibody diluted in TBS-T/3% BSA was added to the plates. 5 μg/mL of biotinylated (Thermo Fisher Scientific) mouse anti-HLA-A2-peptide (SLLMWITQV (SEQ ID NO: 185)) Complex (3M4E5) antibody (RHM03002-100; ProteoGenix) or in-house-generated human anti-HLA-A2-peptide (SLLMWITQV (SEQ ID NO: 185)) Complex (3M4E5) antibody (SEQ ID NO: 34-35) was used as a capture antibody. After another 2-hour incubation at room temperature, plates were washed three times with TBS-T. HRP-conjugated streptavidin (Abcam) was diluted to manufacturer's recommendations in TBS-T/3% BSA and added to plates for 30 minutes at room temperature. After washing three times with TBS-T, plates were developed using 1-Step™ Ultra TMB-ELISA Substrate Solution (Thermo Fisher Scientific) and absorbance was measured at 450 nm. Standard 4-parameter sigmoidal binding curves were calculated using GraphPad Prism 10.3.1 without any further editing.

### ELISPOT

1G4 T cells were centrifuged at 300 g for 10 minutes, and re-suspended in CTL-Test™ media (ImmunoSpot) containing 1% L-glutamine (Gibco) for ELISPOT analysis to evaluate T cell responses. APVs were added to human IFN-g single-color ELISPOT plates (ImmunoSpot) and 10-fold serially diluted. Phytohemagglutinin (Gibco) was added in a separate well at a final concentration of 1.5% as a positive control. 100,000 cells were added per well, and plates were incubated at 37° C. in 8% CO₂ for 24 hours. Biotinylated detection, streptavidin-alkaline phosphatase (AP), and substrate solutions were added according to the manufacturer's guidelines. Plates were gently rinsed with water three times to stop the reactions. Plates were air-dried for two hours in a running laminar flow hood. The number of spots and the mean spot sizes were quantified using a CTL ImmunoSpot S6 Universal-V Analyzer (Immunospot).

### Quantification and Statistical Analysis

ELISA serial dilutions were plotted by fitting a standard sigmoidal four parameter logistic curve without weighting or special handling of outliers using Graphpad Prism 10.3.1. Statistical significance in ELISPOT data was calculated using multiple unpaired t-tests with Welch correction and Holm-Sidik's multiple comparison tests to obtain adjusted p-values in Graphpad Prism 10.3.1. Unless otherwise stated, adjusted p-values are reported for comparisons of the maximum concentrations.

### Example 1

### Recruiting ESCRT to Single-Chain Heterotrimer Peptide-MHCI Releases Antigen-Presenting Vesicles that Stimulate T Cells Selectively

Disclosed in this Example is an engineered assembly and budding of peptide/MHCI-displaying APVs from the cell surface of non-immune cells by DNA transfection of an engineered single-chain heterotrimer (SCT) peptide/MHCI (pMHCI). Building on a method for generating enveloped virus-like particles displaying the SARS-CoV-2 spike protein, an endosomal sorting complex required for transport (ESCRT)- and ALG-2-interacting protein X (ALIX)-binding region (EABR) was fused to the cytoplasmic tail of SCT pMHCI to directly recruit the ESCRT machinery and induce the release of pMHCI-displaying vesicles. Immuno-electron microscopy (immuno-EM) of the supernatant from transfected cells showed halos of black punctate surrounding exosome-like vesicles, confirming the presence of SCT pMHCI on APVs. ELISPOT assays showed that SCT pMHCI/EABR APVs were capable of stimulating IFN-γ release from T cells at a level equivalent to refolded pMHCI tetramer, and that the APVs selectively stimulated T cells presenting their cognate TCR. Altogether, these results demonstrated a new method for generating synthetic, native-like APVs in high copy number and valency with the therapeutic potential for improved cell-free immunoregulation.

EABR Addition to the Cytoplasmic Tail of SCT pMHCI Promotes APV Release

To generate APVs from non-immune cells, an EABR sequence from the human CEP55 protein was attached to the cytoplasmic tail of MHCI (FIG. 1A). To ensure each APV presented the same, intended peptide on MHCI, the complete peptide:MHCI complex was formed into a single-chain heterotrimer. Specifically, the N-terminus of the HLA-A*02:01 alpha chain was extended with a 4×G₄S linker (SEQ ID NO: 31) connecting to the C-terminus of beta-2 microglobulin (β2M), which was itself extended at its N-terminus by a 3×G4S linker (SEQ ID NO: 30) connecting to either the 10-mer MART-1 (MART1) (SEQ ID NO: 184) or 9-mer NY-ESO-1 (NYESO1) (SEQ ID NO: 185) cancer-related peptides. The EABR sequence used here was identical to the sequence used to create an enveloped virus-like particles (eVLPs) displaying the SARS-CoV-2 spike protein, and was attached to the cytoplasmic tail of the HLA-A*02:01 alpha chain by a G3S linker (SEQ ID NO: 9) (FIG. 1). A first SCT pMHCI construct was named “D1/EABR” to denote a “D1” SCT design with an “EABR” sequence appended to the end of its cytoplasmic tail. A second “D9” variant of SCT pMHCI, containing Y84C and A139C substitutions, was also tested given its improvements in thermostability and T cell receptor binding compared to the D1 design.

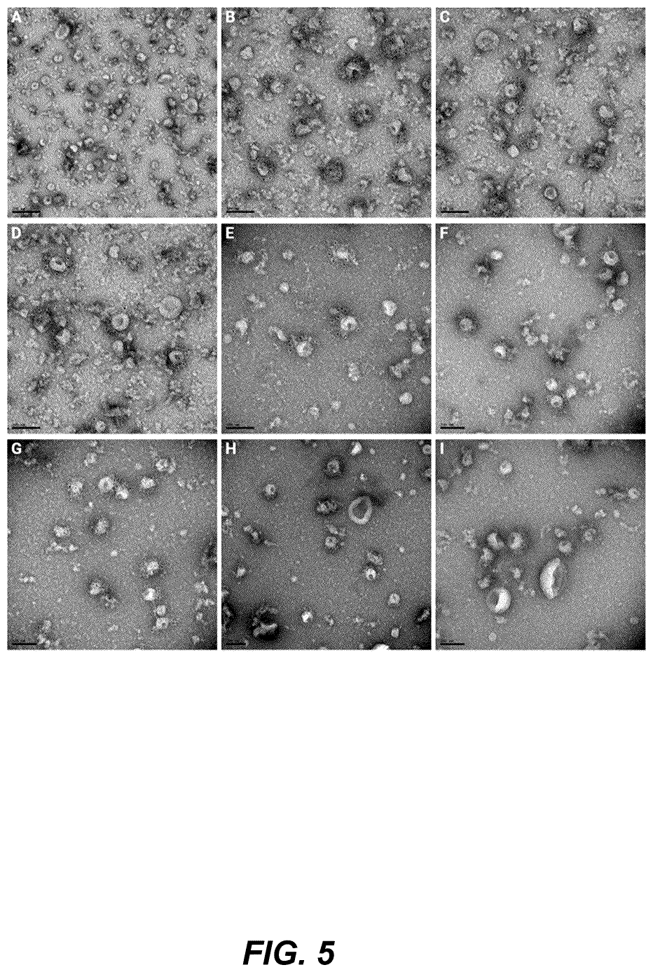

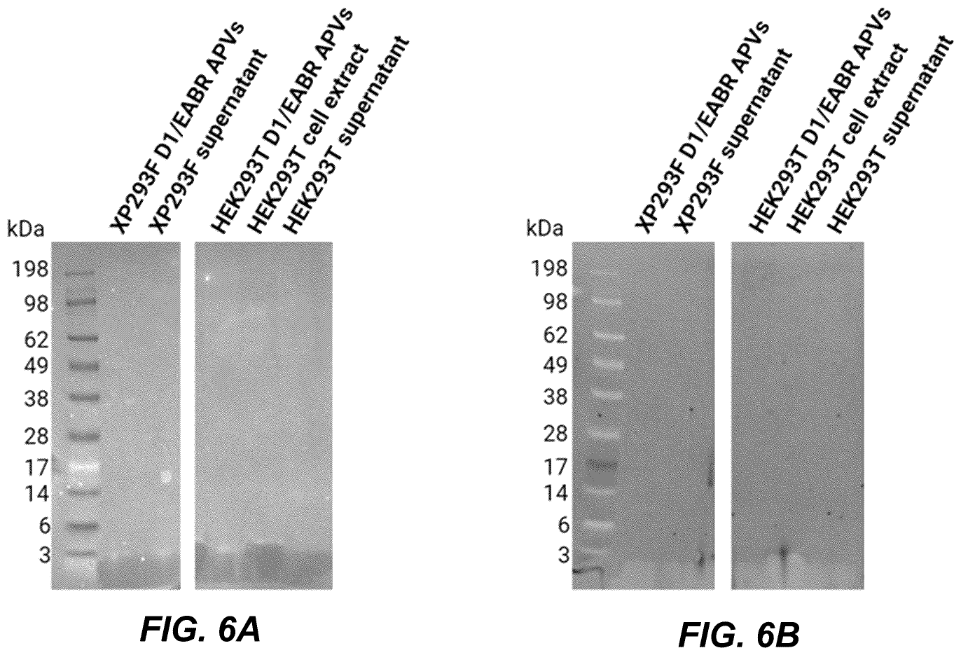

APVs were expressed by transfecting HEK293T or Expi293F cells with DNA plasmids encoding the SCT pMHCI constructs. Vesicles were purified by ultracentrifugation of cell culture supernatant on a 20% sucrose gradient as is typical for the purification of viral particles and exosomes (FIG. 1C). Ultracentrifuged product was re-suspended in pH 7.4 phosphate-buffered saline (PBS) and optionally further purified by size exclusion chromatography (SEC) to yield a high concentration of product in the void volume (FIG. 1D). Western blot analysis of the purified product showed that the addition of the EABR sequence to the cytoplasmic tails of both the MART1/D1/EABR and MART1/D9/EABR constructs generated higher levels of MHCI compared to their non-EABR counterparts (FIG. 1E). Because pMHCI is difficult to resolve with cryo-EM or cryo-ET, immuno-electron microscopy grids of the purified product was prepared to confirm the presence of pMHCI-displaying APVs, which presented as cup-like, collapsed spheres resembling the canonical shape of exosomes that partially collapse upon paraformaldehyde fixation and negative staining (FIG. 1F and FIG. 5). Western blots of these pMHCI vesicles did not stain for the hallmark exosomal tetraspanin proteins CD81 or CD63 (FIG. 6A-FIG. 6B). As a result, the vesicles were denoted as pMHCI-displaying “antigen-presenting vesicles” to differentiate them from traditional “exosomes.”

EABR Addition to SCT pMHCI Promotes Budding of the Complete, Intended pMHCI Complex on APVs

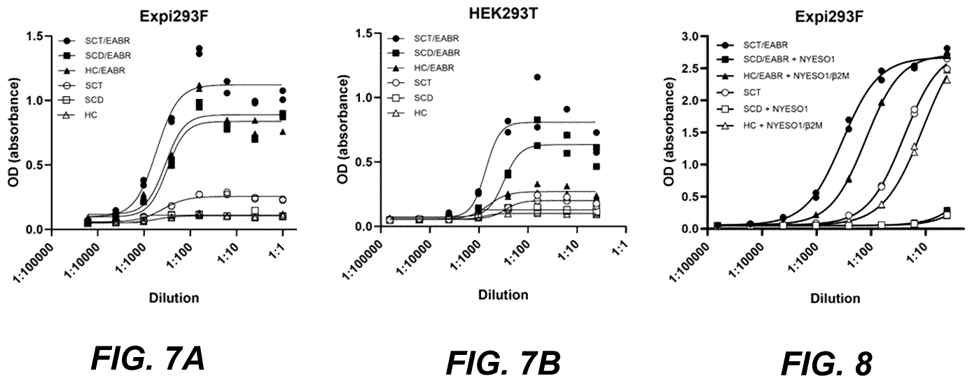

To better quantify the relative improvement in APV formation with EABR addition, ELISA of SCT MART1 and SCT NYESO1 constructs was performed. Both constructs showed over 100-fold improvements in APV formation with the addition of the EABR sequence (FIG. 2A). Subsequent ELISAs showed that appending EABR to MHCI heavy chain (HC) alone (i.e., without covalently linked β2M and presenting peptide) still boosted MHCI yield in the purified transfection product relative to HC without an EABR tag, and an even greater concentration of MHCI resulted from transfection with EABR-tagged single-chain heterodimer (SCD) constructs comprised of MHCI heavy chain covalently linked to β2M without a presenting peptide (FIG. 7A-FIG. 7B).

It was also confirmed that the SCT form of pMHCI was not required to assemble the intended pMHCI complex on released APVs. To compare the impact of different NYESO1/MHCI construct designs on APV assembly and release, the Gly₄Ser linkers were selectively removed from the SCT NYESO1/D9 construct—either removing the 3×G₄S linker (SEQ ID NO: 30) between the peptide and β2M yielding SCD, or the 4×G4S linker (SEQ ID NO: 31) between β2M and the MHCI heavy chain yielding NYESO1/β2M—and performed ELISAs with a TCR-like antibody specific for the fully formed NYESO1:HLA-A*02:01 complex (FIG. 2B and FIG. 2D). Decoupling the NYESO1 peptide from the EABR-tagged SCD MHCI complex led to an over 10-fold reduction in the detected level of expression of NYESO1/MHCI APVs in HEK293T cells (FIG. 2B). Co-transfecting two separate constructs of HC/EABR and NYESO1/β2M generated APVs detectable by sandwich ELISA and provides an alternative design for APVs. However, the SCT form of NYESO1/D9/EABR provided the best expression of pMHCI-displaying APVs out of all constructs tested (FIG. 2B), and it is the only design that ensures the APVs are presenting the construct's intended peptide.

Interestingly, low levels of MHCI protein were still recovered after purification of non-EABR SCT pMHCI, an effect that was amplified by expressing the same constructs in the higher-expressing Expi293F cell line, which has been used previously for exosome production (FIG. 2A and FIG. 8). Dynamic light scattering (DLS) of both the purified SCT MART1/D1 and SCT MART1/D1/EABR constructs showed similar median particle diameters of 60-80 nm (range 30-250 nm) with the SCT MART1/D1/EABR construct, exhibiting a slightly narrower distribution compared to the non-EABR SCT MART1/D1 construct (FIG. 2C). This particle size distribution was consistent with the vesicle sizes seen in immuno-EM images of the EABR constructs. However, to pMHCI vesicles were not observed when imaging immuno-EM grid preparations of the SCT MART1/D1 construct purified from Expi293F supernatant. Instead, the non-EABR SCT MART1/D1 grids showed what appeared to be pMHC aggregates or blebbing of sparsely populated membrane, which was less frequently observed in the grids prepared for the EABR-tagged vesicles (FIG. 2F, FIG. 2G, and FIG. 5). As a direct contrast, identically prepared EM grids for both SARS-CoV-2 S/EPM/EABR and MART1/D1/EABR constructs were densely populated with vesicles displaying their respective proteins, and only the MART1/D1/EABR vesicles stained positively with a halo of anti-HLA-A antibody around each vesicle (FIG. 2E and FIG. 2F). Altogether, the results showed that SCT pMHCI promoted the display of complete pMHCI complexes on APVs, and that addition of the EABR sequence increases APV release.

EABR-Mediated APV Release Generalizes Across SCT pMHCI Variants

To further explore the generalizability of EABR sequence addition and heterotrimerization of pMHCI on APV release, the ability of EABR-tagged SCT constructs of murine MHCI and chimeric human/mouse MHCI to generate APVs displaying pMHCI relevant for in vivo studies of immunoregulation was also tested. SCT constructs of murine MHCI H-2Kd were prepared as described for the human D1 constructs with either an NRP-V7 or IGRP206-214 type 1 diabetes-relevant presenting peptide fused to a 3×(Gly₄Ser) linker (SEQ ID NO: 30), murine β2M, a 4×(Gly₄Ser) linker (SEQ ID NO: 31), and the H-2Kd alpha chain, i.e., NRP-V7/H-2Kd and IGRP206-214/H-2Kd, respectively. The chimeric human/mouse MHCI HLA-A2/H-2Db and HLA-A2/H-2Kb were designed according to the D9 SCT design, with an NYESO1 presenting peptide fused to a 3×(Gly₄Ser) linker (SEQ ID NO: 30), human β2M, a 4×(Gly₄Ser) linker (SEQ ID NO: 31), and a chimeric MHCI alpha chain composed of the extracellular α1 and α2 domains of human Y84A, A139C HLA-A*02:01 heavy chain and the α3, transmembrane and cytoplasmic domains of murine H-2Kb or H-2Db heavy chain, i.e., NYESO1/A2_D9/H-2Db and NYESO1/A2_D9/H-2Kb, respectively.

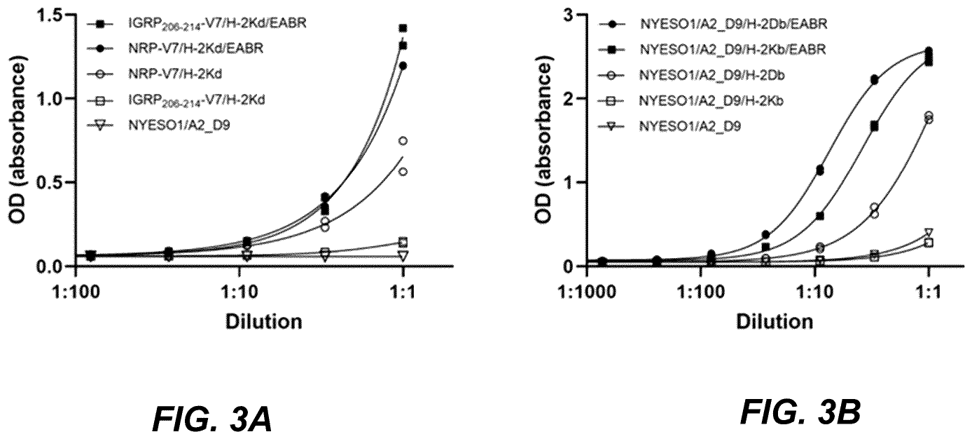

For both SCT NRP-V7/H-2Kd and SCT IGRP206-214-V7/H-2Kd, addition of an EABR sequence to the cytoplasmic tail of MHCI heavy chain amplified the production of APVs as detected by sandwich ELISA (FIG. 3A). However, like the previous SCT MART1/D1, NYESO1/D1, and NYESO1/D9 pMHCI constructs, the SCT NRP-V7/H-2Kd pMHCI construct showed evidence of substantial vesicle production in HEK293T cells regardless of the addition of the EABR sequence. Interestingly, IGRP206-214-V7/H-2Kd vesicle production was noticeably reduced despite the only sequence difference being a change in the presenting peptide. Appending an EABR sequence to this IGRP206-214-V7/H-2Kd construct led to a more dramatic change in APV production relative to EABR's effect on SCT NRP-V7/H-2Kd pMHCI APV production. Nonetheless, addition of an EABR sequence for both constructs amplified vesicle production.

In similar fashion, the SCT form of chimeric human/mouse NYESO1/A2_D9/H-2Db pMHCI expressed substantial levels of APVs without EABR addition, whereas swapping the α3, transmembrane, and cytoplasmic domains to a different MHC haplotype, NYESO1/A2_D9/H-2Kb, led to comparatively reduced APV expression (FIG. 3B). Mirroring the behavior of the lower-expressing murine IGRP206-214-V7/H-2Kd construct, addition of an EABR sequence to the cytoplasmic tail of NYESO1/A2_D9/H-2Kb increased APV expression by more than 10-fold (FIG. 3B). For both chimeric human/mouse pMHCI constructs, however, addition of the EABR sequence increased vesicle production relative to the constructs' non-EABR counterparts.

Collectively, these results suggested that MHC haplotype and the loaded presenting peptide can influence vesicle production for SCT constructs. As shown previously by immuno-EM of the MART1/D9 and MART1/D9/EABR constructs (FIG. 2F and FIG. 2G), the increased yield of pMHCI product observed by ELISA in the non-EABR NRP-V7/H-2Kd and NYESO1/A2_D9/H-2Db constructs may be an artifact of pMHCI aggregation or indiscriminate membrane blebbing rather than intact APV formation. Regardless of this intrinsic vesicle-forming potential, addition of the EABR sequence consistently increased APV release across these different pMHCI complexes, demonstrating the generalizability of the EABR sequence for inducing APV production.

pMHCI/EABR APVs Induce IFN-γ Release from their Cognate T Cells In Vitro

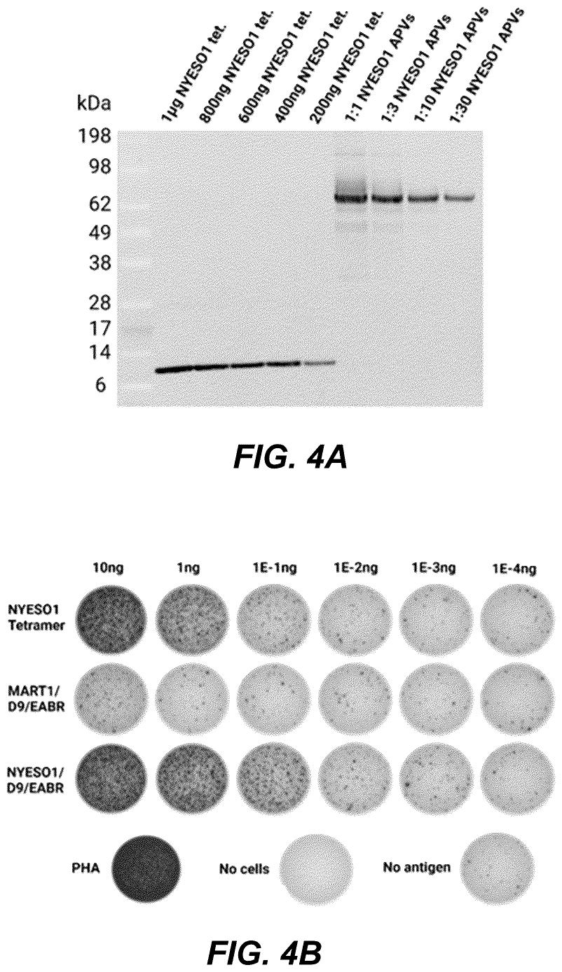

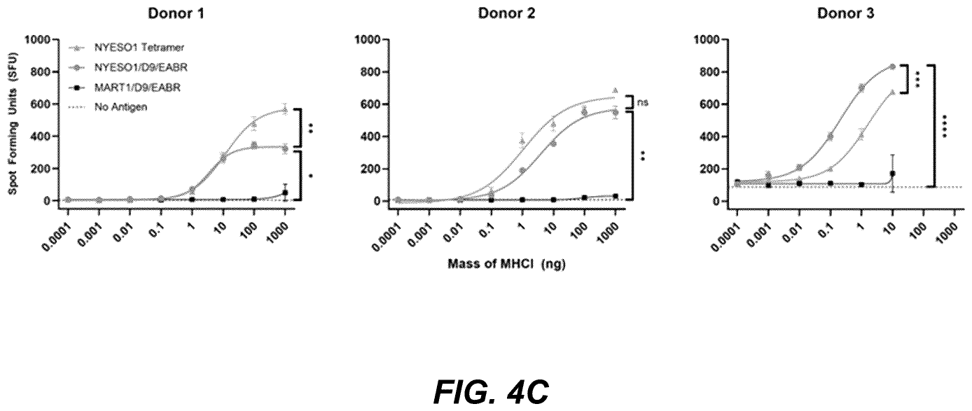

In addition to confirming that the EABR sequence reliably produced pMHCI-displaying APVs, this Example also confirmed that EABR-induced pMHCI-displaying APVs were functional as T cell stimulators. Specifically, SCT NYESO1/D9/EABR APVs were purified from transfected cell supernatants by ultracentrifugation and SEC, and the MHCI protein concentrations in purified APVs were determined by quantitative western blot analysis (FIG. 4A). MHCI for MART1/D9/EABR, NYESO1/D9/EABR, or NYESO1:HLA-A*02:01 tetramers of an equivalent concentration was mixed with donor T cells that were transduced with the 1G4 TCR specific for the NYESO1:HLA-A*02:01 epitope. T cell stimulation was evaluated using an ELISPOT assay measuring IFN-7 release (FIG. 4B).

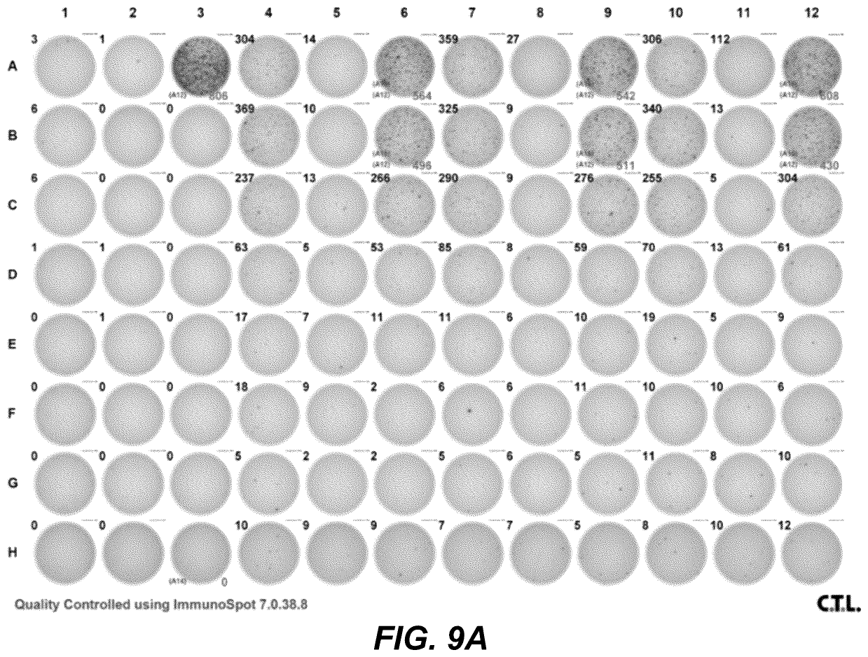

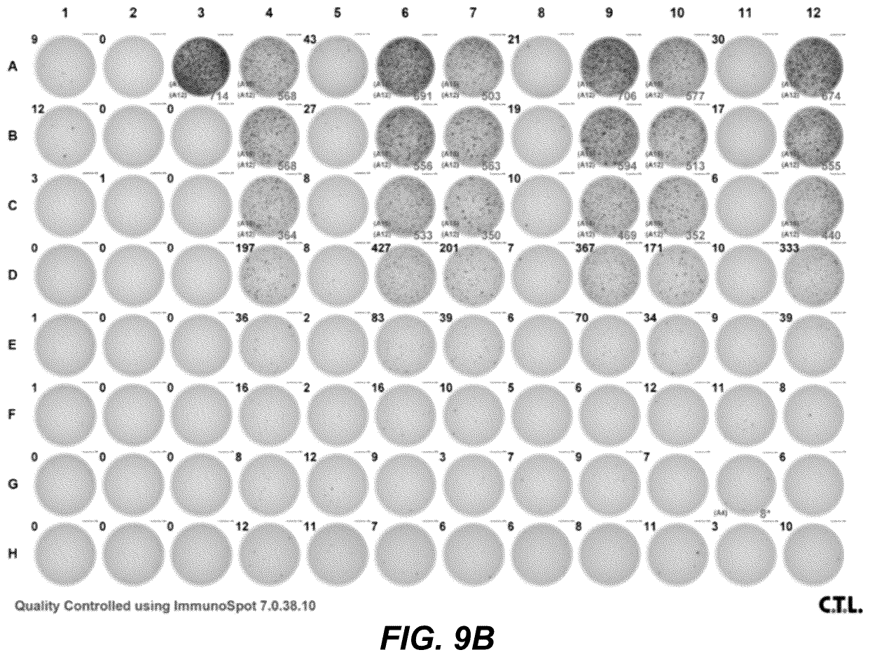

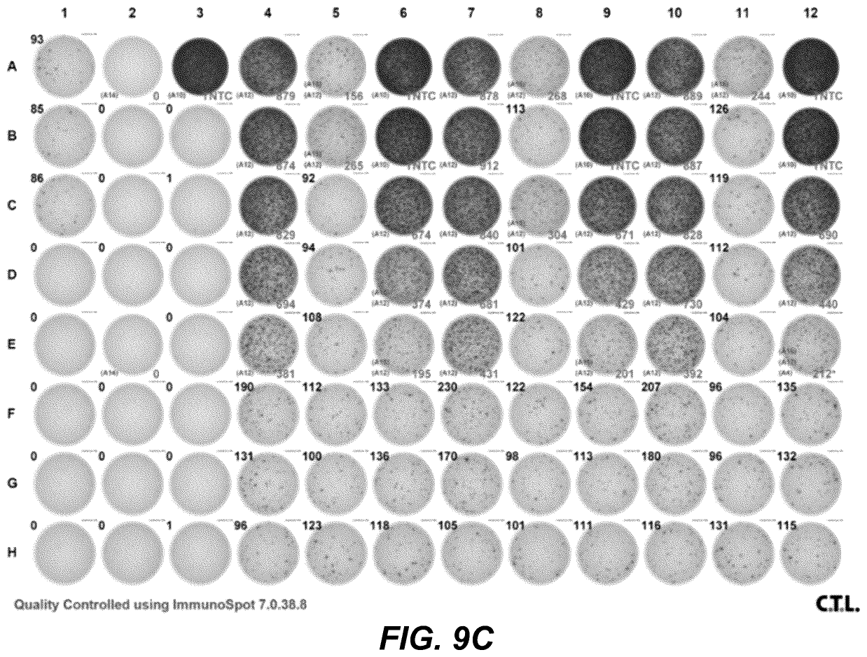

Analysis of ELISPOT assay spot forming units (SFUs) from three different T cell donors showed that both refolded NYESO1:HLA-A*02:01 tetramers and NYESO1/D9/EABR APVs significantly stimulated 1G4 T cells above background (FIG. 4C). This level of activation was specific to the NYESO1 peptide, as 1G4 T cells dosed with MART1/D9/EABR APVs demonstrated a statistically similar response to 1G4 T cells without antigen challenge. Interestingly, the elevated baseline of T cell activation without antigen challenge in “Donor 3” coincided with an increase in the potency of NYESO1/D9/EABR APVs relative to NYESO1:HLA-A*02:01 tetramers for this donor. The apparent association between the baseline activation level of a donor's T cells and the potency of the NYESO1/D9/EABR APVs was also seen in the relatively weakly activated “Donor 1” and moderately activated “Donor 2” T cells, which demonstrated correspondingly weak and moderate receptiveness, respectively, to NYESO1/D9/EABR APV stimulation as compared to NYESO1:HLA-A*02:01 tetramers (FIG. 9A-FIG. 9C). This observed dynamic potency of the APVs can be consistent and statistically significant.

Overall, these data confirmed that the presence of pMHCI/EABR APVs was sufficient to stimulate IFN-7 release from T cells harboring their cognate TCR, and that their stimulation is specific to those T cells that feature their cognate TCR.

### Discussion

Disclosed herein is a new approach for generating immunomodulatory pMHCI-displaying APVs from non-immune cells. SCT pMHCI/EABR constructs containing a cytoplasmic EABR sequence from the human centrosomal protein CEP55 recruited ESCRT proteins involved in cell division and viral budding to drive efficient assembly and release of pMHCI-displaying APVs. The lipid bilayer of the APV allowed for pMHCI reorganization in the membrane and pMHCI clustering, both of which are considered important for the formation of a T cell stimulating immunological synapse. Purified pMHCI/EABR APVs elicited IFN-7 release from T cells, and the response was specific to T cells harboring the cognate TCR.

Unlike previous methods that have used viral coat proteins to induce budding, the pMHCI/EABR construct does not require accessory proteins for vesicle release. Consequently, these pMHCI/EABR APVs can be less immunogenic compared to other APV production methods, as the pMHCI/EABR vesicles were composed of protein sequences already found in the human proteome. Thus, these T cell-targeting APVs, with putatively reduced immunogenicity, can be applicable as a targeted immunosuppressive therapeutic for autoimmune diseases. As an example, the presenting peptides that were tested for SCT murine pMHCI H-2Kd in this study—NRP-V7 and IGRP206-214—are two therapeutically relevant epitopes for a murine type 1 diabetes (T1D) model. These pMHCI complexes have previously been conjugated to iron-oxide polymer nanoparticles for use as a targeted T1D therapy, suggesting a potential clinical application for the APVs described here.

Another advantage of using the EABR sequence for APV budding is that pMHCI APV assembly requires expression of only a single pMHCI/EABR protein and the corresponding gene construct could be delivered as an mRNA therapeutic. Previous work has explored the use of mRNA therapeutics for multiple sclerosis by delivering an mRNA-LNP therapeutic coding for an autogenic peptide with the goal of loading and presenting that peptide on the endogenous MHC that resides on, and is often limited to, the surface of the cell. An mRNA therapeutic encoding pMHCI/EABR can result in increased presentation of the fully formed pMHCI complex on the cell surface in addition to the release of pMHCI-loaded APVs that could distribute throughout the body, mirroring the natural release of endogenous APVs seen during an immune response and potentially increasing their therapeutic effect.

The increase in purified pMHCI product resulting from the single-chain construct may be due to the single-chain heterotrimer's increased stability. The D9 variant, in particular, has previously been shown to have increased traffic to the cell surface and ability to maintain a steady-state presence there by bypassing cellular quality control steps. This increased cell surface expression and stability can act synergistically with the EABR sequence to increase APV budding and release in the comparatively lower-expressing HEK293T cell line, which makes the pMHCI/EABR APV approach particularly powerful as an injectable mRNA therapeutic that would likely be translated in lower-expressing primary cells in vivo. pMHCI/EABR APVs, or lipid nanoparticles encapsulating mRNA encoding a pMHCI/EABR construct, can be effective as an autoimmune therapy in some embodiments.

In summary, a new method for efficiently generating APVs from non-immune cells is disclosed in this Example. These APVs displayed complete pMHCI complexes and were capable of specific stimulation of cognate T cells, and, in some embodiments provided herein, can be employed as a cell-free therapeutic strategy for targeted immunoregulation.

### Example 2

### Immunoregulatory Antigen-Presenting Vesicles

The technology in this example is based on an approach utilizing the endosomal sorting complex required for transport (ESCRT) pathway. Post-mRNA vaccination budding of spike protein containing eVLPs was achieved by inserting an ESCRT and ALIX-binding region (EABR) peptide into the cytoplasmic tail of the spike protein. eVLPs elicited in this way showed superior neutralizing antibody responses in a mouse model. Insertion of the EABR peptide into the cytoplasmic tail of single-chain peptide-MHC (pMHC) constructs led to production of APVs displaying pMHCs for engagement with T cells. APVs constructed in this way have been demonstrated to be approximately 60 nm-80 nm in size (FIG. 2C).

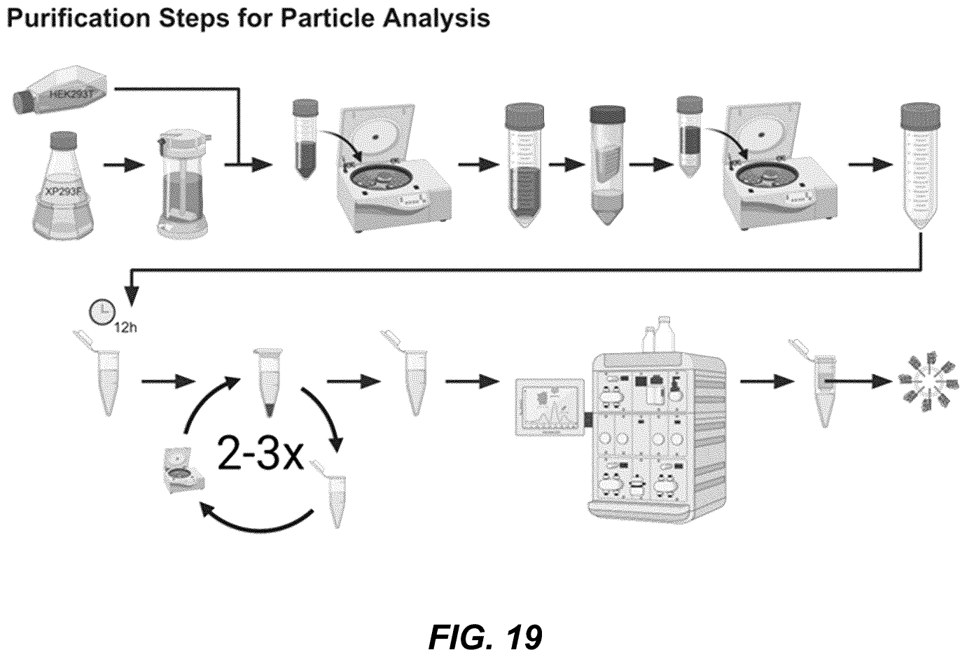

To generate these particles in a lab setting, suspension Expi293F or adherent HEK293T cells were lipotransfected with a DNA plasmid using a CMV promoter. The transfected plasmid coded for a single-chain trimer peptide-MHC complex, such as HLA-A*02:01 with the NY-ESO-1₁₅₇₋₁₆₅ peptide, and an EABR tail sequence (FIG. 16). The single-chain trimer peptide-MHC complex detected using Western blotting in FIG. 16 comprised an EABR sequence of about 7 kDa, linked to a HLA heavy chain of about 45 kDa through a linker, which was, in turn, linked to β2M of about 12 kDa through a linker. The β2M is also linked to a peptide (e.g., NY-ESO-1₁₅₇₋₁₆₅ peptide) through a linker. The total size of the peptide (e.g., NY-ESO-1₁₅₇₋₁₆₅ peptide) and the linker connecting the peptide and β2M was about 3 kDa. The supernatant from either Expi293F or HEK293T cell culture was collected and spun down at 500 g for 10 minutes. The supernatant was decanted from any cell pellet and passed through a 0.45 μm filter to remove any remaining cells from the supernatant. The filtrate was poured into a 100 kDa molecular weight concentrator, which was spun at 3000 g until 3 mL remain in the concentrator. 2.4 mL of the 3 mL of concentrated cell culture supernatant was pipetted gently onto a 600 μL cushion of 20% sucrose in 7.4 pH PBS in a polypropylene ultracentrifuge tube, and centrifuged at 135,000 g for 2 hours at 4° C. Any liquid in the polypropylene ultracentrifuge tube was gently aspirated so as not to disturb an oily pellet that formed at the bottom of the tube. This oily pellet was then resuspended in 200 μL of PBS and left overnight (12 hours) at 4° C. After 12 hours, the pellet was forcibly resuspended in the 200 μL of PBS using repeated pipetting if the pellet has not dissolved into the PBS already. The dissolved pellet in 200 μL of PBS is incubated at 37° C. for 15 minutes, then centrifuged at 10000 g for 10 minutes. The supernatant was then decanted from any visible pellet into a separate tube and centrifuged again at 10000 g for 10 minutes and decanted until no visible pellet forms after centrifugation. This purified PBS, roughly 200 μL in volume, was further purified using size-exclusion chromatography or tangential flow filtration (FIG. 19).

### Example 3

### Immunoregulatory Antigen-Presenting Vesicles Utilizing MHCII

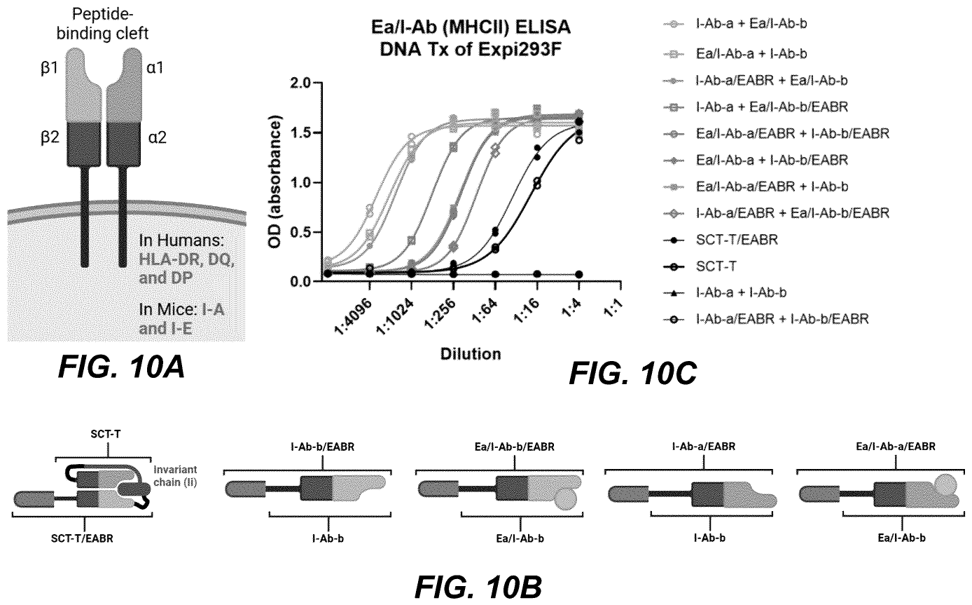

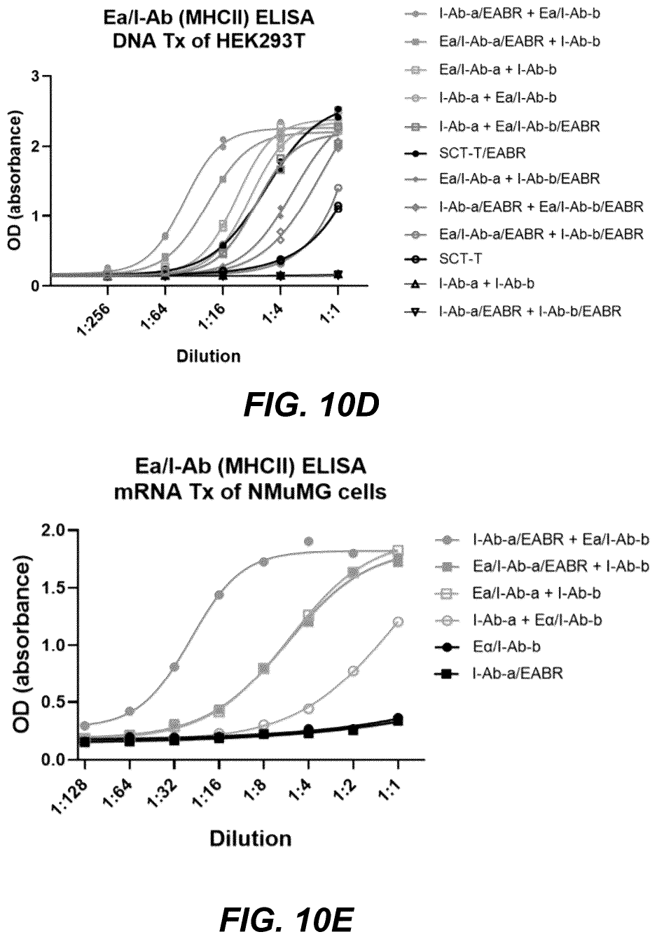

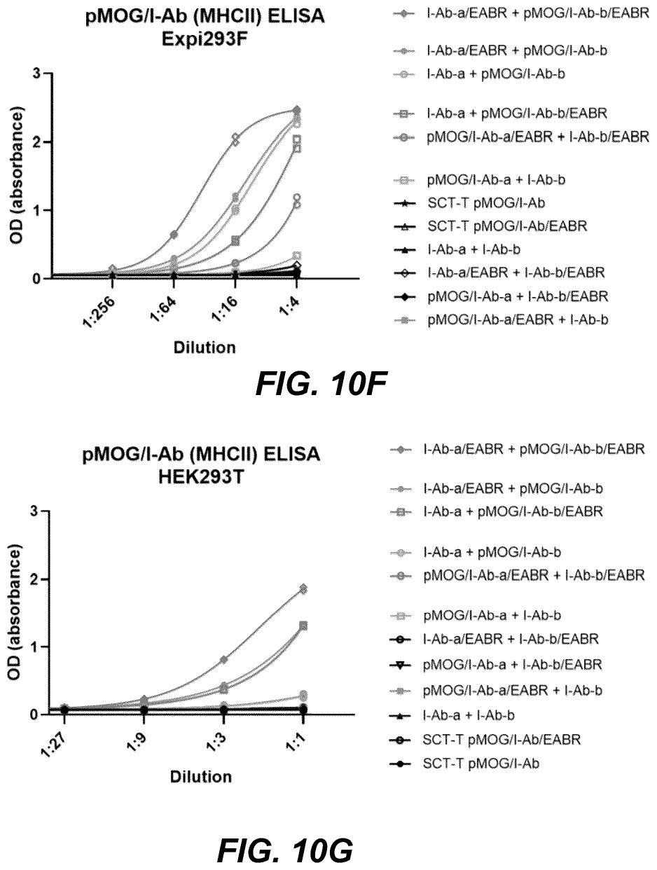

This example shows that EABR addition to pMHCII also promoted budding of the complete, intended pMHCII complex on APVs. Naturally-occurring MHCII complexes include the α and β chains, which form peptide-binding cleft when complexed (FIG. 10A). Production of SCT pMHCII/EABR construct (SCT-T & SCT-T/EABR) and various alternative constructs illustrated in FIG. 10B was detected using ELISA in (C-G). FIG. 10C-FIG. 10G show the results of the ELISA. As shown in FIG. 10C, a natural form of pMHCII (I-Ab-a & Ea/I-Ab-b) without EABR performed best in the high-expressing, engineered Expi293F cell line, transfected with various DNA plasmids encoding a murine version of MHCII, I-Ab, presenting the Ealpha peptide (Ea) or pMHCII single-chain trimer constructs. In FIG. 10D, transfection of the lower-expressing cell line HEK293T (compared to Expi293F) with an EABR-tagged I-Ab-a (I-Ab-a/EABR & Ea/I-Ab-b) outperformed the transfection of the more natural “I-Ab-a & Ea/I-Ab-b” with regard to vesicle production harboring pMHCII. In FIG. 10E, mRNA transfection of the lower-expressing cell line NMuMG (lower than both Expi293F and HEK293T) with an EABR-tagged I-Ab-a (I-Ab-a/EABR & Ea/I-Ab-b) outperformed the transfection with more natural “I-Ab-a & Ea/I-Ab-b” for vesicle production harboring pMHCII. FIG. 10F and FIG. 10G show production of murine version of MHCII, I-Ab, presenting the multiple-sclerosis-relevant myelin oligodendrocyte peptide or pMOG peptide as well as single-chain trimer pMHCII constructs from transfected Expi293F cells and HEK293T cells, respectively.

### Example 4

### Immunoregulatory Antigen-Presenting Vesicles with Co-Stimulatory Molecules

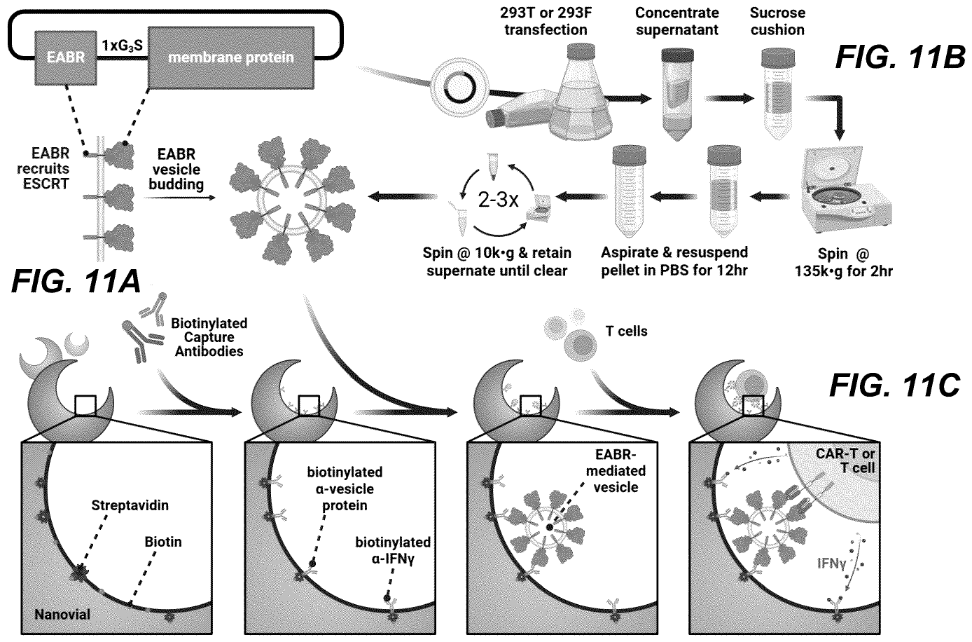

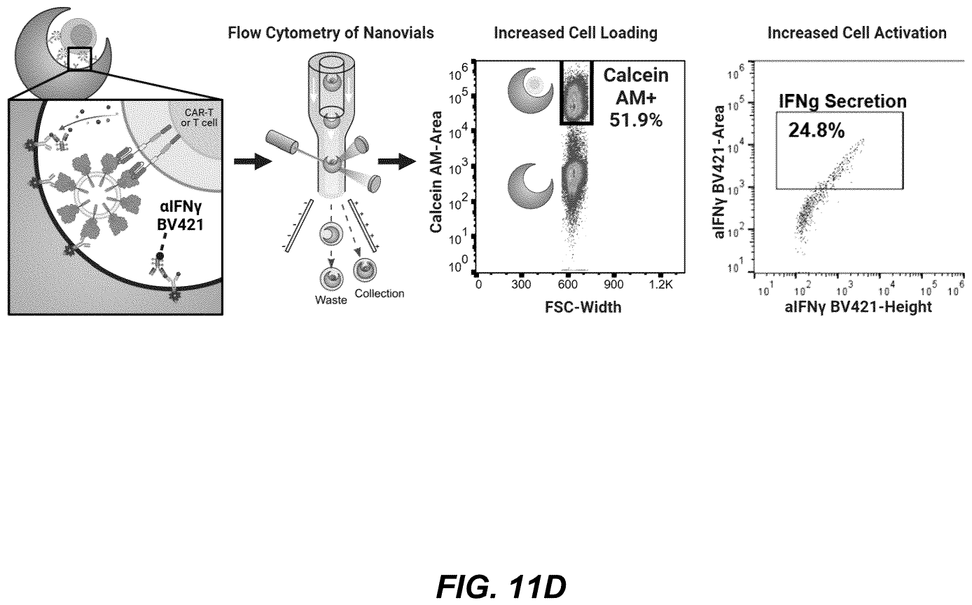

This example shows that engineered extracellular vesicles improved single cell capture and T cell activation on hydrogel nanovials compared to functionalizing the hydrogel surface with recombinant protein. Post-fabrication biotinylation of hydrogel nanovials via gelatin were functionalized with streptavidin, followed by biotinylated capture antibodies. Capture antibodies can be any protein present on the vesicle surface, including the EABR-tagged membrane protein that induces vesicle formation. Cells, such as CAR-T cells, bound to the captured extracellular vesicles within the nanovial cavity, and cell activation cytokines were captured using the biotinylated capture antibodies covering the nanovial surface (FIG. 11C). Using fluorescent sandwich antibodies, flow cytometry results of the nanovials revealed that functionalization of the nanovial cavity with engineered extracellular vesicles improved cell loading and activation (FIG. 11D).

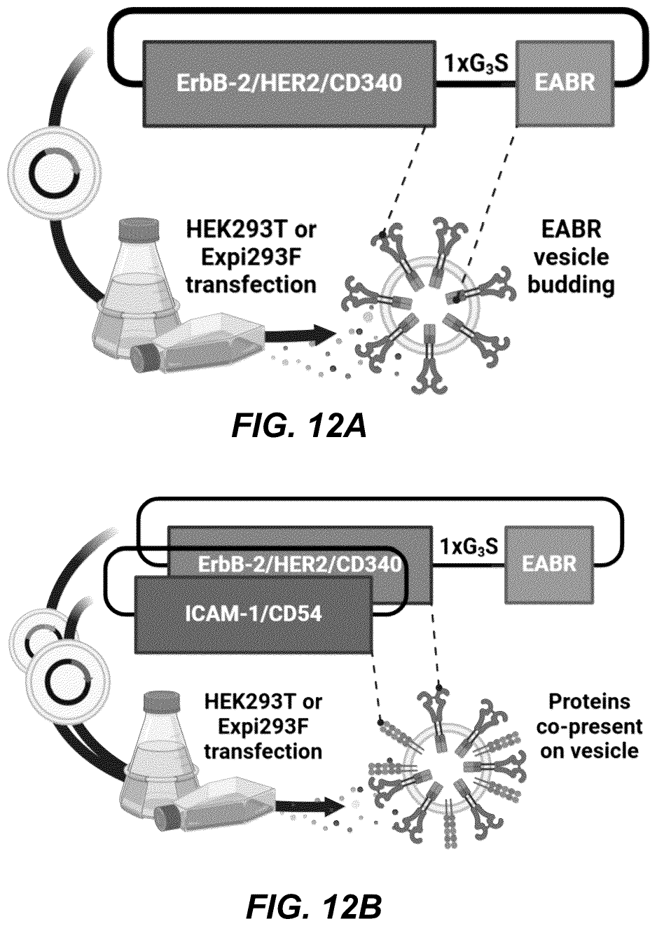

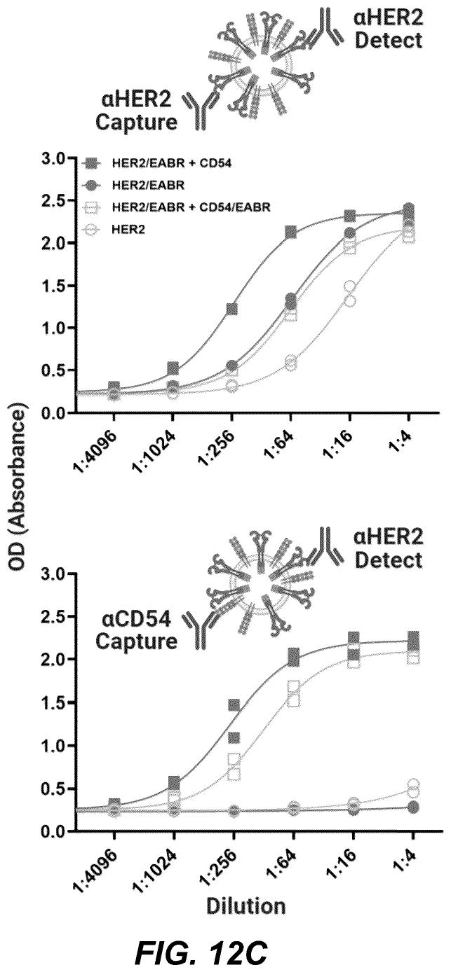

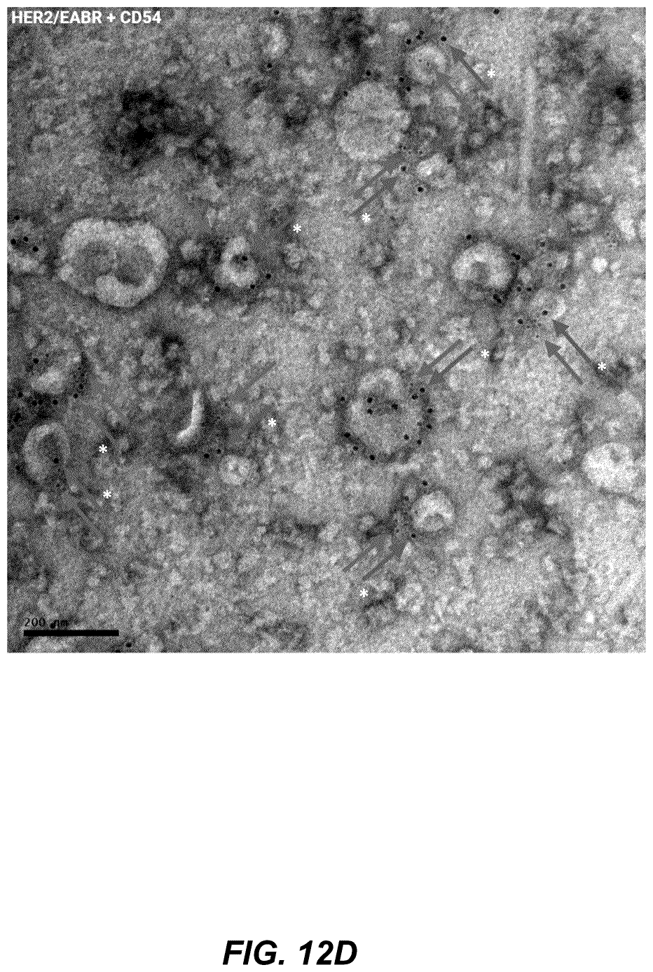

Vesicles produced from co-expression of HER2/EABR and CD54 were, then, examined. Both HER2/EABR and CD54 proteins were presented on purified extracellular vesicles in transfection supernatant (FIG. 12B). Thus, only HER2 required a vesicle-forming “EABR” sequence to be appended for both CD54 and HER2 to appear on the harvested vesicles, which was confirmed by results of sandwich ELISA of APVs purified by ultracentrifugation from transfected Expi293F cells (FIG. 12C) and immuno-EM image (FIG. 12D).

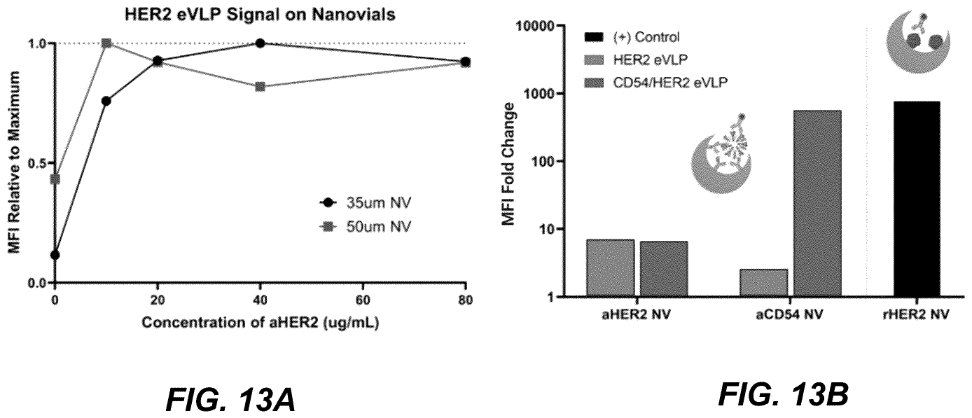

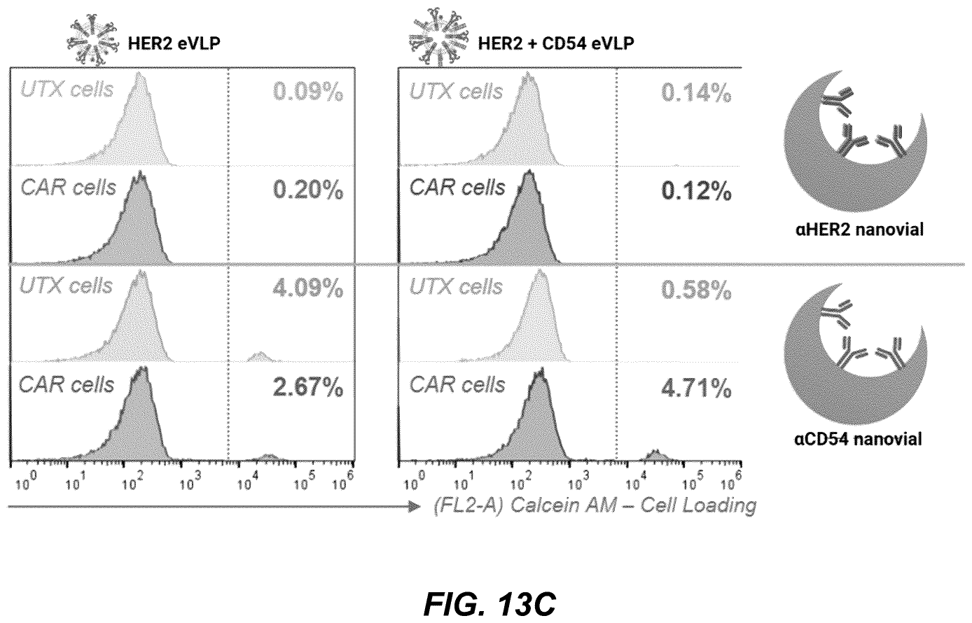

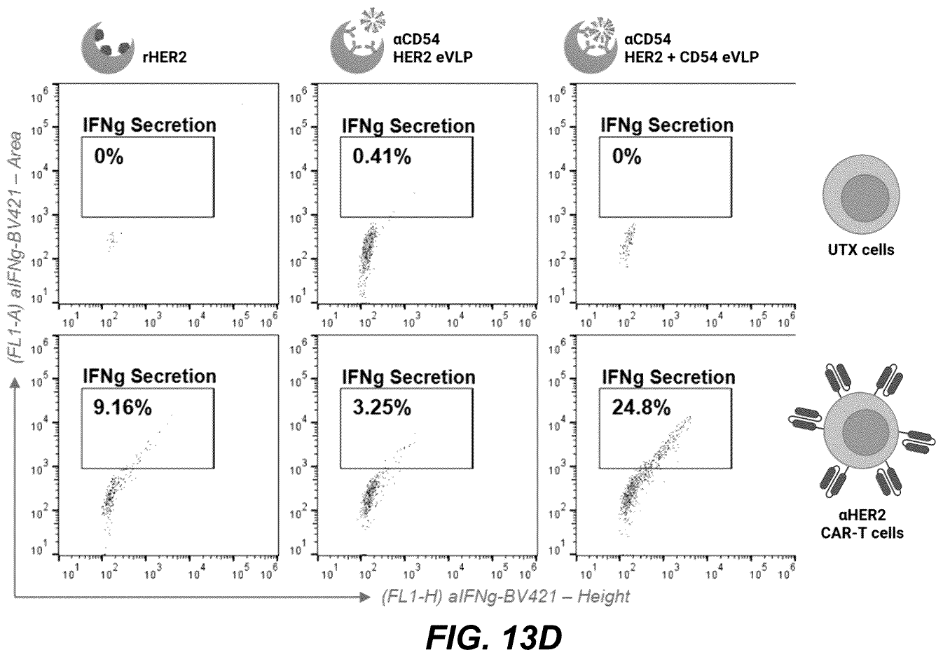

Coating hydrogel nanovials with vesicles co-presenting HER2/EABR and CD54 also improved cell loading and CAR-T activation compared to coating with recombinant HER2. Immobilization of HER2 eVLP on 35 μm and 50 μm nanovials based on concentration of aHER2 capture antibodies on the nanovials was evaluated. Maximum capture signal was detected at around 20 μg/mL aHER2 capture antibodies for both 35 um and 50 um nanovials (FIG. 13A). Compared to the HER2-only Evlp, CD54/HER2 eVLP showed significantly higher capture rate on nanovials, of which signal was comparable to the positive control (FIG. 13B). Then, aHER2 CAR cell and control untransfected (UTX) T cell were loaded in nanovials using both HER2 eVLP and CD54/HER2 eVLP. Nanovials were conjugated with aHER2 antibodies, then incubated with either HER2 or CD54/HER2 eVLP, which cannot bind control UTX cells nor aHER2 CAR T cells (FIG. 13C). Nanovials conjugated with aCD54 antibodies showed non-specific cell loading which may be due to endogenous CD54 expression on T cells (FIG. 13C). This non-specific binding was significantly reduced when CD54/HER2 eVLP were immobilized on nanovials.

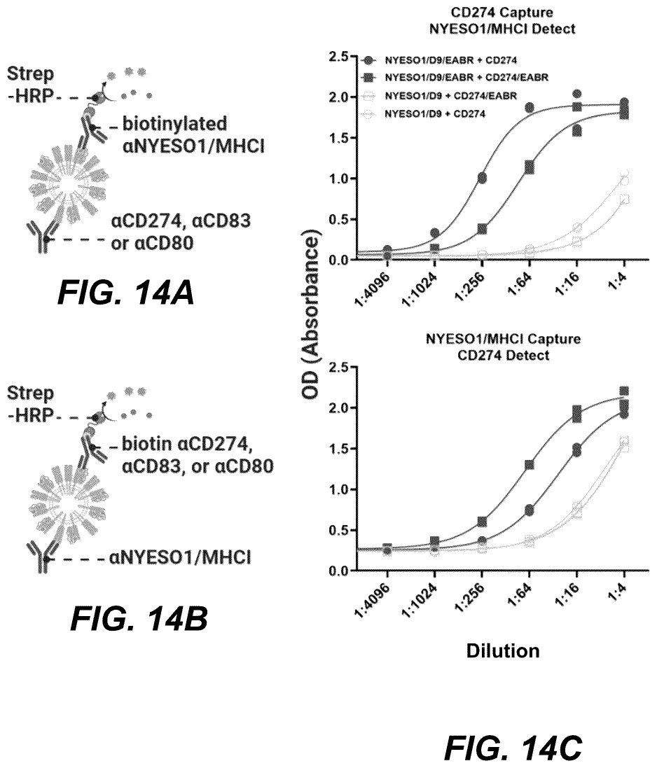

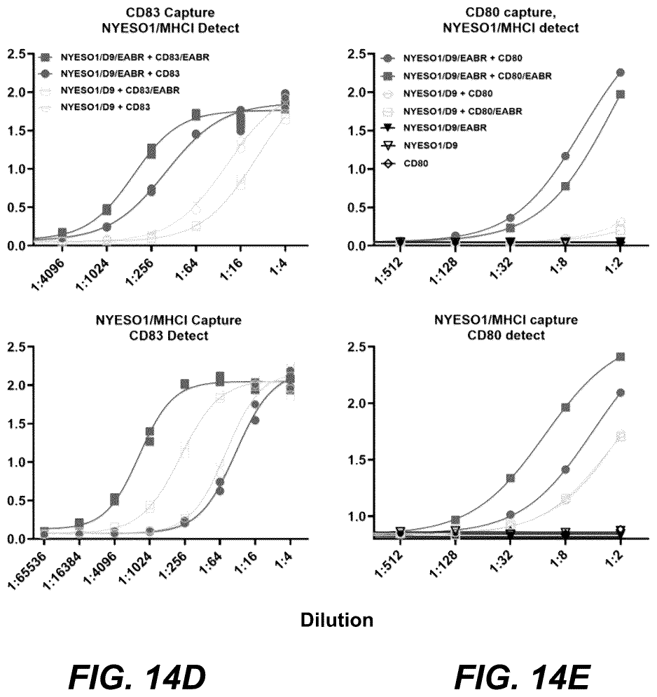

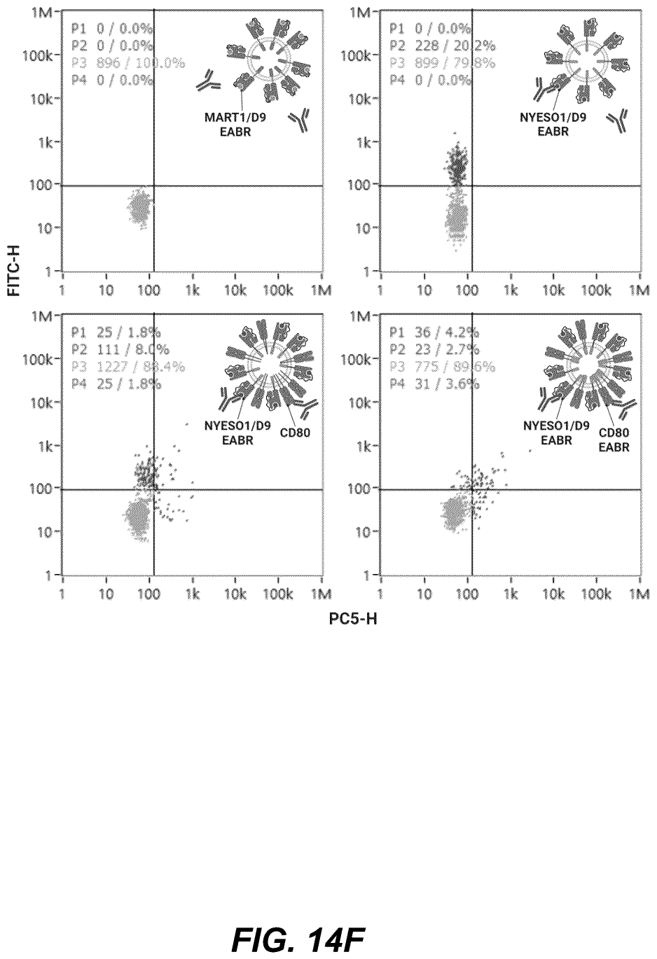

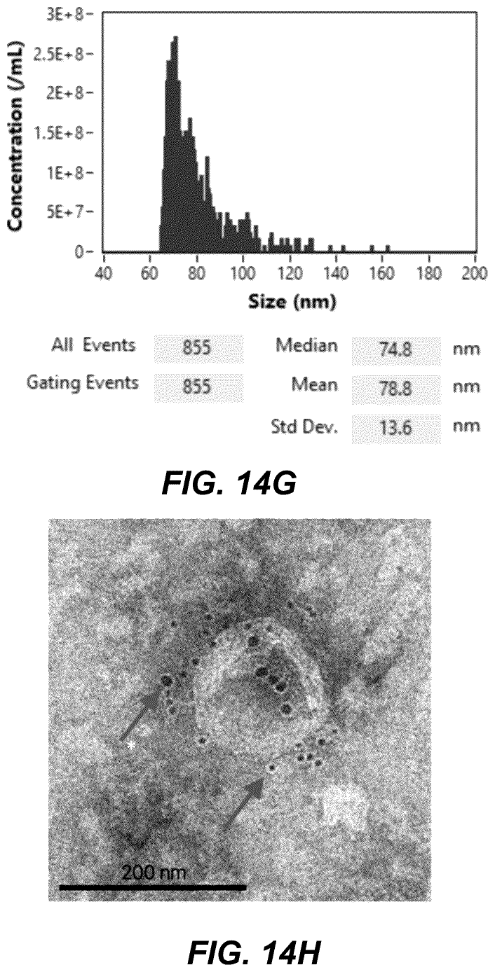

Moreover, co-expression of SCT pMHCI/EABR and CD274, CD83 or CD80 immunoregulatory proteins (co-stimulatory molecules) allowed for fine-tuned synthesis of known endogenous immunoregulatory extracellular vesicles. Expression of immunoregulatory membrane proteins CD274, CD83, or CD80 on engineered extracellular vesicles were captured with plated antibody, then co-presented NYESO1/HLA-A*02:01 were detected and quantified with biotinylated anti-NYESO:HLA-A*02:01 and HRP-conjugated streptavidin (strep-TRP) (FIG. 14A). This assay was also performed with the capture antibody and detection antibody swapped (FIG. 14B). FIG. 14C-FIG. 14E show the results of sandwich ELISA of the APVs The inclusion of the immunoregulatory membrane proteins was also confirmed using nano flow cytometry (NanoFCM) (FIG. 14F), nanoFCM size analysis (FIG. 14G) and immuno-EM image of THE vesicles (FIG. 14H).

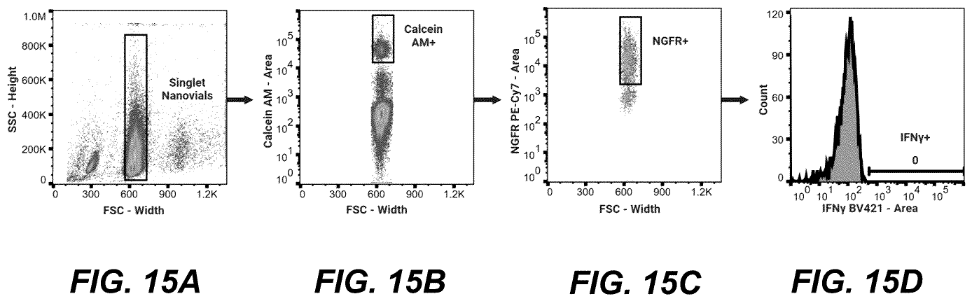

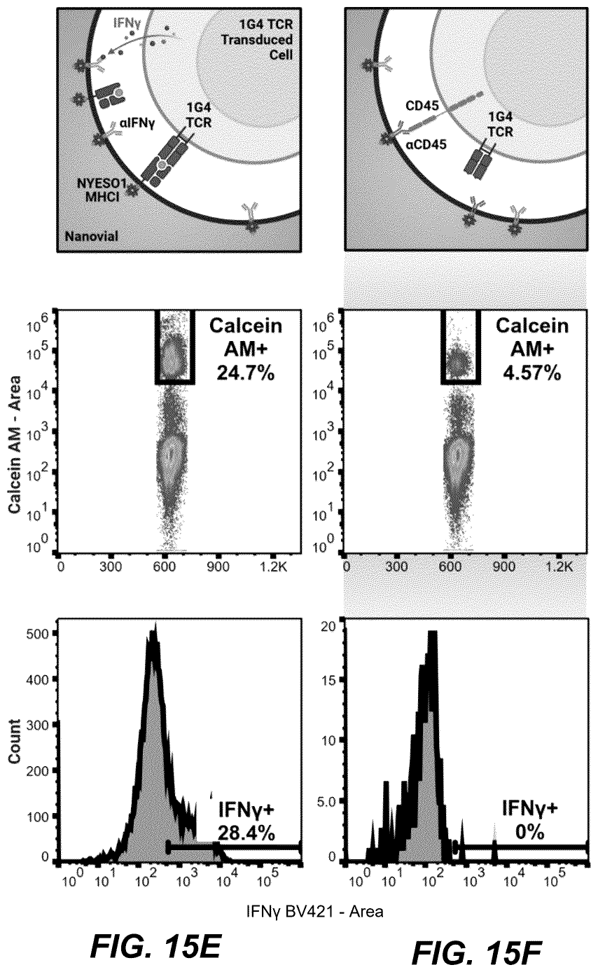

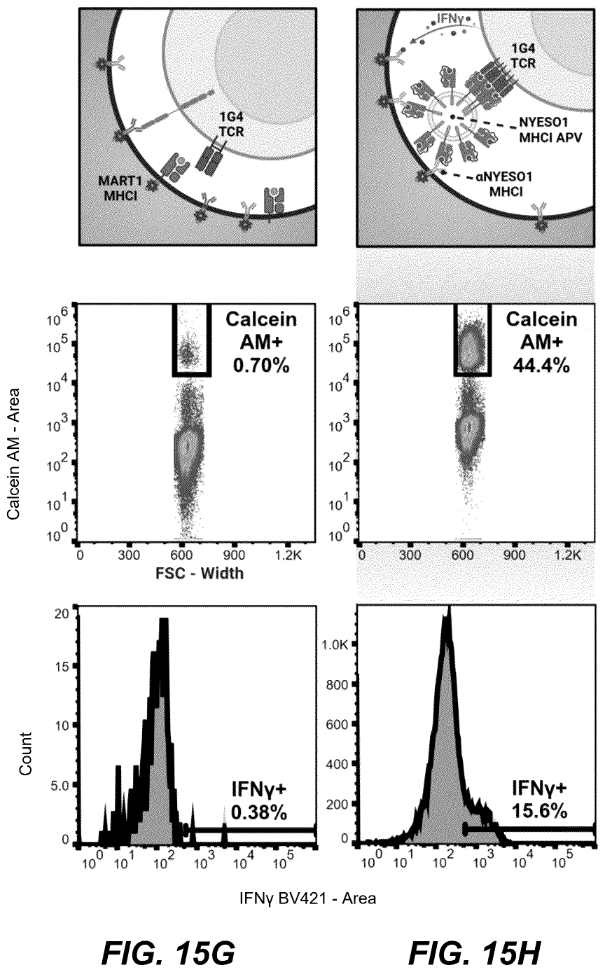

As shown by flow cytometry results, coating hydrogel nanovials with synthetic antigen-presenting vesicles co-presenting pMHCI/EABR and CD80 improved cell loading and T cell activation compared to coating with recombinant pMHCI. T cell loading was significantly improved by using a matched pMHC relative to standard anti-CD45 antibody-based binding. pMHC expression in eVLPs further improves T cell loading rates (middle panels of FIG. 15E-FIG. 15I).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A composition, comprising:
a nucleic acid composition comprising one or more polynucleotides encoding components of an antigen presentation complex (APC),
wherein the components of the APC are capable of assembling into an antigen-presenting vesicle (APV) budded from a cell in which the APC components are expressed,
wherein the APV comprises a plurality of APCs, and
wherein the APC comprises an APC-assembly domain, one or more antigen presentation polypeptide(s) and a presenting peptide.

2. A composition, comprising:
a population of an antigen-presenting vesicles (APVs),
wherein each of the APVs comprises a plurality of antigen presentation complexes (APCs), and
wherein the APC comprises an APC-assembly domain, one or more antigen presentation polypeptide(s) and a presenting peptide.

3. (canceled)

4. The composition of claim 1, wherein the APC comprises all or a portion of a major histocompatibility complex (MHC), a MHC mimetic, or a derivative thereof.

5.-9. (canceled)

10. The composition of claim 1, wherein at least two components of the APC are derived from different species.

11. The composition of claim 1, wherein the APC comprises at least one mutation at positions equivalent to Y84 and A139 of HLA-A.

12. The composition of claim 1, wherein the antigen presentation polypeptide(s) is capable of forming a binding site that binds the presenting peptide.

13. The composition of claim 1, wherein:
the presence of the antigen presentation polypeptide(s) does not induce an immune response in a subject; and/or
wherein the presence of the antigen presentation polypeptide(s) induces an immune response in a subject.

14.-15. (canceled)

16. The composition of claim 1, wherein the cell:
is not an immune cell; or
is an immune cell.

17.-30. (canceled)

31. The composition of claim 1, wherein the APC comprises a single fusion protein or two or more separate polypeptides.

32. The composition of claim 31, wherein:
the single fusion protein comprises one or more linker(s), the presenting peptide, the antigen presentation polypeptide(s), and the APC-assembly domain; or
at least one of the two or more separate polypeptides comprises (i) a linker, a first antigen presentation polypeptide, and the APC-assembly domain, or (ii) a linker, a second antigen presentation polypeptide, and the presenting peptide.

33. (canceled)

34. The composition of claim 31, wherein:
the antigen presentation polypeptide(s) is situated between the APC-assembly domain and the presenting peptide;
at least one of the antigen presentation polypeptide(s) is situated between the APC-assembly domain and the presenting peptide;
the antigen presentation polypeptide(s) comprise two or more antigen presentation polypeptides with the presenting peptide situated between said two or more antigen presentation polypeptides; and/or
the antigen presentation polypeptide(s) comprise MHC-I α chain and beta-2 microglobulin (β2M).

35. The composition of claim 32, wherein the components of the APC are connected in the order from N-terminus to C-terminus:
the presenting peptide, beta-2 microglobulin (β2M), MHC-I α chain and the APC-assembly domain;
the APC-assembly domain, MHC-I α chain, beta-2 microglobulin (β2M) and the presenting peptide;
the APC-assembly domain, MHC-II β chain, the presenting peptide and MHC-II α chain;
the APC-assembly domain, the MHC-II α chain, the presenting peptide and the MHC-II (3 chain;
the MHC-II α chain, the presenting peptide, the MHC-II β chain and the APC-assembly domain; or
the MHC-II β chain, the presenting peptide, the MHC-II α chain and the APC-assembly domain.

36. The composition of claim 31, wherein the antigen presentation polypeptide(s) comprise MHC-II α chain and MHC-II β chain.

37.-42. (canceled)

43. The composition of claim 32, wherein the first antigen presentation polypeptide is capable of binding to the second antigen presentation polypeptide.

44.-46. (canceled)

47. The composition of claim 1, wherein the APC comprises an amino acid sequence having at least 65%, 70%, 75%, 80%, 85%, 90%, 95%, 98%, or 99% sequence identity to an amino acid sequence selected from the group consisting of SEQ ID NOs: 37-52, 55-56, 59-60, 63-80, 114-119, 122-143, 146-151, and 154-159, and wherein the one or more polynucleotides comprise the nucleotide sequence of any one of SEQ ID NOs: 90-111 and 160-183 or a sequence that is at least 80%, 85%, 90%, 95%, 98%, 99%, or 100% identical to any one of SEQ ID NOs: 90-111 and 160-183.

48. The composition of claim 1, wherein the APVs comprise one or more supplemental polypeptide(s) selected from the group comprising immunosuppressive polypeptide(s), immunostimulatory polypeptide(s), costimulatory polypeptide(s), cell adhesion polypeptide(s), anti-phagocytic polypeptide(s), tissue-homing polypeptide(s), cell-surface-molecule binding polypeptide(s), or any combination thereof.

49.-52. (canceled)

53. The composition of claim 1, wherein at least two of the APCs of the plurality of APCs are different from each other with respect to the presenting peptide, and wherein the population of APVs thereby display a plurality of disparate presenting peptides.

54.-57. (canceled)

58. The composition of claim 1, wherein the APV and/or the APC is capable of inducing immunogenic responses against the presenting peptide in a subject without the administration of exogenous antigen presenting cells and/or exogenous antigen presenting components other than the APV and/or the APC.

59.-75. (canceled)

76. A cell comprising the nucleic acid composition of claim 1.

77. A method of stimulating an immune response or treating or preventing a disease or disorder in a subject in need thereof, comprising:
administering to the subject a therapeutically effective amount of the composition of claim 1, thereby stimulating an immune response or treating or preventing the disease or disorder in the subject.

78.-99. (canceled)
